(19) United States
(12) Patent Application Publication
(10) Pub. No.: US 2026/0293519 A1
(43) Pub. Date: Sep. 24, 2026

(54) ORGANIC ELECTROLUMINESCENT MATERIAL AND DEVICE THEREOF

(71) Applicant: BEIJING SUMMER SPROUT TECHNOLOGY CO., LTD., Beijing (CN)

(72) Inventors: Cuifang ZHANG, Beijing (CN); Qi ZHANG, Beijing (CN); Han ZHANG, Beijing (CN); Zhihong DAI, Beijing (CN); Zheng WANG, Beijing (CN); Hongbo LI, Beijing (CN); Weina ZHANG, Beijing (CN); Chi Yuen Raymond Kwong, Beijing (CN); Chuanjun Xia, Beijing (CN)

(21) Appl. No.: 19/565,271

(22) Filed: Mar. 12, 2026

(30) Foreign Application Priority Data
Mar. 13, 2025 (CN) 202510298051.5

(51) Int. Cl. H10K 85/60; C07B 59/00; C07D 209/86; C07D 263/57; C07D 307/91; C07D 401/14; C07D 405/04; C07D 405/14; C07D 409/14; C07D 413/12; C07D 413/14; C07D 417/12; C07D 487/04; C07F 5/02; C09K 11/02; C09K 11/06; H10K 50/12

(52) U.S. Cl. CPC H10K 85/658; C07B 59/002; C07D 209/86; C07D 263/57; C07D 307/91; C07D 401/14; C07D 405/04; C07D 405/14; C07D 409/14; C07D 413/12; C07D 413/14; C07D 417/12; C07D 487/04; C07F 5/027; C09K 11/02; C09K 11/06; C09K 2211/1018; H10K 50/121

(57) ABSTRACT

Provided are an organic electroluminescent material and device thereof. The organic electroluminescent material is a polycyclic compound having a structure of Formula 1, and the compound is usable as a TADF material in an organic electroluminescent device. The polycyclic compound has a modulation effect on an emitted color, can achieve the desired orange to red luminescence, and has deep HOMO and LUMO energy levels, thus possessing a potential to become an excellent light-emitting material and having a very broad industrial application prospect. Further provided are an organic electroluminescent device comprising the compound and a compound composition comprising the compound.

### CROSS-REFERENCE TO RELATED APPLICATION(S)

This application claims priority to Chinese Patent Application No. 202510298051.5 filed on Mar. 13, 2025, the disclosure of which is incorporated herein by reference in its entirety.

### TECHNICAL FIELD

The present disclosure relates to compounds for organic electronic devices such as organic light-emitting devices. Particularly, the present disclosure relates to a polycyclic compound, an organic electroluminescent device comprising the polycyclic compound, and a compound composition comprising the polycyclic compound.

### BACKGROUND

Organic electronic devices include, but are not limited to, the following types: organic light-emitting diodes (OLEDs), organic field-effect transistors (O-FETs), organic light-emitting transistors (OLETs), organic photovoltaic devices (OPVs), dye-sensitized solar cells (DSSCs), organic optical detectors, organic photoreceptors, organic field-quench devices (OFQDs), light-emitting electrochemical cells (LECs), organic laser diodes and organic plasmon emitting devices.

In 1987, Tang and Van Slyke of Eastman Kodak reported a bilayer organic electroluminescent device, which comprises an arylamine hole transporting layer and a tris-8-hydroxyquinolato-aluminum layer as the electron and emitting layer (Applied Physics Letters, 1987, 51 (12): 913-915). Once a bias is applied to the device, green light was emitted from the device. This device laid the foundation for the development of modern organic light-emitting diodes (OLEDs). State-of-the-art OLEDs may comprise multiple layers such as charge injection and transporting layers, charge and exciton blocking layers, and one or multiple emissive layers between the cathode and anode. Since the OLED is a self-emitting solid state device, it offers tremendous potential for display and lighting applications. In addition, the inherent properties of organic materials, such as their flexibility, may make them well suited for particular applications such as fabrication on flexible substrates.

The OLED can be categorized as three different types according to its emitting mechanism. The OLED invented by Tang and van Slyke is a fluorescent OLED. It only utilizes singlet emission. The triplets generated in the device are wasted through nonradiative decay channels. Therefore, the internal quantum efficiency (IQE) of the fluorescent OLED is only 25%. This limitation hindered the commercialization of OLED. In 1997, Forrest and Thompson reported phosphorescent OLED, which uses triplet emission from heavy metal containing complexes as the emitter. As a result, both singlet and triplets can be harvested, achieving 100% IQE. The discovery and development of phosphorescent OLED contributed directly to the commercialization of active-matrix OLED (AMOLED) due to its high efficiency. Recently, Adachi achieved high efficiency through thermally activated delayed fluorescence (TADF) of organic compounds. These emitters have small singlet-triplet gap that makes the transition from triplet back to singlet possible. In the TADF device, the triplet excitons can go through reverse intersystem crossing to generate singlet excitons, resulting in high IQE.

OLEDs can also be classified as small molecule and polymer OLEDs according to the forms of the materials used. A small molecule refers to any organic or organometallic material that is not a polymer. The molecular weight of the small molecule can be large as long as it has well defined structure. Dendrimers with well-defined structures are considered as small molecules. Polymer OLEDs include conjugated polymers and non-conjugated polymers with pendant emitting groups. Small molecule OLED can become the polymer OLED if post polymerization occurred during the fabrication process.

There are various methods for OLED fabrication. Small molecule OLEDs are generally fabricated by vacuum thermal evaporation. Polymer OLEDs are fabricated by solution process such as spin-coating, inkjet printing, and slit printing. If the material can be dissolved or dispersed in a solvent, the small molecule OLED can also be produced by solution process.

The emitting color of the OLED can be achieved by emitter structural design. An OLED may comprise one emitting layer or a plurality of emitting layers to achieve desired spectrum. In the case of green, yellow, and red OLEDs, phosphorescent emitters have successfully reached commercialization. Blue phosphorescent device still suffers from non-saturated blue color, short device lifetime, and high operating voltage. Commercial full-color OLED displays normally adopt a hybrid strategy, using fluorescent blue and phosphorescent yellow, or red and green. At present, efficiency roll-off of phosphorescent OLEDs at high brightness remains a problem. In addition, it is desirable to have more saturated emitting color, higher efficiency, and longer device lifetime.

CN113227107A discloses a polycyclic aromatic compound having a general formula of

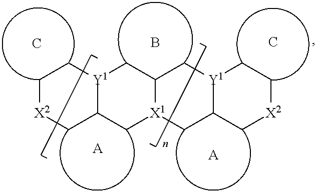

wherein Y¹ is each independently B, P, P═O, P═S, Al, Ga, As, Si—R, or Ge—R, X¹ is each independently N or C—R, and X² is each independently O, N—R, C(—R)₂, S, or Se. It also discloses the following specific compounds:

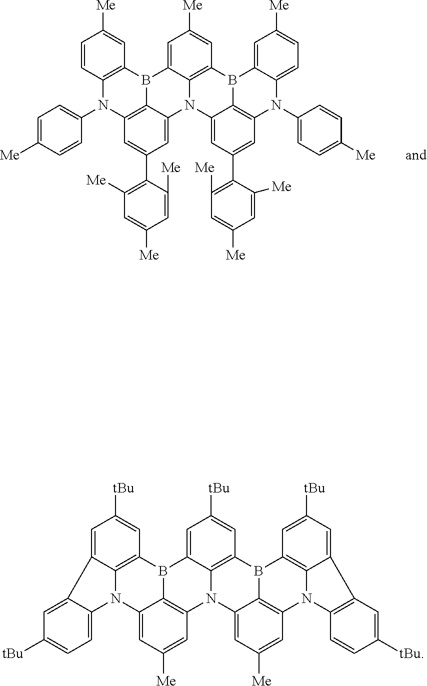 [SMILES: Cc1ccc(N2c3ccc(C)cc3B3c4cc(C)cc5c4N(c4cc(-c6c(C)cc(C)cc6C)cc2c43)c2cc(-c3c(C)cc(C)cc3C)cc3c2B5c2cc(C)ccc2N3c2ccc(C)cc2)cc1 ; Cc1cc2c3c(c1)-n1c4ccc(C(C)(C)C)cc4c4cc(C(C)(C)C)cc(c41)B3c1cc(C(C)(C)C)cc3c1N2c1cc(C)cc2c1B3c1cc(C(C)(C)C)cc3c4cc(C(C)(C)C)ccc4n-2c13]

In compounds disclosed therein, the ring A and the ring C in each of two couples of rings A and rings C have to be joined by X². CN113227107A does not disclose nor teach a compound in which the ring A and the ring C in either one couple of ring A and ring C are not joined, and does not disclose nor teach effects of the compound in modulating a maximum emission wavelength and on device performance.

Polycyclic compounds with boron, nitrogen, etc. as central atoms have been disclosed in the related art. However, when such polycyclic compounds are used as light-emitting materials, the performance of related devices is still insufficient, and particularly the emission wavelength, full width at half maximum, voltage, efficiency, lifetime, and other aspects of the devices are still to be improved. Therefore, further studies are required.

### SUMMARY

The present disclosure aims to provide a new compound to solve at least part of the preceding problems. The compound has a particular structure represented by Formula 1 and may be used as a TADF material in an organic electroluminescent device. The compound has a modulation effect on an emitted color, can achieve the desired orange to red luminescence, and has deep HOMO and LUMO energy levels, thus possessing a potential to become an excellent light-emitting material and having a very broad industrial application prospect.

According to an embodiment of the present disclosure, disclosed is a compound having a structure of Formula 1:

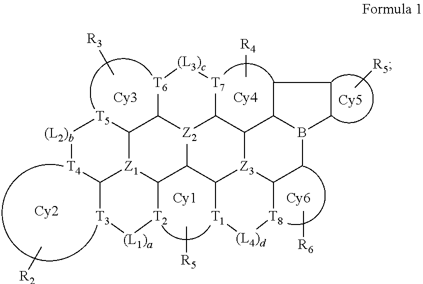

wherein the ring Cy1, the ring Cy2, the ring Cy3, the ring Cy5, and the ring Cy6 are, at each occurrence identically or differently, selected from an unsaturated carbocyclic ring having 5 to 30 carbon atoms or an unsaturated heterocyclic ring having 3 to 30 carbon atoms; and the ring Cy4 is selected from an unsaturated carbocyclic ring having 5 to 30 carbon atoms or an unsaturated heterocyclic ring having 4 to 30 carbon atoms;
Z₁, Z₂, and Z₃ are, at each occurrence identically or differently, selected from B, N, P, P═O, P═S, As, As═O, As═S, SiR, or GeR;
L₁, L₂, L₃, and L₄ are, at each occurrence identically or differently, selected from a single bond, O, S, Se, NR′, PR′, P═OR′, P═SR′, AsR′, As═OR′, As═SR′, CR′R′, SiR′R′, or GeR′R′; when multiple R′ are present at the same time, the multiple R′ are identical or different;
a, b, c, and d are each independently selected from 0, 1, or 2;
T₁ to T₈ are, at each occurrence identically or differently, selected from C, CRₜ, CRₜRₜ, N, NRₜ, O, S, or Se;
R₁, R₂, R₃, R₄, R₅, and R₆ represent, at each occurrence identically or differently, mono-substitution, multiple substitutions, or non-substitution;
R, R′, Rₜ, R¹, R², R³, R⁴, R⁵, and R⁶ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
adjacent substituents R₅ can be optionally joined to form a ring; and
adjacent substituents R′, Rₜ, R¹, R², R³, R⁴, and R⁶ can be optionally joined to form a ring.

According to an embodiment of the present disclosure, disclosed is an organic electroluminescent device, which comprises an anode, a cathode, and an organic layer disposed between the anode and the cathode, wherein the organic layer comprises the compound of any one of the preceding embodiments.

According to another embodiment of the present disclosure, further disclosed is a compound composition comprising the compound of any one of the preceding embodiments.

The present disclosure discloses a series of compounds each having a particular structure represented by Formula 1. The compounds have a modulation effect on the emitted color, can achieve the desired orange to red luminescence, and have deep HOMO and LUMO energy levels, thus possessing a potential to become excellent TADF materials and having a very broad industrial application prospect.

### BRIEF DESCRIPTION OF DRAWINGS

FIG. 1 is a schematic diagram of an organic light-emitting device that may comprise a compound and a compound composition disclosed herein.

FIG. 2 is a schematic diagram of another organic light-emitting device that may comprise a compound and a compound composition disclosed herein.

### DETAILED DESCRIPTION

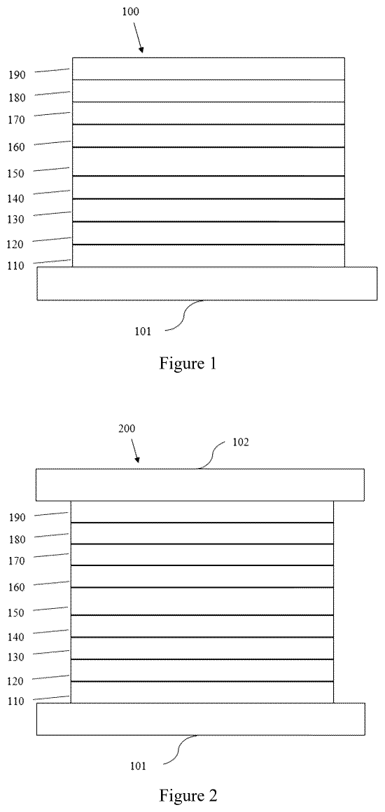

OLEDs can be fabricated on various types of substrates such as glass, plastic, and metal foil. FIG. 1 schematically shows an organic light-emitting device 100 without limitation. The figures are not necessarily drawn to scale. Some of the layers in the figures can also be omitted as needed. Device 100 may include a substrate 101, an anode 110, a hole injection layer 120, a hole transport layer 130, an electron blocking layer 140, an emissive layer 150, a hole blocking layer 160, an electron transport layer 170, an electron injection layer 180 and a cathode 190. Device 100 may be fabricated by depositing the layers described in order. The properties and functions of these various layers, as well as example materials, are described in more detail in U.S. Pat. No. 7,279,704 at cols. 6-10, the contents of which are incorporated by reference herein in its entirety.

More examples for each of these layers are available. For example, a flexible and transparent substrate-anode combination is disclosed in U.S. Pat. No. 5,844,363, which is incorporated by reference herein in its entirety. An example of a p-doped hole transport layer is m-MTDATA doped with F4-TCNQ at a molar ratio of 50:1, as disclosed in U.S. Patent Application Publication No. 2003/0230980, which is incorporated by reference herein in its entirety. Examples of host materials are disclosed in U.S. Pat. No. 6,303,238 to Thompson et al., which is incorporated by reference herein in its entirety. An example of an n-doped electron transport layer is BPhen doped with Li at a molar ratio of 1:1, as disclosed in U.S. Patent Application Publication No. 2003/0230980, which is incorporated by reference herein in its entirety. U.S. Pat. Nos. 5,703,436 and 5,707,745, which are incorporated by reference herein in their entireties, disclose examples of cathodes including composite cathodes having a thin layer of metal such as Mg:Ag with an overlying transparent, electrically-conductive, sputter-deposited ITO layer. The theory and use of blocking layers are described in more detail in U.S. Pat. No. 6,097,147 and U.S. Patent Application Publication No. 2003/0230980, which are incorporated by reference herein in their entireties. Examples of injection layers are provided in U.S. Patent Application Publication No. 2004/0174116, which is incorporated by reference herein in its entirety. A description of protective layers may be found in U.S. Patent Application Publication No. 2004/0174116, which is incorporated by reference herein in its entirety.

The layered structure described above is provided by way of non-limiting examples. Functional OLEDs may be achieved by combining the various layers described in different ways, or layers may be omitted entirely. It may also include other layers not specifically described. Within each layer, a single material or a mixture of multiple materials can be used to achieve optimum performance. Any functional layer may include several sublayers. For example, the emissive layer may have two layers of different emitting materials to achieve desired emission spectrum.

In one embodiment, an OLED may be described as having an “organic layer” disposed between a cathode and an anode. This organic layer may include a single layer or multiple layers.

An OLED can be encapsulated by a barrier layer. FIG. 2 schematically shows an organic light emitting device 200 without limitation. FIG. 2 differs from FIG. 1 in that the organic light emitting device include a barrier layer 102, which is above the cathode 190, to protect it from harmful species from the environment such as moisture and oxygen. Any material that can provide the barrier function can be used as the barrier layer such as glass or organic-inorganic hybrid layers. The barrier layer should be placed directly or indirectly outside of the OLED device. Multilayer thin film encapsulation was described in U.S. Pat. No. 7,968,146, which is incorporated by reference herein in its entirety.

Devices fabricated in accordance with embodiments of the present disclosure can be incorporated into a wide variety of consumer products that have one or more of the electronic component modules (or units) incorporated therein. Some examples of such consumer products include flat panel displays, monitors, medical monitors, televisions, billboards, lights for interior or exterior illumination and/or signaling, heads-up displays, fully or partially transparent displays, flexible displays, smart phones, tablets, phablets, wearable devices, smart watches, laptop computers, digital cameras, camcorders, viewfinders, micro-displays, 3-D displays, vehicles displays, and vehicle tail lights.

The materials and structures described herein may be used in other organic electronic devices listed above.

As used herein, “top” means furthest away from the substrate, while “bottom” means closest to the substrate. Where a first layer is described as “disposed over” a second layer, the first layer is disposed further away from the substrate. There may be other layers between the first and second layers, unless it is specified that the first layer is “in contact with” the second layer. For example, a cathode may be described as “disposed over” an anode, even though there are various organic layers in between.

As used herein, “solution processible” means capable of being dissolved, dispersed, or transported in and/or deposited from a liquid medium, either in solution or suspension form.

A ligand may be referred to as “photoactive” when it is believed that the ligand directly contributes to the photoactive properties of an emissive material. A ligand may be referred to as “ancillary” when it is believed that the ligand does not contribute to the photoactive properties of an emissive material, although an ancillary ligand may alter the properties of a photoactive ligand.

It is believed that the internal quantum efficiency (IQE) of fluorescent OLEDs can exceed the 25% spin statistics limit through delayed fluorescence. As used herein, there are two types of delayed fluorescence, i.e. P-type delayed fluorescence and E-type delayed fluorescence. P-type delayed fluorescence is generated from triplet-triplet annihilation (TTA).

On the other hand, E-type delayed fluorescence does not rely on the collision of two triplets, but rather on the transition between the triplet states and the singlet excited states. Compounds that are capable of generating E-type delayed fluorescence are required to have very small singlet-triplet gaps to convert between energy states. Thermal energy can activate the transition from the triplet state back to the singlet state. This type of delayed fluorescence is also known as thermally activated delayed fluorescence (TADF). A distinctive feature of TADF is that the delayed component increases as temperature rises. If the reverse intersystem crossing (RISC) rate is fast enough to minimize the non-radiative decay from the triplet state, the fraction of back populated singlet excited states can potentially reach 75%. The total singlet fraction can be 100%, far exceeding 25% of the spin statistics limit for electrically generated excitons.

E-type delayed fluorescence characteristics can be found in an exciplex system or in a single compound. Without being bound by theory, it is believed that E-type delayed fluorescence requires the luminescent material to have a small singlet-triplet energy gap (AES-T). Organic, non-metal containing, donor-acceptor luminescent materials may be able to achieve this. The emission in these materials is generally characterized as a donor-acceptor charge-transfer (CT) type emission. The spatial separation of the HOMO and LUMO in these donor-acceptor type compounds generally results in small AES-T. These states may involve CT states. Generally, donor-acceptor luminescent materials are constructed by connecting an electron donor moiety such as amino- or carbazole-derivatives and an electron acceptor moiety such as N-containing six-membered aromatic rings.

### Definition of Terms of Substituents

Halogen or halide—as used herein includes fluorine, chlorine, bromine, and iodine.

Alkyl—as used herein includes both straight and branched chain alkyl groups. Alkyl may be alkyl having 1 to 20 carbon atoms, preferably alkyl having 1 to 12 carbon atoms, and more preferably alkyl having 1 to 6 carbon atoms. Examples of alkyl groups include a methyl group, an ethyl group, a propyl group, an isopropyl group, a n-butyl group, an s-butyl group, an isobutyl group, a t-butyl group, an n-pentyl group, an n-hexyl group, an n-heptyl group, an n-octyl group, an n-nonyl group, an n-decyl group, an n-undecyl group, an n-dodecyl group, an n-tridecyl group, an n-tetradecyl group, an n-pentadecyl group, an n-hexadecyl group, an n-heptadecyl group, an n-octadecyl group, a neopentyl group, a 1-methylpentyl group, a 2-methylpentyl group, a 1-pentylhexyl group, a 1-butylpentyl group, a 1-heptyloctyl group, and a 3-methylpentyl group. Of the above, preferred are a methyl group, an ethyl group, a propyl group, an isopropyl group, a n-butyl group, an s-butyl group, an isobutyl group, a t-butyl group, an n-pentyl group, a neopentyl group, and an n-hexyl group. Additionally, the alkyl group may be optionally substituted.

Cycloalkyl—as used herein includes cyclic alkyl groups. The cycloalkyl groups may be those having 3 to 20 ring carbon atoms, preferably those having 4 to 10 carbon atoms. Examples of cycloalkyl include cyclobutyl, cyclopentyl, cyclohexyl, 4-methylcyclohexyl, 4,4-dimethylcylcohexyl, 1-adamantyl, 2-adamantyl, 1-norbornyl, 2-norbornyl, and the like. Of the above, preferred are cyclopentyl, cyclohexyl, 4-methylcyclohexyl, and 4,4-dimethylcylcohexyl. Additionally, the cycloalkyl group may be optionally substituted.

Heteroalkyl—as used herein, includes a group formed by replacing one or more carbons in an alkyl chain with a hetero-atom(s) selected from the group consisting of a nitrogen atom, an oxygen atom, a sulfur atom, a selenium atom, a phosphorus atom, a silicon atom, a germanium atom, and a boron atom. Heteroalkyl may be those having 1 to 20 carbon atoms, preferably those having 1 to 10 carbon atoms, and more preferably those having 1 to 6 carbon atoms. Examples of heteroalkyl include methoxymethyl, ethoxymethyl, ethoxyethyl, methylthiomethyl, ethylthiomethyl, ethylthioethyl, methoxymethoxymethyl, ethoxymethoxymethyl, ethoxyethoxyethyl, hydroxymethyl, hydroxyethyl, hydroxypropyl, mercaptomethyl, mercaptoethyl, mercaptopropyl, aminomethyl, aminoethyl, aminopropyl, dimethylaminomethyl, trimethylgermanylmethyl, trimethylgermanylethyl, trimethylgermanylisopropyl, dimethylethylgermanylmethyl, dimethylisopropylgermanylmethyl, tert-butyldimethylgermanylmethyl, triethylgermanylmethyl, triethylgermanylethyl, triisopropylgermanylmethyl, triisopropylgermanylethyl, trimethylsilylmethyl, trimethylsilylethyl, trimethylsilylisopropyl, triisopropylsilylmethyl, and triisopropylsilylethyl. Additionally, the heteroalkyl group may be optionally substituted.

Alkenyl—as used herein includes straight chain, branched chain, and cyclic alkene groups. Alkenyl may be those having 2 to 20 carbon atoms, preferably those having 2 to 10 carbon atoms. Examples of alkenyl include vinyl, 1-propenyl group, 1-butenyl, 2-butenyl, 3-butenyl, 1,3-butandienyl, 1-methylvinyl, styryl, 2,2-diphenylvinyl, 1,2-diphenylvinyl, 1-methylallyl, 1,1-dimethylallyl, 2-methylallyl, 1-phenylallyl, 2-phenylallyl, 3-phenylallyl, 3,3-diphenylallyl, 1,2-dimethylallyl, 1-phenyl-1-butenyl, 3-phenyl-1-butenyl, cyclopentenyl, cyclopentadienyl, cyclohexenyl, cycloheptenyl, cycloheptatrienyl, cyclooctenyl, cyclooctatetraenyl, and norbornenyl. Additionally, the alkenyl group may be optionally substituted.

Alkynyl—as used herein includes straight chain alkynyl groups. Alkynyl may be those having 2 to 20 carbon atoms, preferably those having 2 to 10 carbon atoms. Examples of alkynyl groups include ethynyl, propynyl, propargyl, 1-butynyl, 2-butynyl, 3-butynyl, 1-pentynyl, 2-pentynyl, 3,3-dimethyl-1-butynyl, 3-ethyl-3-methyl-1-pentynyl, 3,3-diisopropyl-1-pentynyl, phenylethynyl, phenylpropynyl, etc. Of the above, preferred are ethynyl, propynyl, propargyl, 1-butynyl, 2-butynyl, 3-butynyl, 1-pentynyl, and phenylethynyl. Additionally, the alkynyl group may be optionally substituted.

Aryl or an aromatic group—as used herein includes non-condensed and condensed systems. Aryl may be those having 6 to 30 carbon atoms, preferably those having 6 to 20 carbon atoms, and more preferably those having 6 to 12 carbon atoms. Examples of aryl groups include phenyl, biphenyl, terphenyl, triphenylene, tetraphenylene, naphthalene, anthracene, phenalene, phenanthrene, fluorene, pyrene, chrysene, perylene, and azulene, preferably phenyl, biphenyl, terphenyl, triphenylene, fluorene, and naphthalene. Examples of non-condensed aryl groups include phenyl, biphenyl-2-yl, biphenyl-3-yl, biphenyl-4-yl, p-terphenyl-4-yl, p-terphenyl-3-yl, p-terphenyl-2-yl, m-terphenyl-4-yl, m-terphenyl-3-yl, m-terphenyl-2-yl, o-tolyl, m-tolyl, p-tolyl, p-(2-phenylpropyl)phenyl, 4′-methylbiphenylyl, 4″-t-butyl-p-terphenyl-4-yl, o-cumenyl, m-cumenyl, p-cumenyl, 2,3-xylyl, 3,4-xylyl, 2,5-xylyl, mesityl, and m-quarterphenyl. Additionally, the aryl group may be optionally substituted.

Heterocyclic groups or heterocycle—as used herein include non-aromatic cyclic groups. Non-aromatic heterocyclic groups include saturated heterocyclic groups having 3 to 20 ring atoms and unsaturated non-aromatic heterocyclic groups having 3 to 20 ring atoms, where at least one ring atom is selected from the group consisting of a nitrogen atom, an oxygen atom, a sulfur atom, a selenium atom, a silicon atom, a phosphorus atom, a germanium atom, and a boron atom. Preferred non-aromatic heterocyclic groups are those having 3 to 7 ring atoms, each of which includes at least one hetero-atom such as nitrogen, oxygen, silicon, or sulfur. Examples of non-aromatic heterocyclic groups include oxiranyl, oxetanyl, tetrahydrofuranyl, tetrahydropyranyl, dioxolanyl, dioxanyl, aziridinyl, dihydropyrrolyl, tetrahydropyrrolyl, piperidinyl, oxazolidinyl, morpholinyl, piperazinyl, oxepinyl, thiepinyl, azepinyl, and tetrahydrosilolyl. Additionally, the heterocyclic group may be optionally substituted.

Heteroaryl—as used herein, includes non-condensed and condensed hetero-aromatic groups having 1 to 5 hetero-atoms, where at least one hetero-atom is selected from the group consisting of a nitrogen atom, an oxygen atom, a sulfur atom, a selenium atom, a silicon atom, a phosphorus atom, a germanium atom, and a boron atom. A hetero-aromatic group is also referred to as heteroaryl. Heteroaryl may be those having 3 to 30 carbon atoms, preferably those having 3 to 20 carbon atoms, and more preferably those having 3 to 12 carbon atoms. Suitable heteroaryl groups include dibenzothiophene, dibenzofuran, dibenzoselenophene, furan, thiophene, benzofuran, benzothiophene, benzoselenophene, carbazole, indolocarbazole, pyridoindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, benzofuropyridine, furodipyridine, benzothienopyridine, thienodipyridine, benzoselenophenopyridine, and selenophenodipyridine, preferably dibenzothiophene, dibenzofuran, dibenzoselenophene, carbazole, indolocarbazole, imidazole, pyridine, triazine, benzimidazole, 1,2-azaborine, 1,3-azaborine, 1,4-azaborine, borazine, and aza-analogs thereof. Additionally, the heteroaryl group may be optionally substituted.

Alkoxy—as used herein, is represented by —O-alkyl, —O-cycloalkyl, —O-heteroalkyl, or —O-heterocyclic group. Examples and preferred examples of alkyl, cycloalkyl, heteroalkyl, and heterocyclic groups are the same as those described above. Alkoxy groups may be those having 1 to 20 carbon atoms, preferably those having 1 to 6 carbon atoms. Examples of alkoxy groups include methoxy, ethoxy, propoxy, butoxy, pentyloxy, hexyloxy, cyclopropyloxy, cyclobutyloxy, cyclopentyloxy, cyclohexyloxy, tetrahydrofuranyloxy, tetrahydropyranyloxy, methoxypropyloxy, ethoxyethyloxy, methoxymethyloxy, and ethoxymethyloxy. Additionally, the alkoxy group may be optionally substituted.

Aryloxy—as used herein, is represented by —O-aryl or —O-heteroaryl. Examples and preferred examples of aryl and heteroaryl are the same as those described above. Aryloxy groups may be those having 6 to 30 carbon atoms, preferably those having 6 to 20 carbon atoms. Examples of aryloxy groups include phenoxy and biphenyloxy. Additionally, the aryloxy group may be optionally substituted.

Arylalkyl—as used herein, contemplates alkyl substituted with an aryl group. Arylalkyl may be those having 7 to 30 carbon atoms, preferably those having 7 to 20 carbon atoms, and more preferably those having 7 to 13 carbon atoms. Examples of arylalkyl groups include benzyl, 1-phenylethyl, 2-phenylethyl, 1-phenylisopropyl, 2-phenylisopropyl, phenyl-t-butyl, alpha-naphthylmethyl, 1-alpha-naphthylethyl, 2-alpha-naphthylethyl, 1-alpha-naphthylisopropyl, 2-alpha-naphthylisopropyl, beta-naphthylmethyl, 1-beta-naphthylethyl, 2-beta-naphthylethyl, 1-beta-naphthylisopropyl, 2-beta-naphthylisopropyl, p-methylbenzyl, m-methylbenzyl, o-methylbenzyl, p-chlorobenzyl, m-chlorobenzyl, o-chlorobenzyl, p-bromobenzyl, m-bromobenzyl, o-bromobenzyl, p-iodobenzyl, m-iodobenzyl, o-iodobenzyl, p-hydroxybenzyl, m-hydroxybenzyl, o-hydroxybenzyl, p-aminobenzyl, m-aminobenzyl, o-aminobenzyl, p-nitrobenzyl, m-nitrobenzyl, o-nitrobenzyl, p-cyanobenzyl, m-cyanobenzyl, o-cyanobenzyl, 1-hydroxy-2-phenylisopropyl, and 1-chloro-2-phenylisopropyl. Of the above, preferred are benzyl, p-cyanobenzyl, m-cyanobenzyl, o-cyanobenzyl, 1-phenylethyl, 2-phenylethyl, 1-phenylisopropyl, and 2-phenylisopropyl. Additionally, the arylalkyl group may be optionally substituted.

Alkylsilyl—as used herein, contemplates a silyl group substituted with an alkyl group. Alkylsilyl groups may be those having 3 to 20 carbon atoms, preferably those having 3 to 10 carbon atoms. Examples of alkylsilyl groups include trimethylsilyl, triethylsilyl, methyldiethylsilyl, ethyldimethylsilyl, tripropylsilyl, tributylsilyl, triisopropylsilyl, methyldiisopropylsilyl, dimethylisopropylsilyl, tri-t-butylsilyl, triisobutylsilyl, dimethyl t-butylsilyl, and methyldi-t-butylsilyl. Additionally, the alkylsilyl group may be optionally substituted.

Arylsilyl—as used herein, contemplates a silyl group substituted with an aryl group. Arylsilyl groups may be those having 6 to 30 carbon atoms, preferably those having 8 to 20 carbon atoms. Examples of arylsilyl groups include triphenylsilyl, phenyldibiphenylylsilyl, diphenylbiphenylsilyl, phenyldiethylsilyl, diphenylethylsilyl, phenyldimethylsilyl, diphenylmethylsilyl, phenyldiisopropylsilyl, diphenylisopropylsilyl, diphenylbutylsilyl, diphenylisobutylsilyl, diphenyl t-butylsilyl. Additionally, the arylsilyl group may be optionally substituted.

Alkylgermanyl—as used herein contemplates a germanyl substituted with an alkyl group. The alkylgermanyl may be those having 3 to 20 carbon atoms, preferably those having 3 to 10 carbon atoms. Examples of alkylgermanyl include trimethylgermanyl, triethylgermanyl, methyldiethylgermanyl, ethyldimethylgermanyl, tripropylgermanyl, tributylgermanyl, triisopropylgermanyl, methyldiisopropylgermanyl, dimethylisopropylgermanyl, tri-t-butylgermanyl, triisobutylgermanyl, dimethyl-t-butylgermanyl, and methyldi-t-butylgermanyl. Additionally, the alkylgermanyl may be optionally substituted.

Arylgermanyl—as used herein contemplates a germanyl substituted with at least one aryl group or heteroaryl group. Arylgermanyl may be those having 6 to 30 carbon atoms, preferably those having 8 to 20 carbon atoms. Examples of arylgermanyl include triphenylgermanyl, phenyldibiphenylylgermanyl, diphenylbiphenylgermanyl, phenyldiethylgermanyl, diphenylethylgermanyl, phenyldimethylgermanyl, diphenylmethylgermanyl, phenyldiisopropylgermanyl, diphenylisopropylgermanyl, diphenylbutylgermanyl, diphenylisobutylgermanyl, and diphenyl-t-butylgermanyl. Additionally, the arylgermanyl may be optionally substituted.

The term “aza” in azadibenzofuran, azadibenzothiophene, etc. means that one or more of C—H groups in the respective aromatic fragment are replaced by a nitrogen atom. For example, azatriphenylene encompasses dibenzo[f,h]quinoxaline, dibenzo[f,h]quinoline and other analogs with two or more nitrogens in the ring system. One of ordinary skill in the art can readily envision other nitrogen analogs of the aza-derivatives described above, and all such analogs are intended to be encompassed by the terms as set forth herein.

In the present disclosure, unless otherwise defined, when any term of the group consisting of substituted alkyl, substituted cycloalkyl, substituted heteroalkyl, substituted heterocyclic group, substituted arylalkyl, substituted alkoxy, substituted aryloxy, substituted alkenyl, substituted alkynyl, substituted aryl, substituted heteroaryl, substituted alkylsilyl, substituted arylsilyl, substituted alkylgermanyl, substituted arylgermanyl, substituted amino, substituted acyl, substituted carbonyl, a substituted carboxylic acid group, a substituted ester group, substituted sulfinyl, substituted sulfonyl, and substituted phosphino is used, it means that any group of alkyl, cycloalkyl, heteroalkyl, heterocyclic group, arylalkyl, alkoxy, aryloxy, alkenyl, alkynyl, aryl, heteroaryl, alkylsilyl, arylsilyl, alkylgermanyl, arylgermanyl, amino, acyl, carbonyl, a carboxylic acid group, an ester group, sulfinyl, sulfonyl, and phosphino may be substituted with one or more groups selected from the group consisting of deuterium, halogen, unsubstituted alkyl having 1 to 20 carbon atoms, unsubstituted cycloalkyl having 3 to 20 ring carbon atoms, unsubstituted heteroalkyl having 1 to 20 carbon atoms, an unsubstituted heterocyclic group having 3 to 20 ring atoms, unsubstituted arylalkyl having 7 to 30 carbon atoms, unsubstituted alkoxy having 1 to 20 carbon atoms, unsubstituted aryloxy having 6 to 30 carbon atoms, unsubstituted alkenyl having 2 to 20 carbon atoms, unsubstituted alkynyl having 2 to 20 carbon atoms, unsubstituted aryl having 6 to 30 carbon atoms, unsubstituted heteroaryl having 3 to 30 carbon atoms, unsubstituted alkylsilyl having 3 to 20 carbon atoms, unsubstituted arylsilyl group having 6 to 20 carbon atoms, unsubstituted alkylgermanyl group having 3 to 20 carbon atoms, unsubstituted arylgermanyl group having 6 to 20 carbon atoms, unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof.

It is to be understood that when a molecular fragment is described as being a substituent or otherwise attached to another moiety, its name may be written as if it were a fragment (e.g. phenyl, phenylene, naphthyl, dibenzofuryl) or as if it were the whole molecule (e.g. benzene, naphthalene, dibenzofuran). As used herein, these different ways of designating a substituent or an attached fragment are considered to be equivalent.

In the compounds mentioned in the present disclosure, hydrogen atoms may be partially or fully replaced by deuterium. Other atoms such as carbon and nitrogen may also be replaced by their other stable isotopes. The replacement by other stable isotopes in the compounds may be preferred due to its enhancements of device efficiency and stability.

In the compounds mentioned in the present disclosure, multiple substitutions refer to a range that includes di-substitutions, up to the maximum available substitutions. When substitution in the compounds mentioned in the present disclosure represents multiple substitutions (including di-, tri-, and tetra-substitutions etc.), that means the substituent may exist at a plurality of available substitution positions on its linking structure, the substituents present at a plurality of available substitution positions may have the same structure or different structures.

In the compounds mentioned in the present disclosure, adjacent substituents in the compounds cannot be joined to form a ring unless otherwise explicitly defined, for example, adjacent substituents can be optionally joined to form a ring. In the compounds mentioned in the present disclosure, the expression that adjacent substituents can be optionally joined to form a ring includes a case where adjacent substituents may be joined to form a ring and a case where adjacent substituents are not joined to form a ring. When adjacent substituents can be optionally joined to form a ring, the ring formed may be monocyclic or polycyclic (including spirocyclic, endocyclic, fusedcyclic, and etc.), as well as alicyclic, heteroalicyclic, aromatic, or heteroaromatic. In such expression, adjacent substituents may refer to substituents bonded to the same atom, substituents bonded to carbon atoms which are directly bonded to each other, or substituents bonded to carbon atoms which are more distant from each other. Preferably, adjacent substituents refer to substituents bonded to the same carbon atom and substituents bonded to carbon atoms which are directly bonded to each other.

The expression that adjacent substituents can be optionally joined to form a ring is also intended to mean that two substituents bonded to the same carbon atom are joined to each other via a chemical bond to form a ring, which can be exemplified by the following formula:

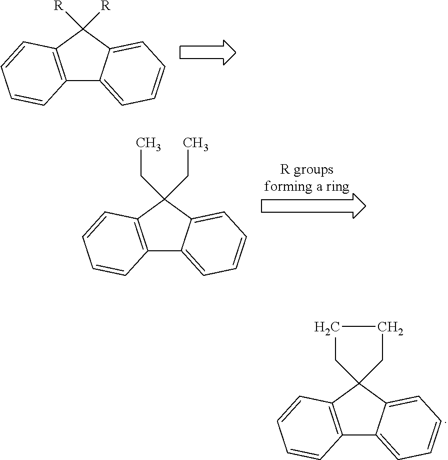

The expression that adjacent substituents can be optionally joined to form a ring is also intended to mean that two substituents bonded to carbon atoms which are directly bonded to each other are joined to each other via a chemical bond to form a ring, which can be exemplified by the following formula:

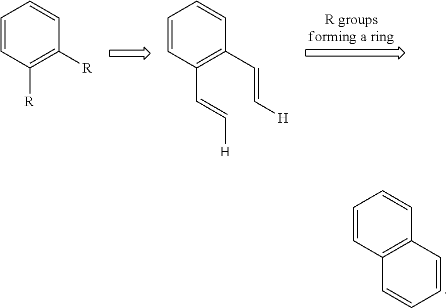

The expression that adjacent substituents can be optionally joined to form a ring is also intended to mean that two substituents bonded to further distant carbon atoms are joined to each other via a chemical bond to form a ring, which can be exemplified by the following formula:

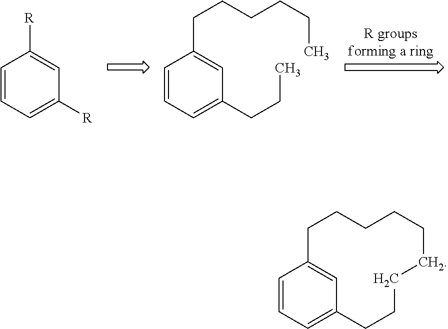

Furthermore, the expression that adjacent substituents can be optionally joined to form a ring is also intended to mean that, in the case where one of the two substituents bonded to carbon atoms which are directly bonded to each other represents hydrogen, the second substituent is bonded at a position at which the hydrogen atom is bonded, thereby forming a ring. This is exemplified by the following formula:

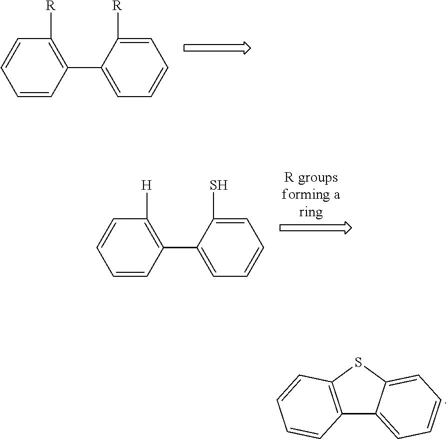

According to an embodiment of the present disclosure, disclosed is a compound having a structure of Formula 1:

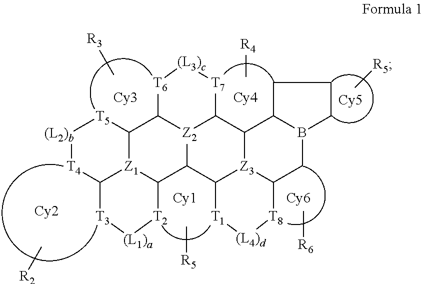

wherein the ring Cy1, the ring Cy2, the ring Cy3, the ring Cy5, and the ring Cy6 are, at each occurrence identically or differently, selected from an unsaturated carbocyclic ring having 5 to 30 carbon atoms or an unsaturated heterocyclic ring having 3 to 30 carbon atoms; and the ring Cy4 is selected from an unsaturated carbocyclic ring having 5 to 30 carbon atoms or an unsaturated heterocyclic ring having 4 to 30 carbon atoms;
Z₁, Z₂, and Z₃ are, at each occurrence identically or differently, selected from B, N, P, P═O, P═S, As, As═O, As═S, SiR, or GeR;
L₁, L₂, L₃, and L₄ are, at each occurrence identically or differently, selected from a single bond, O, S, Se, NR′, PR′, P═OR′, P═SR′, AsR′, As═OR′, As═SR′, CR′R′, SiR′R′, or GeR′R′; when multiple R′ are present at the same time, the multiple R′ are identical or different;
a, b, c, and d are each independently selected from 0, 1, or 2;
T₁ to T₈ are, at each occurrence identically or differently, selected from C, CRₜ, CRₜRₜ, N, NRₜ, O, S, or Se;
R₁, R₂, R₃, R₄, R₅, and R₆ represent, at each occurrence identically or differently, mono-substitution, multiple substitutions, or non-substitution;
R, R′, Rₜ, R₁, R₂, R₃, R₄, R₅, and R₆ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
adjacent substituents R₅ can be optionally joined to form a ring; and
adjacent substituents R′, Rₜ, R₁, R₂, R₃, R₄, and R₆ can be optionally joined to form a ring.

In this embodiment, the “unsaturated carbocyclic ring” comprises an aromatic unsaturated carbocyclic ring (an aromatic ring) and a non-aromatic unsaturated carbocyclic ring; and the “unsaturated heterocyclic ring” comprises an aromatic unsaturated heterocyclic ring (a heteroaromatic ring) and a non-aromatic unsaturated heterocyclic ring.

In this embodiment, when a, b, c, and d are selected from 0, it is intended to mean that L₁, L₂, L₃, and L₄ are absent. For example, when a is 0, L₁ is absent, and T₂ and T₃ are not joined; when b, c, and d are selected from 0, the cases are similar to that of a.

In this embodiment, when a, b, c, and d are selected from 0, T₁ to T₈ are identically or differently, selected from CRₜ, CRₜRₜ, N, NRₜ, O, S, or Se; when a, b, c, and d are selected from 1 or 2, T₁ to T₈ are identically or differently, selected from C or N.

Herein, the expression that “adjacent substituents R₅ can be optionally joined to form a ring” is intended to mean that adjacent substituents R₅ may be joined to form a ring. Obviously, it is also possible that adjacent substituents R₅ are not joined to form a ring.

Herein, the expression that “adjacent substituents R′, Rₜ, R₁, R₂, R₃, R₄, and R₆ can be optionally joined to form a ring” is intended to mean that any one or more of groups of adjacent substituents, such as two substituents R′, two substituents Rₜ, two substituents R₁, two substituents R₂, two substituents R₃, two substituents R₄, two substituents R₆, substituents R₁ and R₂, substituents R₂ and R₃, substituents R₃ and R₄, substituents R₆ and R₁, substituents R₁ and R′, substituents R₂ and R′, substituents R₃ and R′, substituents R₄ and R′, substituents R₆ and R′, substituents R₁ and Rₜ, substituents R₂ and Rₜ, substituents R₃ and Rₜ, substituents R₄ and Rₜ, and substituents R₆ and Rₜ, may be joined to form a ring. Obviously, it is also possible that none of these substituents are joined to form a ring.

According to an embodiment of the present disclosure, the ring Cy1, the ring Cy2, the ring Cy3, the ring Cy5, and the ring Cy6 are, at each occurrence identically or differently, selected from a five-membered unsaturated carbocyclic ring, an aromatic ring having 6 to 18 carbon atoms, or a heteroaromatic ring having 3 to 18 carbon atoms; and the ring Cy4 is selected from a five-membered unsaturated carbocyclic ring, an aromatic ring having 6 to 18 carbon atoms, or a heteroaromatic ring having 4 to 18 carbon atoms.

According to an embodiment of the present disclosure, the ring Cy1, the ring Cy2, the ring Cy3, the ring Cy4, the ring Cy5, and the ring Cy6 are, at each occurrence identically or differently, selected from a benzene ring, a pyridine ring, a naphthalene ring, a phenanthrene ring, an anthracene ring, an indene ring, a fluorene ring, an indole ring, a carbazole ring, a benzofuran ring, a dibenzofuran ring, a benzosilole ring, a dibenzosilole ring, a benzothiophene ring, a dibenzothiophene ring, a dibenzoselenophene ring, a cyclopentadienyl ring, a furan ring, a thiophene ring, a silole ring, or a combination thereof.

According to an embodiment of the present disclosure, the compound has a structure represented by any one of Formula 1-1 to Formula 1-4:

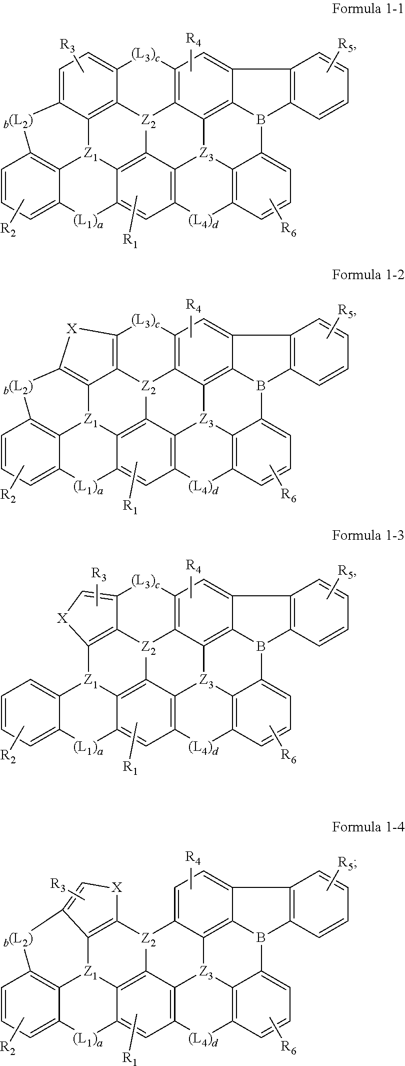

wherein Z₁, Z₂, and Z₃ are, at each occurrence identically or differently, selected from B, N, P, P═O, P═S, As, As═O, As═S, SiR, or GeR;
X is, at each occurrence identically or differently, selected from O, S, Se, or NRx;
L₁, L₂, L₃, and L₄ are, at each occurrence identically or differently, selected from a single bond, O, S, Se, NR′, PR′, P═OR′, P═SR′, AsR′, As═OR′, As═SR′, CR′R′, SiR′R′, or GeR′R′; when multiple R′ are present at the same time, the multiple R′ are identical or different;
a, b, c, and d are each independently selected from 0, 1, or 2;
R₁, R₂, R₃, R₄, R₅, and R₆ represent, at each occurrence identically or differently, mono-substitution, multiple substitutions, or non-substitution;
R, R′, Rₓ, R₁, R₂, R₃, R₄, R₅, and R₆ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
adjacent substituents R₅ can be optionally joined to form a ring; and
adjacent substituents R′, Rₓ, R₁, R₂, R₃, R₄, and R₆ can be optionally joined to form a ring.

Herein, the expression that “adjacent substituents R′, Rₓ, R₁, R₂, R₃, R₄, and R₆ can be optionally joined to form a ring” is intended to mean that any one or more of groups of adjacent substituents, such as two substituents R′, two substituents R₁, two substituents R₂, two substituents R₃, two substituents R₄, two substituents R₆, substituents R₁ and R₂, substituents R₂ and R₃, substituents R₃ and R₄, substituents R₆ and R₁, substituents R₁ and R′, substituents R₂ and R′, substituents R₃ and R′, substituents R₄ and R′, substituents R₆ and R′, substituents Rₓ and R′, substituents Rₓ and R₂, and substituents Rₓ and R₄, may be joined to form a ring. Obviously, it is also possible that none of these substituents are joined to form a ring.

According to an embodiment of the present disclosure, the compound has a structure represented by Formula 1-1.

According to an embodiment of the present disclosure, the L₁, L₂, L₃, and L₄ are, at each occurrence identically or differently, selected from a single bond, O, S, Se, NR′, P═OR′, CR′R′, or SiR′R′; and the R′ is, at each occurrence identically or differently, selected from the group consisting of: substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted aryl having 6 to 30 carbon atoms, substituted or unsubstituted heteroaryl having 3 to 30 carbon atoms, and combinations thereof.

According to an embodiment of the present disclosure, the L₁, L₂, L₃, and L₄ are, at each occurrence identically or differently, selected from a single bond, O, S, NR′, CR′R′, or SiR′R′; and the R′ is, at each occurrence identically or differently, selected from the group consisting of substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, and combinations thereof.

According to an embodiment of the present disclosure, the L₁, L₂, and L₄ are, at each occurrence identically or differently, selected from a single bond, O, S, NR′, CR′R′, or SiR′R′; L₃ is selected from a single bond, O, S, CR′R′, or SiR′R′; and the R′ is, at each occurrence identically or differently, selected from the group consisting of: substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, and combinations thereof.

According to an embodiment of the present disclosure, the L₁, L₂, L₃, and L₄ are, at each occurrence identically or differently, selected from a single bond, O, S, CR′R′, or SiR′R′; and the R′ is, at each occurrence identically or differently, selected from the group consisting of substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, and combinations thereof.

According to an embodiment of the present disclosure, the L₁, L₂, L₃, and L₄ are each selected from a single bond.

According to an embodiment of the present disclosure, a, b, c, and d are each independently selected from 0 or 1.

According to an embodiment of the present disclosure, a+b+c+d=0, 1, or 2.

According to an embodiment of the present disclosure, a+b+c+d=0 or 1.

According to an embodiment of the present disclosure, a+b=0, 1, or 2; and c and d are both 0.

According to an embodiment of the present disclosure, a+b=1; and c and d are both 0.

According to an embodiment of the present disclosure, a is 1, and b, c, and d are all 0.

According to an embodiment of the present disclosure, b is 1, and a, c, and d are all 0.

According to an embodiment of the present disclosure, a+b=1, and L₁ or L₂ is a single bond; and c and d are both 0.

According to an embodiment of the present disclosure, a is 1, and L₁ is a single bond; and b, c, and d are all 0.

According to an embodiment of the present disclosure, b is 1, and L₂ is a single bond; and a, c, and d are all 0.

According to an embodiment of the present disclosure, the Z₁, Z₂, and Z₃ are, at each occurrence identically or differently, selected from B, N, P, P═O, or P═S, and at least one of the Z₁, Z₂, or Z₃ is, at each occurrence identically or differently, selected from N, P, P═O, or P═S.

According to an embodiment of the present disclosure, the Z₁, Z₂, and Z₃ are, at each occurrence identically or differently, selected from B, N, or P═O, and at least two of the Z₁, Z₂, and Z₃ are selected from N.

According to an embodiment of the present disclosure, the Z₁ and Z₃ are, at each occurrence identically or differently, selected from N, P, P═O, or P═S, and the Z₂ is selected from B.

According to an embodiment of the present disclosure, the Z₁ and Z₃ are, at each occurrence identically or differently, selected from N or P, and the Z₂ is selected from B.

According to an embodiment of the present disclosure, the Z₁ and Z₃ are selected from N, and the Z₂ is selected from B.

According to an embodiment of the present disclosure, the compound comprises at most two B atoms.

According to an embodiment of the present disclosure, the R₁, R₂, R₃, R₄, R₅, and R₆ are, at each occurrence identically or differently, selected from the group consisting of hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted alkylgermanyl having 3 to 20 carbon atoms, substituted or unsubstituted amino having 0 to 20 carbon atoms, cyano, isocyano, and combinations thereof; adjacent substituents R₅ can be optionally joined to form a ring; and adjacent substituents R₁, R₂, R₃, R₄, and R₆ can be optionally joined to form a ring.

According to an embodiment of the present disclosure, the R₁, R₂, R₃, R₄, R₅, and R₆ are, at each occurrence identically or differently, selected from the group consisting of hydrogen, deuterium, halogen, substituted or unsubstituted alkyl having 1 to 12 carbon atoms, substituted or unsubstituted cycloalkyl having 3 to 12 ring carbon atoms, substituted or unsubstituted aryl having 6 to 24 carbon atoms, substituted or unsubstituted heteroaryl having 3 to 24 carbon atoms, substituted or unsubstituted alkylsilyl having 3 to 10 carbon atoms, substituted or unsubstituted amino having 0 to 20 carbon atoms, cyano, and combinations thereof; adjacent substituents R₅ can be optionally joined to form a ring; and adjacent substituents R₁, R₂, R₃, R₄, and R₆ can be optionally joined to form a ring.

According to an embodiment of the present disclosure, the R₁, R₂, R₃, R₄, R₅, and R₆ are, at each occurrence identically or differently, selected from the group consisting of hydrogen, deuterium, fluorine, cyano, methyl, ethyl, n-propyl, isopropyl, cyclopropyl, n-butyl, isobutyl, t-butyl, cyclopentyl, neopentyl, cyclohexyl, trimethylsilyl, phenyl, biphenyl, terphenyl, tetraphenyl, triphenylenyl, tetraphenylenyl, naphthyl, phenanthryl, anthryl, indenyl, fluorenyl, indolyl, carbazolyl, benzofuryl, dibenzofuryl, benzosilolyl, dibenzosilolyl, benzothienyl, dibenzothienyl, dibenzoselenophenyl, diphenylamino, dibenzofurylphenylamino, and combinations thereof.

According to an embodiment of the present disclosure, at least one of the R₁, R₂, R₃, R₄, R₅, or R₆ is, at each occurrence identically or differently, selected from the group consisting of: deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof, adjacent substituents R₅ can be optionally joined to form a ring; and adjacent substituents R₁, R₂, R₃, R₄, and R₆ can be optionally joined to form a ring.

According to an embodiment of the present disclosure, at least one of the R₁, R₂, R₃, R₄, R₅, or R₆ is, at each occurrence identically or differently, selected from the group consisting of: deuterium, halogen, substituted or unsubstituted alkyl having 1 to 12 carbon atoms, substituted or unsubstituted cycloalkyl having 3 to 12 ring carbon atoms, substituted or unsubstituted aryl having 6 to 24 carbon atoms, substituted or unsubstituted heteroaryl having 3 to 24 carbon atoms, substituted or unsubstituted alkylsilyl having 3 to 10 carbon atoms, substituted or unsubstituted amino having 0 to 20 carbon atoms, cyano, and combinations thereof, adjacent substituents R₅ can be optionally joined to form a ring; and adjacent substituents R₁, R₂, R₃, R₄, and R₆ can be optionally joined to form a ring.

According to an embodiment of the present disclosure, at least one of the R₁, R₂, R₃, R₄, R₅, or R₆ is, at each occurrence identically or differently, selected from the group consisting of: deuterium, fluorine, cyano, methyl, ethyl, n-propyl, isopropyl, cyclopropyl, n-butyl, isobutyl, t-butyl, cyclopentyl, neopentyl, cyclohexyl, trimethylsilyl, phenyl, biphenyl, terphenyl, tetraphenyl, triphenylenyl, tetraphenylenyl, naphthyl, phenanthryl, anthryl, indenyl, fluorenyl, indolyl, carbazolyl, benzofuryl, dibenzofuryl, benzosilolyl, dibenzosilolyl, benzothienyl, dibenzothienyl, dibenzoselenophenyl, diphenylamino, dibenzofurylphenylamino, and combinations thereof.

According to an embodiment of the present disclosure, at least one of the R₁, R₂, R₃, R₄, R₅, or R₆ is, at each occurrence identically or differently, selected from substituted or unsubstituted alkyl having 1 to 20 carbon atoms.

According to an embodiment of the present disclosure, at least one of the R₁, R₂, R₃, R₄, R₅, or R₆ is, at each occurrence identically or differently, selected from substituted or unsubstituted alkyl having 1 to 12 carbon atoms.

According to an embodiment of the present disclosure, at least one of the R₁, R₂, R₃, R₄, R₅, or R₆ is selected from cyano.

According to an embodiment of the present disclosure, at least one of R₅ is selected from cyano.

According to an embodiment of the present disclosure, the compound has a structure represented by Formula 1-1-1:

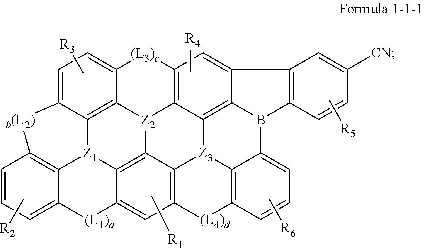

wherein Z₁, Z₂, and Z₃ are, at each occurrence identically or differently, selected from B, N, P, P═O, P═S, As, As═O, As═S, SiR, or GeR;
L₁, L₂, L₃, and L₄ are, at each occurrence identically or differently, selected from a single bond, O, S, Se, NR′, PR′, P═OR′, P═SR′, AsR′, As═OR′, As═SR′, CR′R′, SiR′R′, or GeR′R′; when multiple R′ are present at the same time, the multiple R′ are identical or different;
a, b, c, and d are each independently selected from 0, 1, or 2;
R₁, R₂, R₃, R₄, R₅, and R₆ represent, at each occurrence identically or differently, mono-substitution, multiple substitutions, or non-substitution;
R, R′, R₁, R₂, R₃, R₄, R₅, and R₆ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof,
adjacent substituents R₅ can be optionally joined to form a ring; and
adjacent substituents R′, R₁, R₂, R₃, R₄, and R₆ can be optionally joined to form a ring.

According to an embodiment of the present disclosure, the compound is selected from the group consisting of Compound BN-1-1-h₁ to Compound BN-1-20-h₁ and Compound BN-2-1-h₂ to Compound BN-2-6-h₂; wherein h₁ is selected from an integer from 1 to 127; and h₂ is selected from an integer from 1 to 390. For specific structures of Compound BN-1-1-h₁ to Compound BN-1-20-h₁ and Compound BN-2-1-h₂ to Compound BN-2-6-h₂, refer to claim 9.

In this embodiment, h₁ is selected from an integer from 1 to 127; therefore, Compound BN-1-1-h₁ represents Compounds BN-1-1-1 to BN-1-1-127, Compound BN-1-2-h₁ represents Compounds BN-1-2-1 to BN-1-2-127, and it is true for Compound BN-1-3-h₁ to Compound BN-1-20-h₁. For example, Compound BN-1-5-h₁ represents Compounds BN-1-5-1 to BN-1-5-127, and Compounds BN-1-5-1 to BN-1-5-127 all have a skeleton structure of

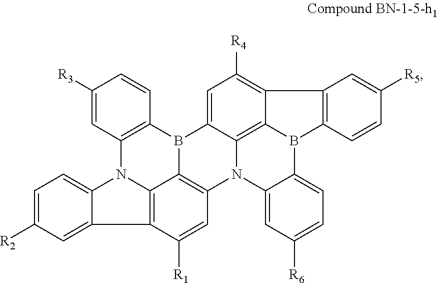

wherein R₁, R₂, R₃, R₄, R₅, and R₆ in Compound BN-1-5-73 are selected from atoms or groups corresponding to h₁ of 73, that is, the structure of Compound BN-1-5-73 is

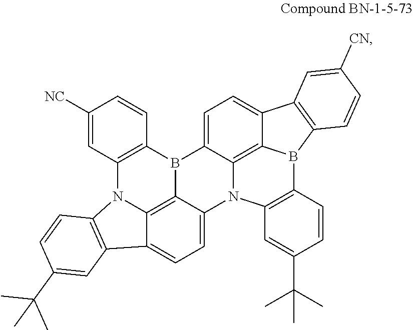 [SMILES: CC(C)(C)c1ccc2c(c1)N1c3ccc4c5cc(C(C)(C)C)ccc5n5c4c3B(c3ccc(C#N)cc3-5)c3ccc4c(c31)B2c1ccc(C#N)cc1-4]

and R₁, R₂, R₃, R₄, R₅, and R₆ in Compound BN-1-5-94 are selected from atoms or groups corresponding to h₁ of 94, that is, the structure of Compound BN-1-5-94 is

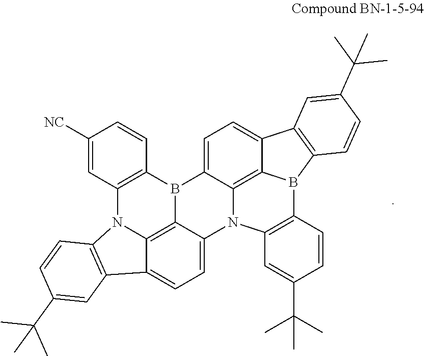 [SMILES: CC(C)(C)c1ccc2c(c1)-c1ccc3c4c1B2c1ccc(C(C)(C)C)cc1N4c1ccc2c4cc(C(C)(C)C)ccc4n4c2c1B3c1ccc(C#N)cc1-4]

In another example, Compound BN-1-7-h₁ represents Compounds BN-1-7-1 to BN-1-7-127, and Compounds BN-1-7-1 to BN-1-7-127 all have a skeleton structure of

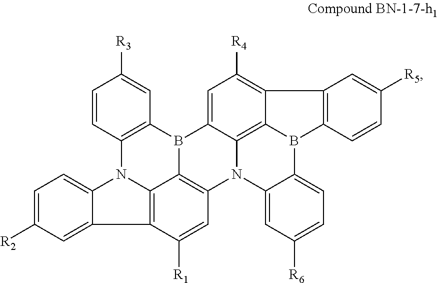

wherein R₁, R₂, R₃, R₄, R₅, and R₆ in Compound BN-1-7-53 are selected from atoms or groups corresponding to h₁ of 53, that is, the structure of Compound BN-1-7-53 is

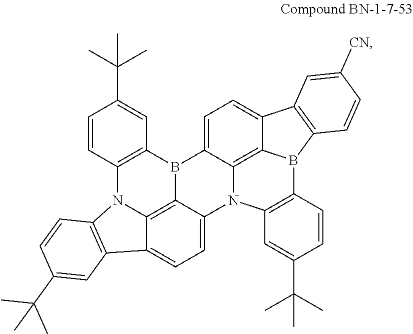 [SMILES: CC(C)(C)c1ccc2c(c1)B1c3ccc4c5c3N(c3cc(C(C)(C)C)ccc3B5c3ccc(C#N)cc3-4)c3ccc4c5cc(C(C)(C)C)ccc5n-2c4c31]

and R₁, R₂, R₃, R₄, R₅, and R₆ in Compound BN-1-7-80 are selected from atoms or groups corresponding to h₁ of 80, that is, the structure of Compound BN-1-7-80 is

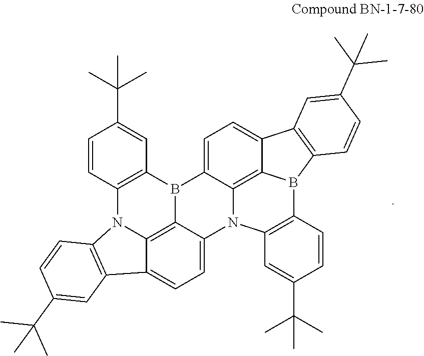 [SMILES: CC(C)(C)c1ccc2c(c1)B1c3ccc4c5c3N(c3cc(C(C)(C)C)ccc3B5c3ccc(C(C)(C)C)cc3-4)c3ccc4c5cc(C(C)(C)C)ccc5n-2c4c31]

In another example, Compound BN-1-11-h₁ represents Compounds BN-1-11-1 to BN-1-11-127, and Compounds BN-1-11-1 to BN-1-11-127 all have a skeleton structure of

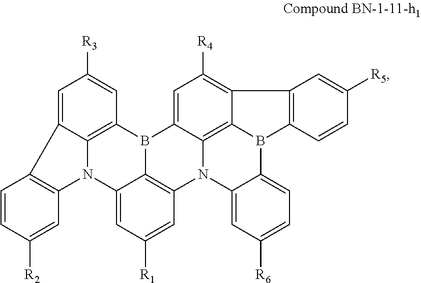

wherein R₁, R₂, R₃, R₄, R₅, and R₆ in Compound BN-1-11-101 are selected from atoms or groups corresponding to h₁ of 101, that is, the structure of Compound BN-1-11-101 is

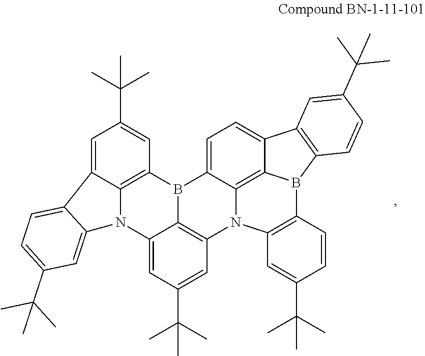 [SMILES: CC(C)(C)c1ccc2c(c1)-c1ccc3c4c1B2c1ccc(C(C)(C)C)cc1N4c1cc(C(C)(C)C)cc2c1B3c1cc(C(C)(C)C)cc3c4ccc(C(C)(C)C)cc4n-2c13]

and R₁, R₂, R₃, R₄, R₅, and R₆ in Compound BN-1-11-115 are selected from atoms or groups corresponding to h₁ of 115, that is, the structure of Compound BN-1-11-115 is

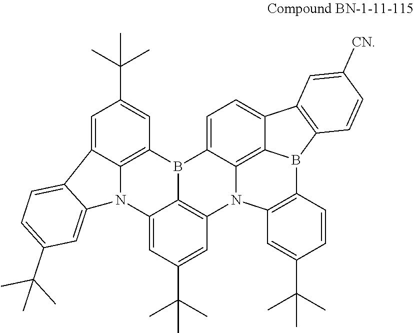 [SMILES: CC(C)(C)c1ccc2c(c1)N1c3cc(C(C)(C)C)cc4c3B(c3ccc5c(c31)B2c1ccc(C#N)cc1-5)c1cc(C(C)(C)C)cc2c3ccc(C(C)(C)C)cc3n-4c12]

Similarly, h₂ is selected from an integer from 1 to 390; therefore, Compound BN-2-1-h₂ to Compound BN-2-6-h₂ represent Compounds BN-2-1-1 to BN-2-1-390, Compounds BN-2-2-1 to BN-2-2-390, Compounds BN-2-3-1 to BN-2-3-390, Compounds BN-2-4-1 to BN-2-4-390, Compounds BN-2-5-1 to BN-2-5-390, and Compounds BN-2-6-1 to BN-2-6-390.

According to an embodiment of the present disclosure, hydrogens in the structures of Compound BN-1-1-h₁ to Compound BN-1-20-h₁ and Compound BN-2-1-h₂ to Compound BN-2-6-h₂ can be partially or fully substituted with deuterium.

According to an embodiment of the present disclosure, disclosed is an organic electroluminescent device, which comprises an anode, a cathode, and an organic layer disposed between the anode and the cathode, wherein the organic layer comprises the compound of any one of the preceding embodiments.

According to an embodiment of the present disclosure, the organic layer is an emissive layer and the compound is a light-emitting material.

According to an embodiment of the present disclosure, the compound is a fluorescent material.

According to an embodiment of the present disclosure, the compound is a delayed fluorescent material.

According to an embodiment of the present disclosure, the device emits red light.

According to an embodiment of the present disclosure, the device emits orange light.

According to an embodiment of the present disclosure, the device emits white light.

According to an embodiment of the present disclosure, the emissive layer comprises at least one host material.

According to an embodiment of the present disclosure, the emissive layer comprises at least two host materials.

According to an embodiment of the present disclosure, the host material comprises at least one chemical group selected from the group consisting of benzene, pyridine, pyrimidine, triazine, carbazole, azacarbazole, indolocarbazole, dibenzothiophene, azadibenzothiophene, dibenzofuran, azadibenzofuran, dibenzoselenophene, triphenylene, azatriphenylene, fluorene, silafluorene, naphthalene, quinoline, isoquinoline, quinazoline, quinoxaline, phenanthrene, azaphenanthrene, and combinations thereof.

According to an embodiment of the present disclosure, the emissive layer comprises at least a first host material.

According to an embodiment of the present disclosure, the first host material has a structure represented by Formula PH-1, Formula PH-2, or Formula PH-3:

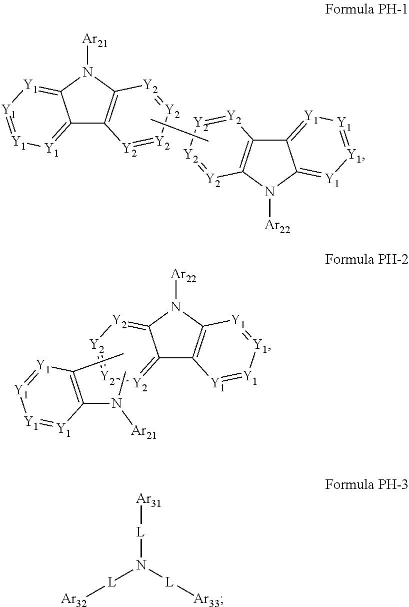

wherein Y₁ is, at each occurrence identically or differently, selected from CR_{y} or N;
Y₂ is, at each occurrence identically or differently, selected from C, CR_{y}, or N;
L is, at each occurrence identically or differently, selected from a single bond, substituted or unsubstituted arylene having 6 to 30 carbon atoms, substituted or unsubstituted heteroarylene having 3 to 30 carbon atoms, or a combination thereof;
Ar₂₁, Ar₂₂, Ar₃₁, Ar₃₂, and Ar₃₃ are, at each occurrence identically or differently, selected from substituted or unsubstituted aryl having 6 to 30 carbon atoms or substituted or unsubstituted heteroaryl having 3 to 30 carbon atoms;
R_{y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof; and
adjacent substituents R_{y} can be optionally joined to form a ring.

Herein, the expression that “adjacent substituents R_{y} can be optionally joined to form a ring” is intended to mean that any adjacent substituents R_{y} may be joined to form a ring. Obviously, it is also possible that any adjacent substituents R_{y} are not joined to form a ring.

According to an embodiment of the present disclosure, the first host material has a structure represented by Formula PH-1-1, Formula PH-2-1, or Formula PH-3-1:

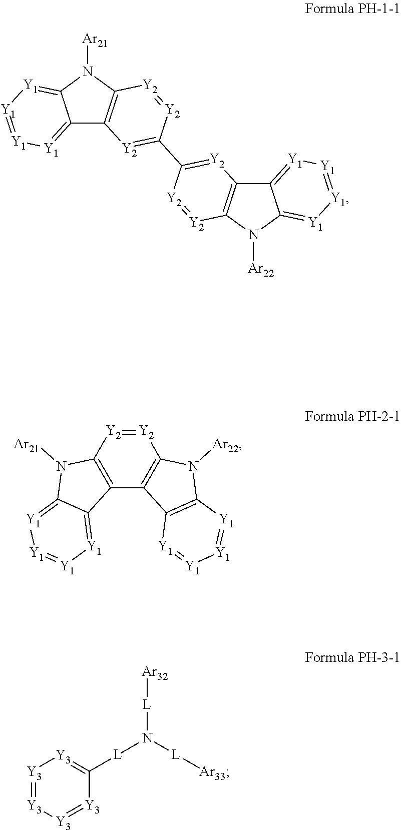

wherein Y₁, Y₂, and Y₃ are, at each occurrence identically or differently, selected from CR_{y} or N;
A₂₁, Ar₂₂, Ar₃₂, and A₃₃ are, at each occurrence identically or differently, selected from substituted or unsubstituted aryl having 6 to 30 carbon atoms or substituted or unsubstituted heteroaryl having 3 to 30 carbon atoms;
L is, at each occurrence identically or differently, selected from a single bond, substituted or unsubstituted arylene having 6 to 30 carbon atoms, substituted or unsubstituted heteroarylene having 3 to 30 carbon atoms, or a combination thereof;
R_{y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof; and
adjacent substituents R_{y} can be optionally joined to form a ring.

According to an embodiment of the present disclosure, the first host material is selected from the group consisting of Compound 1-1-1 to Compound 1-1-104, Compound 1-2-1 to Compound 1-2-100, and Compound 1-3-1 to Compound 1-3-110, wherein for specific structures of Compound 1-1-1 to Compound 1-1-104, Compound 1-2-1 to Compound 1-2-100, and Compound 1-3-1 to Compound 1-3-110, refer to claim 12.

According to an embodiment of the present disclosure, hydrogens in Compound 1-1-1 to Compound 1-1-104, Compound 1-2-1 to Compound 1-2-100, and Compound 1-3-1 to Compound 1-3-100 can be partially or fully substituted with deuterium.

According to an embodiment of the present disclosure, in the electroluminescent device, the emissive layer further comprises a second host material.

According to an embodiment of the present disclosure, the second host material has a structure represented by Formula NH-1:

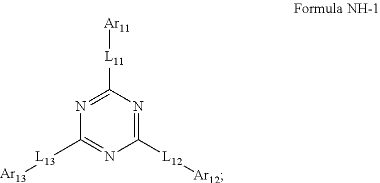

wherein L₁₁ to L₁₃ are, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 30 carbon atoms, substituted or unsubstituted heteroarylene having 3 to 30 carbon atoms, or a combination thereof; and
Ar₁₁ to Ar₁₃ are, at each occurrence identically or differently, selected from substituted or unsubstituted aryl having 6 to 30 carbon atoms, substituted or unsubstituted heteroaryl having 3 to 30 carbon atoms, or a combination thereof.

According to an embodiment of the present disclosure, the second host material has a structure represented by Formula NH-1-1 or Formula NH-1-2:

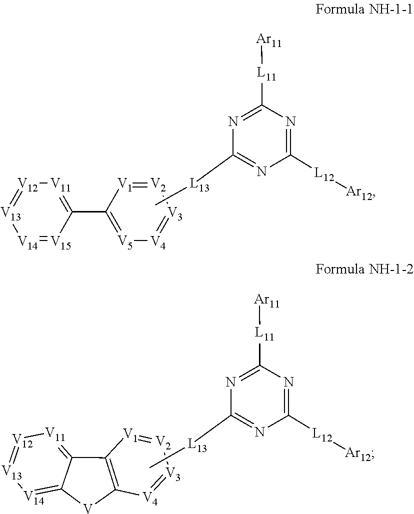

wherein in Formula NH-1-1, V₁ to V₅ are, at each occurrence identically or differently, selected from C, N, or CRᵥ, V₁₁ to V₁₅ are, at each occurrence identically or differently, selected from N or CRᵥ₁, and one of V₁ to V₅ is C and joined to L₁₃;
in Formula NH-1-2, V₁ to V₄ are, at each occurrence identically or differently, selected from C, N or CRᵥ, V₁₁ to V₁₄ are, at each occurrence identically or differently, selected from N or CRᵥ₁, and one of V₁ to V₄ is C and joined to L₁₃;
V is selected from O, S, or Se;
L₁₁ to L₁₃ are, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 30 carbon atoms, substituted or unsubstituted heteroarylene having 3 to 30 carbon atoms, or a combination thereof;
Ar₁₁ and Ar₁₂ are, at each occurrence identically or differently, selected from substituted or unsubstituted aryl having 6 to 30 carbon atoms, substituted or unsubstituted heteroaryl having 3 to 30 carbon atoms, or a combination thereof;
Rᵥ and Rᵥ₁ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof; and
adjacent substituents Rᵥ and Rᵥ₁ can be optionally joined to form a ring.

Herein, the expression that “adjacent substituents Rᵥ and Rᵥ₁ can be optionally joined to form a ring” is intended to mean that any one or more of groups of adjacent substituents, such as adjacent substituents Rᵥ, adjacent substituents Rᵥ₁, and adjacent substituents Rᵥ and Rᵥ₁, may be joined to form a ring. Obviously, it is also possible that none of these substituents are joined to form a ring.

According to an embodiment of the present disclosure, at least one of Ar₁₁ or Ar₁₂ is a structure having two or three fused rings.

According to an embodiment of the present disclosure, Ar₁₁ and Ar₁₂ are, at each occurrence identically or differently, selected from substituted or unsubstituted phenyl, substituted or unsubstituted biphenyl, substituted or unsubstituted terphenyl, substituted or unsubstituted naphthyl, substituted or unsubstituted phenanthryl, substituted or unsubstituted triphenylenyl, substituted or unsubstituted chrysenyl, substituted or unsubstituted fluorenyl, substituted or unsubstituted carbazolyl, substituted or unsubstituted dibenzofuryl, substituted or unsubstituted dibenzothienyl, substituted or unsubstituted pyridyl, substituted or unsubstituted pyrimidinyl, substituted or unsubstituted quinolyl, substituted or unsubstituted indolocarbazolyl, or a combination thereof.

According to an embodiment of the present disclosure, L₁₁ to L₁₃ are, at each occurrence identically or differently, selected from a single bond, substituted or unsubstituted phenylene, substituted or unsubstituted naphthylene, substituted or unsubstituted biphenylylene, or a combination thereof.

According to an embodiment of the present disclosure, the second host material is selected from the group consisting of Compound B-1 to Compound B-243, wherein for specific structures of Compound B-1 to Compound B-243, refer to claim 13.

According to an embodiment of the present disclosure, hydrogens in Compound B-1 to Compound B-243 can be partially or fully substituted with deuterium.

According to an embodiment of the present disclosure, the emissive layer further comprises at least one metal complex.

According to an embodiment of the present disclosure, the metal complex is a phosphorescent sensitizer.

According to an embodiment of the present disclosure, a maximum emission wavelength in a photoluminescence spectrum of the metal complex is 570 nm to 800 nm.

According to an embodiment of the present disclosure, the metal complex has a general formula of M(Lₐ)ₘ(L_{b})ₙ(L_{c})_{q};
M is selected from a metal with a relative atomic mass greater than 40;
Lₐ, L_{b}, and L_{c} are a first ligand, a second ligand, and a third ligand coordinated to M, respectively, and Lₐ, L_{b}, and L_{c} can be optionally joined to form a multidentate ligand;
Lₐ, L_{b}, and L_{c} are identical or different; m is 1, 2, or 3, n is 0, 1, or 2, q is 0, 1, or 2, and m+n+q is equal to the oxidation state of M; when m is greater than or equal to 2, multiple Lₐ are identical or different; when n is 2, two L_{b} are identical or different; when q is 2, two L_{c} are identical or different;
Lₐ is, at each occurrence identically or differently, selected from a structure represented by Formula 2:

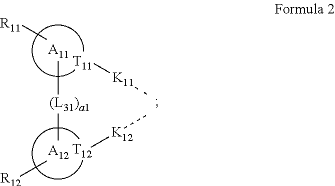

wherein
the ring A₁₁ and the ring A₁₂ are each selected from an aromatic ring having 6 to 30 ring atoms, a heteroaromatic ring having 5 to 30 ring atoms, or a combination thereof;
T₁₁ and T₁₂ are, at each occurrence identically or differently, selected from C or N;
K₁₁ and K₁₂ are, at each occurrence identically or differently, selected from a single bond, O, S, or NRₖ;
L₃₁ is, at each occurrence identically or differently, selected from the group consisting of: a single bond, BR_{L11}, CR_{L11}R_{L11}, NR_{L11}, O, SiR_{L11}R_{L11}, PR_{L11}, S, GeR_{L11}R_{L11}, Se, substituted or unsubstituted vinylene, ethynylene, substituted or unsubstituted arylene having 6 to 30 carbon atoms, substituted or unsubstituted heteroarylene having 3 to 30 carbon atoms, and combinations thereof; when two R_{L11} are present at the same time, the two R_{L11} are identical or different;
a₁ is selected from 0 or 1;
R₁₁ and R₁₂ represent, at each occurrence identically or differently, mono-substitution, multiple substitutions, or non-substitution;
R_{L11}, R₁₁, R₁₂, and Rₖ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof; and adjacent substituents R_{L11}, R₁₁, and R₁₂ can be optionally joined to form a ring;
L_{b} and L_{c} are, at each occurrence identically or differently, selected from any one of the following structures:

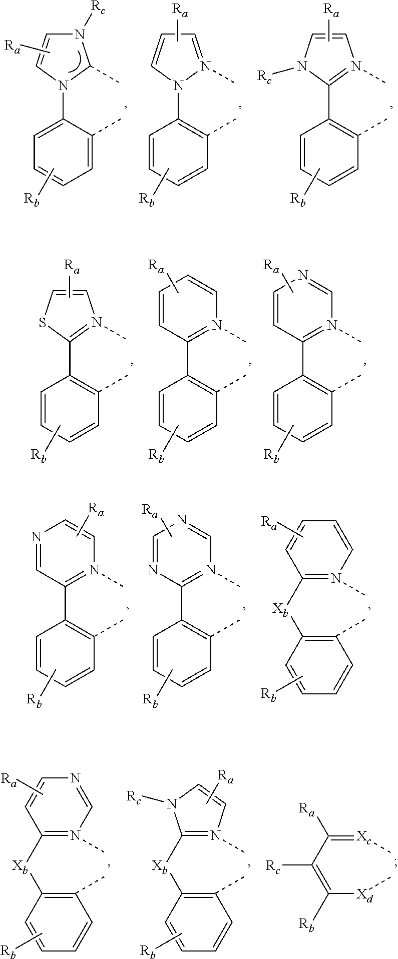

wherein
Rₐ, R_{b}, and R_{c} represent, at each occurrence identically or differently, mono-substitution, multiple substitutions, or non-substitution;
X_{b} is, at each occurrence identically or differently, selected from the group consisting of: O, S, Se, NR_{N1}, and CR_{C1}R_{C2};
X_{c} and X_{d} are, at each occurrence identically or differently, selected from the group consisting of: O, S, Se, and NR_{N2};
Rₐ, R_{b}, R_{c}, R_{N1}, R_{N2}, R_{C1}, and R_{C2} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof; and
adjacent substituents Rₐ, R_{b}, R_{c}, R_{N1}, R_{N2}, R_{C1}, and R_{C2} can be optionally joined to form a ring.

Herein, the expression that “adjacent substituents Rₐ, R_{b}, R_{c}, R_{N1}, R_{N2}, R_{C1}, and R_{C2} can be optionally joined to form a ring” is intended to mean that any one or more of groups of adjacent substituents, such as two substituents Rₐ, two substituents R_{b}, two substituents R_{c}, substituents Rₐ and R_{b}, substituents Rₐ and R_{c}, substituents R_{b} and R_{c}, substituents Rₐ and R_{N1}, substituents R_{b} and R_{N1}, substituents Rₐ and R_{C1}, substituents Rₐ and R_{C2}, substituents R_{b} and R_{C1}, substituents R_{b} and R_{C2}, substituents Rₐ and R_{N2}, substituents R_{b} and R_{N2}, and substituents R_{C1} and R_{C2}, may be joined to form a ring. For example, adjacent substituents Rₐ and R_{b} in

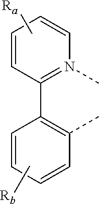

can be optionally joined to forma ring, which can form one or more of the following structures including, but not limited to,

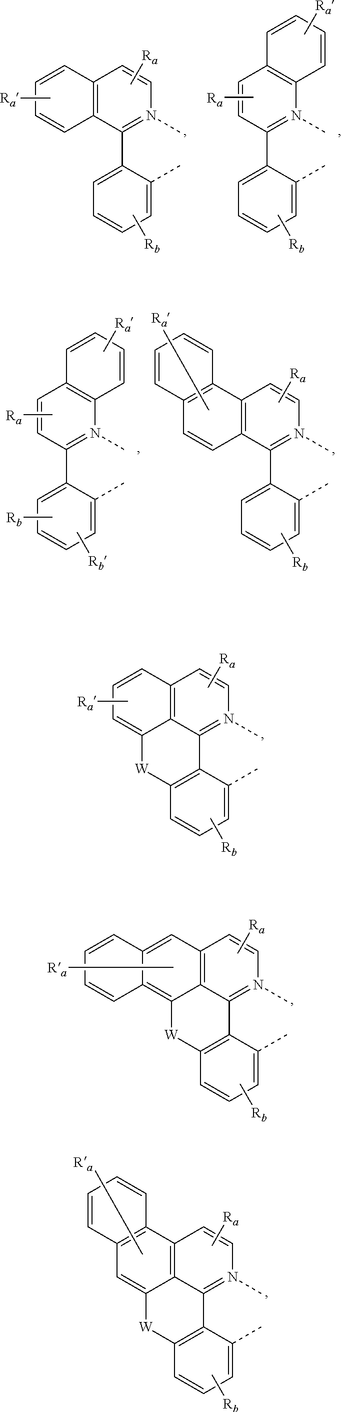

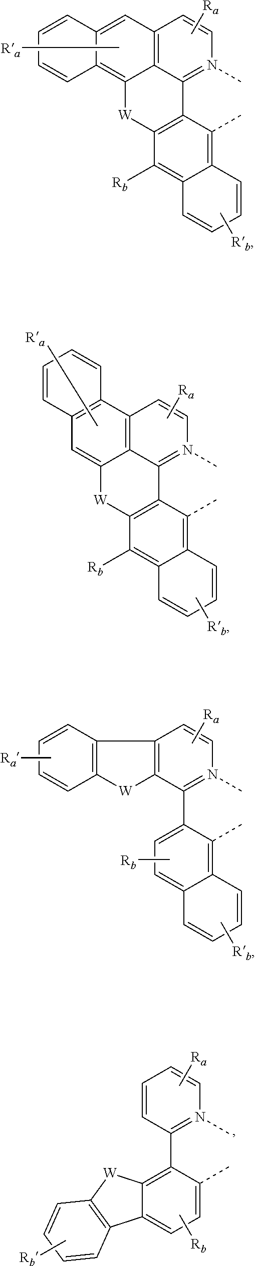

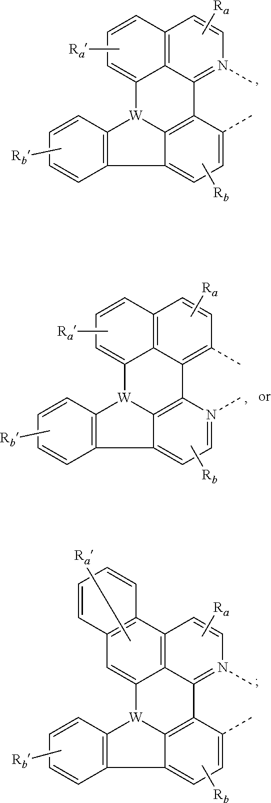

wherein W is selected from O, S, Se, NR_{w1}, or CR_{w1}R_{w1}, and R_{w1}, Rₐ′, and R_{b}′ are defined the same as Rₐ. Obviously, it is also possible that none of these substituents are joined to form a ring.

According to an embodiment of the present disclosure, the ligand Lₐ is, at each occurrence identically or differently, selected from a structure represented by Formula 3:

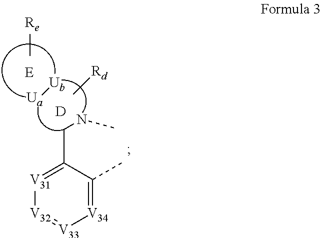

wherein
the ring D is selected from a five-membered heteroaromatic ring or a six-membered heteroaromatic ring;
the ring E is selected from a five-membered unsaturated carbocyclic ring, a benzene ring, a five-membered heteroaromatic ring, or a six-membered heteroaromatic ring;
the ring D and the ring E are fused via Uₐ and U_{b};
Uₐ and U_{b} are, at each occurrence identically or differently, selected from C or N;
R_{d} and Rₑ represent, at each occurrence identically or differently, mono-substitution, multiple substitutions, or non-substitution;
V₃₁ to V₃₄ are, at each occurrence identically or differently, selected from CRᵥ₃ or N;
R_{d}, Rₑ, and Rᵥ₃ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof; and adjacent substituents R_{d}, Rₑ, and Rᵥ₃ can be optionally joined to form a ring.

Herein, the expression that “adjacent substituents R_{d}, Rₑ, and Rᵥ₃ can be optionally joined to form a ring” is intended to mean that in the presence of substituents R_{d}, Rₑ, and Rᵥ₃, any one or more of groups of adjacent substituents, such as adjacent substituents R_{d}, adjacent substituents Rₑ, adjacent substituents Rᵥ₃, adjacent substituents R_{d} and Rₑ, adjacent substituents R_{d} and Rᵥ₃, and adjacent substituents Rₑ and Rᵥ₃, can be joined to form a ring. Obviously, in the presence of the substituents R_{d}, Rₑ, and Rᵥ₃, it is also possible that none of these groups of substituents are joined to form a ring.

According to an embodiment of the present disclosure, in Formula 3, two adjacent substituents Rₑ are joined to form a ring.

According to an embodiment of the present disclosure, in Formula 3, two adjacent substituents Rₑ are joined to form a five-membered unsaturated carbocyclic ring, a five-membered heteroaromatic ring, or a benzene ring.

According to an embodiment of the present disclosure, in Formula 3, the ring D is a six-membered heteroaromatic ring, and the ring E is a benzene ring or a six-membered heteroaromatic ring.

According to an embodiment of the present disclosure, in Formula 3, the ring D is a six-membered heteroaromatic ring, and the ring E is a five-membered heteroaromatic ring or a five-membered unsaturated carbocyclic ring.

According to an embodiment of the present disclosure, in Formula 3, the ring D is a six-membered heteroaromatic ring, the ring E is a benzene ring or a six-membered heteroaromatic ring, and two adjacent substituents Rₑ are joined to form a benzene ring or a six-membered heteroaromatic ring.

According to an embodiment of the present disclosure, in Formula 3, the ring D is a six-membered heteroaromatic ring, the ring E is a five-membered heteroaromatic ring or a five-membered unsaturated carbocyclic ring, and two adjacent substituents Rₑ are joined to form a benzene ring or a six-membered heteroaromatic ring.

According to an embodiment of the present disclosure, in Formula 3, at least one or two of groups of adjacent substituents among R_{d}, Rₑ, and Rᵥ₃ are joined to form a ring. For example, two substituents R_{d} are joined to form a ring, two substituents Rₑ are joined to form a ring, two substituents Rᵥ₃ are joined to form a ring, substituents R_{d} and Rₑ are joined to form a ring, substituents R_{d} and Rᵥ₃ are joined to form a ring, substituents Rₑ and Rᵥ₃ are joined to form a ring, two substituents Rₑ are joined to form a ring while two substituents R_{d} are joined to form a ring, two substituents Rᵥ₃ are joined to form a ring while two substituents R_{d} are joined to form a ring, two substituents Rᵥ₃ are joined to form a ring while two substituents Rₑ are joined to form a ring, two substituents Rᵥ₃ are joined to form a ring while substituents Rₑ and Rᵥ₃ are joined to form a ring, or two substituents Rᵥ₃ are joined to form a ring while substituents R_{d} and Rᵥ₃ are joined to form a ring; more groups of adjacent substituents among R_{d}, Rₑ, and Rᵥ₃ are joined to form a ring similarly.

According to an embodiment of the present disclosure, in the organic electroluminescent device, the metal complex has a general formula of M(Lₐ)ₘ(L_{b})ₙ;
the metal M is selected from a metal with a relative atomic mass greater than 40;
Lₐ and L_{b} are a first ligand and a second ligand coordinated to the metal M, respectively, and Lₐ and L_{b} can be optionally joined to form a multidentate ligand;
m is 1, 2, or 3, n is 0, 1, or 2, and m+n is equal to the oxidation state of the metal M; when m is greater than or equal to 2, multiple Lₐ may be identical or different; when n is 2, two L_{b} may be identical or different;
Lₐ is, at each occurrence identically or differently, selected from a structure represented by Formula 3:

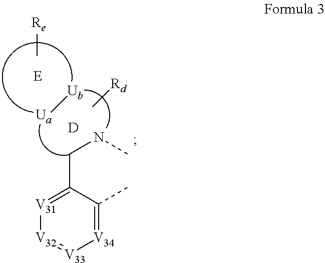

wherein
the ring D is selected from a five-membered heteroaromatic ring or a six-membered heteroaromatic ring;
the ring E is selected from a five-membered unsaturated carbocyclic ring, a benzene ring, a five-membered heteroaromatic ring, or a six-membered heteroaromatic ring;
the ring D and the ring E are fused via Uₐ and U_{b};
Uₐ and U_{b} are, at each occurrence identically or differently, selected from C or N;
R_{d} and Rₑ represent, at each occurrence identically or differently, mono-substitution, multiple substitutions, or non-substitution;
V₃₁ to V₃₄ are, at each occurrence identically or differently, selected from CRᵥ₃ or N;
R_{d}, Rₑ, and Rᵥ₃ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof; and
adjacent substituents R_{d}, Rₑ, and Rᵥ₃ can be optionally joined to form a ring;
the ligand L_{b} is, at each occurrence identically or differently, selected from the following structure:

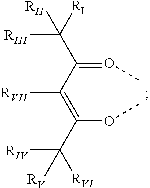

wherein R₁ to R_{V11} are each independently selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a sulfanyl group, a sulfinyl group, a sulfonyl group, a phosphino group, and combinations thereof.

According to an embodiment of the present disclosure, at least one of R_{I} to R_{III} is selected from substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, or a combination thereof, and/or at least one of R_{IV} to R_{VI} is selected from substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, or a combination thereof.

According to an embodiment of the present disclosure, at least two of R_{I} to R_{III} are, at each occurrence identically or differently, selected from substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, or a combination thereof, and/or at least two of R_{IV} to R_{VI} are, at each occurrence identically or differently, selected from substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, or a combination thereof.

According to an embodiment of the present disclosure, at least two of R_{I} to R_{III} are, at each occurrence identically or differently, selected from substituted or unsubstituted alkyl having 2 to 20 carbon atoms, substituted or unsubstituted cycloalkyl having 3 to 20 ring carbon atoms, substituted or unsubstituted heteroalkyl having 2 to 20 carbon atoms, or a combination thereof, and/or at least two of R_{IV} to R_{VI} are, at each occurrence identically or differently, selected from substituted or unsubstituted alkyl having 2 to 20 carbon atoms, substituted or unsubstituted cycloalkyl having 3 to 20 ring carbon atoms, substituted or unsubstituted heteroalkyl having 2 to 20 carbon atoms, or a combination thereof.

According to an embodiment of the present disclosure, Lₐ has a structure represented by Formula 3 and comprises at least one structural unit selected from the group consisting of an aromatic ring formed by fusing a six-membered ring to a six-membered ring, a heteroaromatic ring formed by fusing a six-membered ring to a six-membered ring, an aromatic ring formed by fusing a six-membered ring to a five-membered ring, and a heteroaromatic ring formed by fusing a six-membered ring to a five-membered ring.

According to an embodiment of the present disclosure, in the organic electroluminescent device, Lₐ has the structure represented by Formula 3 and comprises at least one structural unit selected from the group consisting of naphthalene, phenanthrene, quinoline, isoquinoline, and azaphenanthrene.

According to an embodiment of the present disclosure, in the organic electroluminescent device, the metal complex is an Ir complex, a Pt complex, or an Os complex.

According to an embodiment of the present disclosure, in the organic electroluminescent device, the metal complex is an Ir complex and has a structure represented by any one of Ir(Lₐ)(L_{b})(L_{c}), Ir(Lₐ)₂(L_{b}), Ir(Lₐ)(L_{b})₂, Ir(Lₐ)₂(L₄), or Ir(Lₐ)(L₄)₂.

According to an embodiment of the present disclosure, the metal complex has a general formula of M(Lₐ)ₘ(L_{b})₃₋ₘ and has a structure represented by Formula M-a:

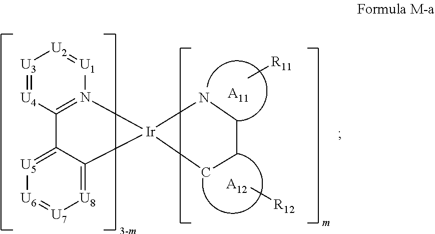

wherein
m is selected from 1, 2, or 3; when m is selected from 1, two L_{b} are identical or different; when m is selected from 2 or 3, multiple Lₐ are identical or different;
the ring A₁₁ and the ring A₁₂ are each selected from an aromatic ring having 6 to 24 ring atoms, a heteroaromatic ring having 5 to 24 ring atoms, or a combination thereof;
U₁ to U₈ are, at each occurrence identically or differently, selected from CRᵤ₁ or N;
R₁₁ and R₁₂ represent, at each occurrence identically or differently, mono-substitution, multiple substitutions, or non-substitution;
R₁₁, R₁₂, and Rᵤ₁ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof; and
adjacent substituents R₁₁, R₁₂, and Rᵤ₁ can be optionally joined to form a ring.

Herein, the expression that “adjacent substituents R₁₁, R₁₂, and Rᵤ₁ can be optionally joined to form a ring” is intended to mean that any one or more of groups of adjacent substituents, such as two substituents R₁₁, two substituents R₁₂, two substituents Rᵤ₁, and two substituents R₁₁ and R₁₂, may be joined to form a ring. Obviously, it is also possible that none of these substituents are joined to form a ring.

According to an embodiment of the present disclosure, the ring A₁₁ is, at each occurrence identically or differently, selected from any one of the following structures:

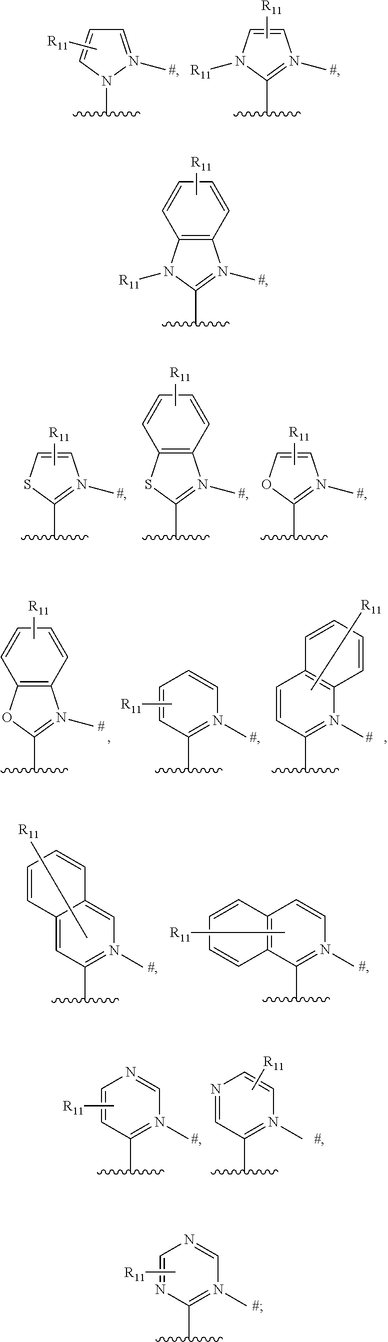

wherein
R₁₁ represents, at each occurrence identically or differently, mono-substitution, multiple substitutions, or non-substitution; when multiple R₁₁ are present at the same time in any structure, the multiple R₁₁ are identical or different;
R₁₁ is, at each occurrence identically or differently, selected from the group consisting of hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
adjacent substituents R₁₁ can be optionally joined to form a ring; and
“#” represents a position where the ring A₁₁ is joined to the metal Ir, and

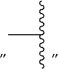

represents a position where the ring A₁₁ is joined to the ring A₁₂.

Herein, the expression that “adjacent substituents R₁₁ can be optionally joined to form a ring” is intended to mean that two adjacent substituents R₁₁ may be joined to form a ring. Obviously, it is also possible that two adjacent substituents R₁₁ are not joined to form a ring.

According to an embodiment of the present disclosure, the ring A₁₁ is selected from

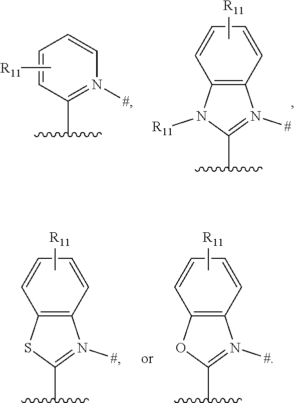

According to an embodiment of the present disclosure, the ring A₁₁ is selected from

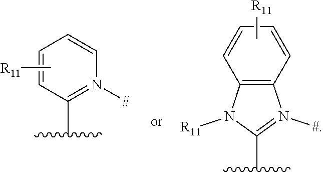

According to an embodiment of the present disclosure, the ring A₁₂ is, at each occurrence identically or differently, selected from any one of the following structures:

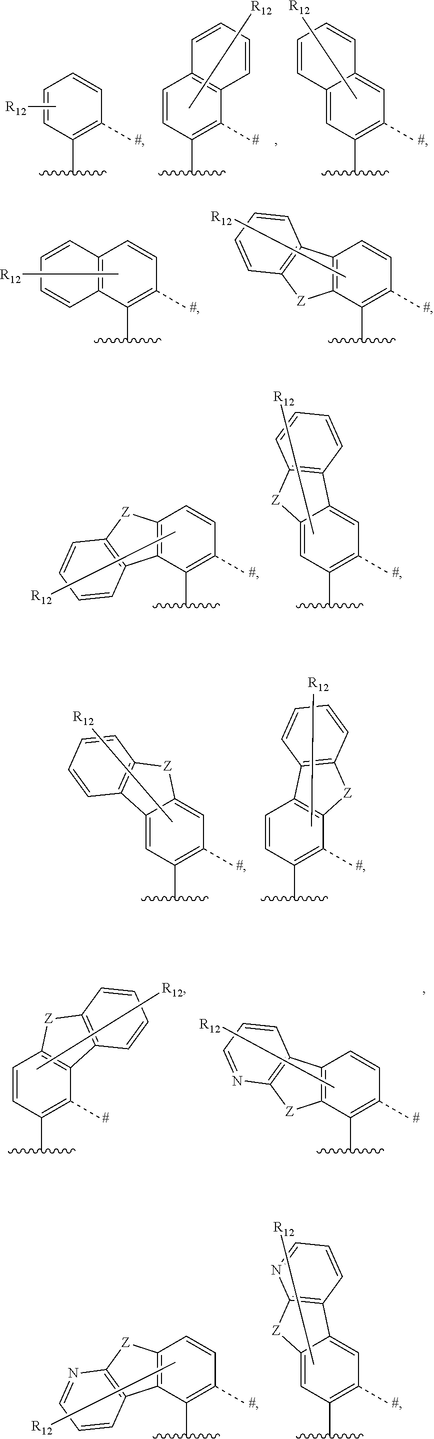

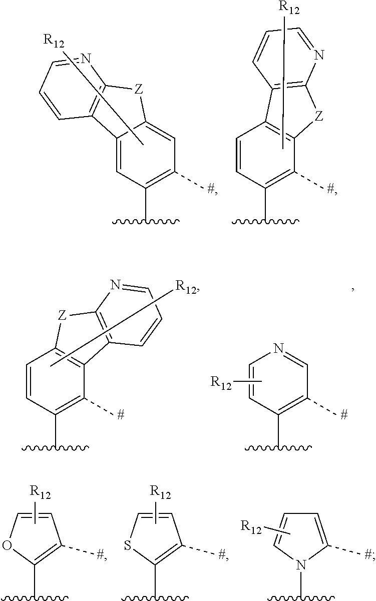

wherein
Z is, at each occurrence identically or differently, selected from the group consisting of: O, S, Se, NR_{z}, CR_{z}R_{z}, SiR_{z}R_{z}, and GeR_{z}R_{z};
R₁₂ represents, at each occurrence identically or differently, mono-substitution, multiple substitutions, or non-substitution; when multiple R₁₂ are present at the same time in any structure, the multiple R₁₂ are identical or different;
R_{z} and R₁₂ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
adjacent substituents R_{z} and R₁₂ can be optionally joined to form a ring; and
“#” represents a position where the ring A₁₂ is joined to the metal Ir, and

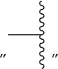

represents a position where the ring A₁₂ is joined to the ring A₁₁.

Herein, the expression that “adjacent substituents R_{z} and R₁₂ can be optionally joined to form a ring” is intended to mean that two adjacent substituents R_{z} may be joined to form a ring, and two adjacent substituents R₁₂ may be joined to form a ring. Obviously, it is also possible that two adjacent substituents R_{z} are not joined to form a ring, and two adjacent substituents R₁₂ are not joined to form a ring.

According to an embodiment of the present disclosure, the ring A₁₂ is selected from

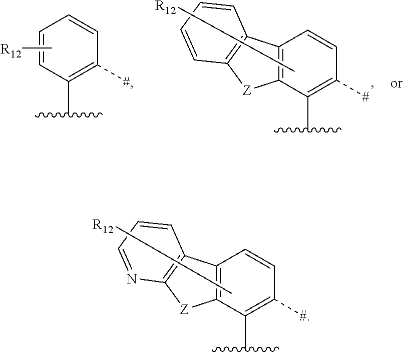

According to an embodiment of the present disclosure, the metal complex has a general formula of Ir(Lₐ)ₘ(L_{b})₃₋ₘ and has a structure represented by Formula M-a-0:

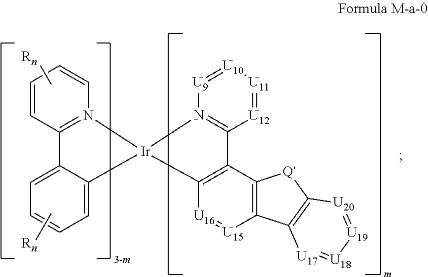

wherein
m is selected from 1, 2, or 3; when m is selected from 1, two L_{b} are identical or different; when m is selected from 2 or 3, two or three Lₐ are identical or different;
Q′ is selected from the group consisting of O, S, Se, NR_{Q}, CR_{Q}R_{Q}, SiR_{Q}R_{Q}, and GeR_{Q}R_{Q}; when multiple R_{Q} are present, the multiple R_{Q} are identical or different;
U₁₅ to U₂₀ are, at each occurrence identically or differently, selected from CRₙ₁ or N;
U₉ to U₁₂ are, at each occurrence identically or differently, selected from CRₙ₂ or N;
Rₙ represents, at each occurrence identically or differently, mono-substitution, multiple substitutions, or non-substitution;
Rₙ₁, Rₙ₂, R_{Q}, and Rₙ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof; and
adjacent substituents Rₙ₁, Rₙ₂, R_{Q}, and Rₙ can be optionally joined to form a ring.

According to an embodiment of the present disclosure, the Q′ is selected from O, S, Se, NR_{Q}, or CR_{Q}R_{Q}.

According to an embodiment of the present disclosure, the Q′ is selected from O or S.

According to an embodiment of the present disclosure, U₉ to U₁₂ are, at each occurrence identically or differently, selected from CRₙ₂, U₁₅ to U₁₉ are, at each occurrence identically or differently, selected from CRₙ₁, and U₂₀ is selected from CRₙ₁ or N; and Rₙ₁ and Rₙ₂ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, cyano, hydroxyl, sulfanyl, and combinations thereof.

According to an embodiment of the present disclosure, at least two of U₁₅ to U₂₀ are selected from CRₙ₁, one of the Rₙ₁ is cyano or fluorine, and another of the Rₙ₁ is selected from the group consisting of deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof.

According to an embodiment of the present disclosure, at least two of U₁₅ to U₂₀ are selected from CRₙ₁, one of the Rₙ₁ is cyano or fluorine, and another of the Rₙ₁ is selected from the group consisting of deuterium, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, and combinations thereof.

According to an embodiment of the present disclosure, U₁₉ is selected from CRₙ₁, and the Rₙ₁ is cyano or fluorine; U₂₀ is selected from CRₙ₁, and the Rₙ₁ is selected from deuterium, substituted or unsubstituted aryl having 6 to 18 carbon atoms, substituted or unsubstituted heteroaryl having 3 to 18 carbon atoms, or a combination thereof.

According to an embodiment of the present disclosure, Rₙ is, at each occurrence identically or differently, selected from the group consisting of hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted alkylgermanyl having 3 to 20 carbon atoms, cyano, and combinations thereof.

According to an embodiment of the present disclosure, Rₙ is, at each occurrence identically or differently, selected from the group consisting of: hydrogen, deuterium, fluorine, substituted or unsubstituted alkyl having 1 to 6 carbon atoms, substituted or unsubstituted cycloalkyl having 3 to 6 ring carbon atoms, substituted or unsubstituted aryl having 6 to 12 carbon atoms, substituted or unsubstituted heteroaryl having 3 to 12 carbon atoms, substituted or unsubstituted alkylsilyl having 3 to 12 carbon atoms, substituted or unsubstituted alkylgermanyl having 3 to 12 carbon atoms, and combinations thereof.

According to an embodiment of the present disclosure, the metal complex is selected from the group consisting of Compound RD1 to Compound RD214 and Compound GD1 to Compound GD184, wherein for specific structures of Compound RD1 to Compound RD214 and Compound GD1 to Compound GD184, refer to claim 14.

According to an embodiment of the present disclosure, hydrogens in Compound RD1 to Compound RD214 and Compound GD1 to Compound GD184 can be partially or fully substituted with deuterium.

According to another embodiment of the present disclosure, further disclosed is a compound composition comprising the compound of any one of the preceding embodiments.

Combination with Other Materials

The materials described in the present disclosure for a particular layer in an organic light emitting device can be used in combination with various other materials present in the device. The combinations of these materials are described in more detail in U.S. Pat. App. No. 20160359122 at paragraphs 0132-0161, which is incorporated by reference herein in its entirety. The materials described or referred to the disclosure are non-limiting examples of materials that may be useful in combination with the compounds disclosed herein, and one of skill in the art can readily consult the literature to identify other materials that may be useful in combination.

The materials described herein as useful for a particular layer in an organic light emitting device may be used in combination with a variety of other materials present in the device. For example, compounds disclosed herein may be used in combination with a wide variety of light-emitting dopants, hosts, transporting layers, blocking layers, injection layers, electrodes, and other layers that may be present. The combination of these materials is described in detail in paragraphs 0080-0101 of U.S. Pat. App. No. 20150349273, which is incorporated by reference herein in its entirety. The materials described or referred to the disclosure are non-limiting examples of materials that may be useful in combination with the compounds disclosed herein, and one of skill in the art can readily consult the literature to identify other materials that may be useful in combination.

In the embodiments of material synthesis, all reactions were performed under nitrogen protection unless otherwise stated. All reaction solvents were anhydrous and used as received from commercial sources. Synthetic products were structurally confirmed and tested for properties using one or more conventional equipment in the art (including, but not limited to, nuclear magnetic resonance instrument produced by BRUKER, liquid chromatograph produced by SHIMADZU, liquid chromatograph-mass spectrometry produced by SHIMADZU, gas chromatograph-mass spectrometry produced by SHIMADZU, differential Scanning calorimeters produced by SHIMADZU, fluorescence spectrophotometer produced by SHANGHAI LENGGUANG TECH., electrochemical workstation produced by WUHAN CORRTEST, and sublimation apparatus produced by ANHUI BEQ, etc.) by methods well known to the persons skilled in the art. In the embodiments of the device, the characteristics of the device were also tested using conventional equipment in the art (including, but not limited to, evaporator produced by ANGSTROM ENGINEERING, optical testing system produced by SUZHOU FSTAR, life testing system produced by SUZHOU FSTAR, and ellipsometer produced by BEIJING ELLITOP, etc.) by methods well known to the persons skilled in the art. As the persons skilled in the art are aware of the above-mentioned equipment use, test methods and other related contents, the inherent data of the sample can be obtained with certainty and without influence, so the above related contents are not further described in this patent.

### Material Synthesis Example

A method for preparing the compound of the present disclosure is not limited. Those skilled in the art may select appropriate raw materials and process routes according to synthesis targets. Alternatively, those skilled in the art may prepare the compound of the present disclosure according to the synthesis route illustrated below.

### Step 1:

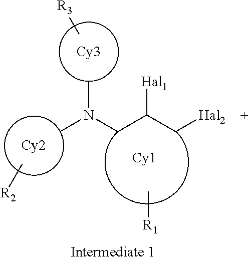

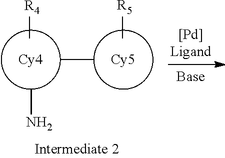

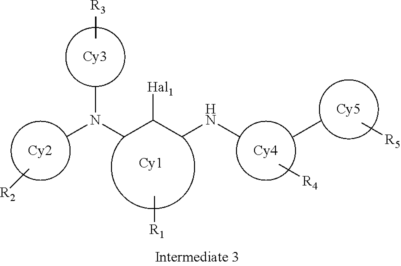

In step 1, Intermediate 1 and Intermediate 2 are subjected to a carbon-nitrogen coupling reaction such as the Ullmann reaction or the Buchwald reaction to obtain Intermediate 3, wherein Hal₁ is H, Cl, Br, etc. and Hal₂ is Cl, Br, I, OTf, etc. Reaction conditions of the Buchwald reaction are well-known to those skilled in the art. For example, as illustrated in the above formula, a common metal palladium reagent [Pd] is typically used, such as palladium acetate or Pd₂(dba)₃; the ligand is typically an organophosphine ligand, such as P(tBu)₃, X-phos, Xantphos, S-phos, DPPF, BINAP, or Ruphos; and the base is typically an organic or inorganic base, such as sodium t-butoxide, lithium t-butoxide, potassium t-butoxide, potassium carbonate, sodium carbonate, or cesium carbonate. The solvent is typically a commonly used organic solvent, such as toluene, t-butylbenzene, or xylene. Typically, the reaction concentration is 0.01-0.5 [M], and the reaction temperature is between 90° C. and 180° C.

### Step 2:

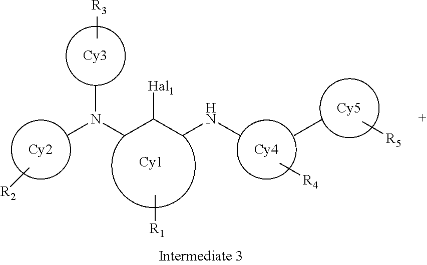

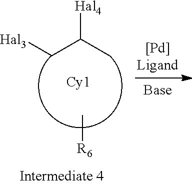

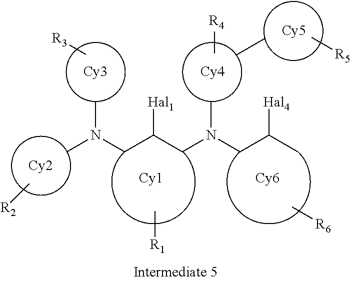

In step 2, Intermediate 3 and Intermediate 4 are subjected to a carbon-nitrogen coupling reaction such as the Ullmann reaction or the Buchwald reaction to obtain Intermediate 5, wherein Hal₃ and Hal₄ are Br, I, OTf, etc.

### Step 3:

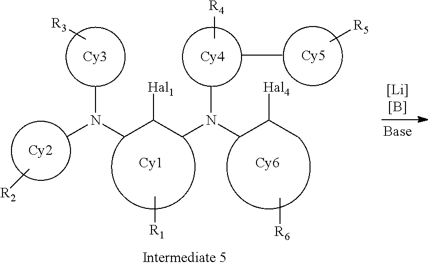

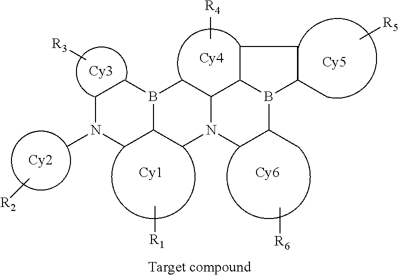

In step 3, Intermediate 5 is subjected to a metalation reaction at a particular position by using a metal lithium reagent [Li], a lithium-boron metal exchange by adding a boron reagent [B], and a tandem bora-Friedel-Crafts reaction by adding a Brønsted base to obtain a target compound. Alternatively, a Lewis acid may be added to promote the reaction.

Reaction conditions of the metalation reaction and the tandem bora-Friedel-Crafts reaction are well-known to those skilled in the art. For example, the metalation reaction may be carried out by directly subjecting Hal₁ and Hal₄ positions to lithium-halogen exchange using the metal lithium reagent, thereby achieving lithiation. The metal lithium reagent is typically an alkyllithium reagent such as methyllithium, n-butyllithium, sec-butyllithium, or t-butyllithium or a lithium amide reagent such as lithium diisopropylamide or lithium 2,2,6,6-tetramethylpiperidide. The metal lithium reagent is typically used in an amount of 2-5 equivalents. The boron reagent is typically boron trichloride, boron tribromide, etc. The boron reagent is typically used in an amount of 3-6 equivalents. The Brønsted base is typically an organic base, such as N,N-diisopropylethylamine, triethylamine, or 2,2,6,6-tetramethylpiperidine. The Brønsted base is typically used in an amount of 5-10 equivalents. Typically, the reaction concentration is 0.01-0.5 [M], and the reaction temperature is between −78° C. and 180° C.

To promote the reaction, the Lewis acid may be added, such as AlCl₃, BF₃·OEt₂, BCl₃, BBr₃, InCl₃, InBr₃, In(OTf)₃, SnCl₄, SnBr₄, AgOTf, ScCl₃, or Sc(OTf)₃.

Those skilled in the art will appreciate that the above preparation method is merely exemplary. Those skilled in the art can obtain other compound structures selected in the present disclosure through modifications of the preparation method.

Due to a particular polycyclic structural design, the compounds of the present disclosure modulate the emitted color to different degrees and can achieve the desired orange to red light emission. To further verify the luminescence effect, the maximum emission wavelengths and energy levels of some compounds of the present disclosure were calculated by a density functional theory (DFT).

DFT calculation method used in the present disclosure:

The compounds of the present disclosure and comparative compounds were subjected to DFT calculations by using a B3LYP hybrid functional and a CEP-31G effective core potential basis set in a Gaussian software package in a tetrahydrofuran (THF) solvent environment simulated by a solvation model based on density (SMD). The HOMO values, LUMO values, and singlet energy levels (S1) of the compounds were obtained, and the maximum emission wavelengths λₘₐₓ (nm) of the compounds were obtained through conversion according to the formula λₘₐₓ (nm)=1240/S1. The data are recorded and listed in Table 1.

TABLE 1
Calculated data

| | HOMO | LUMO | S1 | λₘₐₓ |
|---|---|---|---|---|
| Compound | (eV) | (eV) | (eV) | (nm) |
| Compound BN-1-11-101 | −5.46 | −2.60 | 2.08 | 595 |
| Compound BN-1-7-80 | −5.51 | −2.63 | 2.01 | 616 |
| Compound BN-1-11-115 | −5.55 | −2.88 | 1.89 | 656 |
| Compound BN-1-7-53 | −5.60 | −2.92 | 1.84 | 673 |
| Compound BN-1-5-94 | −5.64 | −2.78 | 2.04 | 608 |
| Compound BN-1-5-73 | −5.75 | −3.03 | 1.86 | 666 |
| Compound A | −5.44 | −2.40 | 2.34 | 529 |
| Compound B | −5.08 | −2.09 | 2.29 | 541 |

The related compounds have the following structures:

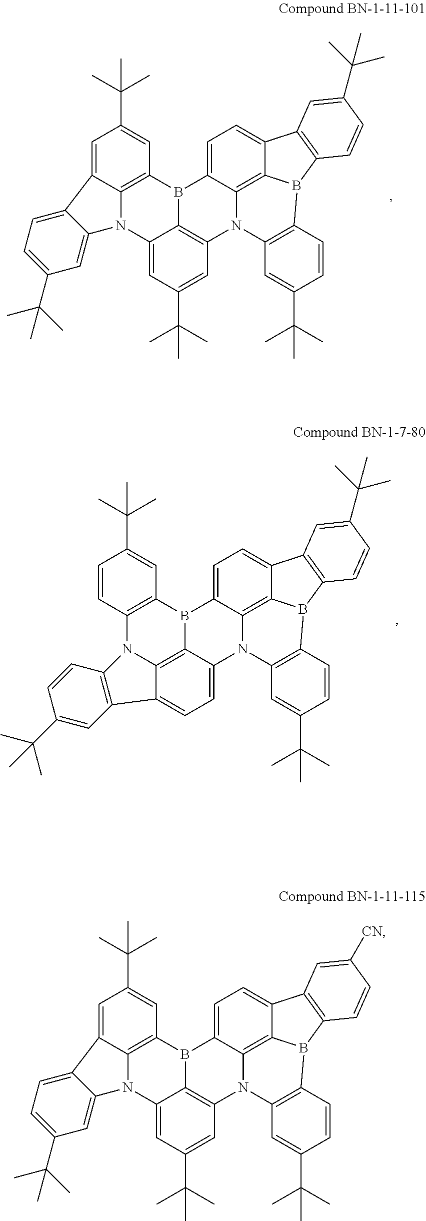

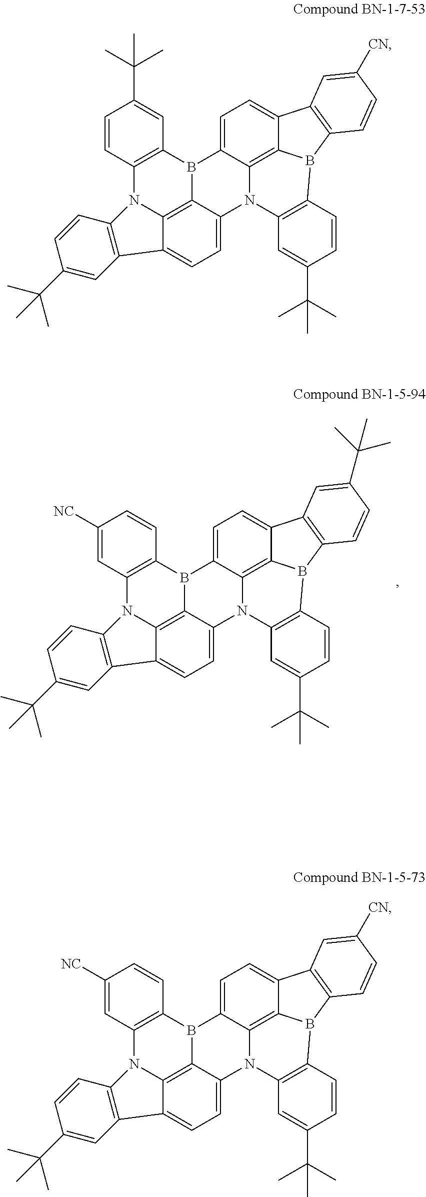

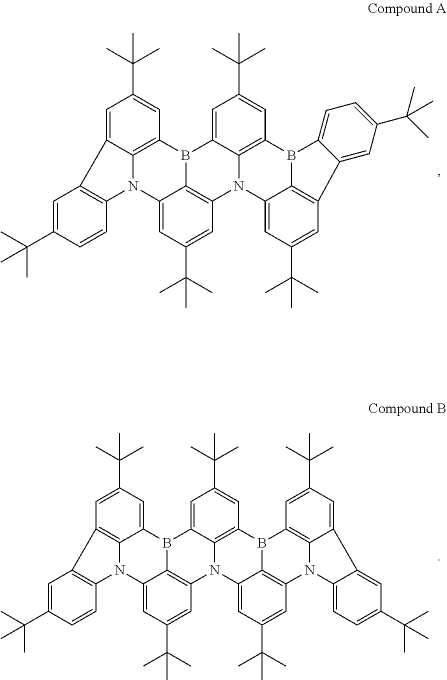

### Discussion:

Compound BN-1-11-101, Compound BN-1-7-80, Compound BN-1-11-115, Compound BN-1-7-53, Compound BN-1-5-94, and Compound BN-1-5-73 of the present disclosure differ from Comparative Compound A mainly in that: in the compounds of the present disclosure, the ring Cy4 and the ring Cy5 are joined by a single bond, and the ring Cy5 and the ring Cy6 are not joined; while in Comparative Compound A, two rings corresponding to the ring Cy4 and the ring Cy5 are not joined, and two rings corresponding to the ring Cy5 and the ring Cy6 are joined by a single bond. As can be seen from the data in Table 1, compared with that of Comparative Compound A, the maximum emission wavelengths of Compound BN-1-11-101, Compound BN-1-7-80, Compound BN-1-11-115, Compound BN-1-7-53, Compound BN-1-5-94, and Compound BN-1-5-73 of the present disclosure are unexpectedly red-shifted by 66 nm, 87 nm, 127 nm, 144 nm, 79 nm, and 137 nm, respectively. Compared with Comparative Compound B in the related art, the compounds of the present disclosure also exhibit unexpected significant red-shifts. As can be seen from the data in Table 1, compared with that of Comparative Compound B, the maximum emission wavelengths of Compound BN-1-11-101, Compound BN-1-7-80, Compound BN-1-11-115, Compound BN-1-7-53, Compound BN-1-5-94, and Compound BN-1-5-73 of the present disclosure are significantly red-shifted by 54 nm, 75 nm, 115 nm, 132 nm, 67 nm, and 125 nm, respectively. It can be seen that due to a particular polycyclic structure represented by Formula 1, the compound of the present disclosure can effectively adjust the emission wavelength and modulate the emitted color to a desired orange to red band, and the compounds of the present disclosure adjust the maximum emission wavelength to different degrees and can very conveniently implement fine adjustments to different wavelengths or colors, greatly enriching a light-emitting material system and an emitted color range.

Additionally, the compound of the present disclosure has deep HOMO and LUMO energy levels and a relatively strong hole or electron trapping capability so that the compound of the present disclosure, as a light-emitting material, has a potential to achieve high device efficiency.

To conclude, the compound of the present disclosure having a particular structure represented by Formula 1 can adjust the maximum emission wavelength to different degrees and achieve the desired orange to red light emission, greatly enriching a fluorescent material system and the emitted color range, and meanwhile, the deep HOMO and LUMO energy levels can ensure that the compound has the potential to achieve high device efficiency and become an excellent light-emitting material, proving the excellent performance and broad potential application prospect of the compound of the present disclosure having the particular structure of Formula 1.

It should be understood that various embodiments described herein are merely embodiments and not intended to limit the scope of the present disclosure. Therefore, it is apparent to those skilled in the art that the present disclosure as claimed may include variations of specific embodiments and preferred embodiments described herein. Many of the materials and structures described herein may be replaced with other materials and structures without departing from the spirit of the present disclosure. It should be understood that various theories as to why the present disclosure works are not intended to be limitative.

What is claimed is:

1. A compound having a structure represented by Formula 1: 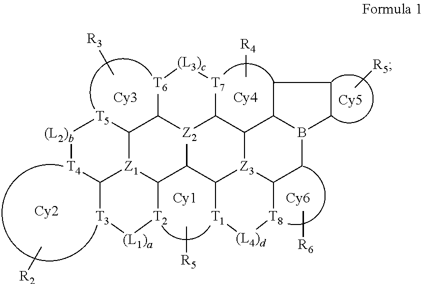
wherein the ring Cy1, the ring Cy2, the ring Cy3, the ring Cy5, and the ring Cy6 are, at each occurrence identically or differently, selected from an unsaturated carbocyclic ring having 5 to 30 carbon atoms or an unsaturated heterocyclic ring having 3 to 30 carbon atoms; and the ring Cy4 is selected from an unsaturated carbocyclic ring having 5 to 30 carbon atoms or an unsaturated heterocyclic ring having 4 to 30 carbon atoms;
Z₁, Z₂, and Z₃ are, at each occurrence identically or differently, selected from B, N, P, P═O, P═S, As, As═O, As═S, SiR, or GeR;
L₁, L₂, L₃, and L₄ are, at each occurrence identically or differently, selected from a single bond, O, S, Se, NR′, PR′, P═OR′, P═SR′, AsR′, As═OR′, As═SR′, CR′R′, SiR′R′, or GeR′R′; when multiple R′ are present at the same time, the multiple R′ are identical or different;
a, b, c, and d are each independently selected from 0, 1, or 2;
T₁ to T₈ are, at each occurrence identically or differently, selected from C, CRₜ, CRₜRₜ, N, NRₜ, O, S, or Se;
R₁, R₂, R₃, R₄, R₅, and R₆ represent, at each occurrence identically or differently, mono-substitution, multiple substitutions, or non-substitution;
R, R′, Rₜ, R₁, R₂, R₃, R₄, R₅, and R₆ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
adjacent substituents R₅ can be optionally joined to form a ring; and
adjacent substituents R′, Rₜ, R₁, R₂, R₃, R₄, and R₆ can be optionally joined to form a ring.

2. The compound according to claim 1, wherein the ring Cy1, the ring Cy2, the ring Cy3, the ring Cy5, and the ring Cy6 are, at each occurrence identically or differently, selected from a five-membered unsaturated carbocyclic ring, an aromatic ring having 6 to 18 carbon atoms, or a heteroaromatic ring having 3 to 18 carbon atoms; and the ring Cy4 is selected from a five-membered unsaturated carbocyclic ring, an aromatic ring having 6 to 18 carbon atoms, or a heteroaromatic ring having 4 to 18 carbon atoms;
preferably, the ring Cy1, the ring Cy2, the ring Cy3, the ring Cy4, the ring Cy5, and the ring Cy6 are, at each occurrence identically or differently, selected from a benzene ring, a pyridine ring, a naphthalene ring, a phenanthrene ring, an anthracene ring, an indene ring, a fluorene ring, an indole ring, a carbazole ring, a benzofuran ring, a dibenzofuran ring, a benzosilole ring, a dibenzosilole ring, a benzothiophene ring, a dibenzothiophene ring, a dibenzoselenophene ring, a cyclopentadienyl ring, a furan ring, a thiophene ring, a silole ring, or a combination thereof.

3. The compound according to claim 1, wherein the compound has a structure represented by any one of Formula 1-1 to Formula 1-4: 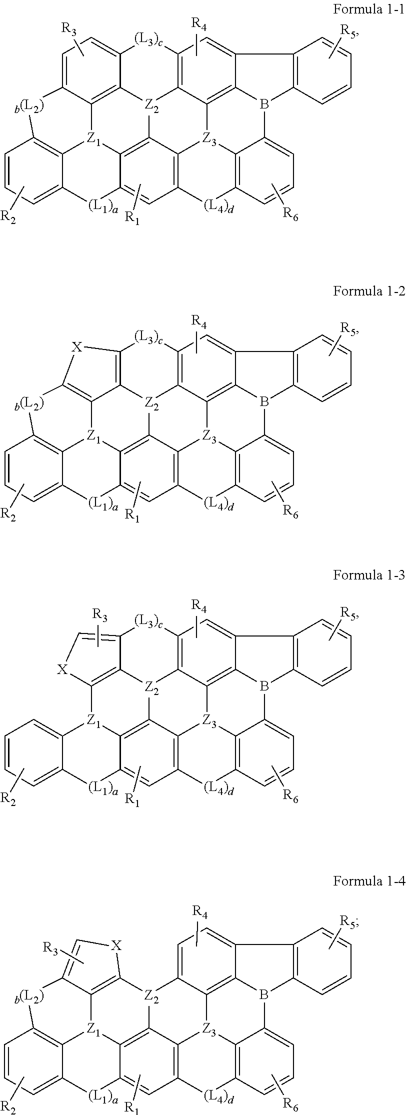
wherein Z₁, Z₂, and Z₃ are, at each occurrence identically or differently, selected from B, N, P, P═O, P═S, As, As═O, As═S, SiR, or GeR;
X is, at each occurrence identically or differently, selected from O, S, Se, or NRx;
L₁, L₂, L₃, and L₄ are, at each occurrence identically or differently, selected from a single bond, O, S, Se, NR′, PR′, P═OR′, P═SR′, AsR′, As═OR′, As═SR′, CR′R′, SiR′R′, or GeR′R′; when multiple R′ are present at the same time, the multiple R′ are identical or different;
a, b, c, and d are each independently selected from 0, 1, or 2;
R₁, R₂, R₃, R₄, R₅, and R₆ represent, at each occurrence identically or differently, mono-substitution, multiple substitutions, or non-substitution;
R, R′, Rₓ, R₁, R₂, R₃, R₄, R₅, and R₆ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
adjacent substituents R₅ can be optionally joined to form a ring; and
adjacent substituents R′, Rₓ, R₁, R₂, R₃, R₄, and R₆ can be optionally joined to form a ring;
preferably, the compound has a structure represented by Formula 1-1.

4. The compound according to claim 1, wherein the L₁, L₂, L₃, and L₄ are, at each occurrence identically or differently, selected from a single bond, O, S, Se, NR′, P═OR′, CR′R′, or SiR′R′; and the R′ is, at each occurrence identically or differently, selected from the group consisting of substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted aryl having 6 to 30 carbon atoms, substituted or unsubstituted heteroaryl having 3 to 30 carbon atoms, and combinations thereof;
preferably, the L₁, L₂, L₃, and L₄ are, at each occurrence identically or differently, selected from a single bond, O, S, NR′, CR′R′, or SiR′R′; and the R′ is, at each occurrence identically or differently, selected from the group consisting of substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, and combinations thereof.

5. The compound according to claim 1, wherein a, b, c, and d are each independently selected from 0 or 1;
preferably, a+b+c+d=0, 1, or 2;
more preferably, a+b=0, 1, or 2; and c and d are both 0.

6. The compound according to claim 1, wherein Z₁, Z₂, and Z₃ are, at each occurrence identically or differently, selected from B, N, P, P═O, or P═S, and at least one of Z₁, Z₂, or Z₃ is, at each occurrence identically or differently, selected from N, P, P═O, or P═S;
preferably, Z₁, Z₂, and Z₃ are, at each occurrence identically or differently, selected from B, N, or P═O, and at least two of Z₁, Z₂, and Z₃ are selected from N;
more preferably, Z₁ and Z₃ are selected from N, and Z₂ is selected from B.

7. The compound according to claim 1, wherein the R₁, R₂, R₃, R₄, R₅, and R₆ are, at each occurrence identically or differently, selected from the group consisting of hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted alkylgermanyl having 3 to 20 carbon atoms, substituted or unsubstituted amino having 0 to 20 carbon atoms, cyano, isocyano, and combinations thereof; adjacent substituents R₅ can be optionally joined to form a ring; and adjacent substituents R₁, R₂, R₃, R₄, and R₆ can be optionally joined to form a ring;
preferably, the R₁, R₂, R₃, R₄, R₅, and R₆ are, at each occurrence identically or differently, selected from the group consisting of hydrogen, deuterium, halogen, substituted or unsubstituted alkyl having 1 to 12 carbon atoms, substituted or unsubstituted cycloalkyl having 3 to 12 ring carbon atoms, substituted or unsubstituted aryl having 6 to 24 carbon atoms, substituted or unsubstituted heteroaryl having 3 to 24 carbon atoms, substituted or unsubstituted alkylsilyl having 3 to 10 carbon atoms, substituted or unsubstituted amino having 0 to 20 carbon atoms, cyano, and combinations thereof;
more preferably, the R₁, R₂, R₃, R₄, R₅, and R₆ are, at each occurrence identically or differently, selected from the group consisting of: hydrogen, deuterium, fluorine, cyano, methyl, ethyl, n-propyl, isopropyl, cyclopropyl, n-butyl, isobutyl, t-butyl, cyclopentyl, neopentyl, cyclohexyl, trimethylsilyl, phenyl, biphenyl, terphenyl, tetraphenyl, triphenylenyl, tetraphenylenyl, naphthyl, phenanthryl, anthryl, indenyl, fluorenyl, indolyl, carbazolyl, benzofuryl, dibenzofuryl, benzosilolyl, dibenzosilolyl, benzothienyl, dibenzothienyl, dibenzoselenophenyl, diphenylamino, dibenzofurylphenylamino, and combinations thereof.

8. The compound according to claim 1, wherein at least one of the R₁, R₂, R₃, R₄, R₅, or R₆ is, at each occurrence identically or differently, selected from the group consisting of: deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof, adjacent substituents R₅ can be optionally joined to form a ring; and adjacent substituents R₁, R₂, R₃, R₄, and R₆ can be optionally joined to form a ring;
preferably, at least one of the R₁, R₂, R₃, R₄, R₅, or R₆ is, at each occurrence identically or differently, selected from the group consisting of: deuterium, halogen, substituted or unsubstituted alkyl having 1 to 12 carbon atoms, substituted or unsubstituted cycloalkyl having 3 to 12 ring carbon atoms, substituted or unsubstituted aryl having 6 to 24 carbon atoms, substituted or unsubstituted heteroaryl having 3 to 24 carbon atoms, substituted or unsubstituted alkylsilyl having 3 to 10 carbon atoms, substituted or unsubstituted amino having 0 to 20 carbon atoms, cyano, and combinations thereof;
more preferably, at least one of the R₁, R₂, R₃, R₄, R₅, or R₆ is, at each occurrence identically or differently, selected from the group consisting of deuterium, fluorine, cyano, methyl, ethyl, n-propyl, isopropyl, cyclopropyl, n-butyl, isobutyl, t-butyl, cyclopentyl, neopentyl, cyclohexyl, trimethylsilyl, phenyl, biphenyl, terphenyl, tetraphenyl, triphenylenyl, tetraphenylenyl, naphthyl, phenanthryl, anthryl, indenyl, fluorenyl, indolyl, carbazolyl, benzofuryl, dibenzofuryl, benzosilolyl, dibenzosilolyl, benzothienyl, dibenzothienyl, dibenzoselenophenyl, diphenylamino, dibenzofurylphenylamino, and combinations thereof.

9. The compound according to claim 1, wherein the compound is selected from the group consisting of Compound BN-1-1-h₁ to Compound BN-1-20-h₁ and Compound BN-2-1-h₂ to Compound BN-2-6-h₂; h₁ is selected from an integer from 1 to 127; and h₂ is selected from an integer from 1 to 390;
Compound BN-1-1-h₁ to Compound BN-1-20-h₁ have the following structures: 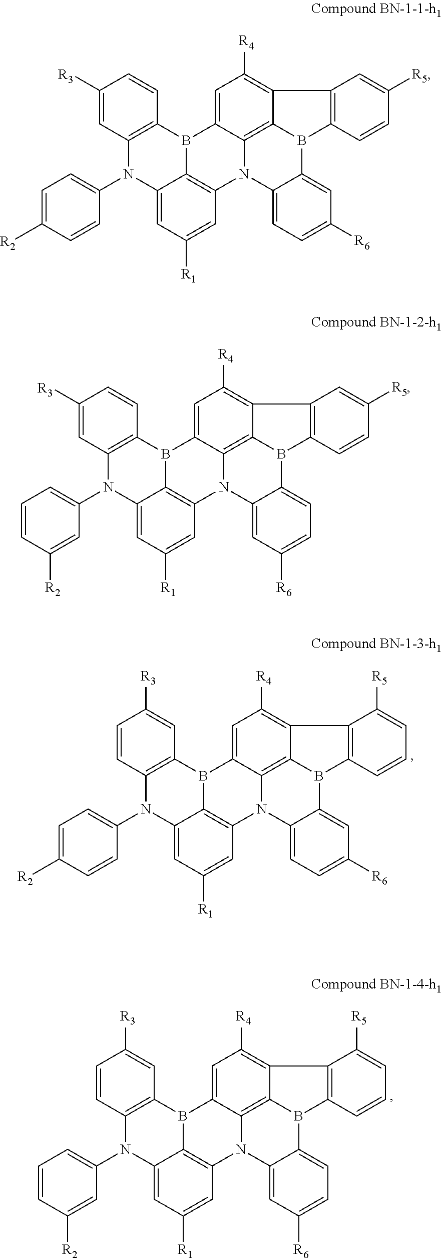 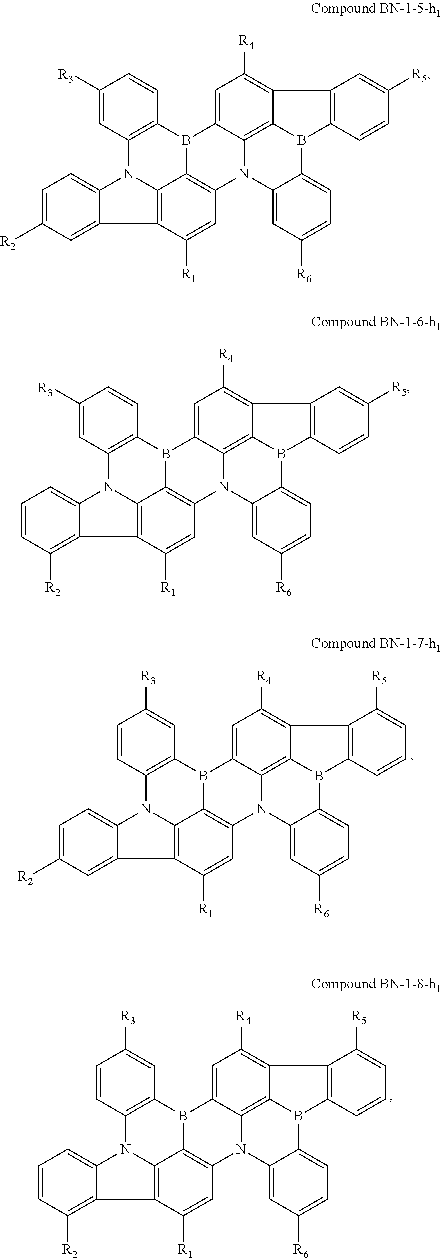 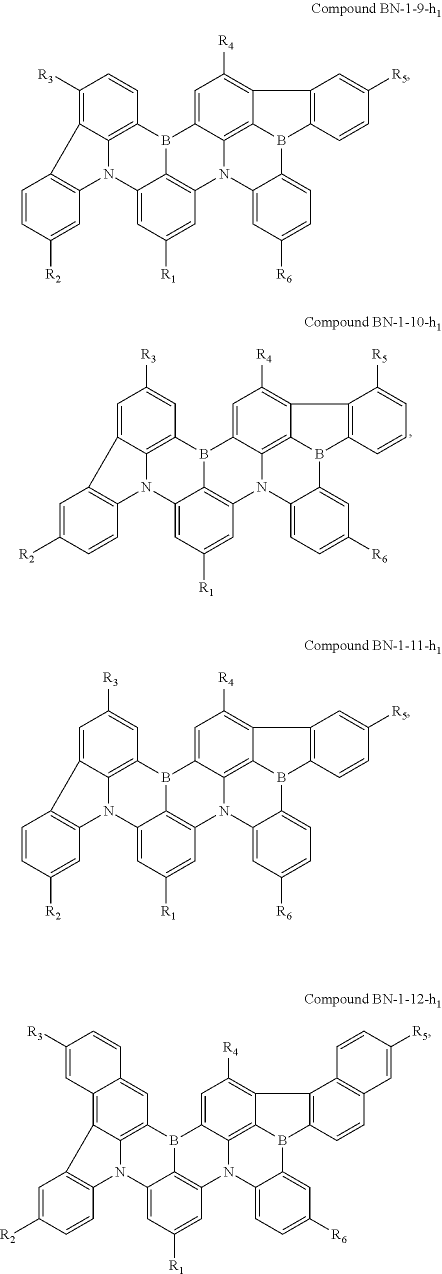 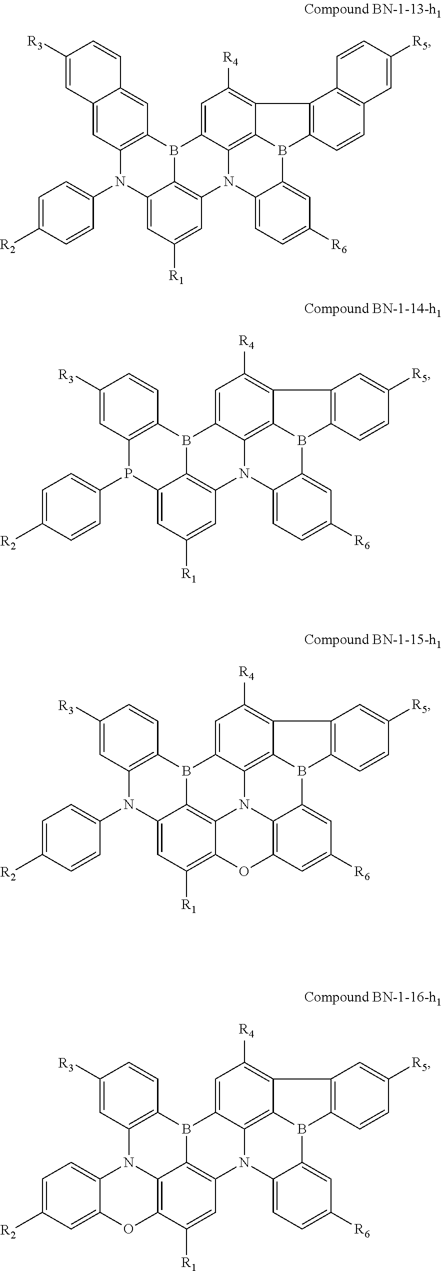 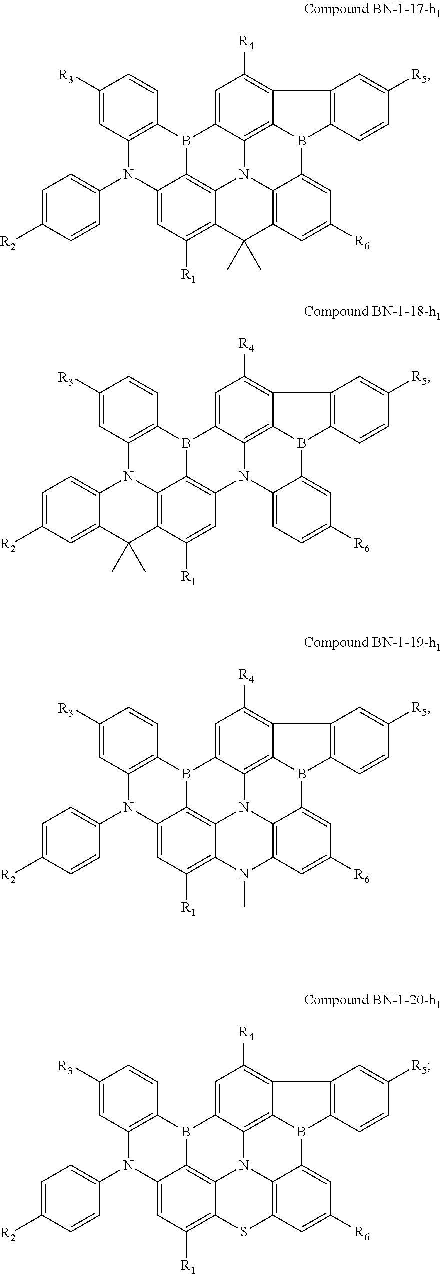
wherein R₁, R₂, R₃, R₄, R₅, R₆ and h₁ are selected from atoms or groups in the following table, respectively:

| h₁ | R₁ | R₂ | R₃ | R₄ | R₅ | R₆ |
|---|---|---|---|---|---|---|
| 1 | H | H | H | H | H | H |
| 2 | H | P1 | P1 | H | P1 | P1 |
| 3 | H | P2 | P2 | H | P2 | P2 |
| 4 | H | P3 | P3 | H | P3 | P3 |
| 5 | H | P4 | P4 | H | P4 | P4 |
| 6 | H | P5 | P5 | H | P5 | P5 |
| 7 | H | P6 | P6 | H | P6 | P6 |
| 8 | H | P7 | P7 | H | P7 | P7 |
| 9 | H | P8 | P8 | H | P8 | P8 |
| 10 | H | P9 | P9 | H | P9 | P9 |
| 11 | H | P10 | P10 | H | P10 | P10 |
| 12 | H | P11 | P11 | H | P11 | P11 |
| 13 | P12 | H | H | H | H | H |
| 14 | P13 | H | H | H | H | H |
| 15 | P14 | H | H | H | H | H |
| 16 | P15 | H | H | H | H | H |
| 17 | P16 | H | H | H | H | H |
| 18 | P17 | H | H | H | H | H |
| 19 | P18 | H | H | H | H | H |
| 20 | P19 | H | H | H | H | H |
| 21 | P20 | H | H | H | H | H |
| 22 | F | H | H | H | H | H |
| 23 | H | H | H | P12 | H | H |
| 24 | H | H | H | P13 | H | H |
| 25 | H | H | H | P14 | H | H |
| 26 | H | H | H | P15 | H | H |
| 27 | H | H | H | P16 | H | H |
| 28 | H | H | H | P17 | H | H |
| 29 | H | H | H | P18 | H | H |
| 30 | H | H | H | P19 | H | H |
| 31 | H | H | H | P20 | H | H |
| 32 | H | H | H | F | H | H |
| 33 | D | D | D | D | D | D |
| 34 | P1 | P1 | P1 | P1 | P1 | P1 |
| 35 | P2 | P2 | P2 | P2 | P2 | P2 |
| 36 | P3 | P3 | P3 | P3 | P3 | P3 |
| 37 | P4 | P4 | P4 | P4 | P4 | P4 |
| 38 | P5 | P5 | P5 | P5 | P5 | P5 |
| 39 | P6 | P6 | P6 | P6 | P6 | P6 |
| 40 | P7 | P7 | P7 | P7 | P7 | P7 |
| 41 | P8 | P8 | P8 | P8 | P8 | P8 |
| 42 | P9 | P9 | P9 | P9 | P9 | P9 |
| 43 | P10 | P10 | P10 | P10 | P10 | P10 |
| 44 | P11 | P11 | P11 | P11 | P11 | P11 |
| 45 | P12 | P12 | P12 | P12 | P12 | P12 |
| 46 | P13 | P13 | P13 | P13 | P13 | P13 |
| 47 | P14 | P14 | P14 | P14 | P14 | P14 |
| 48 | P15 | P15 | P15 | P15 | P15 | P15 |
| 49 | P16 | P16 | P16 | P16 | P16 | P16 |
| 50 | P17 | P17 | P17 | P17 | P17 | P17 |
| 51 | P18 | P18 | P18 | P18 | P18 | P18 |
| 52 | P19 | P19 | P19 | P19 | P19 | P19 |
| 53 | H | P6 | P6 | H | P20 | P6 |
| 54 | P6 | H | P1 | H | P1 | P6 |
| 55 | P6 | H | P2 | H | P2 | P6 |
| 56 | P6 | H | P3 | H | P3 | P6 |
| 57 | P6 | H | P4 | H | P4 | P6 |
| 58 | P6 | H | P5 | H | P5 | P6 |
| 59 | P6 | H | P6 | H | P6 | P6 |
| 60 | P6 | H | P7 | H | P7 | P6 |
| 61 | P6 | H | P8 | H | P8 | P6 |
| 62 | P6 | H | P9 | H | P9 | P6 |
| 63 | P6 | H | P10 | H | P10 | P6 |
| 64 | P6 | H | P11 | H | P11 | P6 |
| 65 | P6 | H | P12 | H | P12 | P6 |
| 66 | P6 | H | P13 | H | P13 | P6 |
| 67 | P6 | H | P14 | H | P14 | P6 |
| 68 | P6 | H | P15 | H | P15 | P6 |
| 69 | P6 | H | P16 | H | P16 | P6 |
| 70 | P6 | H | P17 | H | P17 | P6 |
| 71 | P6 | H | P18 | H | P18 | P6 |
| 72 | P6 | H | P19 | H | P19 | P6 |
| 73 | H | P6 | P20 | H | P20 | P6 |
| 74 | P6 | H | F | H | F | P6 |
| 75 | H | P6 | P1 | H | P6 | P6 |
| 76 | H | P6 | P2 | H | P6 | P6 |
| 77 | H | P6 | P3 | H | P6 | P6 |
| 78 | H | P6 | P4 | H | P6 | P6 |
| 79 | H | P6 | P5 | H | P6 | P6 |
| 80 | H | P6 | P6 | H | P6 | P6 |
| 81 | H | P6 | P7 | H | P6 | P6 |
| 82 | H | P6 | P8 | H | P6 | P6 |
| 83 | H | P6 | P9 | H | P6 | P6 |
| 84 | H | P6 | P10 | H | P6 | P6 |
| 85 | H | P6 | P11 | H | P6 | P6 |
| 86 | H | P6 | P12 | H | P6 | P6 |
| 87 | H | P6 | P13 | H | P6 | P6 |
| 88 | H | P6 | P14 | H | P6 | P6 |
| 89 | H | P6 | P15 | H | P6 | P6 |
| 90 | H | P6 | P16 | H | P6 | P6 |
| 91 | H | P6 | P17 | H | P6 | P6 |
| 92 | H | P6 | P18 | H | P6 | P6 |
| 93 | H | P6 | P19 | H | P6 | P6 |
| 94 | H | P6 | P20 | H | P6 | P6 |
| 95 | H | P6 | F | H | P6 | P6 |
| 96 | P6 | P6 | P6 | H | P1 | P6 |
| 97 | P6 | P6 | P6 | H | P2 | P6 |
| 98 | P6 | P6 | P6 | H | P3 | P6 |
| 99 | P6 | P6 | P6 | H | P4 | P6 |
| 100 | P6 | P6 | P6 | H | P5 | P6 |
| 101 | P6 | P6 | P6 | H | P6 | P6 |
| 102 | P6 | P6 | P6 | H | P7 | P6 |
| 103 | P6 | P6 | P6 | H | P8 | P6 |
| 104 | P6 | P6 | P6 | H | P9 | P6 |
| 105 | P6 | P6 | P6 | H | P10 | P6 |
| 106 | P6 | P6 | P6 | H | P11 | P6 |
| 107 | P6 | P6 | P6 | H | P12 | P6 |
| 108 | P6 | P6 | P6 | H | P13 | P6 |
| 109 | P6 | P6 | P6 | H | P14 | P6 |
| 110 | P6 | P6 | P6 | H | P15 | P6 |
| 111 | P6 | P6 | P6 | H | P16 | P6 |
| 112 | P6 | P6 | P6 | H | P17 | P6 |
| 113 | P6 | P6 | P6 | H | P18 | P6 |
| 114 | P6 | P6 | P6 | H | P19 | P6 |
| 115 | P6 | P6 | P6 | H | P20 | P6 |
| 116 | P6 | P6 | P6 | P6 | P20 | P6 |
| 117 | P1 | P1 | P1 | H | P1 | P1 |
| 118 | P2 | P2 | P2 | H | P2 | P2 |
| 119 | P3 | P3 | P3 | H | P3 | P3 |
| 120 | P4 | P4 | P4 | H | P4 | P4 |
| 121 | P5 | P5 | P5 | H | P5 | P5 |
| 122 | P6 | P6 | P6 | H | P6 | P6 |
| 123 | P7 | P7 | P7 | H | P7 | P7 |
| 124 | P8 | P8 | P8 | H | P8 | P8 |
| 125 | P9 | P9 | P9 | H | P9 | P9 |
| 126 | P10 | P10 | P10 | H | P10 | P10 |
| 127 | P11 | P11 | P11 | H | P11 | P11 |

Compound BN-2-1-h₂ to Compound BN-2-6-h₂ have the following structures: 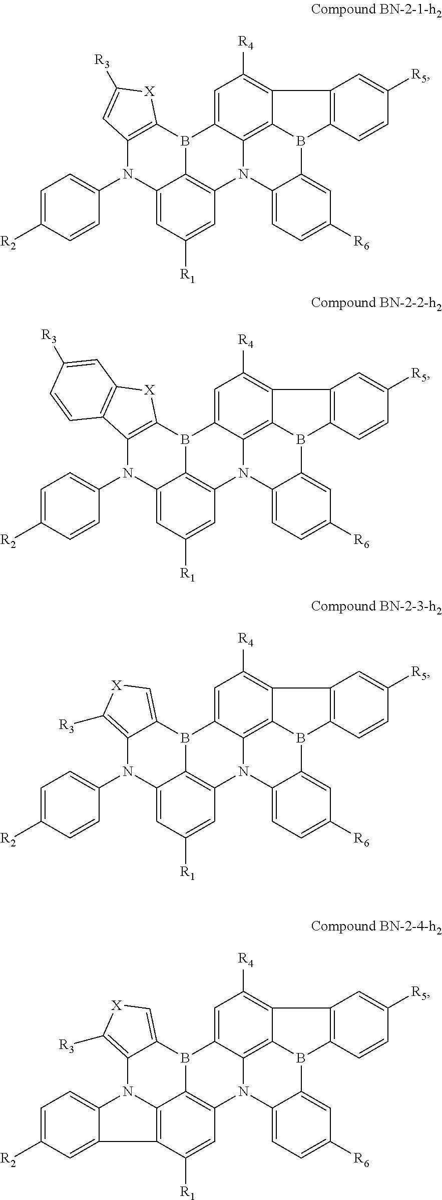 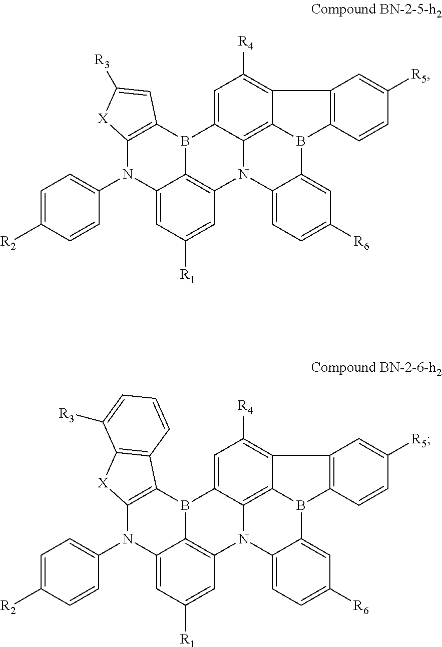
wherein X, R₁, R₂, R₃, R₄, R₅, R₆ and h₂ are selected from atoms or groups in the following table, respectively:

| h₂ | R₁ | R₂ | R₃ | R₄ | R₅ | R₆ | X |
|---|---|---|---|---|---|---|---|
| 1 | H | H | H | H | H | H | O |
| 2 | H | P1 | P1 | H | P1 | P1 | O |
| 3 | H | P2 | P2 | H | P2 | P2 | O |
| 4 | H | P3 | P3 | H | P3 | P3 | O |
| 5 | H | P4 | P4 | H | P4 | P4 | O |
| 6 | H | P5 | P5 | H | P5 | P5 | O |
| 7 | H | P6 | P6 | H | P6 | P6 | O |
| 8 | H | P7 | P7 | H | P7 | P7 | O |
| 9 | H | P8 | P8 | H | P8 | P8 | O |
| 10 | H | P9 | P9 | H | P9 | P9 | O |
| 11 | H | P10 | P10 | H | P10 | P10 | O |
| 12 | H | P11 | P11 | H | P11 | P11 | O |
| 13 | P12 | H | H | H | H | H | O |
| 14 | P13 | H | H | H | H | H | O |
| 15 | P14 | H | H | H | H | H | O |
| 16 | P15 | H | H | H | H | H | O |
| 17 | P16 | H | H | H | H | H | O |
| 18 | P17 | H | H | H | H | H | O |
| 19 | P18 | H | H | H | H | H | O |
| 20 | P19 | H | H | H | H | H | O |
| 21 | P20 | H | H | H | H | H | O |
| 22 | F | H | H | H | H | H | O |
| 23 | H | H | H | P12 | H | H | O |
| 24 | H | H | H | P13 | H | H | O |
| 25 | H | H | H | P14 | H | H | O |
| 26 | H | H | H | P15 | H | H | O |
| 27 | H | H | H | P16 | H | H | O |
| 28 | H | H | H | P17 | H | H | O |
| 29 | H | H | H | P18 | H | H | O |
| 30 | H | H | H | P19 | H | H | O |
| 31 | H | H | H | P20 | H | H | O |
| 32 | H | H | H | F | H | H | O |
| 33 | D | D | D | D | D | D | O |
| 34 | P1 | P1 | P1 | P1 | P1 | P1 | O |
| 35 | P2 | P2 | P2 | P2 | P2 | P2 | O |
| 36 | P3 | P3 | P3 | P3 | P3 | P3 | O |
| 37 | P4 | P4 | P4 | P4 | P4 | P4 | O |
| 38 | P5 | P5 | P5 | P5 | P5 | P5 | O |
| 39 | P6 | P6 | P6 | P6 | P6 | P6 | O |
| 40 | P7 | P7 | P7 | P7 | P7 | P7 | O |
| 41 | P8 | P8 | P8 | P8 | P8 | P8 | O |
| 42 | P9 | P9 | P9 | P9 | P9 | P9 | O |
| 43 | P10 | P10 | P10 | P10 | P10 | P10 | O |
| 44 | P11 | P11 | P11 | P11 | P11 | P11 | O |
| 45 | P12 | P12 | P12 | P12 | P12 | P12 | O |
| 46 | P13 | P13 | P13 | P13 | P13 | P13 | O |
| 47 | P14 | P14 | P14 | P14 | P14 | P14 | O |
| 48 | P15 | P15 | P15 | P15 | P15 | P15 | O |
| 49 | P16 | P16 | P16 | P16 | P16 | P16 | O |
| 50 | P17 | P17 | P17 | P17 | P17 | P17 | O |
| 51 | P18 | P18 | P18 | P18 | P18 | P18 | O |
| 52 | P19 | P19 | P19 | P19 | P19 | P19 | O |
| 53 | P20 | P20 | P20 | P20 | P20 | P20 | O |
| 54 | P6 | H | P1 | H | P1 | P6 | O |
| 55 | P6 | H | P2 | H | P2 | P6 | O |
| 56 | P6 | H | P3 | H | P3 | P6 | O |
| 57 | P6 | H | P4 | H | P4 | P6 | O |
| 58 | P6 | H | P5 | H | P5 | P6 | O |
| 59 | P6 | H | P6 | H | P6 | P6 | O |
| 60 | P6 | H | P7 | H | P7 | P6 | O |
| 61 | P6 | H | P8 | H | P8 | P6 | O |
| 62 | P6 | H | P9 | H | P9 | P6 | O |
| 63 | P6 | H | P10 | H | P10 | P6 | O |
| 64 | P6 | H | P11 | H | P11 | P6 | O |
| 65 | P6 | H | P12 | H | P12 | P6 | O |
| 66 | P6 | H | P13 | H | P13 | P6 | O |
| 67 | P6 | H | P14 | H | P14 | P6 | O |
| 68 | P6 | H | P15 | H | P15 | P6 | O |
| 69 | P6 | H | P16 | H | P16 | P6 | O |
| 70 | P6 | H | P17 | H | P17 | P6 | O |
| 71 | P6 | H | P18 | H | P18 | P6 | O |
| 72 | P6 | H | P19 | H | P19 | P6 | O |
| 73 | P6 | H | P20 | H | P20 | P6 | O |
| 74 | P6 | H | F | H | F | P6 | O |
| 75 | H | P6 | P1 | H | P6 | P6 | O |
| 76 | H | P6 | P2 | H | P6 | P6 | O |
| 77 | H | P6 | P3 | H | P6 | P6 | O |
| 78 | H | P6 | P4 | H | P6 | P6 | O |
| 79 | H | P6 | P5 | H | P6 | P6 | O |
| 80 | H | P6 | P6 | H | P6 | P6 | O |
| 81 | H | P6 | P7 | H | P6 | P6 | O |
| 82 | H | P6 | P8 | H | P6 | P6 | O |
| 83 | H | P6 | P9 | H | P6 | P6 | O |
| 84 | H | P6 | P10 | H | P6 | P6 | O |
| 85 | H | P6 | P11 | H | P6 | P6 | O |
| 86 | H | P6 | P12 | H | P6 | P6 | O |
| 87 | H | P6 | P13 | H | P6 | P6 | O |
| 88 | H | P6 | P14 | H | P6 | P6 | O |
| 89 | H | P6 | P15 | H | P6 | P6 | O |
| 90 | H | P6 | P16 | H | P6 | P6 | O |
| 91 | H | P6 | P17 | H | P6 | P6 | O |
| 92 | H | P6 | P18 | H | P6 | P6 | O |
| 93 | H | P6 | P19 | H | P6 | P6 | O |
| 94 | H | P6 | P20 | H | P6 | P6 | O |
| 95 | H | P6 | F | H | P6 | P6 | O |
| 96 | P6 | P6 | P6 | H | P1 | P6 | O |
| 97 | P6 | P6 | P6 | H | P2 | P6 | O |
| 98 | P6 | P6 | P6 | H | P3 | P6 | O |
| 99 | P6 | P6 | P6 | H | P4 | P6 | O |
| 100 | P6 | P6 | P6 | H | P5 | P6 | O |
| 101 | P6 | P6 | P6 | H | P6 | P6 | O |
| 102 | P6 | P6 | P6 | H | P7 | P6 | O |
| 103 | P6 | P6 | P6 | H | P8 | P6 | O |
| 104 | P6 | P6 | P6 | H | P9 | P6 | O |
| 105 | P6 | P6 | P6 | H | P10 | P6 | O |
| 106 | P6 | P6 | P6 | H | P11 | P6 | O |
| 107 | P6 | P6 | P6 | H | P12 | P6 | O |
| 108 | P6 | P6 | P6 | H | P13 | P6 | O |
| 109 | P6 | P6 | P6 | H | P14 | P6 | O |
| 110 | P6 | P6 | P6 | H | P15 | P6 | O |
| 111 | P6 | P6 | P6 | H | P16 | P6 | O |
| 112 | P6 | P6 | P6 | H | P17 | P6 | O |
| 113 | P6 | P6 | P6 | H | P18 | P6 | O |
| 114 | P6 | P6 | P6 | H | P19 | P6 | O |
| 115 | P6 | P6 | P6 | H | P20 | P6 | O |
| 116 | P6 | P6 | P6 | H | F | P6 | O |
| 117 | P1 | P1 | P1 | H | P1 | P1 | O |
| 118 | P2 | P2 | P2 | H | P2 | P2 | O |
| 119 | P3 | P3 | P3 | H | P3 | P3 | O |
| 120 | P4 | P4 | P4 | H | P4 | P4 | O |
| 121 | P5 | P5 | P5 | H | P5 | P5 | O |
| 122 | P6 | P6 | P6 | H | P6 | P6 | O |
| 123 | P7 | P7 | P7 | H | P7 | P7 | O |
| 124 | P8 | P8 | P8 | H | P8 | P8 | O |
| 125 | P9 | P9 | P9 | H | P9 | P9 | O |
| 126 | P10 | P10 | P10 | H | P10 | P10 | O |
| 127 | P11 | P11 | P11 | H | P11 | P11 | O |
| 128 | H | H | H | H | H | H | S |
| 129 | H | P1 | P1 | H | P1 | P1 | S |
| 130 | H | P2 | P2 | H | P2 | P2 | S |
| 131 | H | P3 | P3 | H | P3 | P3 | S |
| 132 | H | P4 | P4 | H | P4 | P4 | S |
| 133 | H | P5 | P5 | H | P5 | P5 | S |
| 134 | H | P6 | P6 | H | P6 | P6 | S |
| 135 | H | P7 | P7 | H | P7 | P7 | S |
| 136 | H | P8 | P8 | H | P8 | P8 | S |
| 137 | H | P9 | P9 | H | P9 | P9 | S |
| 138 | H | P10 | P10 | H | P10 | P10 | S |
| 139 | H | P11 | P11 | H | P11 | P11 | S |
| 140 | P12 | H | H | H | H | H | S |
| 141 | P13 | H | H | H | H | H | S |
| 142 | P14 | H | H | H | H | H | S |
| 143 | P15 | H | H | H | H | H | S |
| 144 | P16 | H | H | H | H | H | S |
| 145 | P17 | H | H | H | H | H | S |
| 146 | P18 | H | H | H | H | H | S |
| 147 | P19 | H | H | H | H | H | S |
| 148 | P20 | H | H | H | H | H | S |
| 149 | F | H | H | H | H | H | S |
| 150 | H | H | H | P12 | H | H | S |
| 151 | H | H | H | P13 | H | H | S |
| 152 | H | H | H | P14 | H | H | S |
| 153 | H | H | H | P15 | H | H | S |
| 154 | H | H | H | P16 | H | H | S |
| 155 | H | H | H | P17 | H | H | S |
| 156 | H | H | H | P18 | H | H | S |
| 157 | H | H | H | P19 | H | H | S |
| 158 | H | H | H | P20 | H | H | S |
| 159 | H | H | H | F | H | H | S |
| 160 | D | D | D | D | D | D | S |
| 161 | P1 | P1 | P1 | P1 | P1 | P1 | S |
| 162 | P2 | P2 | P2 | P2 | P2 | P2 | S |
| 163 | P3 | P3 | P3 | P3 | P3 | P3 | S |
| 164 | P4 | P4 | P4 | P4 | P4 | P4 | S |
| 165 | P5 | P5 | P5 | P5 | P5 | P5 | S |
| 166 | P6 | P6 | P6 | P6 | P6 | P6 | S |
| 167 | P7 | P7 | P7 | P7 | P7 | P7 | S |
| 168 | P8 | P8 | P8 | P8 | P8 | P8 | S |
| 169 | P9 | P9 | P9 | P9 | P9 | P9 | S |
| 170 | P10 | P10 | P10 | P10 | P10 | P10 | S |
| 171 | P11 | P11 | P11 | P11 | P11 | P11 | S |
| 172 | P12 | P12 | P12 | P12 | P12 | P12 | S |
| 173 | P13 | P13 | P13 | P13 | P13 | P13 | S |
| 174 | P14 | P14 | P14 | P14 | P14 | P14 | S |
| 175 | P15 | P15 | P15 | P15 | P15 | P15 | S |
| 176 | P16 | P16 | P16 | P16 | P16 | P16 | S |
| 177 | P17 | P17 | P17 | P17 | P17 | P17 | S |
| 178 | P18 | P18 | P18 | P18 | P18 | P18 | S |
| 179 | P19 | P19 | P19 | P19 | P19 | P19 | S |
| 180 | P20 | P20 | P20 | P20 | P20 | P20 | S |
| 181 | P6 | H | P1 | H | P1 | P6 | S |
| 182 | P6 | H | P2 | H | P2 | P6 | S |
| 183 | P6 | H | P3 | H | P3 | P6 | S |
| 184 | P6 | H | P4 | H | P4 | P6 | S |
| 185 | P6 | H | P5 | H | P5 | P6 | S |
| 186 | P6 | H | P6 | H | P6 | P6 | S |
| 187 | P6 | H | P7 | H | P7 | P6 | S |
| 188 | P6 | H | P8 | H | P8 | P6 | S |
| 189 | P6 | H | P9 | H | P9 | P6 | S |
| 190 | P6 | H | P10 | H | P10 | P6 | S |
| 191 | P6 | H | P11 | H | P11 | P6 | S |
| 192 | P6 | H | P12 | H | P12 | P6 | S |
| 193 | P6 | H | P13 | H | P13 | P6 | S |
| 194 | P6 | H | P14 | H | P14 | P6 | S |
| 195 | P6 | H | P15 | H | P15 | P6 | S |
| 196 | P6 | H | P16 | H | P16 | P6 | S |
| 197 | P6 | H | P17 | H | P17 | P6 | S |
| 198 | P6 | H | P18 | H | P18 | P6 | S |
| 199 | P6 | H | P19 | H | P19 | P6 | S |
| 200 | P6 | H | P20 | H | P20 | P6 | S |
| 201 | P6 | H | F | H | F | P6 | S |
| 202 | H | P6 | P1 | H | P6 | P6 | S |
| 203 | H | P6 | P2 | H | P6 | P6 | S |
| 204 | H | P6 | P3 | H | P6 | P6 | S |
| 205 | H | P6 | P4 | H | P6 | P6 | S |
| 206 | H | P6 | P5 | H | P6 | P6 | S |
| 207 | H | P6 | P6 | H | P6 | P6 | S |
| 208 | H | P6 | P7 | H | P6 | P6 | S |
| 209 | H | P6 | P8 | H | P6 | P6 | S |
| 210 | H | P6 | P9 | H | P6 | P6 | S |
| 211 | H | P6 | P10 | H | P6 | P6 | S |
| 212 | H | P6 | P11 | H | P6 | P6 | S |
| 213 | H | P6 | P12 | H | P6 | P6 | S |
| 214 | H | P6 | P13 | H | P6 | P6 | S |
| 215 | H | P6 | P14 | H | P6 | P6 | S |
| 216 | H | P6 | P15 | H | P6 | P6 | S |
| 217 | H | P6 | P16 | H | P6 | P6 | S |
| 218 | H | P6 | P17 | H | P6 | P6 | S |
| 219 | H | P6 | P18 | H | P6 | P6 | S |
| 220 | H | P6 | P19 | H | P6 | P6 | S |
| 221 | H | P6 | P20 | H | P6 | P6 | S |
| 222 | H | P6 | F | H | P6 | P6 | S |
| 223 | P6 | P6 | P6 | H | P1 | P6 | S |
| 224 | P6 | P6 | P6 | H | P2 | P6 | S |
| 225 | P6 | P6 | P6 | H | P3 | P6 | S |
| 226 | P6 | P6 | P6 | H | P4 | P6 | S |
| 227 | P6 | P6 | P6 | H | P5 | P6 | S |
| 228 | P6 | P6 | P6 | H | P6 | P6 | S |
| 229 | P6 | P6 | P6 | H | P7 | P6 | S |
| 230 | P6 | P6 | P6 | H | P8 | P6 | S |
| 231 | P6 | P6 | P6 | H | P9 | P6 | S |
| 232 | P6 | P6 | P6 | H | P10 | P6 | S |
| 233 | P6 | P6 | P6 | H | P11 | P6 | S |
| 234 | P6 | P6 | P6 | H | P12 | P6 | S |
| 235 | P6 | P6 | P6 | H | P13 | P6 | S |
| 236 | P6 | P6 | P6 | H | P14 | P6 | S |
| 237 | P6 | P6 | P6 | H | P15 | P6 | S |
| 238 | P6 | P6 | P6 | H | P16 | P6 | S |
| 239 | P6 | P6 | P6 | H | P17 | P6 | S |
| 240 | P6 | P6 | P6 | H | P18 | P6 | S |
| 241 | P6 | P6 | P6 | H | P19 | P6 | S |
| 242 | P6 | P6 | P6 | H | P20 | P6 | S |
| 243 | P6 | P6 | P6 | H | F | P6 | S |
| 244 | P1 | P1 | P1 | H | P1 | P1 | S |
| 245 | P2 | P2 | P2 | H | P2 | P2 | S |
| 246 | P3 | P3 | P3 | H | P3 | P3 | S |
| 247 | P4 | P4 | P4 | H | P4 | P4 | S |
| 248 | P5 | P5 | P5 | H | P5 | P5 | S |
| 249 | P6 | P6 | P6 | H | P6 | P6 | S |
| 250 | P7 | P7 | P7 | H | P7 | P7 | S |
| 251 | P8 | P8 | P8 | H | P8 | P8 | S |
| 252 | P9 | P9 | P9 | H | P9 | P9 | S |
| 253 | P10 | P10 | P10 | H | P10 | P10 | S |
| 254 | P11 | P11 | P11 | H | P11 | P11 | S |
| 255 | H | H | H | H | H | H | NMe |
| 256 | H | P1 | P1 | H | P1 | P1 | NMe |
| 257 | H | P2 | P2 | H | P2 | P2 | NMe |
| 258 | H | P3 | P3 | H | P3 | P3 | NMe |
| 259 | H | P4 | P4 | H | P4 | P4 | NMe |
| 260 | H | P5 | P5 | H | P5 | P5 | NMe |
| 261 | H | P6 | P6 | H | P6 | P6 | NMe |
| 262 | H | P7 | P7 | H | P7 | P7 | NMe |
| 263 | H | P8 | P8 | H | P8 | P8 | NMe |
| 264 | H | P9 | P9 | H | P9 | P9 | NMe |
| 265 | H | P10 | P10 | H | P10 | P10 | NMe |
| 266 | H | P11 | P11 | H | P11 | P11 | NMe |
| 267 | P12 | H | H | H | H | H | NMe |
| 268 | P13 | H | H | H | H | H | NMe |
| 269 | P14 | H | H | H | H | H | NMe |
| 270 | P15 | H | H | H | H | H | NMe |
| 271 | P16 | H | H | H | H | H | NMe |
| 272 | P17 | H | H | H | H | H | NMe |
| 273 | P18 | H | H | H | H | H | NMe |
| 274 | P19 | H | H | H | H | H | NMe |
| 275 | P20 | H | H | H | H | H | NMe |
| 276 | F | H | H | H | H | H | NMe |
| 277 | H | H | H | P12 | H | H | NMe |
| 278 | H | H | H | P13 | H | H | NMe |
| 279 | H | H | H | P14 | H | H | NMe |
| 280 | H | H | H | P15 | H | H | NMe |
| 281 | H | H | H | P16 | H | H | NMe |
| 282 | H | H | H | P17 | H | H | NMe |
| 283 | H | H | H | P18 | H | H | NMe |
| 284 | H | H | H | P19 | H | H | NMe |
| 285 | H | H | H | P20 | H | H | NMe |
| 286 | H | H | H | F | H | H | NMe |
| 287 | D | D | D | D | D | D | NMe |
| 288 | P1 | P1 | P1 | P1 | P1 | P1 | NMe |
| 289 | P2 | P2 | P2 | P2 | P2 | P2 | NMe |
| 290 | P3 | P3 | P3 | P3 | P3 | P3 | NMe |
| 291 | P4 | P4 | P4 | P4 | P4 | P4 | NMe |
| 292 | P5 | P5 | P5 | P5 | P5 | P5 | NMe |
| 293 | P6 | P6 | P6 | P6 | P6 | P6 | NMe |
| 294 | P7 | P7 | P7 | P7 | P7 | P7 | NMe |
| 295 | P8 | P8 | P8 | P8 | P8 | P8 | NMe |
| 296 | P9 | P9 | P9 | P9 | P9 | P9 | NMe |
| 297 | P10 | P10 | P10 | P10 | P10 | P10 | NMe |
| 298 | P11 | P11 | P11 | P11 | P11 | P11 | NMe |
| 299 | P12 | P12 | P12 | P12 | P12 | P12 | NMe |
| 300 | P13 | P13 | P13 | P13 | P13 | P13 | NMe |
| 301 | P14 | P14 | P14 | P14 | P14 | P14 | NMe |
| 302 | P15 | P15 | P15 | P15 | P15 | P15 | NMe |
| 303 | P16 | P16 | P16 | P16 | P16 | P16 | NMe |
| 304 | P17 | P17 | P17 | P17 | P17 | P17 | NMe |
| 305 | P18 | P18 | P18 | P18 | P18 | P18 | NMe |
| 306 | P19 | P19 | P19 | P19 | P19 | P19 | NMe |
| 307 | P20 | P20 | P20 | P20 | P20 | P20 | NMe |
| 308 | P6 | H | P1 | H | P1 | P6 | NMe |
| 309 | P6 | H | P2 | H | P2 | P6 | NMe |
| 310 | P6 | H | P3 | H | P3 | P6 | NMe |
| 311 | P6 | H | P4 | H | P4 | P6 | NMe |
| 312 | P6 | H | P5 | H | P5 | P6 | NMe |
| 313 | P6 | H | P6 | H | P6 | P6 | NMe |
| 314 | P6 | H | P7 | H | P7 | P6 | NMe |
| 315 | P6 | H | P8 | H | P8 | P6 | NMe |
| 316 | P6 | H | P9 | H | P9 | P6 | NMe |
| 317 | P6 | H | P10 | H | P10 | P6 | NMe |
| 318 | P6 | H | P11 | H | P11 | P6 | NMe |
| 319 | P6 | H | P12 | H | P12 | P6 | NMe |
| 320 | P6 | H | P13 | H | P13 | P6 | NMe |
| 321 | P6 | H | P14 | H | P14 | P6 | NMe |
| 322 | P6 | H | P15 | H | P15 | P6 | NMe |
| 323 | P6 | H | P16 | H | P16 | P6 | NMe |
| 324 | P6 | H | P17 | H | P17 | P6 | NMe |
| 325 | P6 | H | P18 | H | P18 | P6 | NMe |
| 326 | P6 | H | P19 | H | P19 | P6 | NMe |
| 327 | P6 | H | P20 | H | P20 | P6 | NMe |
| 328 | P6 | H | F | H | F | P6 | NMe |
| 329 | H | P6 | P1 | H | P6 | P6 | NMe |
| 330 | H | P6 | P2 | H | P6 | P6 | NMe |
| 331 | H | P6 | P3 | H | P6 | P6 | NMe |
| 332 | H | P6 | P4 | H | P6 | P6 | NMe |
| 333 | H | P6 | P5 | H | P6 | P6 | NMe |
| 334 | H | P6 | P6 | H | P6 | P6 | NMe |
| 335 | H | P6 | P7 | H | P6 | P6 | NMe |
| 336 | H | P6 | P8 | H | P6 | P6 | NMe |
| 337 | H | P6 | P9 | H | P6 | P6 | NMe |
| 338 | H | P6 | P10 | H | P6 | P6 | NMe |
| 339 | H | P6 | P11 | H | P6 | P6 | NMe |
| 340 | H | P6 | P12 | H | P6 | P6 | NMe |
| 341 | H | P6 | P13 | H | P6 | P6 | NMe |
| 342 | H | P6 | P14 | H | P6 | P6 | NMe |
| 343 | H | P6 | P15 | H | P6 | P6 | NMe |
| 344 | H | P6 | P16 | H | P6 | P6 | NMe |
| 345 | H | P6 | P17 | H | P6 | P6 | NMe |
| 346 | H | P6 | P18 | H | P6 | P6 | NMe |
| 347 | H | P6 | P19 | H | P6 | P6 | NMe |
| 348 | H | P6 | P20 | H | P6 | P6 | NMe |
| 349 | H | P6 | F | H | P6 | P6 | NMe |
| 350 | P6 | P6 | P6 | H | P1 | P6 | NMe |
| 351 | P6 | P6 | P6 | H | P2 | P6 | NMe |
| 352 | P6 | P6 | P6 | H | P3 | P6 | NMe |
| 353 | P6 | P6 | P6 | H | P4 | P6 | NMe |
| 354 | P6 | P6 | P6 | H | P5 | P6 | NMe |
| 355 | P6 | P6 | P6 | H | P6 | P6 | NMe |
| 356 | P6 | P6 | P6 | H | P7 | P6 | NMe |
| 357 | P6 | P6 | P6 | H | P8 | P6 | NMe |
| 358 | P6 | P6 | P6 | H | P9 | P6 | NMe |
| 359 | P6 | P6 | P6 | H | P10 | P6 | NMe |
| 360 | P6 | P6 | P6 | H | P11 | P6 | NMe |
| 361 | P6 | P6 | P6 | H | P12 | P6 | NMe |
| 362 | P6 | P6 | P6 | H | P13 | P6 | NMe |
| 363 | P6 | P6 | P6 | H | P14 | P6 | NMe |
| 364 | P6 | P6 | P6 | H | P15 | P6 | NMe |
| 365 | P6 | P6 | P6 | H | P16 | P6 | NMe |
| 366 | P6 | P6 | P6 | H | P17 | P6 | NMe |
| 367 | P6 | P6 | P6 | H | P18 | P6 | NMe |
| 368 | P6 | P6 | P6 | H | P19 | P6 | NMe |
| 369 | P6 | P6 | P6 | H | P20 | P6 | NMe |
| 370 | P6 | P6 | P6 | H | F | P6 | NMe |
| 371 | P1 | P1 | P1 | H | P1 | P1 | NMe |
| 372 | P2 | P2 | P2 | H | P2 | P2 | NMe |
| 373 | P3 | P3 | P3 | H | P3 | P3 | NMe |
| 374 | P4 | P4 | P4 | H | P4 | P4 | NMe |
| 375 | P5 | P5 | P5 | H | P5 | P5 | NMe |
| 376 | P6 | P6 | P6 | H | P6 | P6 | NMe |
| 377 | P7 | P7 | P7 | H | P7 | P7 | NMe |
| 378 | P8 | P8 | P8 | H | P8 | P8 | NMe |
| 379 | P9 | P9 | P9 | H | P9 | P9 | NMe |
| 380 | P10 | P10 | P10 | H | P10 | P10 | NMe |
| 381 | P11 | P11 | P11 | H | P11 | P11 | NMe |
| 382 | H | H | H | H | H | H | Se |
| 383 | P6 | P6 | P6 | P6 | P6 | P6 | Se |
| 384 | P6 | P6 | P20 | P6 | P6 | P6 | Se |
| 385 | P6 | P6 | P19 | P6 | P6 | P6 | Se |
| 386 | P6 | P6 | F | P6 | P6 | P6 | Se |
| 387 | P6 | P6 | P6 | P20 | P6 | P6 | Se |
| 388 | P6 | P6 | P6 | P19 | P6 | P6 | Se |
| 389 | P6 | P6 | P6 | F | P6 | P6 | Se |
| 390 | P6 | P6 | P17 | P6 | P6 | P6 | Se |

wherein P1 to P20 have the following structures: 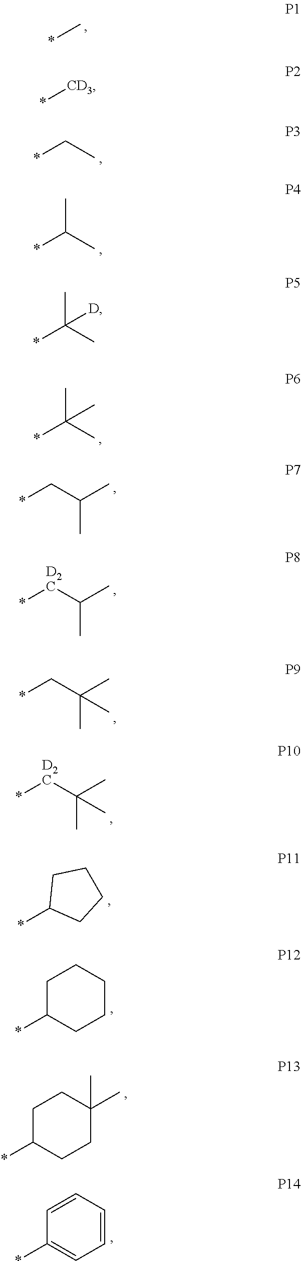 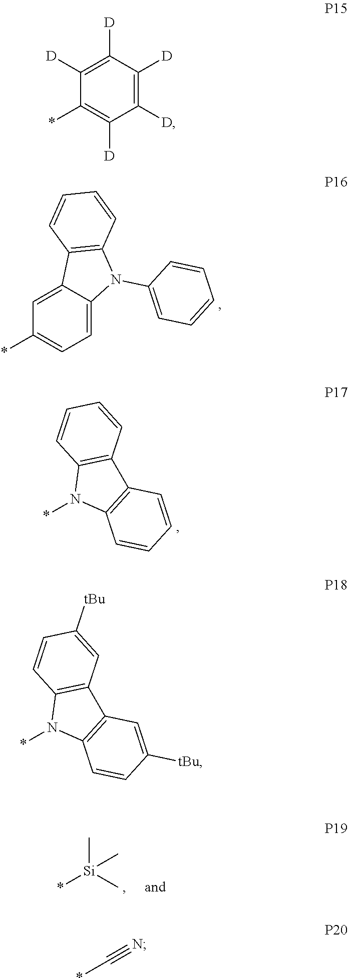
optionally, hydrogens in the above compounds can be partially or fully substituted with deuterium.

10. An organic electroluminescent device, comprising:
an anode,
a cathode, and
an organic layer disposed between the anode and the cathode, wherein the organic layer comprises the compound according to claim 1.

11. The organic electroluminescent device according to claim 10, wherein the organic layer is an emissive layer, and the compound is a light-emitting material;
preferably, the organic electroluminescent device emits red or white light.

12. The organic electroluminescent device according to claim 11, wherein the emissive layer comprises at least a first host material;
preferably, the first host material has a structure represented by Formula PH-1, Formula PH-2, or Formula PH-3: 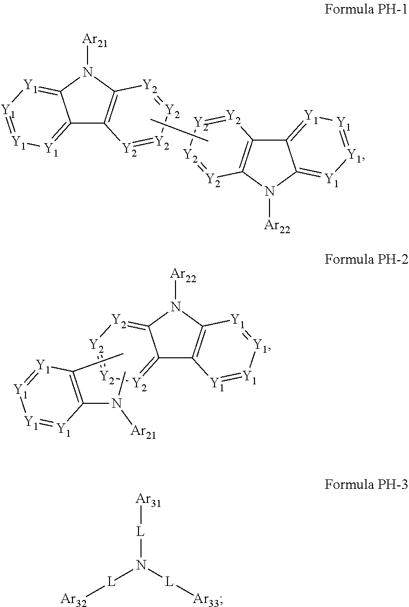
wherein Y₁ is, at each occurrence identically or differently, selected from CR_{y} or N;
Y₂ is, at each occurrence identically or differently, selected from C, CR_{y}, or N;
L is, at each occurrence identically or differently, selected from a single bond, substituted or unsubstituted arylene having 6 to 30 carbon atoms, substituted or unsubstituted heteroarylene having 3 to 30 carbon atoms, or a combination thereof;
Ar₂₁, Ar₂₂, Ar₃₁, Ar₃₂, and Ar₃₃ are, at each occurrence identically or differently, selected from substituted or unsubstituted aryl having 6 to 30 carbon atoms or substituted or unsubstituted heteroaryl having 3 to 30 carbon atoms;
R_{y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof; and
adjacent substituents R_{y} can be optionally joined to form a ring;
more preferably, the first host material is selected from the group consisting of Compound 1-1-1 to Compound 1-1-104, Compound 1-2-1 to Compound 1-2-100, and Compound 1-3-1 to Compound 1-3-110: 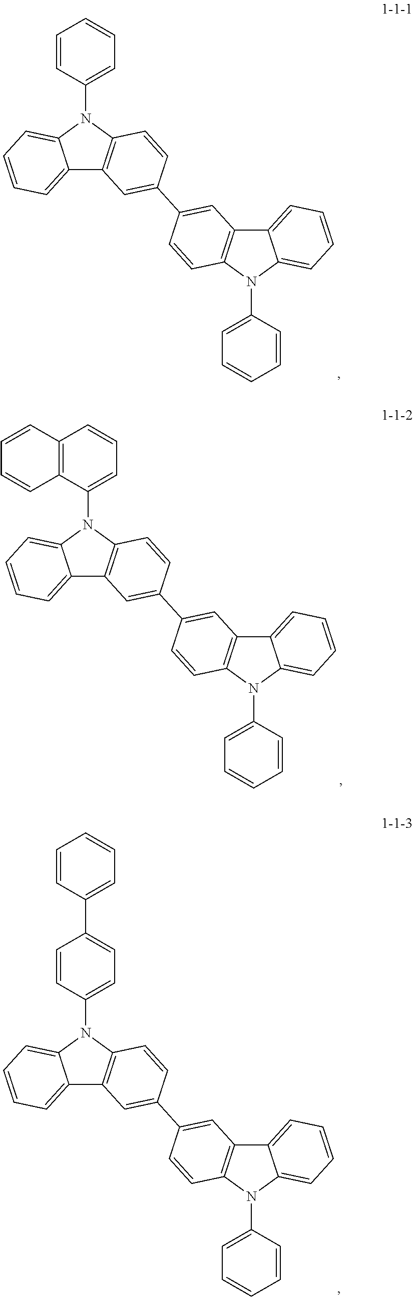 [SMILES: c1ccc(-n2c3ccccc3c3cc(-c4ccc5c(c4)c4ccccc4n5-c4ccccc4)ccc32)cc1 ; c1ccc(-n2c3ccccc3c3cc(-c4ccc5c(c4)c4ccccc4n5-c4cccc5ccccc45)ccc32)cc1 ; c1ccc(-c2ccc(-n3c4ccccc4c4cc(-c5ccc6c(c5)c5ccccc5n6-c5ccccc5)ccc43)cc2)cc1] 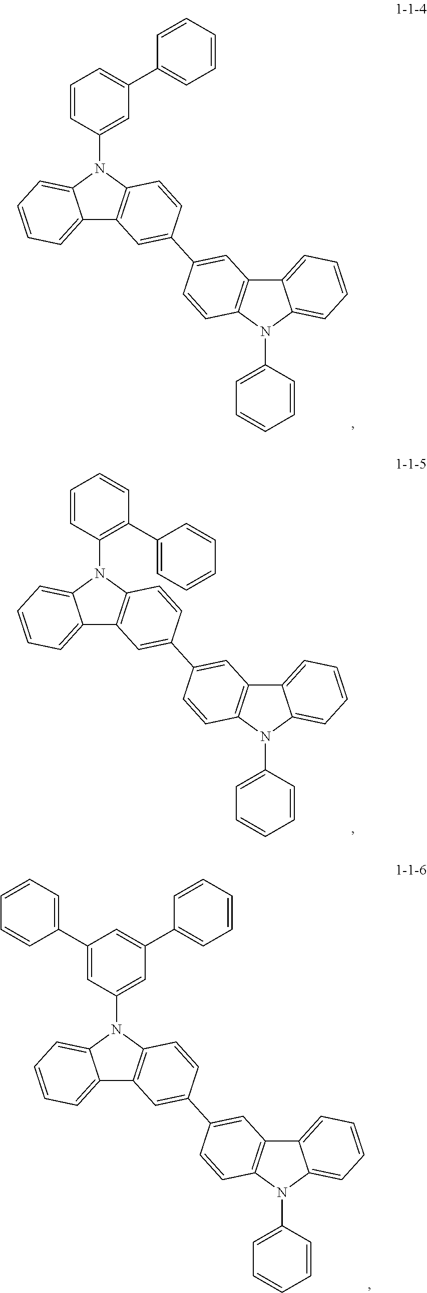 [SMILES: c1ccc(-c2cccc(-n3c4ccccc4c4cc(-c5ccc6c(c5)c5ccccc5n6-c5ccccc5)ccc43)c2)cc1 ; c1ccc(-c2ccccc2-n2c3ccccc3c3cc(-c4ccc5c(c4)c4ccccc4n5-c4ccccc4)ccc32)cc1 ; c1ccc(-c2cc(-c3ccccc3)cc(-n3c4ccccc4c4cc(-c5ccc6c(c5)c5ccccc5n6-c5ccccc5)ccc43)c2)cc1] 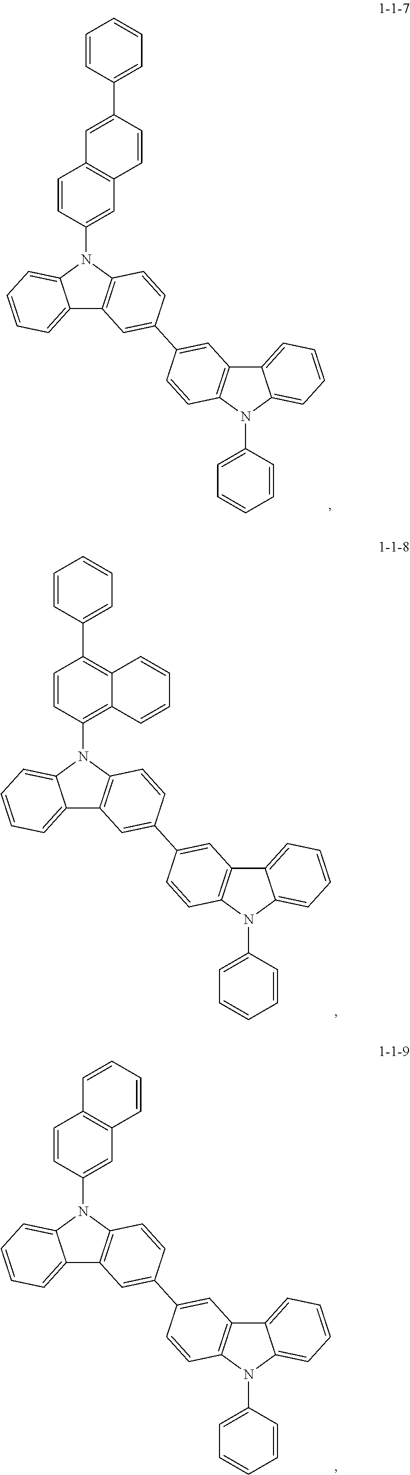 [SMILES: c1ccc(-c2ccc3cc(-n4c5ccccc5c5cc(-c6ccc7c(c6)c6ccccc6n7-c6ccccc6)ccc54)ccc3c2)cc1 ; c1ccc(-c2ccc(-n3c4ccccc4c4cc(-c5ccc6c(c5)c5ccccc5n6-c5ccccc5)ccc43)c3ccccc23)cc1 ; c1ccc(-n2c3ccccc3c3cc(-c4ccc5c(c4)c4ccccc4n5-c4ccc5ccccc5c4)ccc32)cc1] 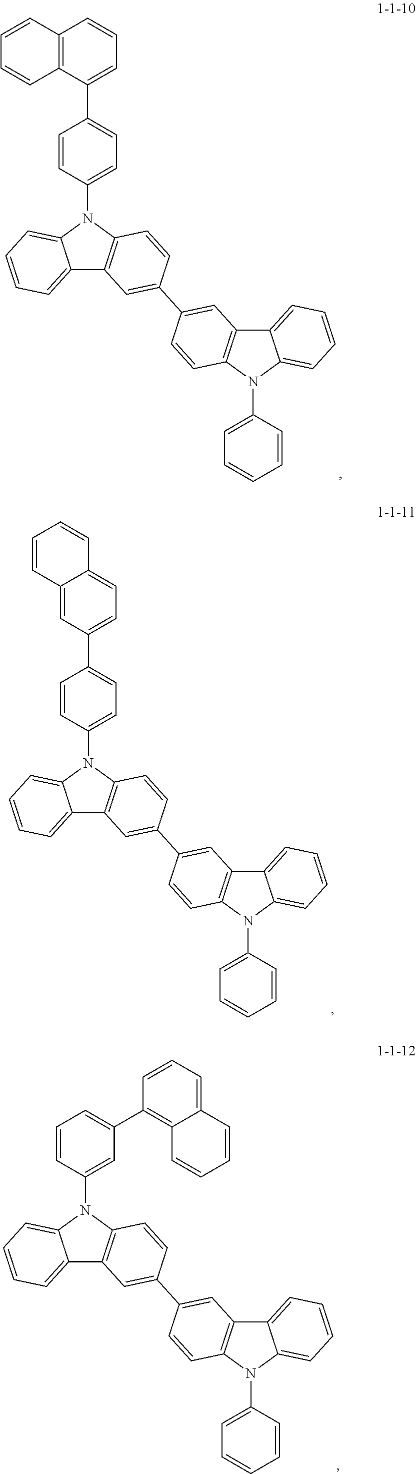 [SMILES: c1ccc(-n2c3ccccc3c3cc(-c4ccc5c(c4)c4ccccc4n5-c4ccc(-c5cccc6ccccc56)cc4)ccc32)cc1 ; c1ccc(-n2c3ccccc3c3cc(-c4ccc5c(c4)c4ccccc4n5-c4ccc(-c5ccc6ccccc6c5)cc4)ccc32)cc1 ; c1ccc(-n2c3ccccc3c3cc(-c4ccc5c(c4)c4ccccc4n5-c4cccc(-c5cccc6ccccc56)c4)ccc32)cc1] 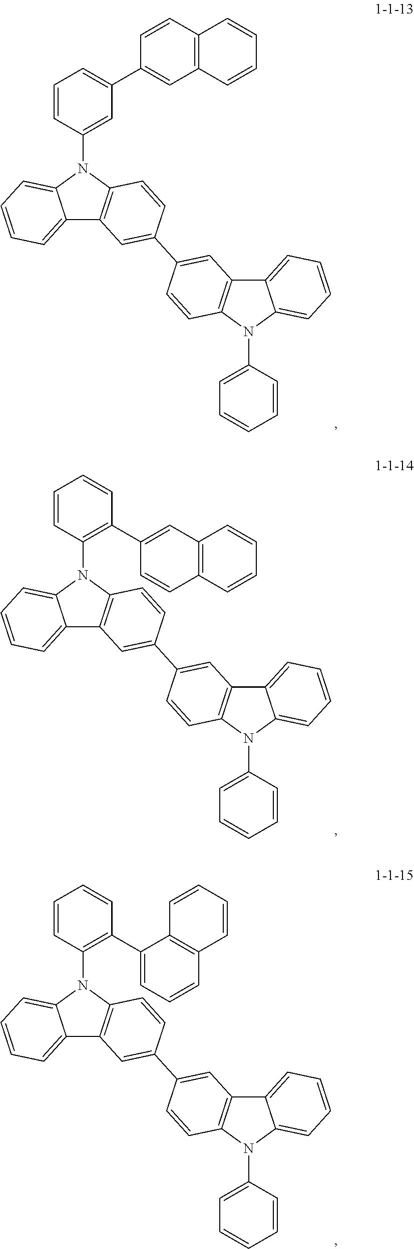 [SMILES: c1ccc(-n2c3ccccc3c3cc(-c4ccc5c(c4)c4ccccc4n5-c4cccc(-c5ccc6ccccc6c5)c4)ccc32)cc1 ; c1ccc(-n2c3ccccc3c3cc(-c4ccc5c(c4)c4ccccc4n5-c4ccccc4-c4ccc5ccccc5c4)ccc32)cc1 ; c1ccc(-n2c3ccccc3c3cc(-c4ccc5c(c4)c4ccccc4n5-c4ccccc4-c4cccc5ccccc45)ccc32)cc1] 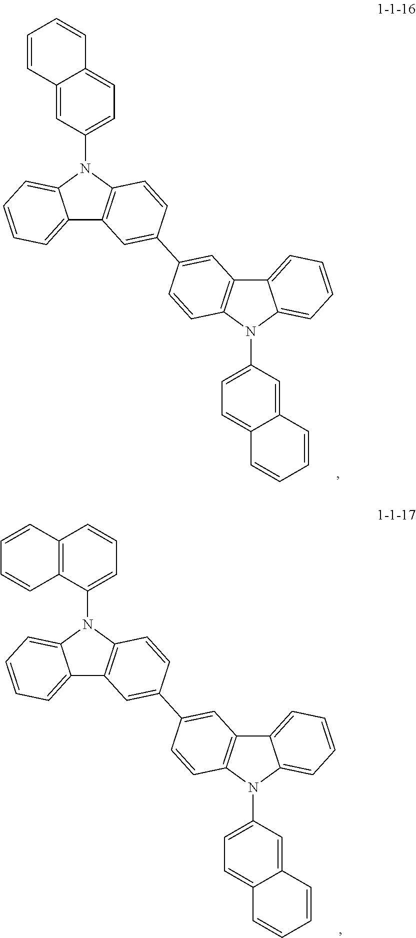 [SMILES: c1ccc2cc(-n3c4ccccc4c4cc(-c5ccc6c(c5)c5ccccc5n6-c5ccc6ccccc6c5)ccc43)ccc2c1 ; c1ccc2cc(-n3c4ccccc4c4cc(-c5ccc6c(c5)c5ccccc5n6-c5cccc6ccccc56)ccc43)ccc2c1] 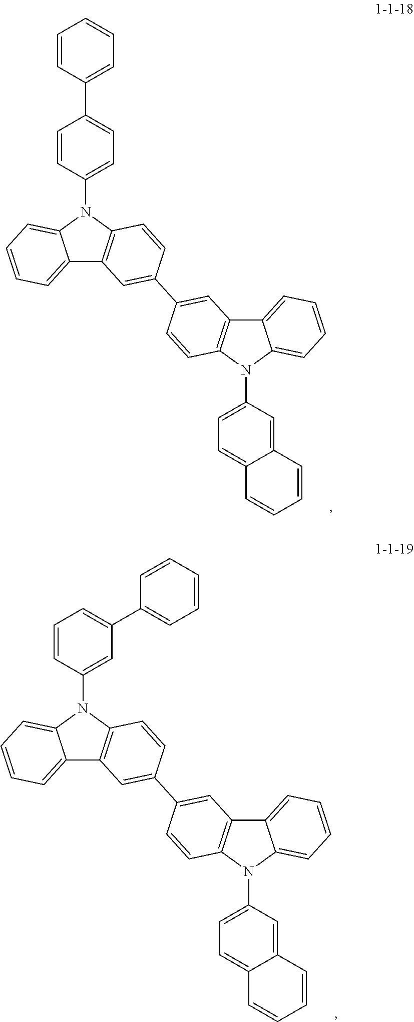 [SMILES: c1ccc(-c2ccc(-n3c4ccccc4c4cc(-c5ccc6c(c5)c5ccccc5n6-c5ccc6ccccc6c5)ccc43)cc2)cc1 ; c1ccc(-c2cccc(-n3c4ccccc4c4cc(-c5ccc6c(c5)c5ccccc5n6-c5ccc6ccccc6c5)ccc43)c2)cc1] 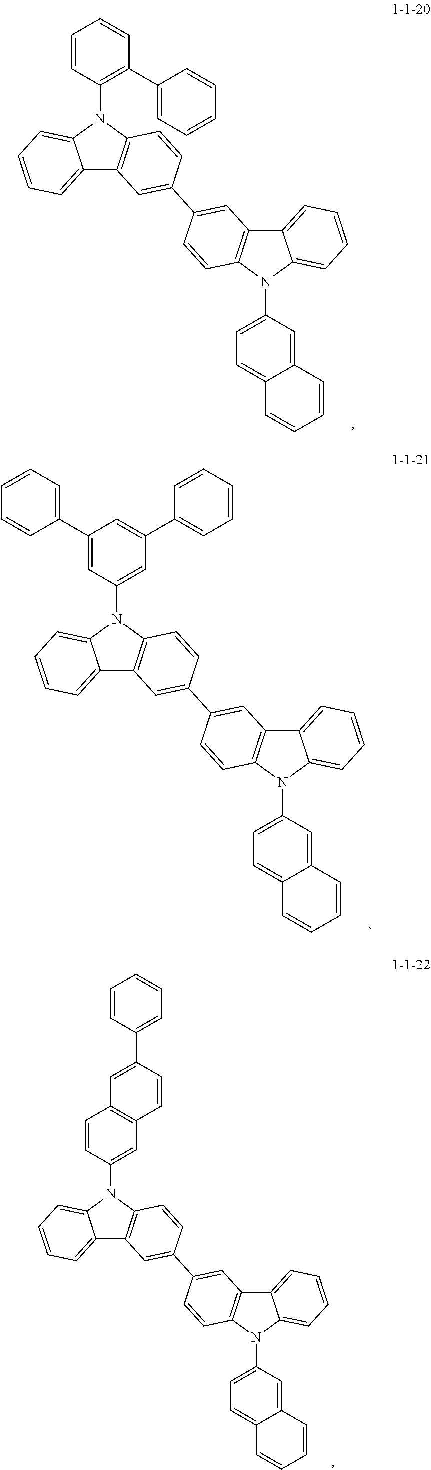 [SMILES: c1ccc(-c2ccccc2-n2c3ccccc3c3cc(-c4ccc5c(c4)c4ccccc4n5-c4ccc5ccccc5c4)ccc32)cc1 ; c1ccc(-c2cc(-c3ccccc3)cc(-n3c4ccccc4c4cc(-c5ccc6c(c5)c5ccccc5n6-c5ccc6ccccc6c5)ccc43)c2)cc1 ; c1ccc(-c2ccc3cc(-n4c5ccccc5c5cc(-c6ccc7c(c6)c6ccccc6n7-c6ccc7ccccc7c6)ccc54)ccc3c2)cc1] 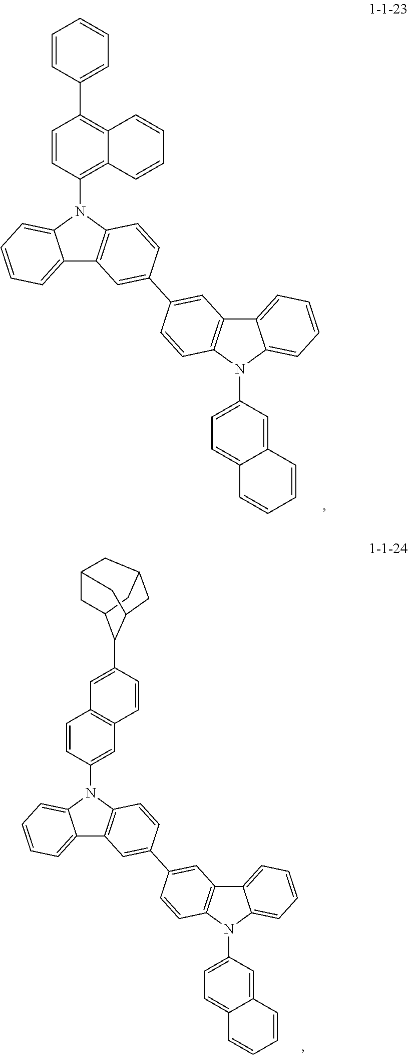 [SMILES: c1ccc(-c2ccc(-n3c4ccccc4c4cc(-c5ccc6c(c5)c5ccccc5n6-c5ccc6ccccc6c5)ccc43)c3ccccc23)cc1 ; c1ccc2cc(-n3c4ccccc4c4cc(-c5ccc6c(c5)c5ccccc5n6-c5ccc6cc(C7C8CC9CC(C8)CC7C9)ccc6c5)ccc43)ccc2c1] 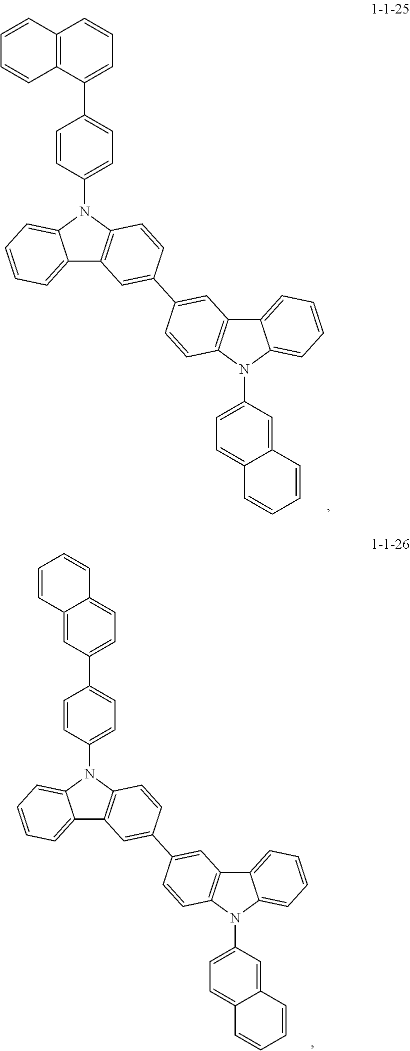 [SMILES: c1ccc2cc(-n3c4ccccc4c4cc(-c5ccc6c(c5)c5ccccc5n6-c5ccc(-c6cccc7ccccc67)cc5)ccc43)ccc2c1 ; c1ccc2cc(-c3ccc(-n4c5ccccc5c5cc(-c6ccc7c(c6)c6ccccc6n7-c6ccc7ccccc7c6)ccc54)cc3)ccc2c1] 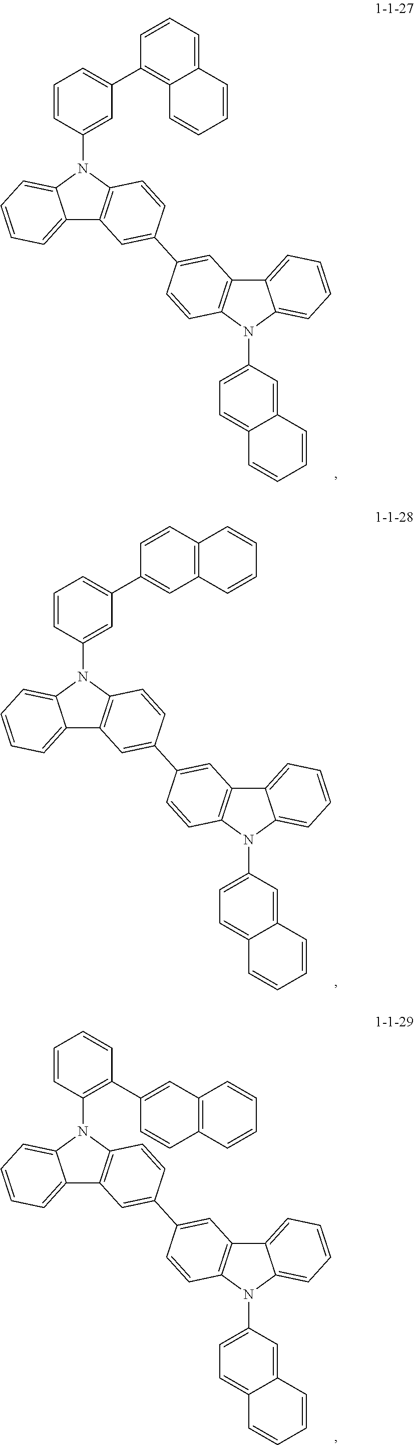 [SMILES: c1cc(-c2cccc3ccccc23)cc(-n2c3ccccc3c3cc(-c4ccc5c(c4)c4ccccc4n5-c4ccc5ccccc5c4)ccc32)c1 ; c1cc(-c2ccc3ccccc3c2)cc(-n2c3ccccc3c3cc(-c4ccc5c(c4)c4ccccc4n5-c4ccc5ccccc5c4)ccc32)c1 ; c1ccc(-n2c3ccccc3c3cc(-c4ccc5c(c4)c4ccccc4n5-c4ccc5ccccc5c4)ccc32)c(-c2ccc3ccccc3c2)c1] 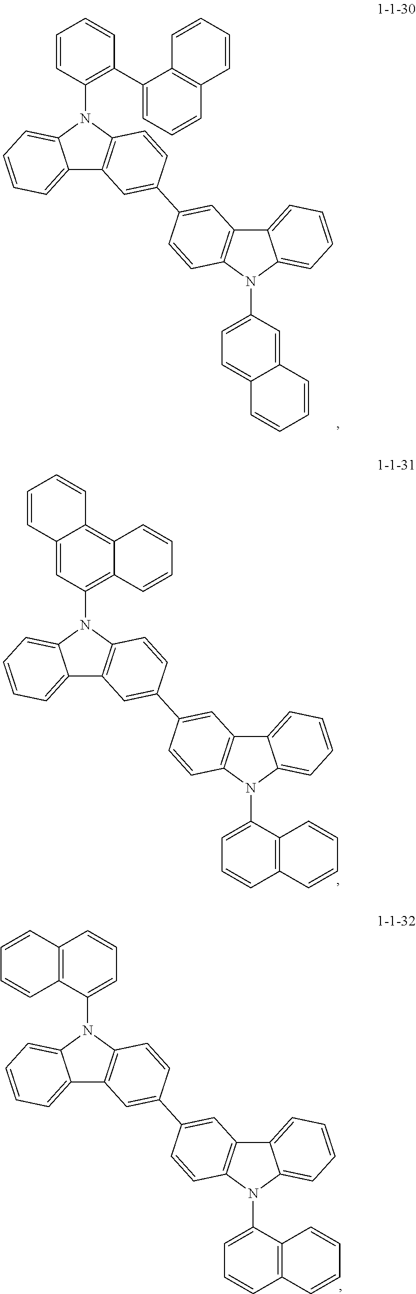 [SMILES: c1ccc(-n2c3ccccc3c3cc(-c4ccc5c(c4)c4ccccc4n5-c4ccc5ccccc5c4)ccc32)c(-c2cccc3ccccc23)c1 ; c1ccc2c(-n3c4ccccc4c4cc(-c5ccc6c(c5)c5ccccc5n6-c5cc6ccccc6c6ccccc56)ccc43)cccc2c1 ; c1ccc2c(-n3c4ccccc4c4cc(-c5ccc6c(c5)c5ccccc5n6-c5cccc6ccccc56)ccc43)cccc2c1] 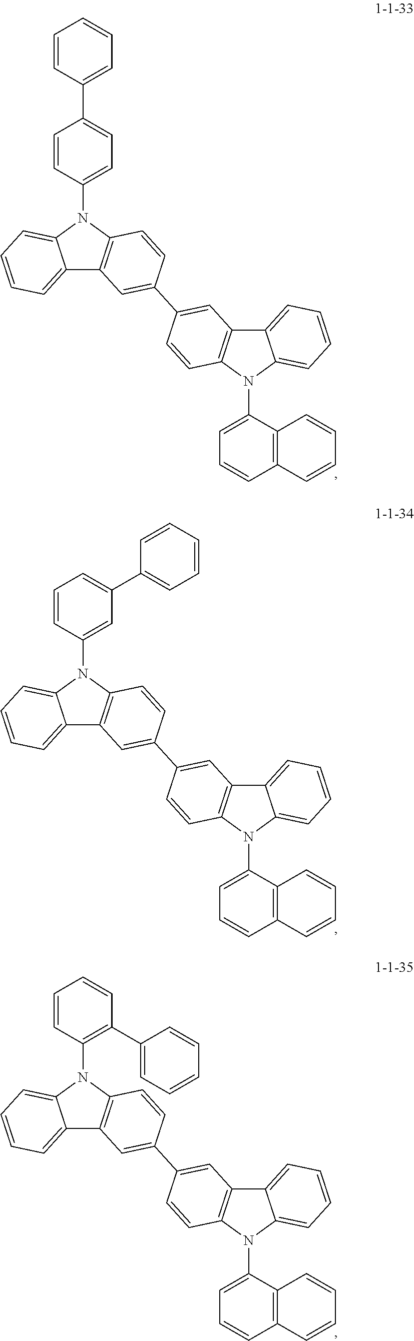 [SMILES: c1ccc(-c2ccc(-n3c4ccccc4c4cc(-c5ccc6c(c5)c5ccccc5n6-c5cccc6ccccc56)ccc43)cc2)cc1 ; c1ccc(-c2cccc(-n3c4ccccc4c4cc(-c5ccc6c(c5)c5ccccc5n6-c5cccc6ccccc56)ccc43)c2)cc1 ; c1ccc(-c2ccccc2-n2c3ccccc3c3cc(-c4ccc5c(c4)c4ccccc4n5-c4cccc5ccccc45)ccc32)cc1] 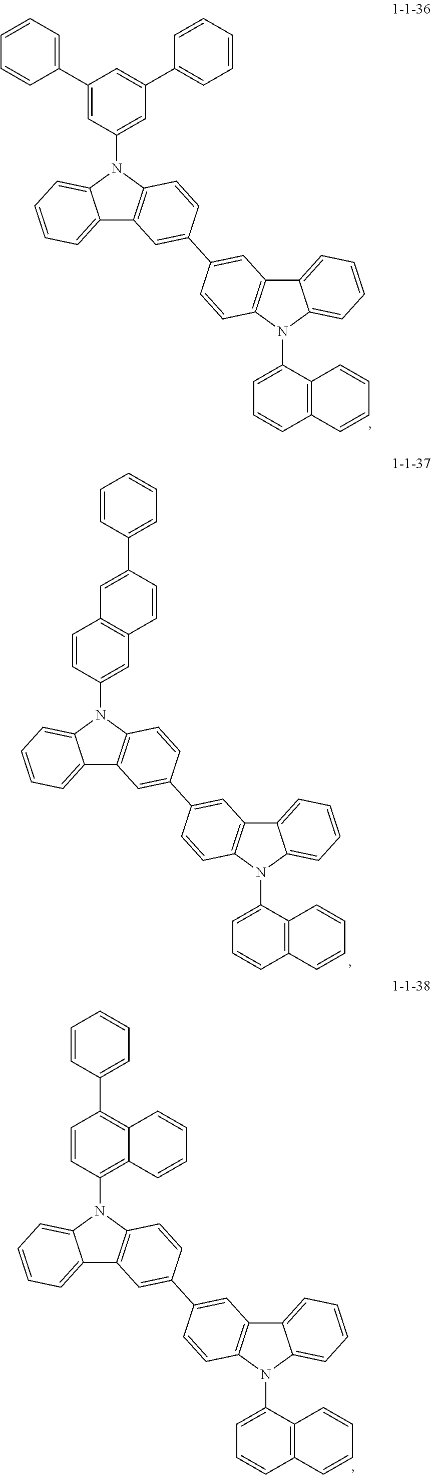 [SMILES: c1ccc(-c2cc(-c3ccccc3)cc(-n3c4ccccc4c4cc(-c5ccc6c(c5)c5ccccc5n6-c5cccc6ccccc56)ccc43)c2)cc1 ; c1ccc(-c2ccc3cc(-n4c5ccccc5c5cc(-c6ccc7c(c6)c6ccccc6n7-c6cccc7ccccc67)ccc54)ccc3c2)cc1 ; c1ccc(-c2ccc(-n3c4ccccc4c4cc(-c5ccc6c(c5)c5ccccc5n6-c5cccc6ccccc56)ccc43)c3ccccc23)cc1] 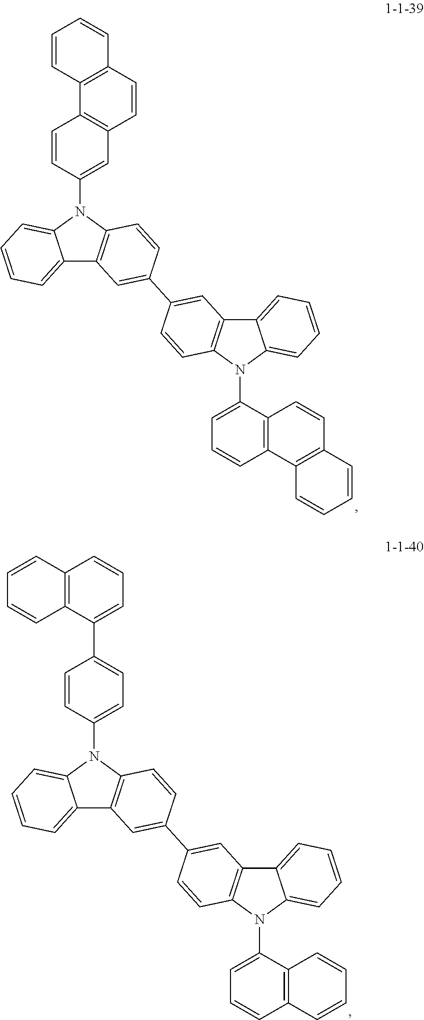 [SMILES: c1ccc2c(c1)ccc1cc(-n3c4ccccc4c4cc(-c5ccc6c(c5)c5ccccc5n6-c5cccc6c5ccc5ccccc56)ccc43)ccc12 ; c1ccc2c(-c3ccc(-n4c5ccccc5c5cc(-c6ccc7c(c6)c6ccccc6n7-c6cccc7ccccc67)ccc54)cc3)cccc2c1] 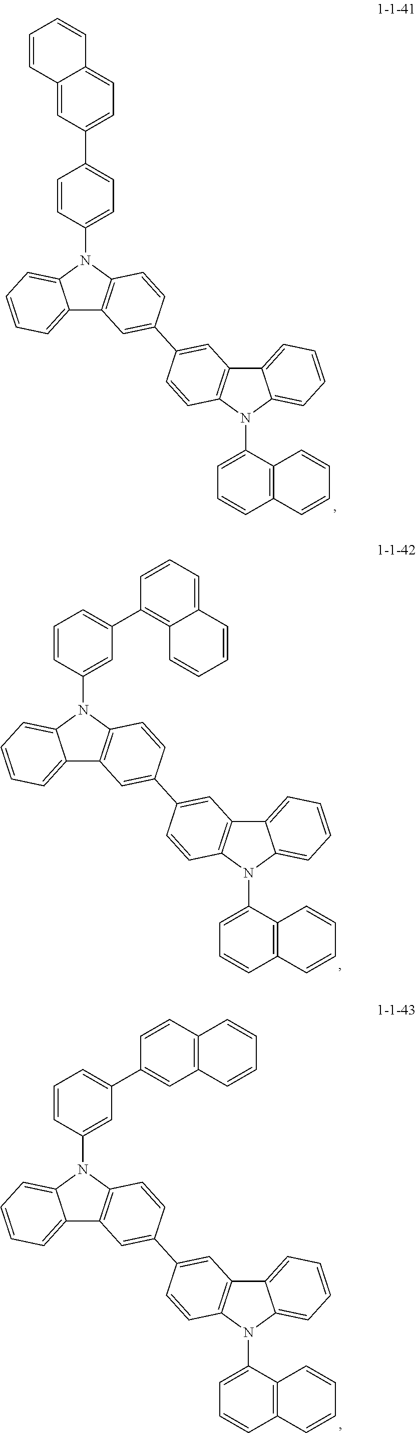 [SMILES: c1ccc2cc(-c3ccc(-n4c5ccccc5c5cc(-c6ccc7c(c6)c6ccccc6n7-c6cccc7ccccc67)ccc54)cc3)ccc2c1 ; c1cc(-c2cccc3ccccc23)cc(-n2c3ccccc3c3cc(-c4ccc5c(c4)c4ccccc4n5-c4cccc5ccccc45)ccc32)c1 ; c1cc(-c2ccc3ccccc3c2)cc(-n2c3ccccc3c3cc(-c4ccc5c(c4)c4ccccc4n5-c4cccc5ccccc45)ccc32)c1] 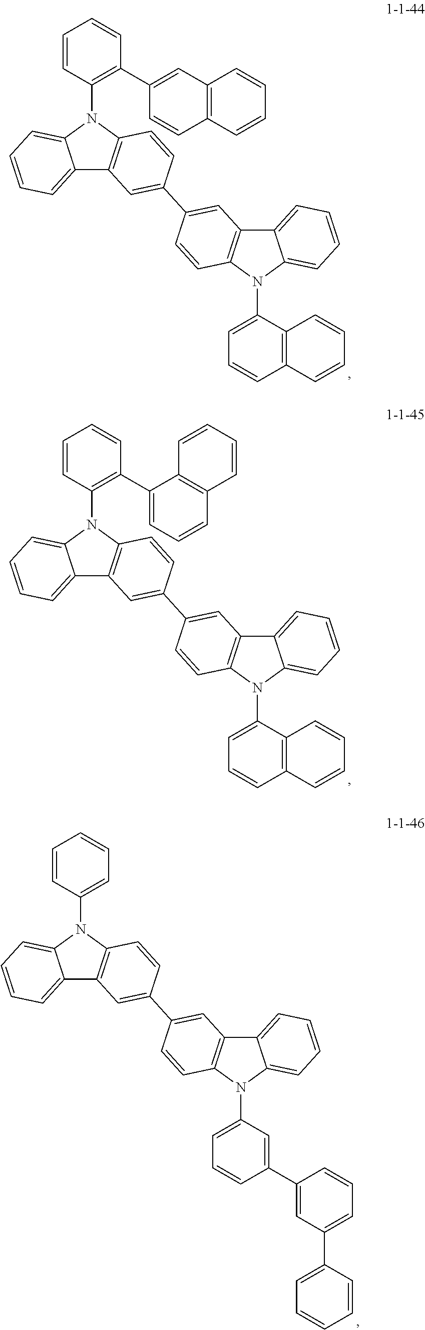 [SMILES: c1ccc(-n2c3ccccc3c3cc(-c4ccc5c(c4)c4ccccc4n5-c4cccc5ccccc45)ccc32)c(-c2ccc3ccccc3c2)c1 ; c1ccc(-n2c3ccccc3c3cc(-c4ccc5c(c4)c4ccccc4n5-c4cccc5ccccc45)ccc32)c(-c2cccc3ccccc23)c1 ; c1ccc(-c2cccc(-c3cccc(-n4c5ccccc5c5cc(-c6ccc7c(c6)c6ccccc6n7-c6ccccc6)ccc54)c3)c2)cc1] 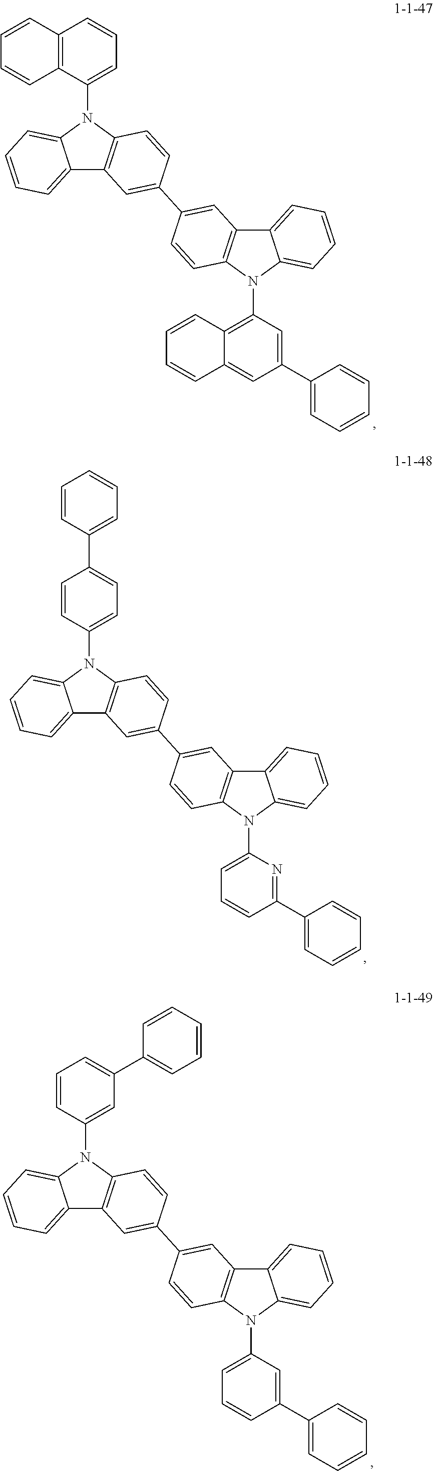 [SMILES: c1ccc(-c2cc(-n3c4ccccc4c4cc(-c5ccc6c(c5)c5ccccc5n6-c5cccc6ccccc56)ccc43)c3ccccc3c2)cc1 ; c1ccc(-c2ccc(-n3c4ccccc4c4cc(-c5ccc6c(c5)c5ccccc5n6-c5cccc(-c6ccccc6)n5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1] 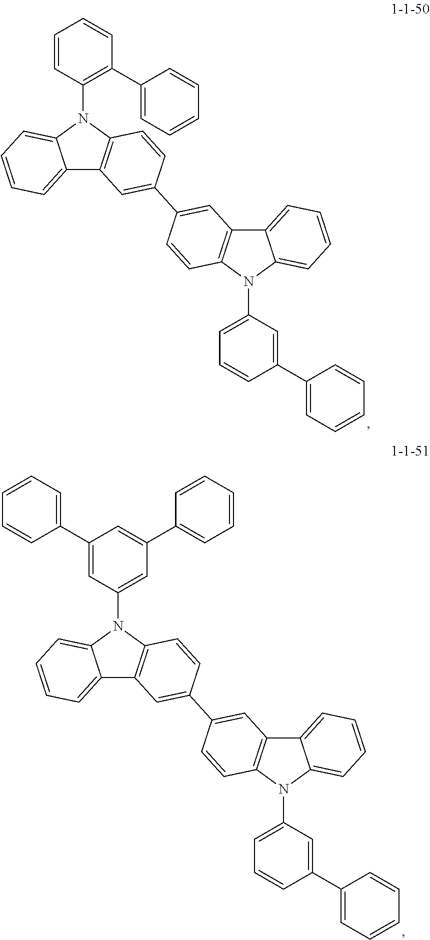 [SMILES: c1ccc(-c2cccc(-n3c4ccccc4c4cc(-c5ccc6c(c5)c5ccccc5n6-c5ccccc5-c5ccccc5)ccc43)c2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1] 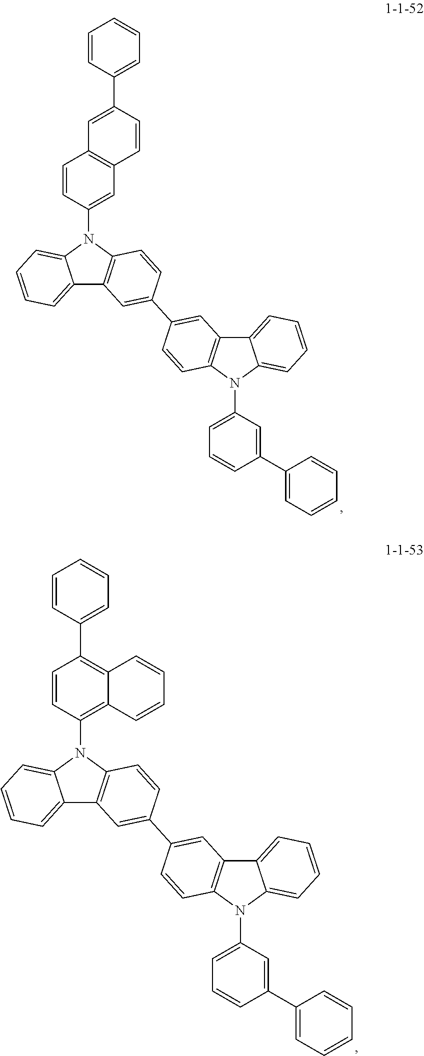 [SMILES: c1ccc(-c2cccc(-n3c4ccccc4c4cc(-c5ccc6c(c5)c5ccccc5n6-c5ccc6cc(-c7ccccc7)ccc6c5)ccc43)c2)cc1 ; c1ccc(-c2cccc(-n3c4ccccc4c4cc(-c5ccc6c(c5)c5ccccc5n6-c5ccc(-c6ccccc6)c6ccccc56)ccc43)c2)cc1] 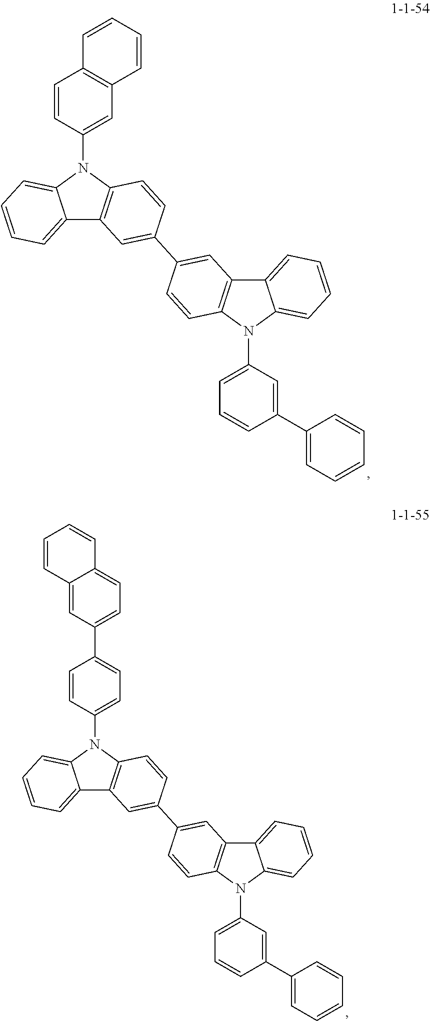 [SMILES: c1ccc(-c2cccc(-n3c4ccccc4c4cc(-c5ccc6c(c5)c5ccccc5n6-c5ccc6ccccc6c5)ccc43)c2)cc1 ; c1ccc(-c2cccc(-n3c4ccccc4c4cc(-c5ccc6c(c5)c5ccccc5n6-c5ccc(-c6ccc7ccccc7c6)cc5)ccc43)c2)cc1] 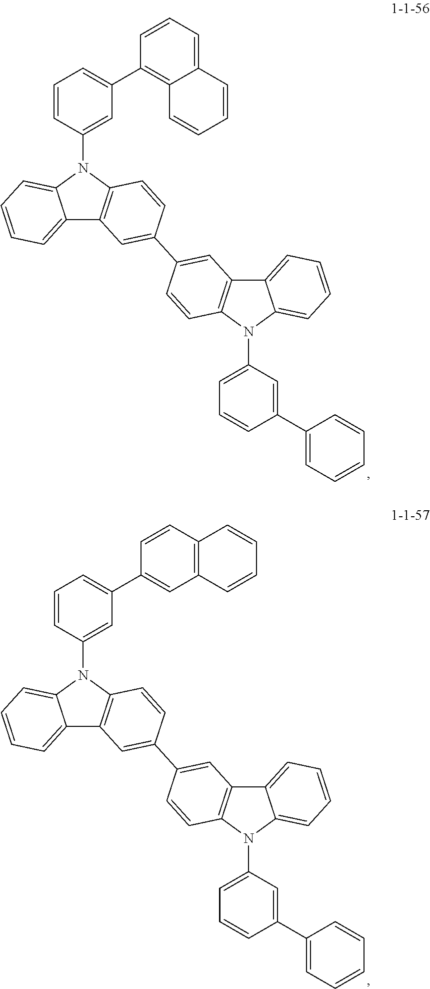 [SMILES: c1ccc(-c2cccc(-n3c4ccccc4c4cc(-c5ccc6c(c5)c5ccccc5n6-c5cccc(-c6cccc7ccccc67)c5)ccc43)c2)cc1 ; c1ccc(-c2cccc(-n3c4ccccc4c4cc(-c5ccc6c(c5)c5ccccc5n6-c5cccc(-c6ccc7ccccc7c6)c5)ccc43)c2)cc1] 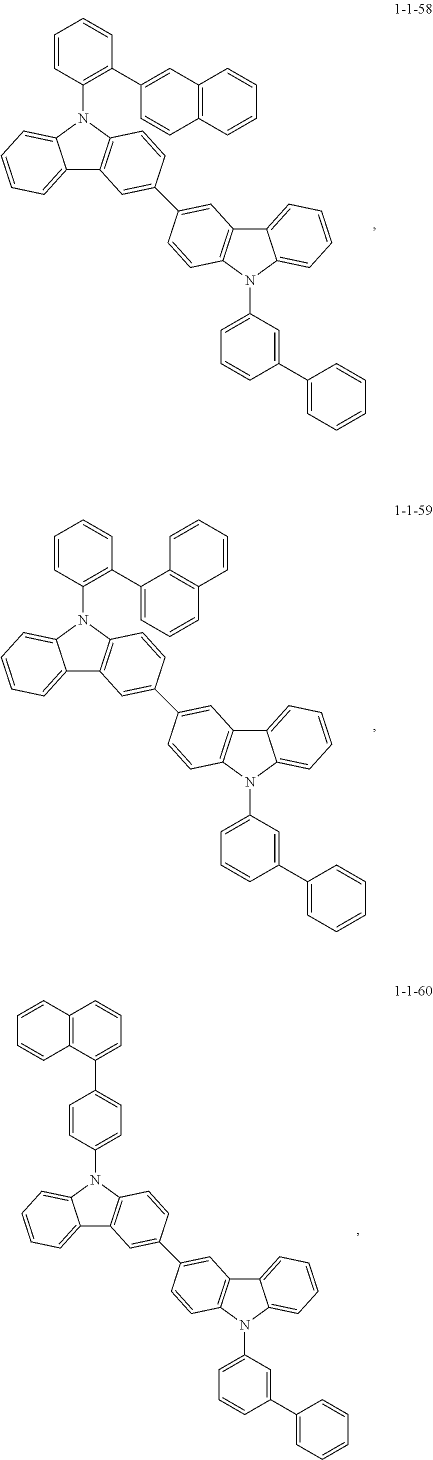 [SMILES: c1ccc(-c2cccc(-n3c4ccccc4c4cc(-c5ccc6c(c5)c5ccccc5n6-c5ccccc5-c5ccc6ccccc6c5)ccc43)c2)cc1 ; c1ccc(-c2cccc(-n3c4ccccc4c4cc(-c5ccc6c(c5)c5ccccc5n6-c5ccccc5-c5cccc6ccccc56)ccc43)c2)cc1 ; c1ccc(-c2cccc(-n3c4ccccc4c4cc(-c5ccc6c(c5)c5ccccc5n6-c5ccc(-c6cccc7ccccc67)cc5)ccc43)c2)cc1] 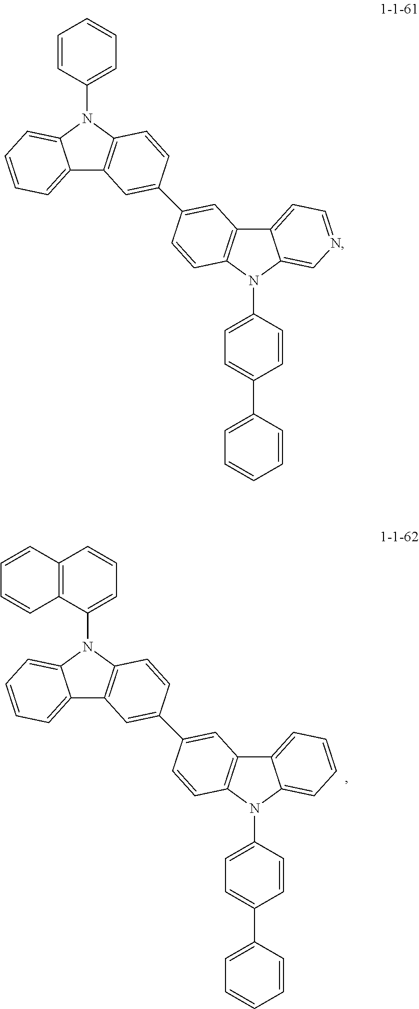 [SMILES: c1ccc(-c2ccc(-n3c4ccc(-c5ccc6c(c5)c5ccccc5n6-c5ccccc5)cc4c4ccncc43)cc2)cc1 ; c1ccc(-c2ccc(-n3c4ccccc4c4cc(-c5ccc6c(c5)c5ccccc5n6-c5cccc6ccccc56)ccc43)cc2)cc1] 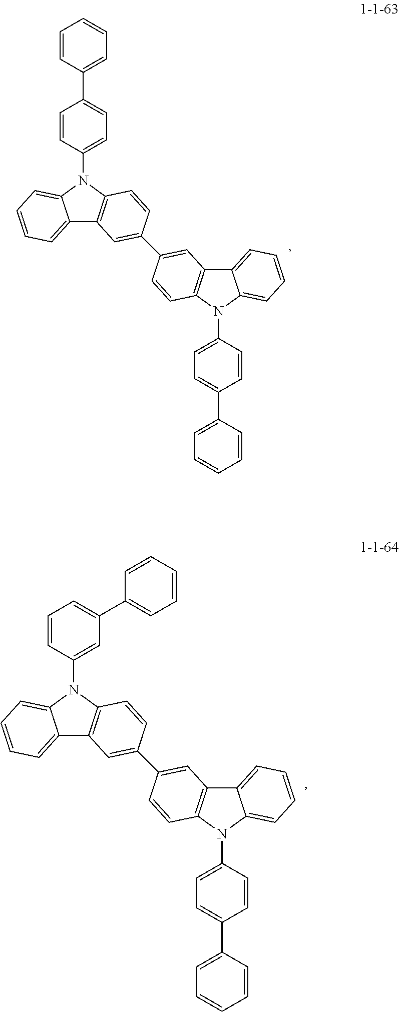 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-n3c4ccccc4c4cc(-c5ccc6c(c5)c5ccccc5n6-c5cccc(-c6ccccc6)c5)ccc43)cc2)cc1] 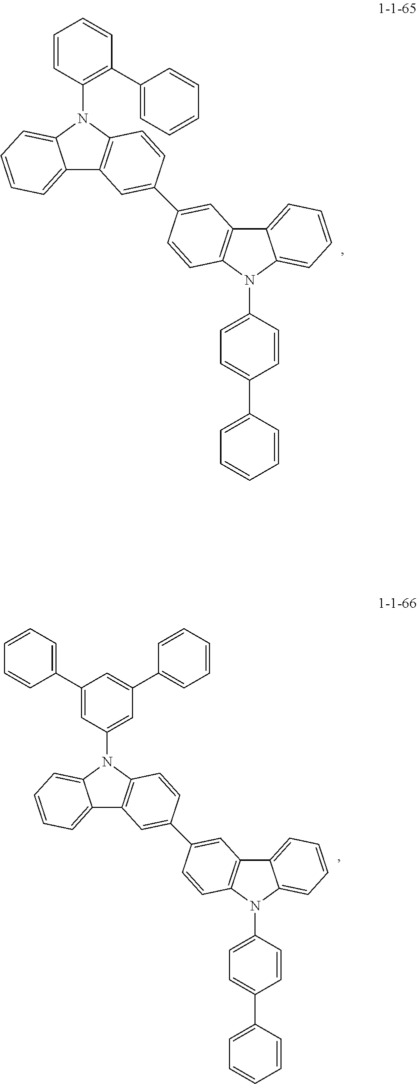 [SMILES: c1ccc(-c2ccc(-n3c4ccccc4c4cc(-c5ccc6c(c5)c5ccccc5n6-c5ccccc5-c5ccccc5)ccc43)cc2)cc1 ; c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1] 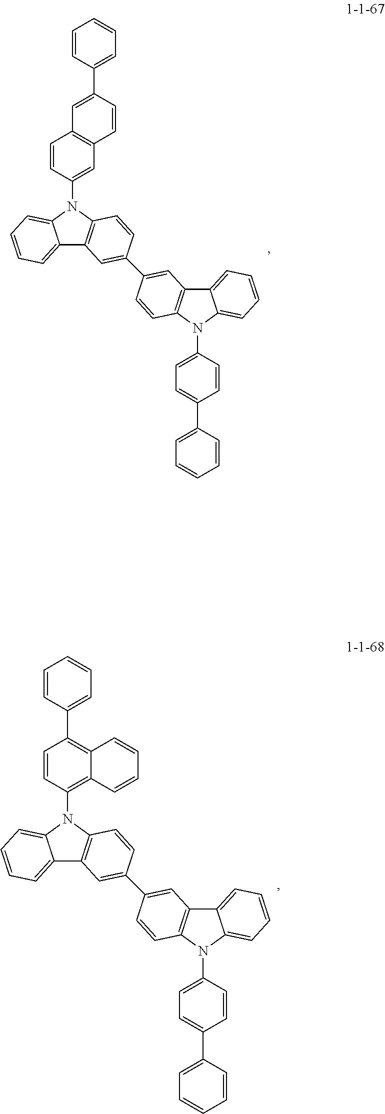 [SMILES: c1ccc(-c2ccc(-n3c4ccccc4c4cc(-c5ccc6c(c5)c5ccccc5n6-c5ccc6cc(-c7ccccc7)ccc6c5)ccc43)cc2)cc1 ; c1ccc(-c2ccc(-n3c4ccccc4c4cc(-c5ccc6c(c5)c5ccccc5n6-c5ccc(-c6ccccc6)c6ccccc56)ccc43)cc2)cc1] 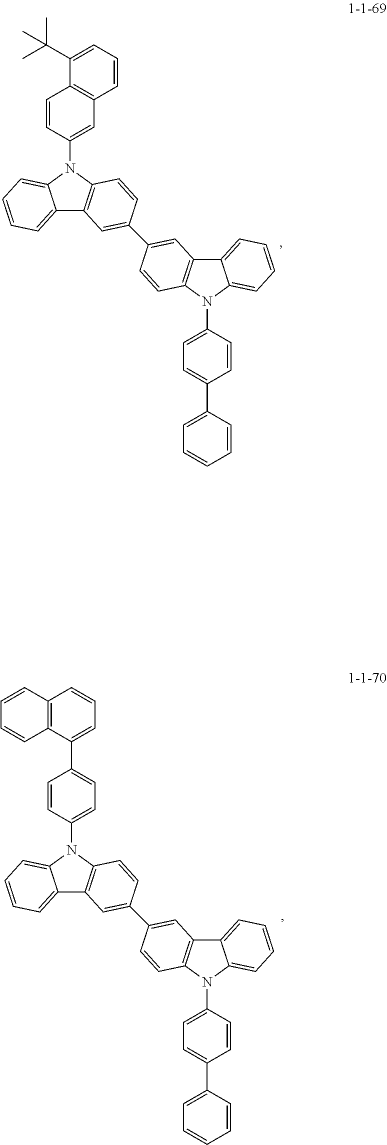 [SMILES: CC(C)(C)c1cccc2cc(-n3c4ccccc4c4cc(-c5ccc6c(c5)c5ccccc5n6-c5ccc(-c6ccccc6)cc5)ccc43)ccc12 ; c1ccc(-c2ccc(-n3c4ccccc4c4cc(-c5ccc6c(c5)c5ccccc5n6-c5ccc(-c6cccc7ccccc67)cc5)ccc43)cc2)cc1] 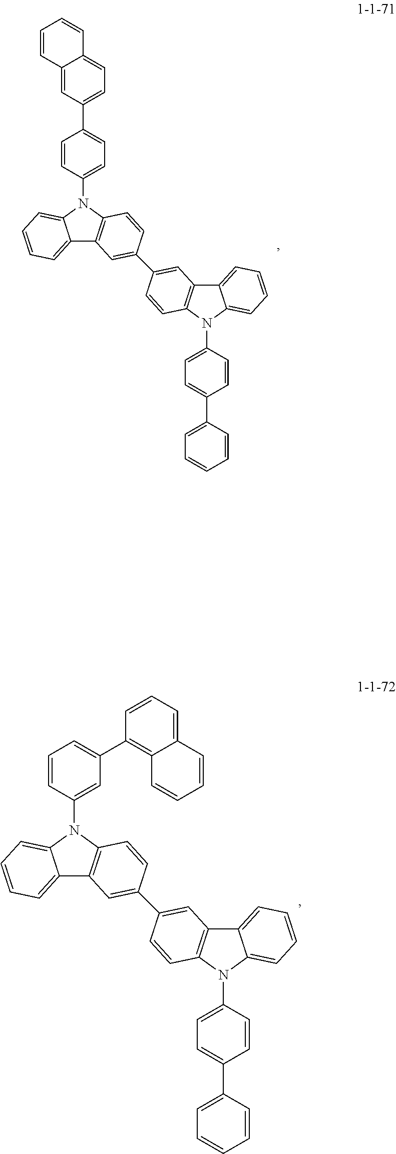 [SMILES: c1ccc(-c2ccc(-n3c4ccccc4c4cc(-c5ccc6c(c5)c5ccccc5n6-c5ccc(-c6ccc7ccccc7c6)cc5)ccc43)cc2)cc1 ; c1ccc(-c2ccc(-n3c4ccccc4c4cc(-c5ccc6c(c5)c5ccccc5n6-c5cccc(-c6cccc7ccccc67)c5)ccc43)cc2)cc1] 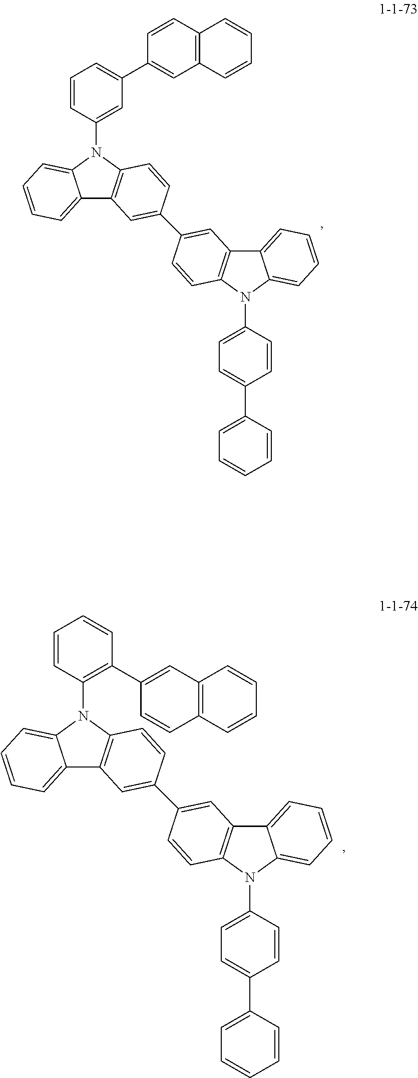 [SMILES: c1ccc(-c2ccc(-n3c4ccccc4c4cc(-c5ccc6c(c5)c5ccccc5n6-c5cccc(-c6ccc7ccccc7c6)c5)ccc43)cc2)cc1 ; c1ccc(-c2ccc(-n3c4ccccc4c4cc(-c5ccc6c(c5)c5ccccc5n6-c5ccccc5-c5ccc6ccccc6c5)ccc43)cc2)cc1] 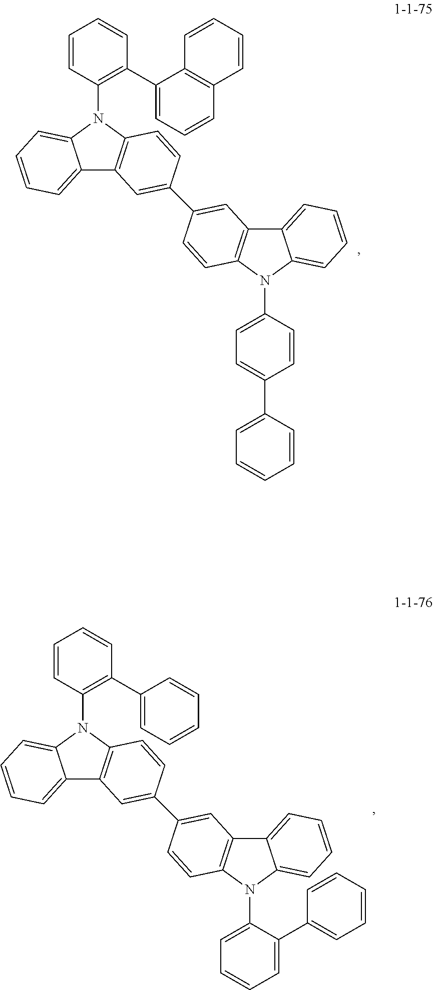 [SMILES: c1ccc(-c2ccc(-n3c4ccccc4c4cc(-c5ccc6c(c5)c5ccccc5n6-c5ccccc5-c5cccc6ccccc56)ccc43)cc2)cc1 ; c1ccc(-c2ccccc2-n2c3ccccc3c3cc(-c4ccc5c(c4)c4ccccc4n5-c4ccccc4-c4ccccc4)ccc32)cc1] 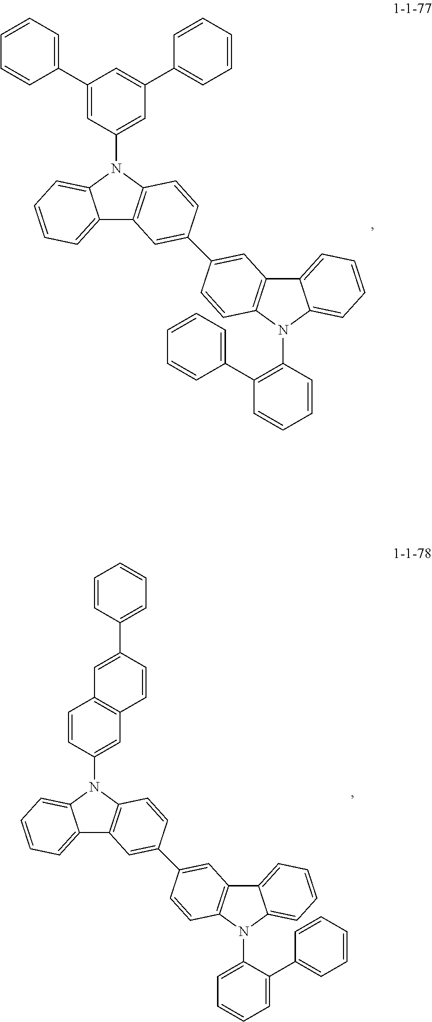 [SMILES: c1ccc(-c2cc(-c3ccccc3)cc(-n3c4ccccc4c4cc(-c5ccc6c(c5)c5ccccc5n6-c5ccccc5-c5ccccc5)ccc43)c2)cc1 ; c1ccc(-c2ccc3cc(-n4c5ccccc5c5cc(-c6ccc7c(c6)c6ccccc6n7-c6ccccc6-c6ccccc6)ccc54)ccc3c2)cc1] 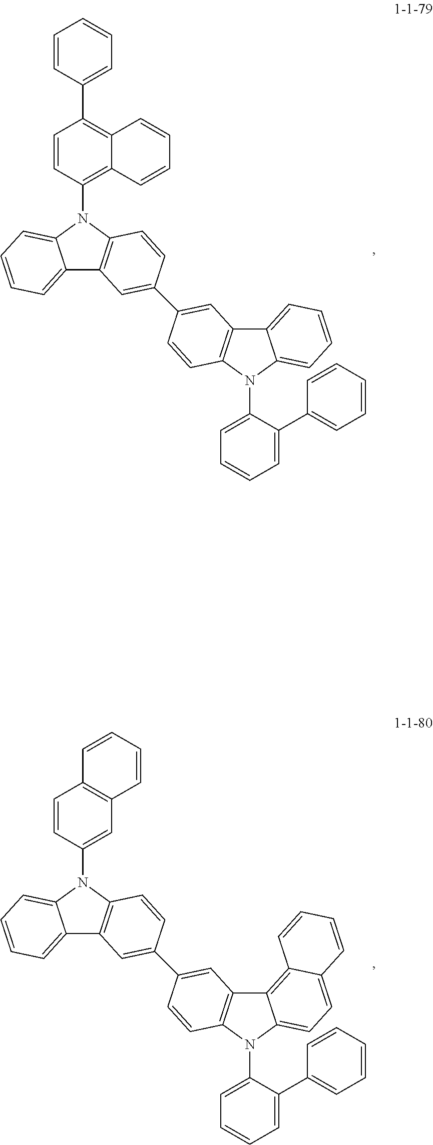 [SMILES: c1ccc(-c2ccccc2-n2c3ccccc3c3cc(-c4ccc5c(c4)c4ccccc4n5-c4ccc(-c5ccccc5)c5ccccc45)ccc32)cc1 ; c1ccc(-c2ccccc2-n2c3ccc(-c4ccc5c(c4)c4ccccc4n5-c4ccc5ccccc5c4)cc3c3c4ccccc4ccc32)cc1] 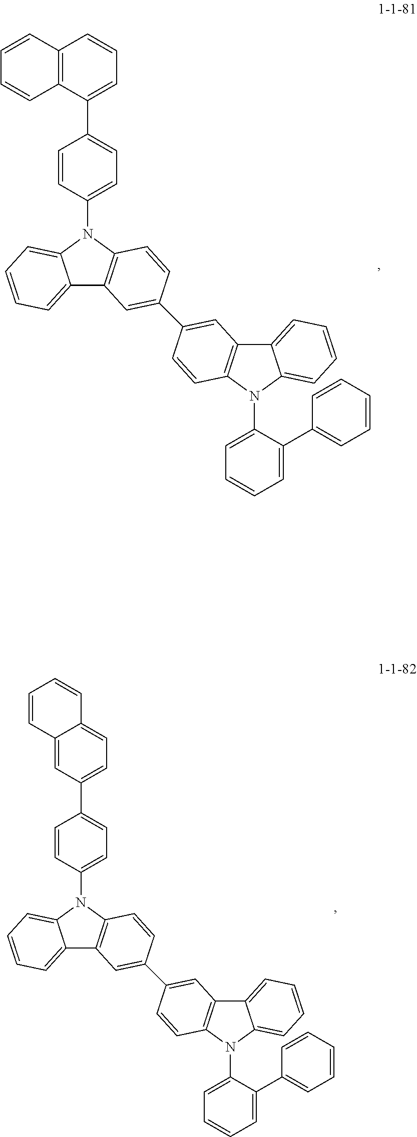 [SMILES: c1ccc(-c2ccccc2-n2c3ccccc3c3cc(-c4ccc5c(c4)c4ccccc4n5-c4ccc(-c5cccc6ccccc56)cc4)ccc32)cc1 ; c1ccc(-c2ccccc2-n2c3ccccc3c3cc(-c4ccc5c(c4)c4ccccc4n5-c4ccc(-c5ccc6ccccc6c5)cc4)ccc32)cc1] 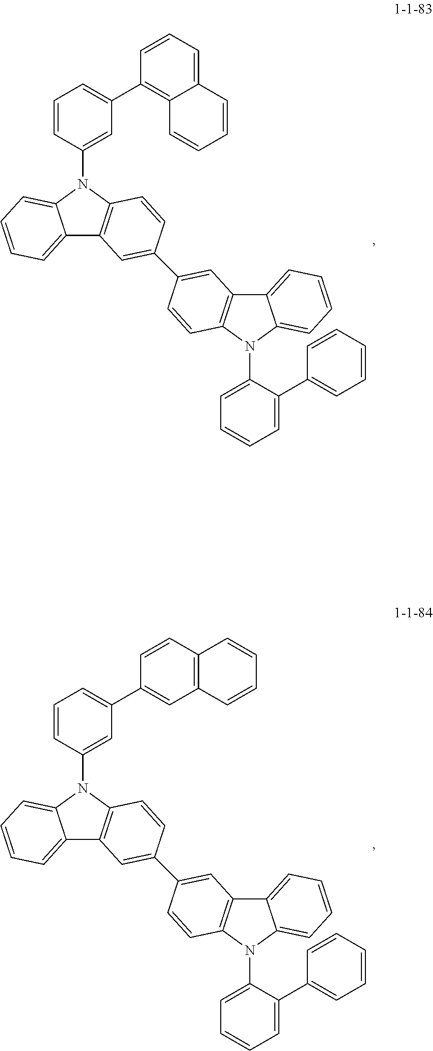 [SMILES: c1ccc(-c2ccccc2-n2c3ccccc3c3cc(-c4ccc5c(c4)c4ccccc4n5-c4cccc(-c5cccc6ccccc56)c4)ccc32)cc1 ; c1ccc(-c2ccccc2-n2c3ccccc3c3cc(-c4ccc5c(c4)c4ccccc4n5-c4cccc(-c5ccc6ccccc6c5)c4)ccc32)cc1] 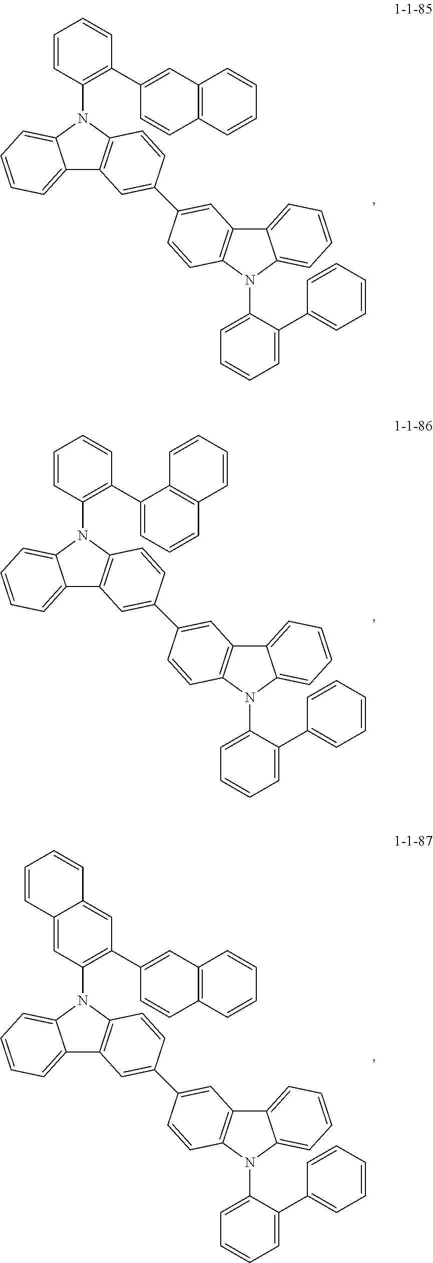 [SMILES: c1ccc(-c2ccccc2-n2c3ccccc3c3cc(-c4ccc5c(c4)c4ccccc4n5-c4ccccc4-c4ccc5ccccc5c4)ccc32)cc1 ; c1ccc(-c2ccccc2-n2c3ccccc3c3cc(-c4ccc5c(c4)c4ccccc4n5-c4ccccc4-c4cccc5ccccc45)ccc32)cc1 ; c1ccc(-c2ccccc2-n2c3ccccc3c3cc(-c4ccc5c(c4)c4ccccc4n5-c4cc5ccccc5cc4-c4ccc5ccccc5c4)ccc32)cc1] 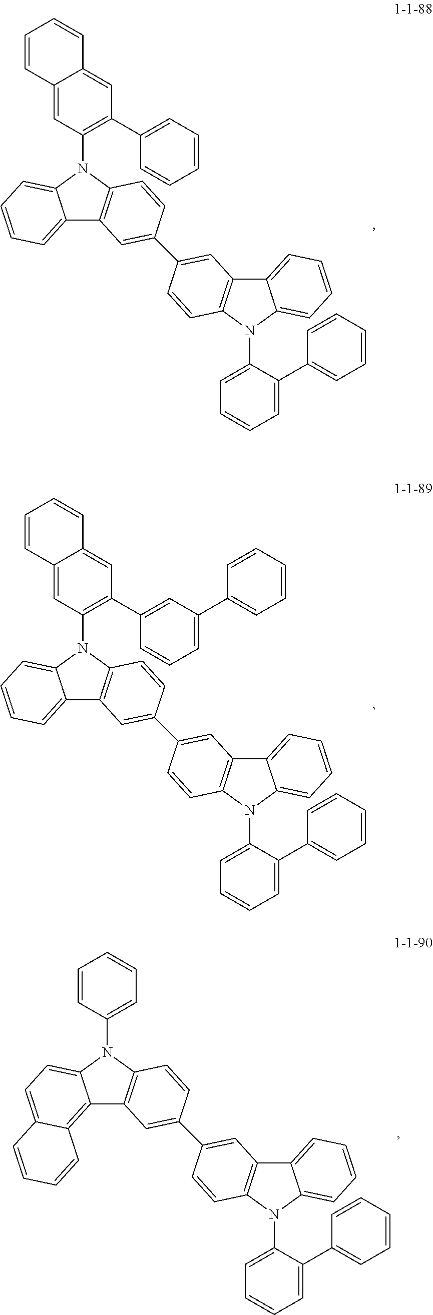 [SMILES: c1ccc(-c2ccccc2-n2c3ccccc3c3cc(-c4ccc5c(c4)c4ccccc4n5-c4cc5ccccc5cc4-c4ccccc4)ccc32)cc1 ; c1ccc(-c2cccc(-c3cc4ccccc4cc3-n3c4ccccc4c4cc(-c5ccc6c(c5)c5ccccc5n6-c5ccccc5-c5ccccc5)ccc43)c2)cc1 ; c1ccc(-c2ccccc2-n2c3ccccc3c3cc(-c4ccc5c(c4)c4c6ccccc6ccc4n5-c4ccccc4)ccc32)cc1] 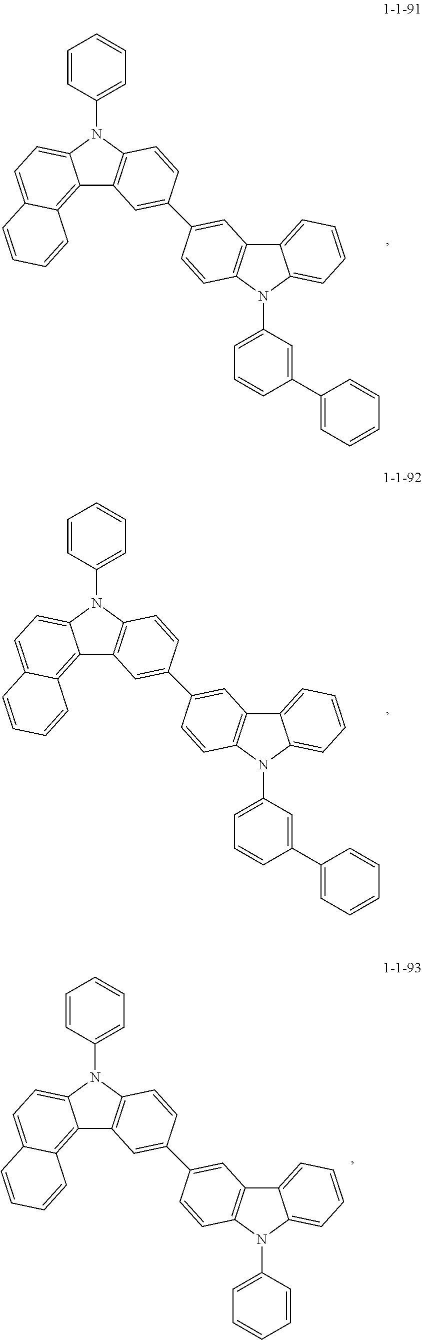 [SMILES: c1ccc(-c2cccc(-n3c4ccccc4c4cc(-c5ccc6c(c5)c5c7ccccc7ccc5n6-c5ccccc5)ccc43)c2)cc1 ; c1ccc(-c2cccc(-n3c4ccccc4c4cc(-c5ccc6c(c5)c5c7ccccc7ccc5n6-c5ccccc5)ccc43)c2)cc1 ; c1ccc(-n2c3ccccc3c3cc(-c4ccc5c(c4)c4c6ccccc6ccc4n5-c4ccccc4)ccc32)cc1] 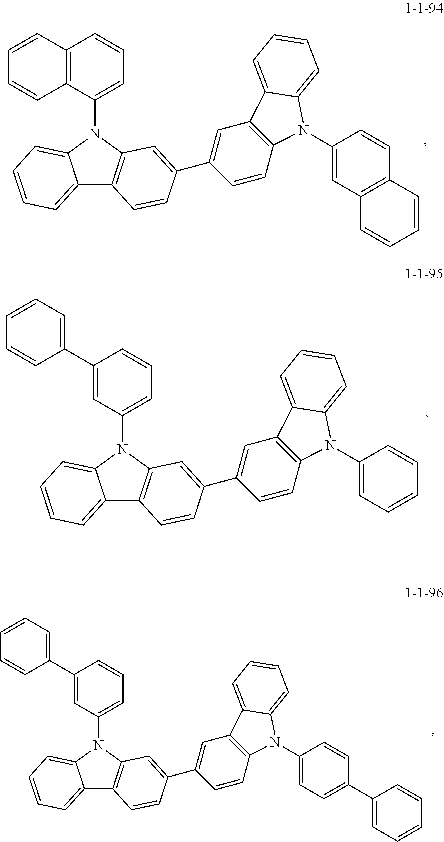 [SMILES: c1ccc2cc(-n3c4ccccc4c4cc(-c5ccc6c7ccccc7n(-c7cccc8ccccc78)c6c5)ccc43)ccc2c1 ; c1ccc(-c2cccc(-n3c4ccccc4c4ccc(-c5ccc6c(c5)c5ccccc5n6-c5ccccc5)cc43)c2)cc1 ; c1ccc(-c2ccc(-n3c4ccccc4c4cc(-c5ccc6c7ccccc7n(-c7cccc(-c8ccccc8)c7)c6c5)ccc43)cc2)cc1] 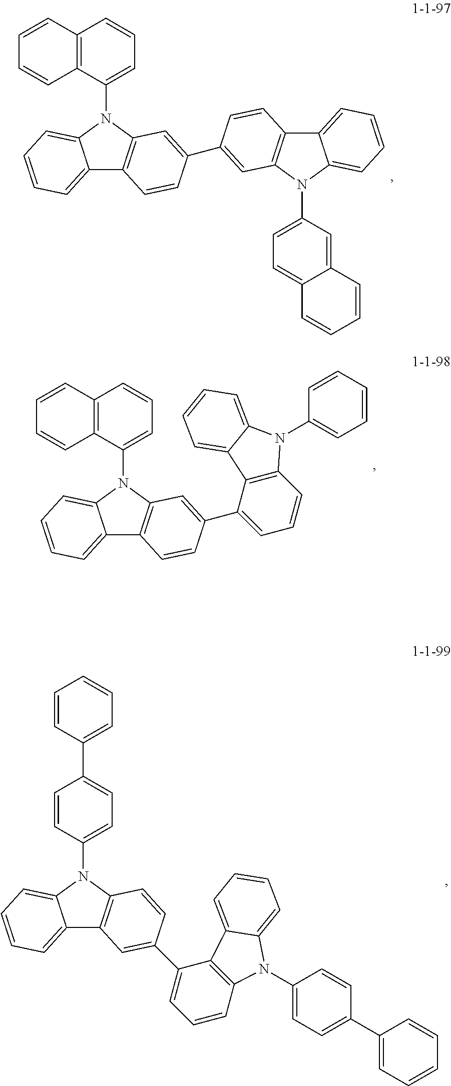 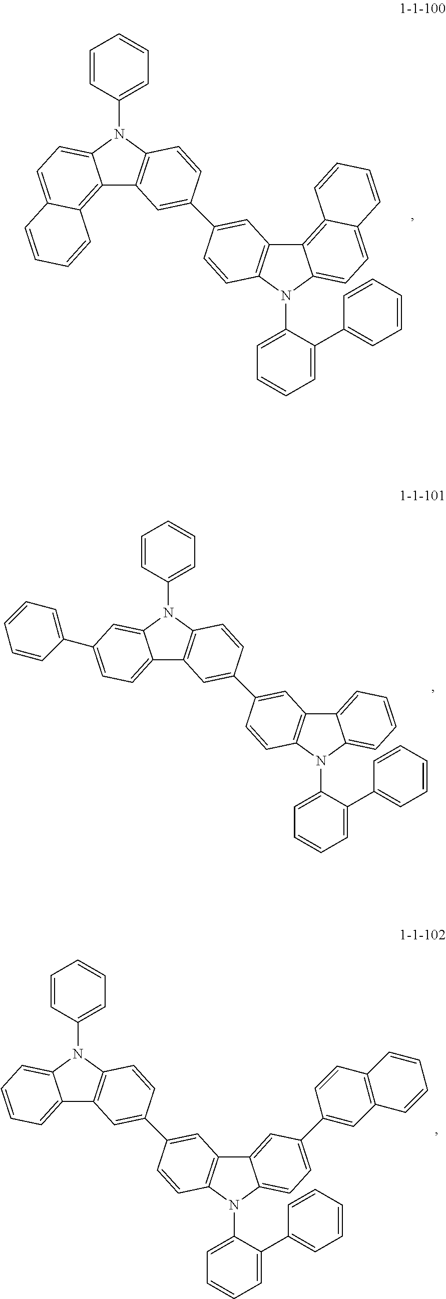 [SMILES: c1ccc(-c2ccccc2-n2c3ccc(-c4ccc5c(c4)c4c6ccccc6ccc4n5-c4ccccc4)cc3c3c4ccccc4ccc32)cc1 ; c1ccc(-c2ccc3c4cc(-c5ccc6c(c5)c5ccccc5n6-c5ccccc5-c5ccccc5)ccc4n(-c4ccccc4)c3c2)cc1 ; c1ccc(-c2ccccc2-n2c3ccc(-c4ccc5ccccc5c4)cc3c3cc(-c4ccc5c(c4)c4ccccc4n5-c4ccccc4)ccc32)cc1] 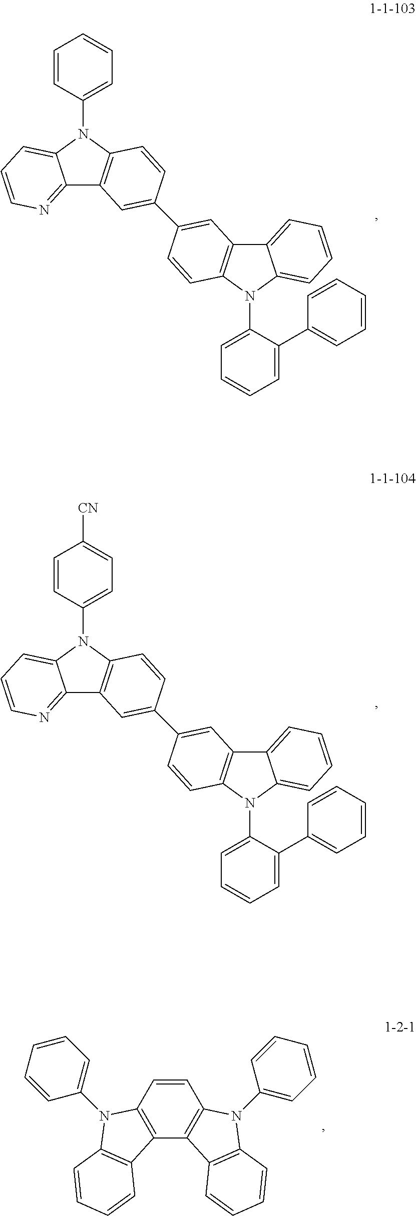 [SMILES: c1ccc(-c2ccccc2-n2c3ccccc3c3cc(-c4ccc5c(c4)c4ncccc4n5-c4ccccc4)ccc32)cc1 ; N#Cc1ccc(-n2c3ccc(-c4ccc5c(c4)c4ccccc4n5-c4ccccc4-c4ccccc4)cc3c3ncccc32)cc1 ; c1ccc(-n2c3ccccc3c3c4c5ccccc5n(-c5ccccc5)c4ccc32)cc1] 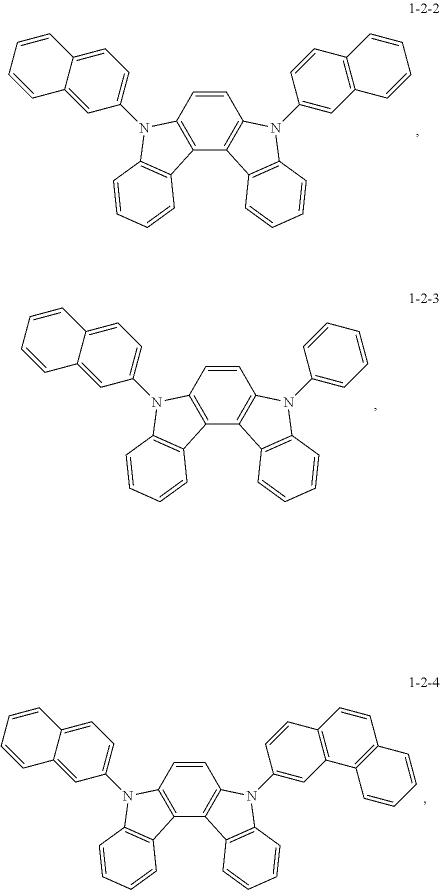 [SMILES: c1ccc2cc(-n3c4ccccc4c4c5c6ccccc6n(-c6ccc7ccccc7c6)c5ccc43)ccc2c1 ; c1ccc(-n2c3ccccc3c3c4c5ccccc5n(-c5ccc6ccccc6c5)c4ccc32)cc1 ; c1ccc2cc(-n3c4ccccc4c4c5c6ccccc6n(-c6ccc7ccc8ccccc8c7c6)c5ccc43)ccc2c1] 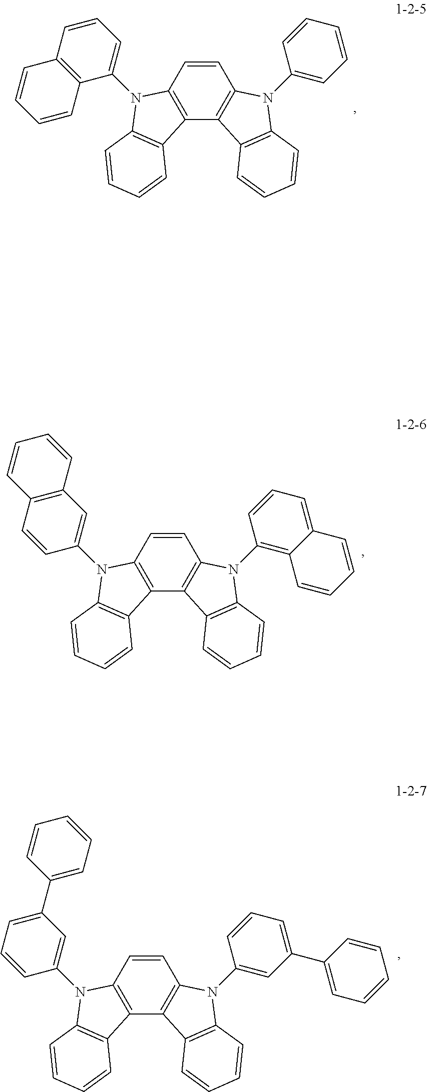 [SMILES: c1ccc(-n2c3ccccc3c3c4c5ccccc5n(-c5cccc6ccccc56)c4ccc32)cc1 ; c1ccc2cc(-n3c4ccccc4c4c5c6ccccc6n(-c6cccc7ccccc67)c5ccc43)ccc2c1 ; c1ccc(-c2cccc(-n3c4ccccc4c4c5c6ccccc6n(-c6cccc(-c7ccccc7)c6)c5ccc43)c2)cc1] 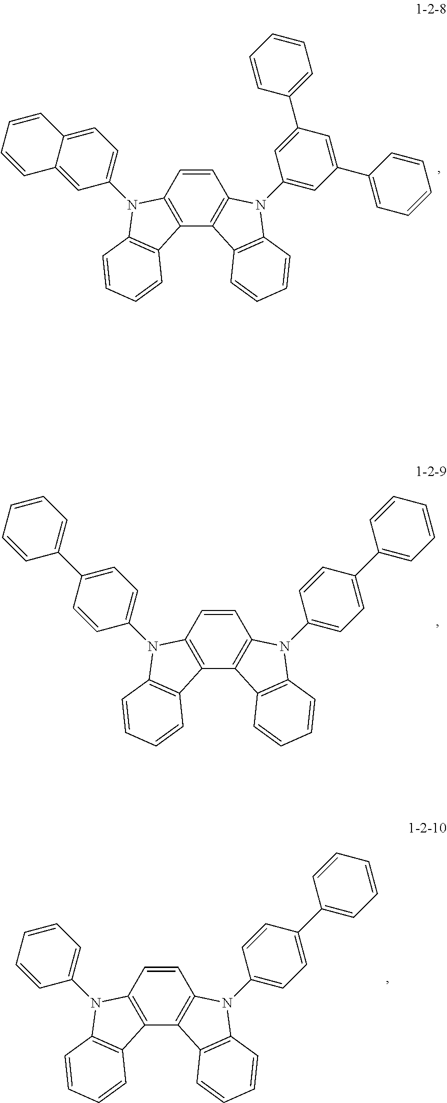 [SMILES: c1ccc(-c2cc(-c3ccccc3)cc(-n3c4ccccc4c4c5c6ccccc6n(-c6ccc7ccccc7c6)c5ccc43)c2)cc1 ; c1ccc(-c2ccc(-n3c4ccccc4c4c5c6ccccc6n(-c6ccc(-c7ccccc7)cc6)c5ccc43)cc2)cc1 ; c1ccc(-c2ccc(-n3c4ccccc4c4c5c6ccccc6n(-c6ccccc6)c5ccc43)cc2)cc1] 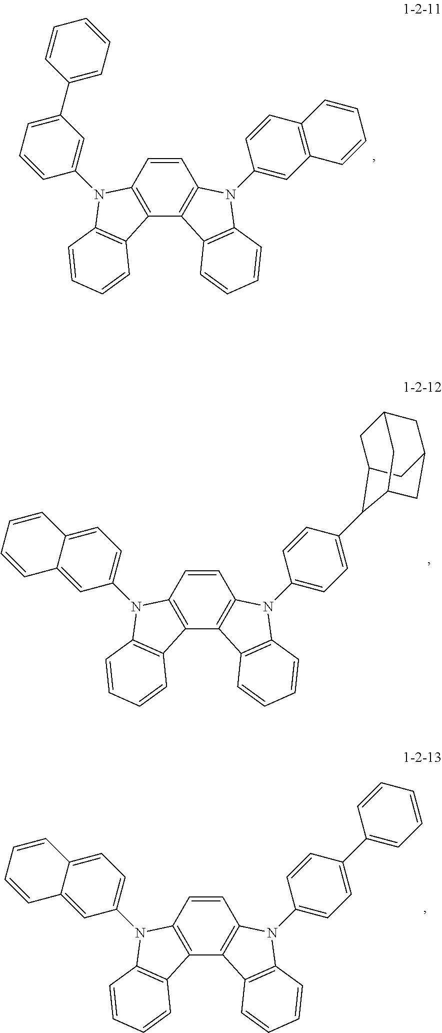 [SMILES: c1ccc(-c2cccc(-n3c4ccccc4c4c5c6ccccc6n(-c6ccc7ccccc7c6)c5ccc43)c2)cc1 ; c1ccc2cc(-n3c4ccccc4c4c5c6ccccc6n(-c6ccc(C7C8CC9CC(C8)CC7C9)cc6)c5ccc43)ccc2c1 ; c1ccc(-c2ccc(-n3c4ccccc4c4c5c6ccccc6n(-c6ccc7ccccc7c6)c5ccc43)cc2)cc1] 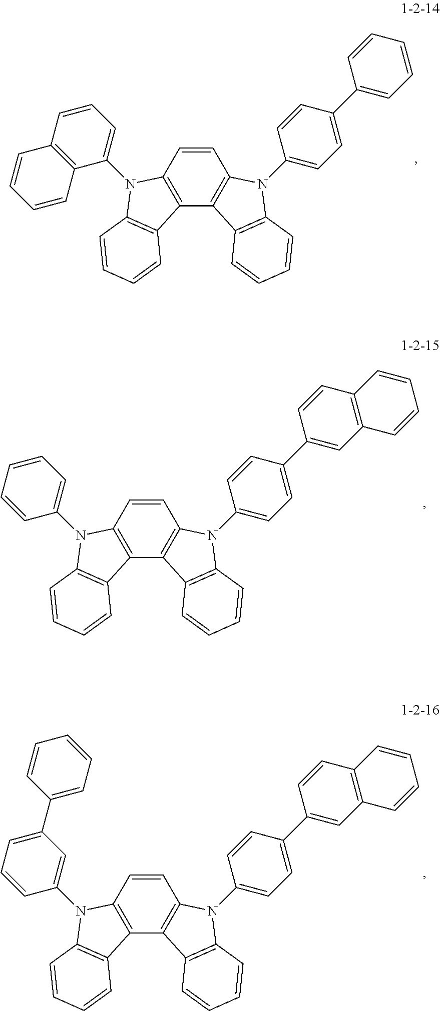 [SMILES: c1ccc(-c2ccc(-n3c4ccccc4c4c5c6ccccc6n(-c6cccc7ccccc67)c5ccc43)cc2)cc1 ; c1ccc(-n2c3ccccc3c3c4c5ccccc5n(-c5ccc(-c6ccc7ccccc7c6)cc5)c4ccc32)cc1 ; c1ccc(-c2cccc(-n3c4ccccc4c4c5c6ccccc6n(-c6ccc(-c7ccc8ccccc8c7)cc6)c5ccc43)c2)cc1] 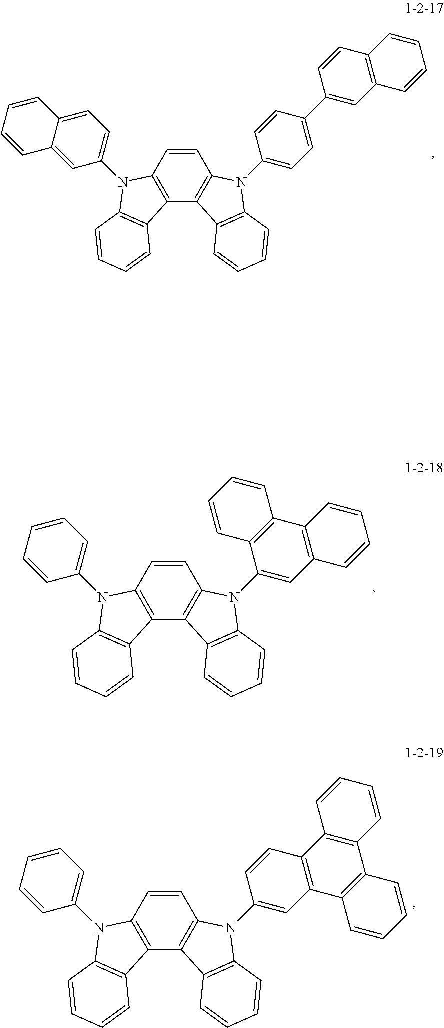 [SMILES: c1ccc2cc(-c3ccc(-n4c5ccccc5c5c6c7ccccc7n(-c7ccc8ccccc8c7)c6ccc54)cc3)ccc2c1 ; c1ccc(-n2c3ccccc3c3c4c5ccccc5n(-c5cc6ccccc6c6ccccc56)c4ccc32)cc1 ; c1ccc(-n2c3ccccc3c3c4c5ccccc5n(-c5ccc6c7ccccc7c7ccccc7c6c5)c4ccc32)cc1] 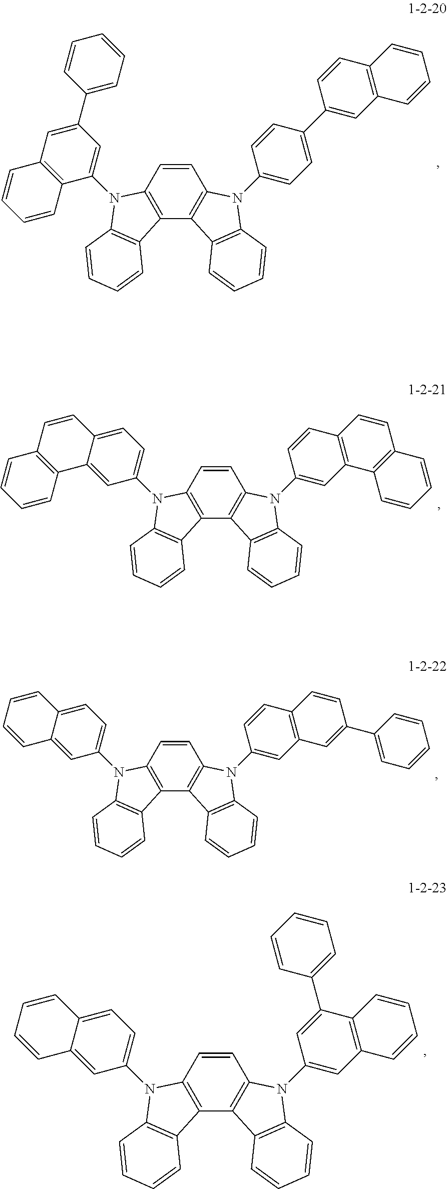 [SMILES: c1ccc(-c2cc(-n3c4ccccc4c4c5c6ccccc6n(-c6ccc(-c7ccc8ccccc8c7)cc6)c5ccc43)c3ccccc3c2)cc1 ; c1ccc2c(c1)ccc1ccc(-n3c4ccccc4c4c5c6ccccc6n(-c6ccc7ccc8ccccc8c7c6)c5ccc43)cc12 ; c1ccc(-c2ccc3ccc(-n4c5ccccc5c5c6c7ccccc7n(-c7ccc8ccccc8c7)c6ccc54)cc3c2)cc1 ; c1ccc(-c2cc(-n3c4ccccc4c4c5c6ccccc6n(-c6ccc7ccccc7c6)c5ccc43)cc3ccccc23)cc1] 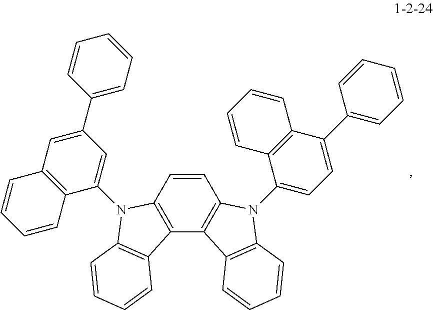 [SMILES: c1ccc(-c2cc(-n3c4ccccc4c4c5c6ccccc6n(-c6ccc(-c7ccccc7)c7ccccc67)c5ccc43)c3ccccc3c2)cc1] 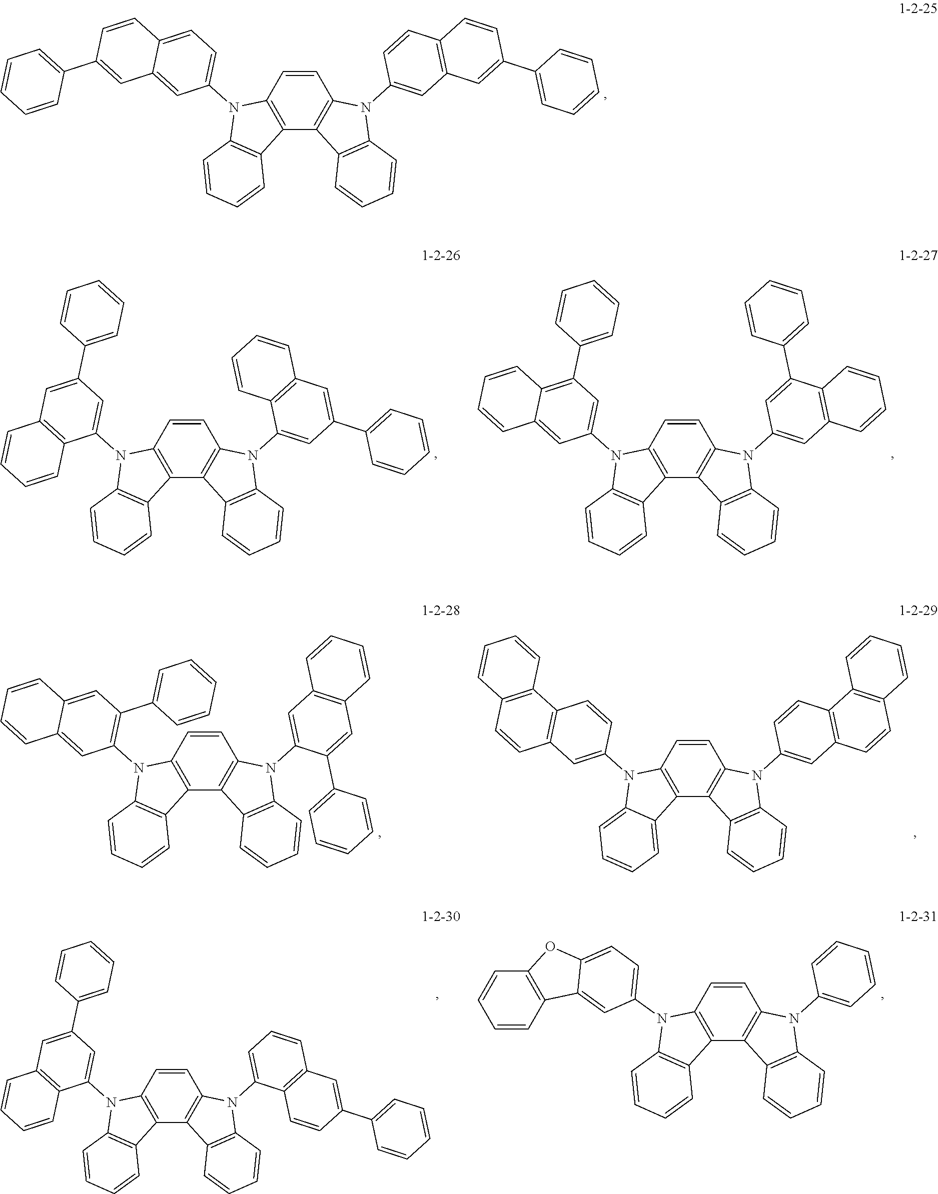 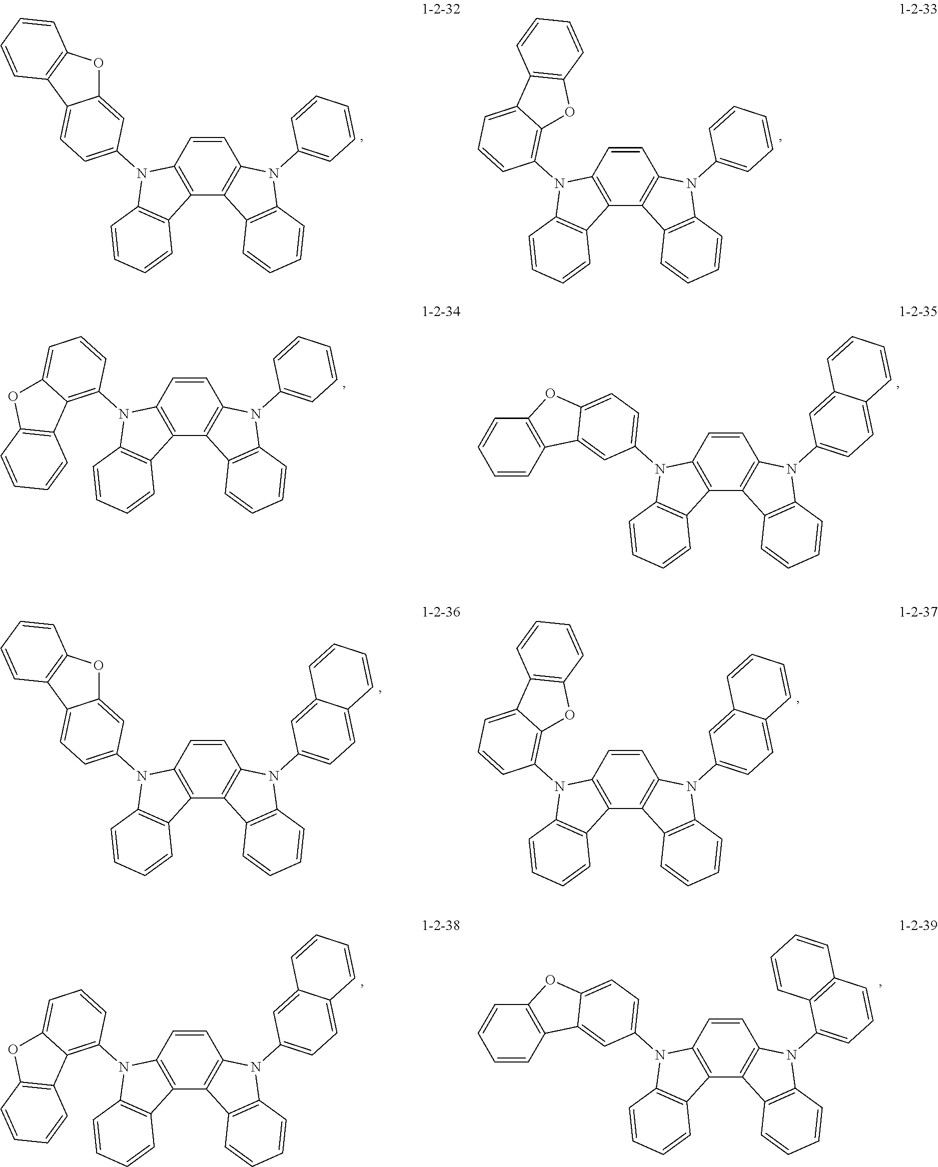 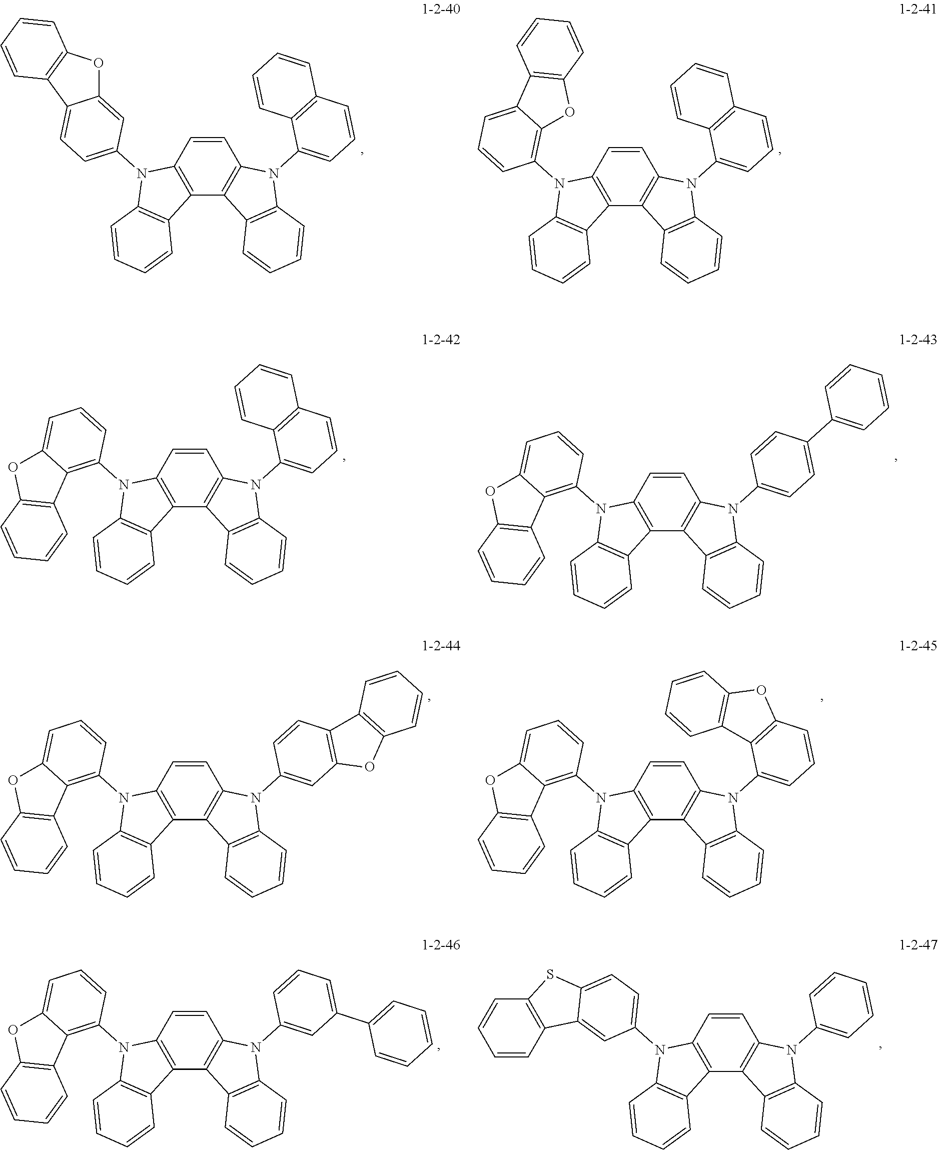 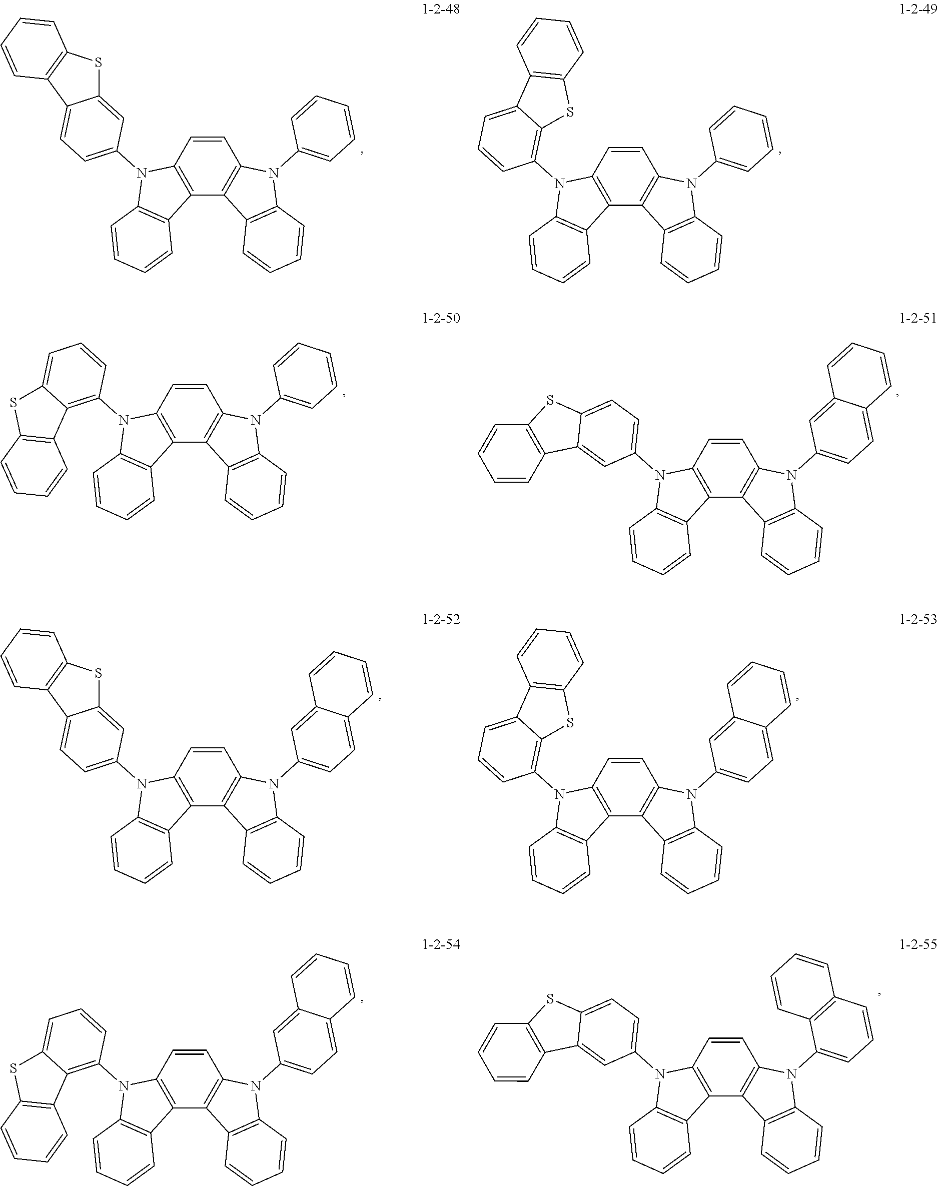 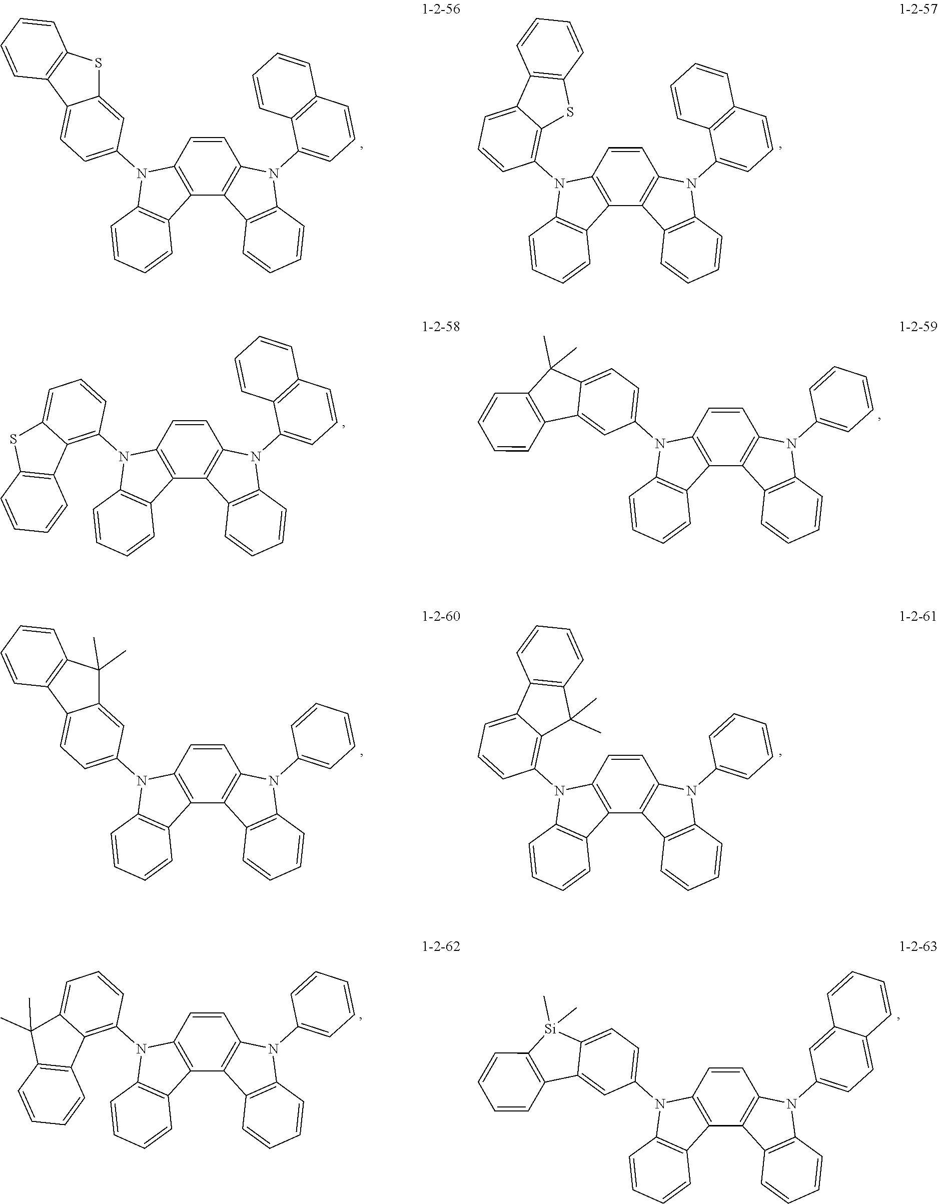 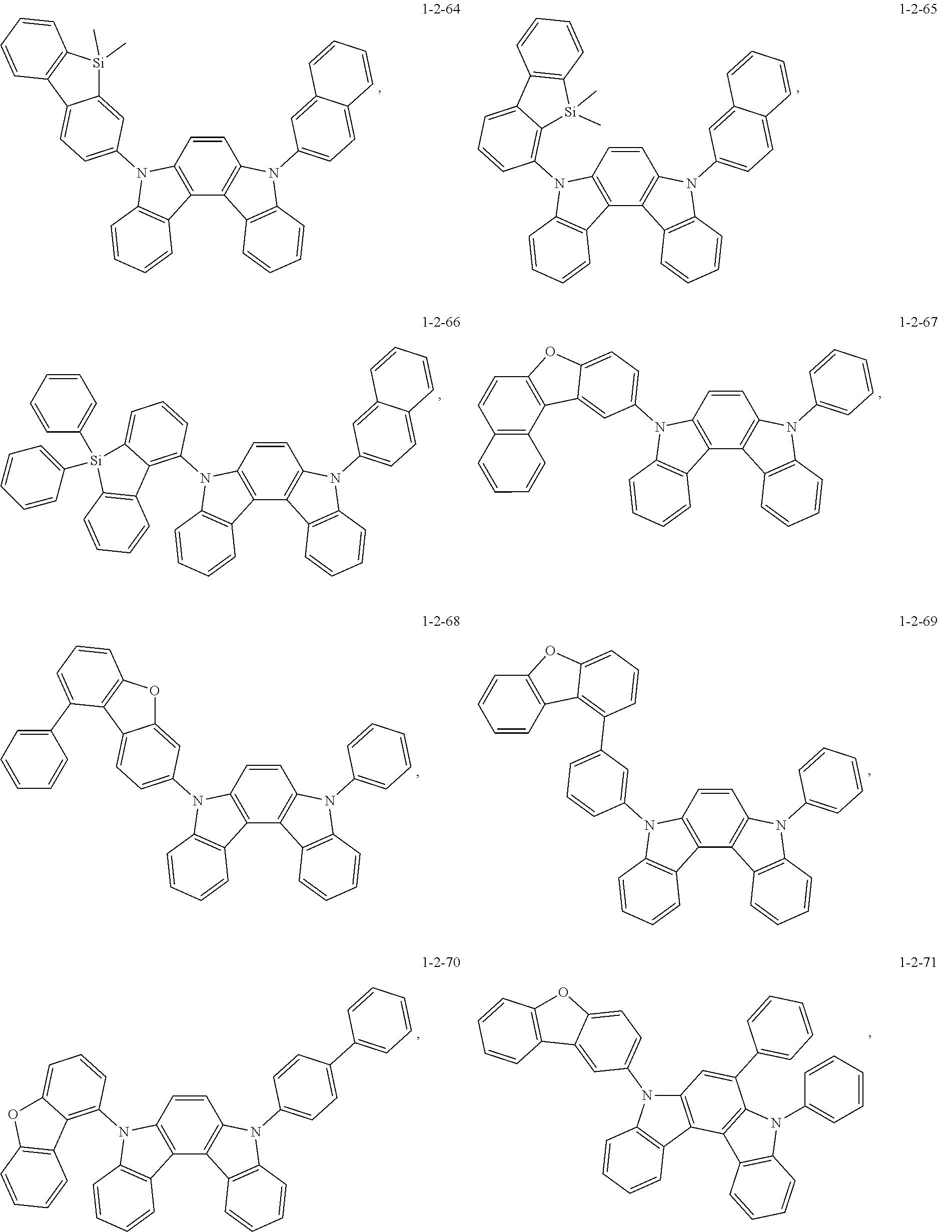 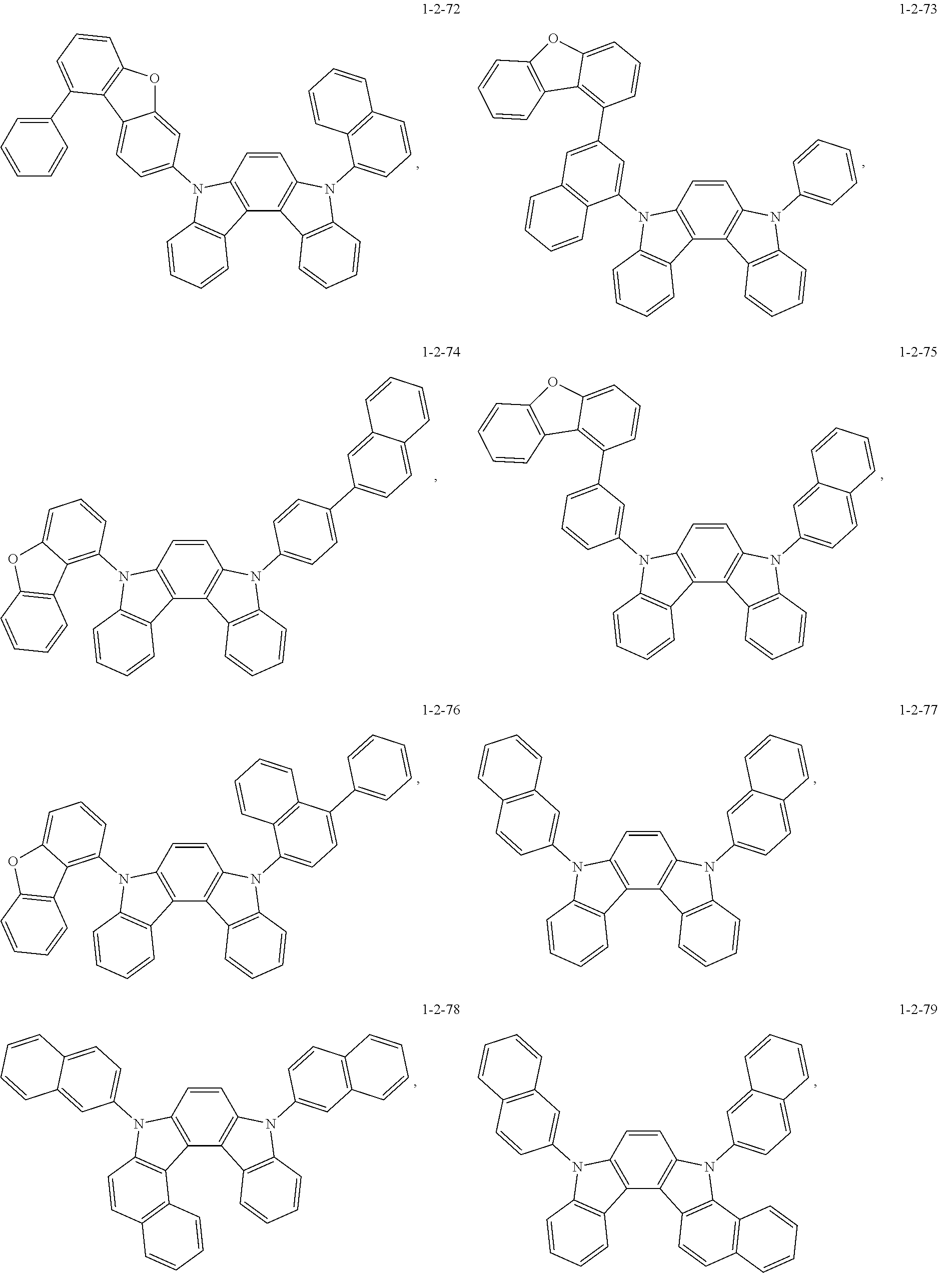 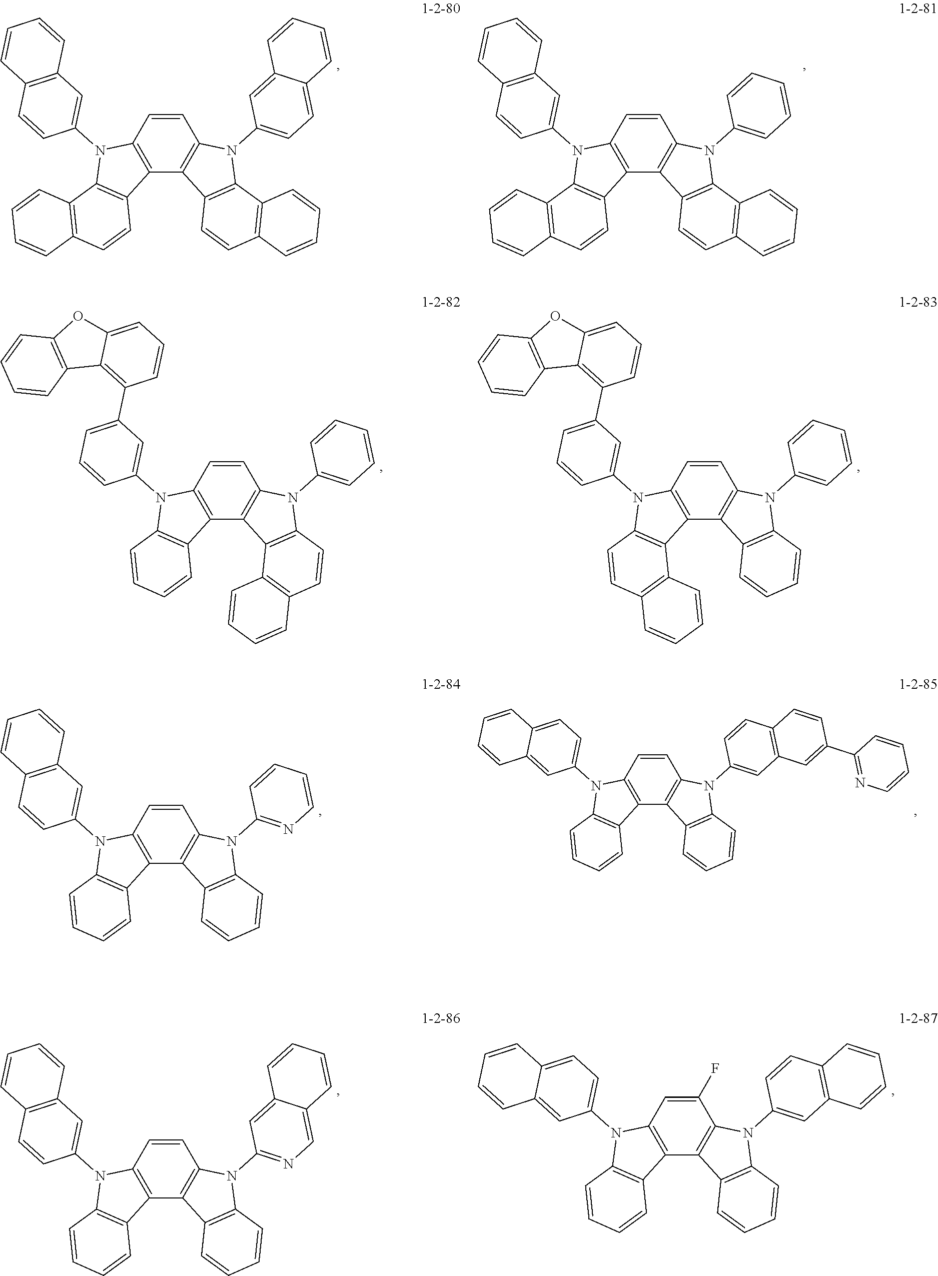 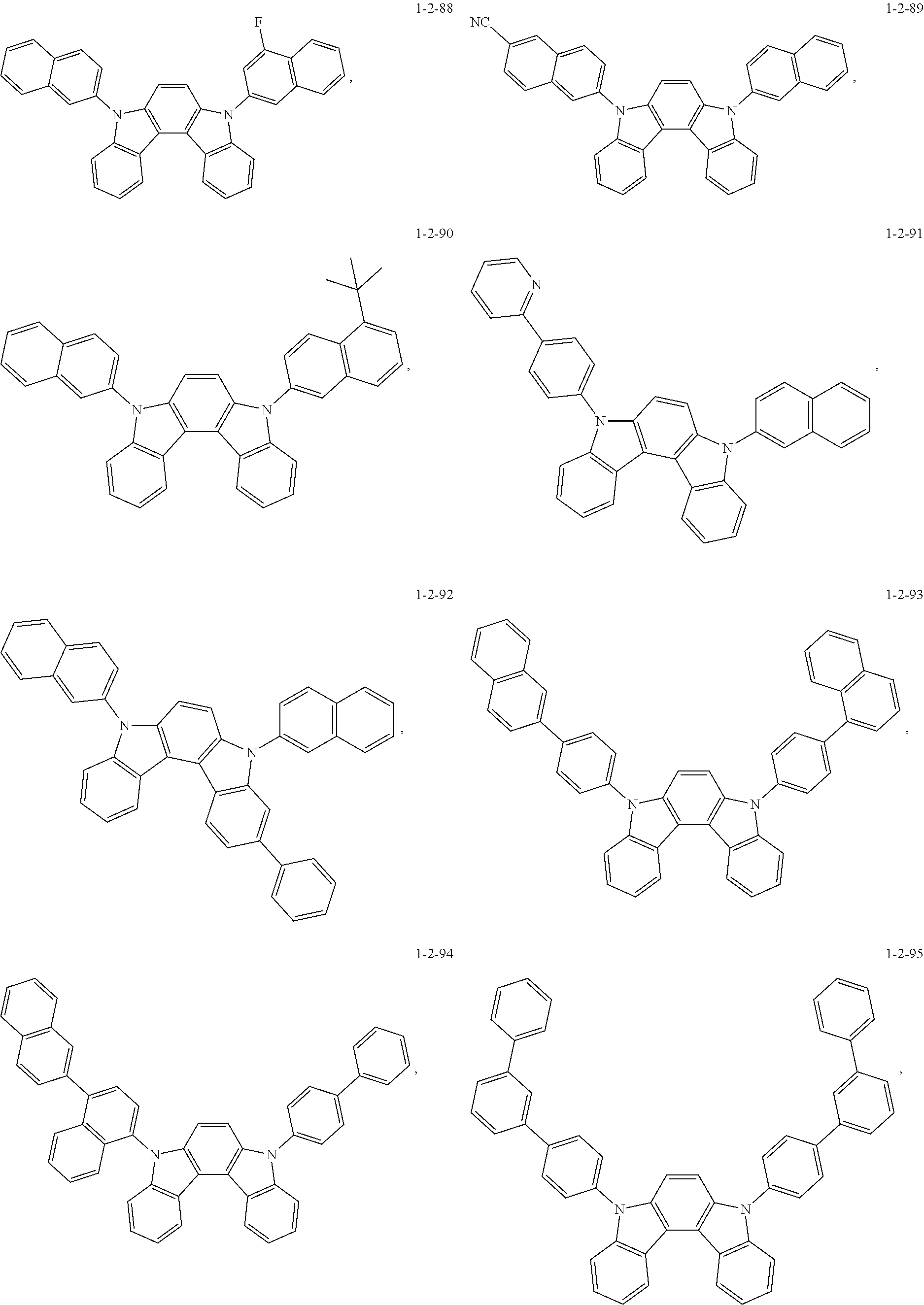 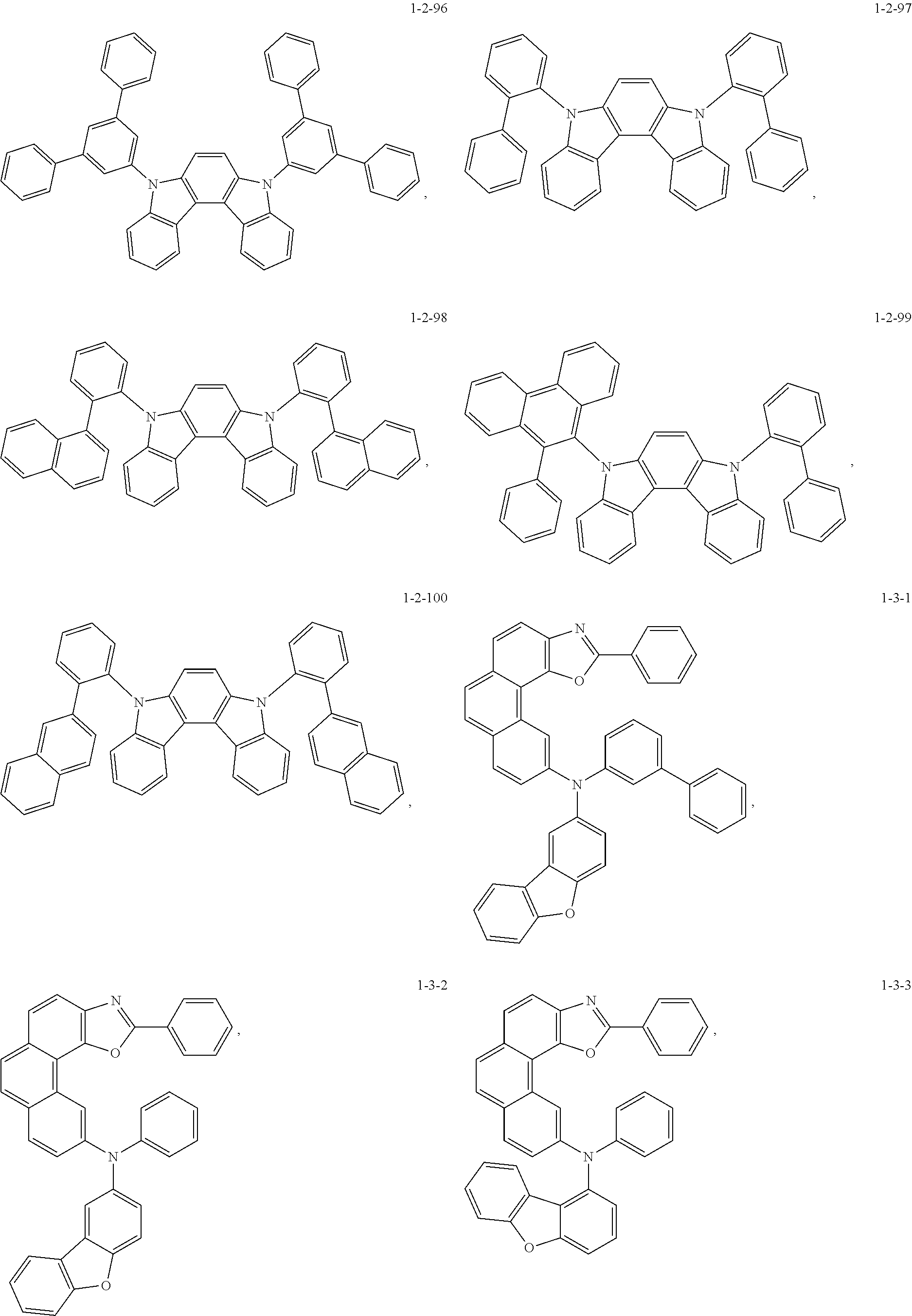 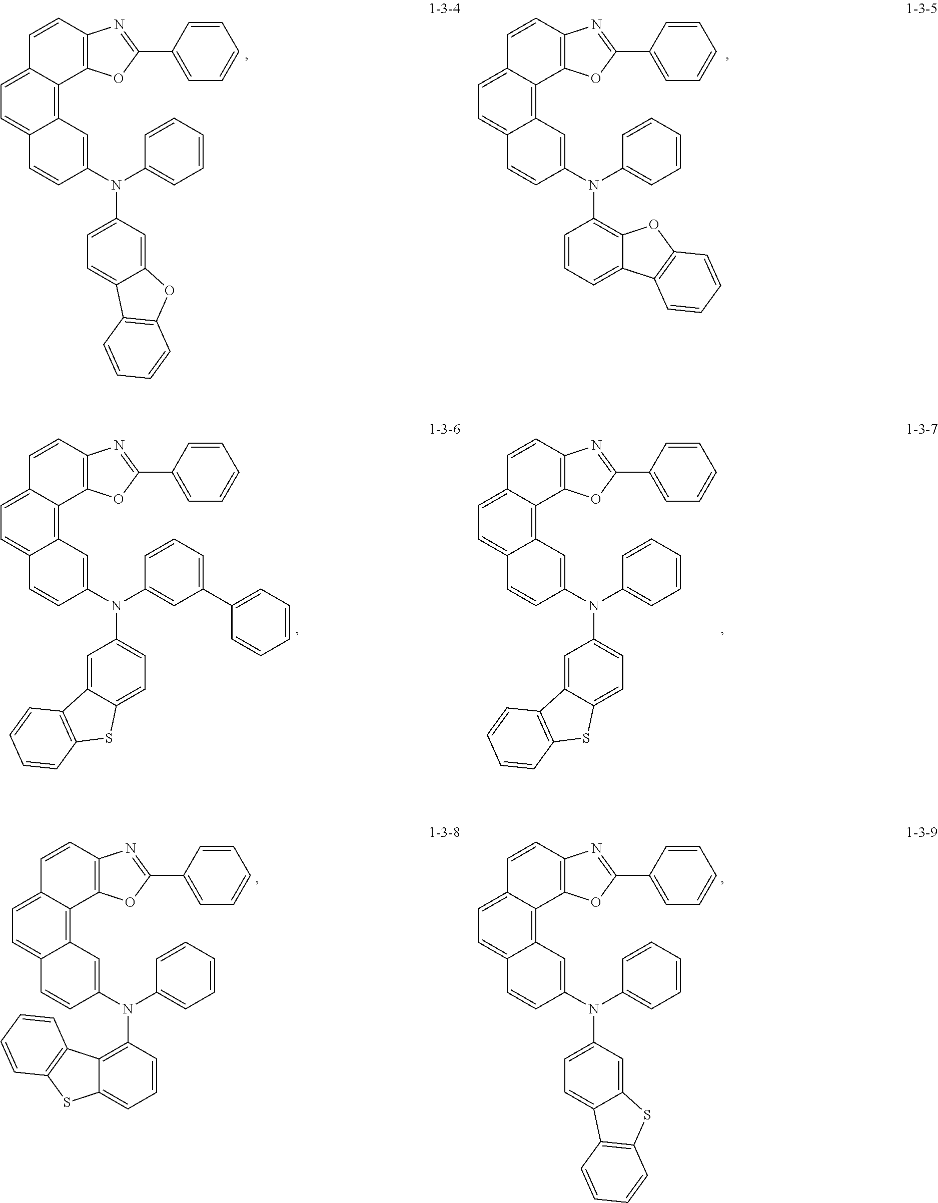 [SMILES: c1ccc(-c2nc3ccc4ccc5ccc(N(c6ccccc6)c6ccc7c(c6)oc6ccccc67)cc5c4c3o2)cc1 ; c1ccc(-c2nc3ccc4ccc5ccc(N(c6ccccc6)c6cccc7c6oc6ccccc67)cc5c4c3o2)cc1 ; c1ccc(-c2cccc(N(c3ccc4sc5ccccc5c4c3)c3ccc4ccc5ccc6nc(-c7ccccc7)oc6c5c4c3)c2)cc1 ; c1ccc(-c2nc3ccc4ccc5ccc(N(c6ccccc6)c6ccc7sc8ccccc8c7c6)cc5c4c3o2)cc1 ; c1ccc(-c2nc3ccc4ccc5ccc(N(c6ccccc6)c6cccc7sc8ccccc8c67)cc5c4c3o2)cc1 ; c1ccc(-c2nc3ccc4ccc5ccc(N(c6ccccc6)c6ccc7c(c6)sc6ccccc67)cc5c4c3o2)cc1] 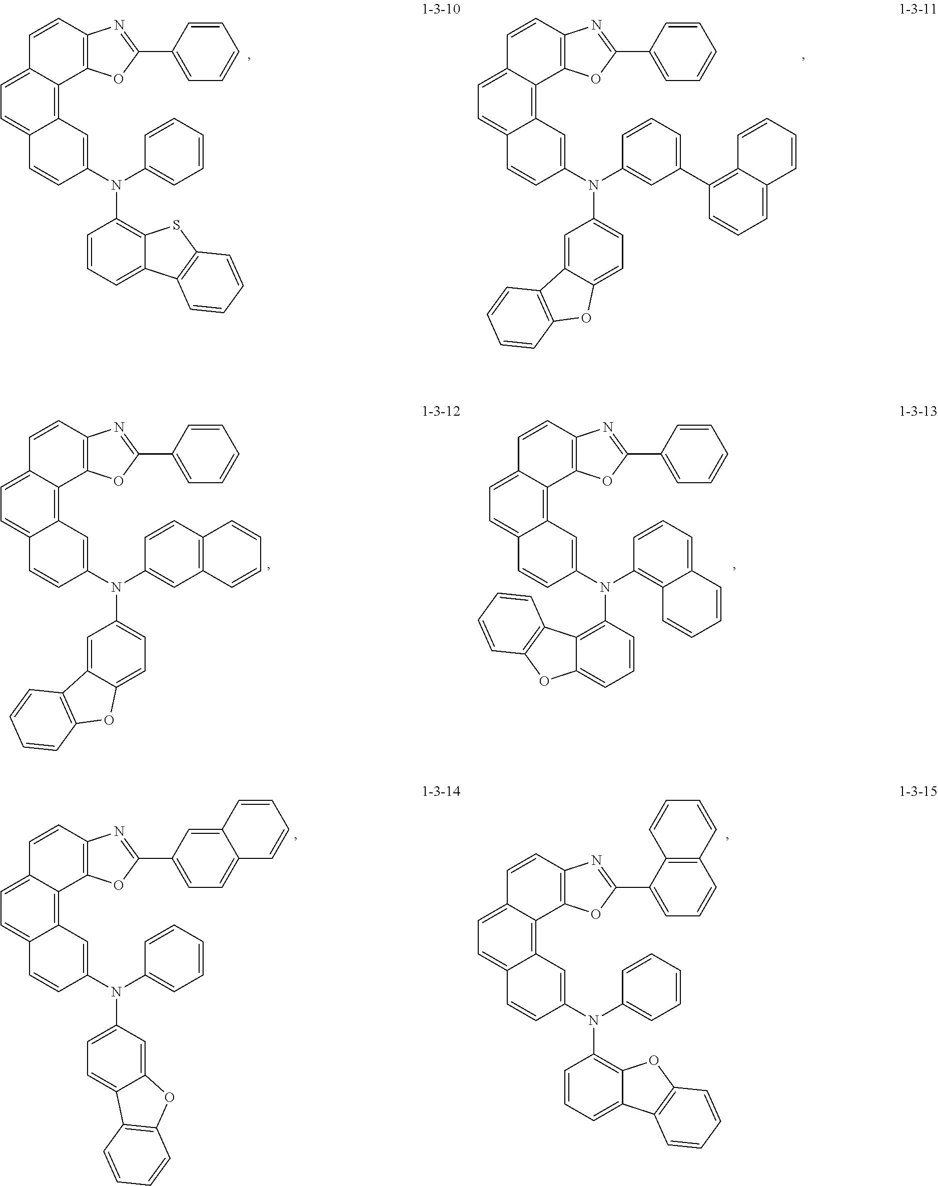 [SMILES: c1ccc(-c2nc3ccc4ccc5ccc(N(c6ccccc6)c6cccc7c6sc6ccccc67)cc5c4c3o2)cc1 ; c1ccc(-c2nc3ccc4ccc5ccc(N(c6cccc(-c7cccc8ccccc78)c6)c6ccc7oc8ccccc8c7c6)cc5c4c3o2)cc1 ; c1ccc(-c2nc3ccc4ccc5ccc(N(c6ccc7ccccc7c6)c6ccc7oc8ccccc8c7c6)cc5c4c3o2)cc1 ; c1ccc(-c2nc3ccc4ccc5ccc(N(c6cccc7ccccc67)c6cccc7oc8ccccc8c67)cc5c4c3o2)cc1 ; c1ccc(N(c2ccc3c(c2)oc2ccccc23)c2ccc3ccc4ccc5nc(-c6ccc7ccccc7c6)oc5c4c3c2)cc1 ; c1ccc(N(c2ccc3ccc4ccc5nc(-c6cccc7ccccc67)oc5c4c3c2)c2cccc3c2oc2ccccc23)cc1] 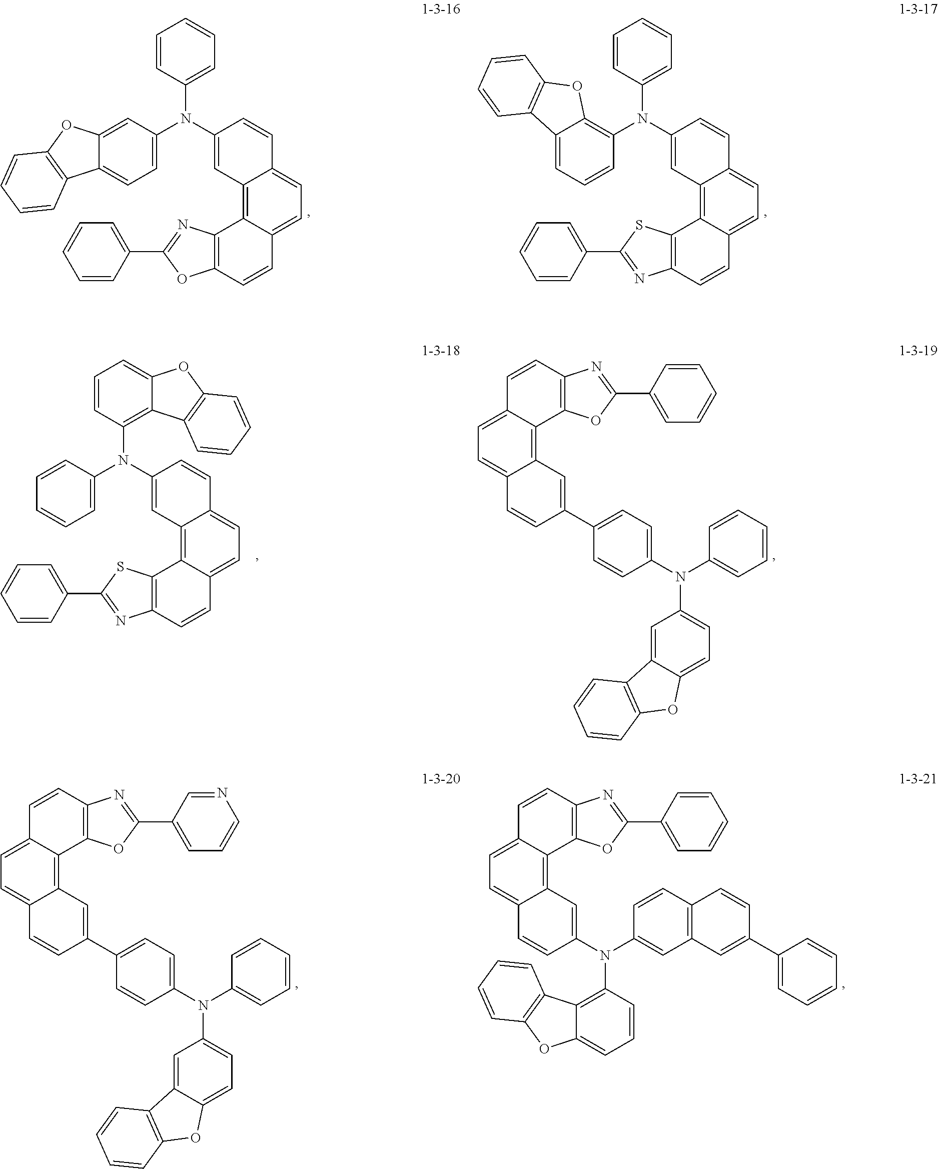 [SMILES: c1ccc(-c2nc3c(ccc4ccc5ccc(N(c6ccccc6)c6ccc7c(c6)oc6ccccc67)cc5c43)o2)cc1 ; c1ccc(-c2nc3ccc4ccc5ccc(N(c6ccccc6)c6cccc7c6oc6ccccc67)cc5c4c3s2)cc1 ; c1ccc(-c2nc3ccc4ccc5ccc(N(c6ccccc6)c6cccc7oc8ccccc8c67)cc5c4c3s2)cc1 ; c1ccc(-c2nc3ccc4ccc5ccc(-c6ccc(N(c7ccccc7)c7ccc8oc9ccccc9c8c7)cc6)cc5c4c3o2)cc1 ; c1ccc(N(c2ccc(-c3ccc4ccc5ccc6nc(-c7cccnc7)oc6c5c4c3)cc2)c2ccc3oc4ccccc4c3c2)cc1 ; c1ccc(-c2ccc3ccc(N(c4ccc5ccc6ccc7nc(-c8ccccc8)oc7c6c5c4)c4cccc5oc6ccccc6c45)cc3c2)cc1] 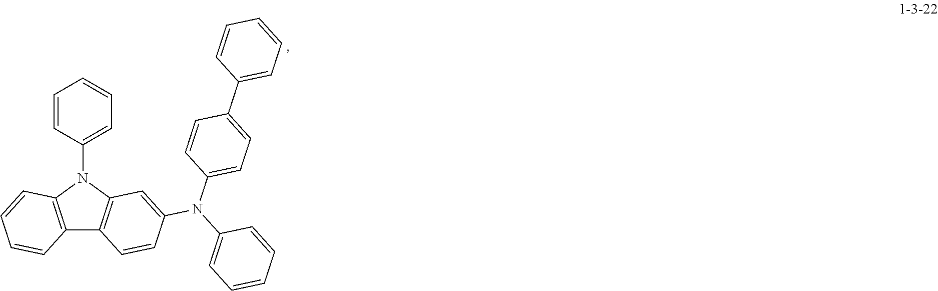 [SMILES: c1ccc(-c2ccc(N(c3ccccc3)c3ccc4c5ccccc5n(-c5ccccc5)c4c3)cc2)cc1] 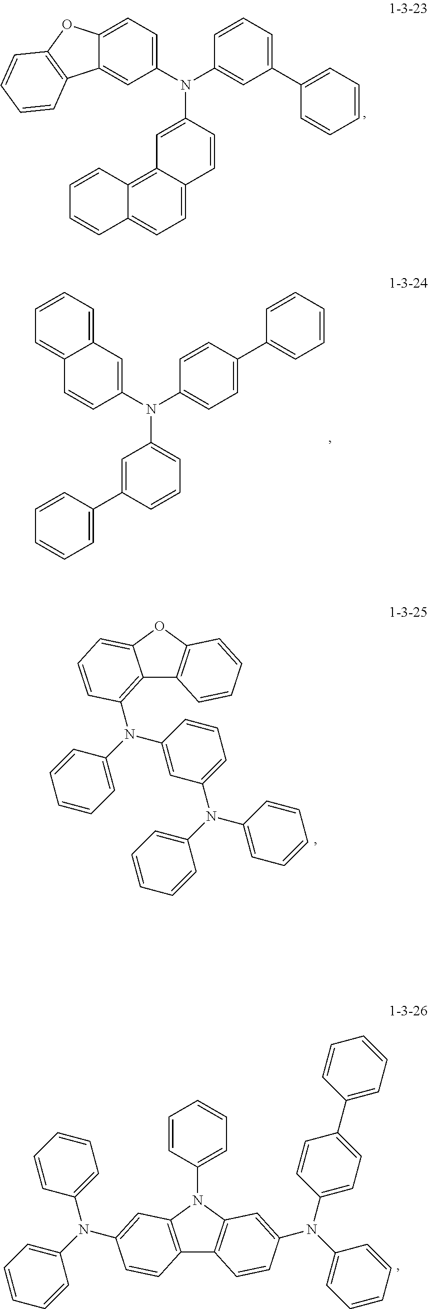 [SMILES: c1ccc(-c2cccc(N(c3ccc4ccc5ccccc5c4c3)c3ccc4oc5ccccc5c4c3)c2)cc1 ; c1ccc(-c2ccc(N(c3cccc(-c4ccccc4)c3)c3ccc4ccccc4c3)cc2)cc1 ; c1ccc(N(c2ccccc2)c2cccc(N(c3ccccc3)c3cccc4oc5ccccc5c34)c2)cc1 ; c1ccc(-c2ccc(N(c3ccccc3)c3ccc4c5ccc(N(c6ccccc6)c6ccccc6)cc5n(-c5ccccc5)c4c3)cc2)cc1] 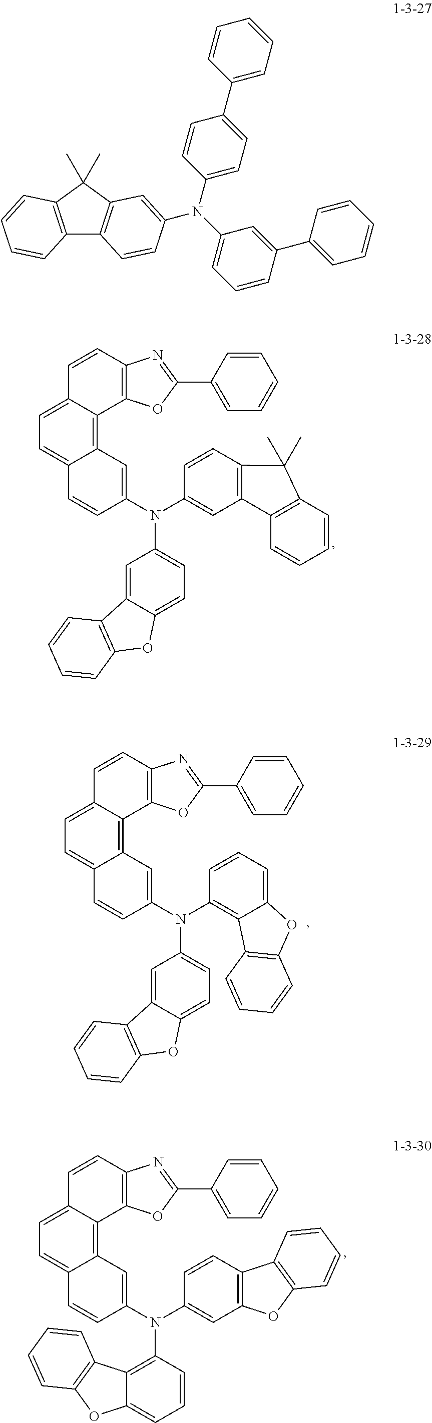 [SMILES: CC1(C)c2ccccc2-c2ccc(N(c3ccc(-c4ccccc4)cc3)c3cccc(-c4ccccc4)c3)cc21 ; CC1(C)c2ccccc2-c2cc(N(c3ccc4oc5ccccc5c4c3)c3ccc4ccc5ccc6nc(-c7ccccc7)oc6c5c4c3)ccc21 ; c1ccc(-c2nc3ccc4ccc5ccc(N(c6ccc7oc8ccccc8c7c6)c6cccc7oc8ccccc8c67)cc5c4c3o2)cc1 ; c1ccc(-c2nc3ccc4ccc5ccc(N(c6ccc7c(c6)oc6ccccc67)c6cccc7oc8ccccc8c67)cc5c4c3o2)cc1] 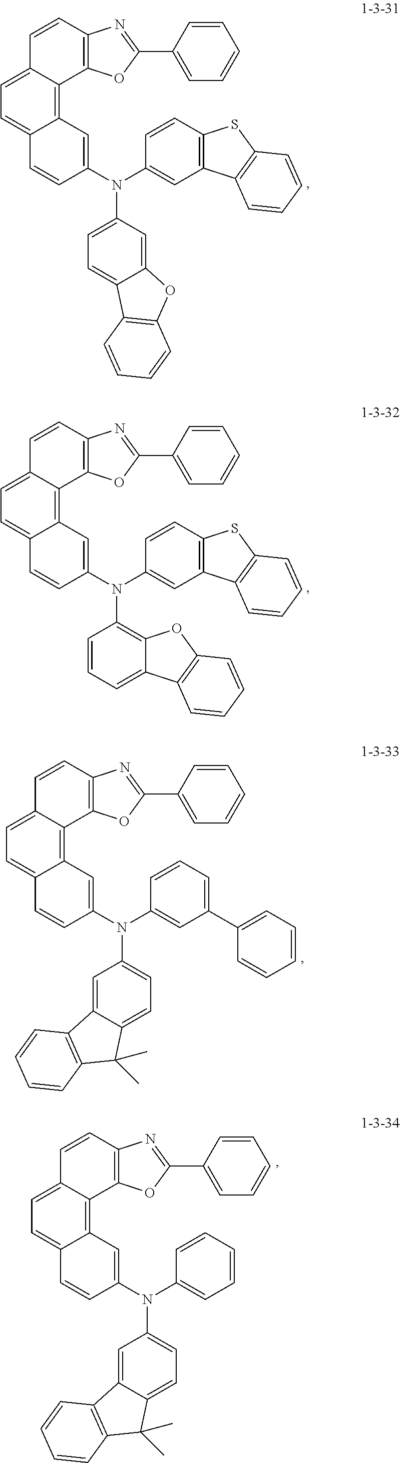 [SMILES: c1ccc(-c2nc3ccc4ccc5ccc(N(c6ccc7c(c6)oc6ccccc67)c6ccc7sc8ccccc8c7c6)cc5c4c3o2)cc1 ; c1ccc(-c2nc3ccc4ccc5ccc(N(c6ccc7sc8ccccc8c7c6)c6cccc7c6oc6ccccc67)cc5c4c3o2)cc1 ; CC1(C)c2ccccc2-c2cc(N(c3cccc(-c4ccccc4)c3)c3ccc4ccc5ccc6nc(-c7ccccc7)oc6c5c4c3)ccc21 ; CC1(C)c2ccccc2-c2cc(N(c3ccccc3)c3ccc4ccc5ccc6nc(-c7ccccc7)oc6c5c4c3)ccc21] 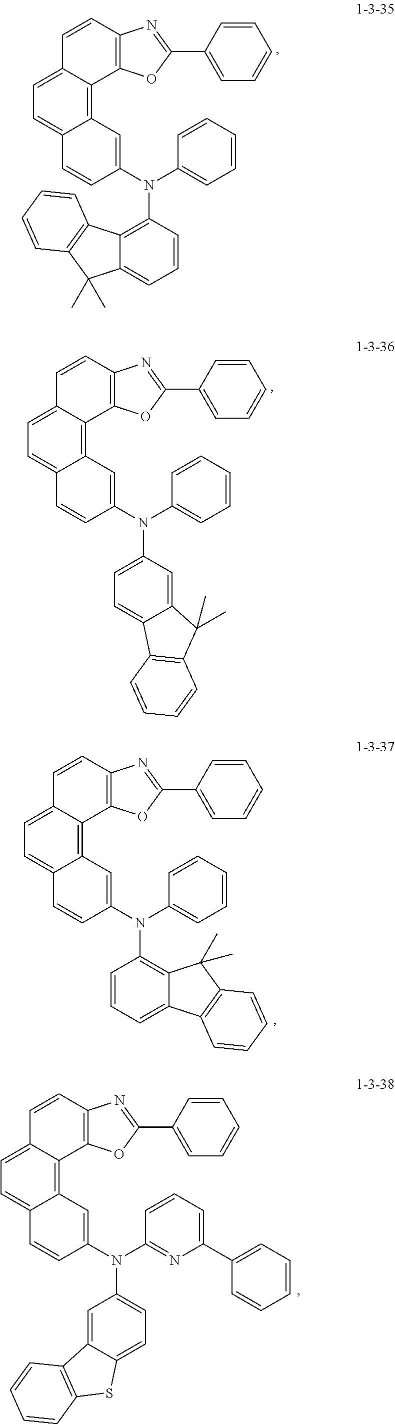 [SMILES: CC1(C)c2ccccc2-c2c(N(c3ccccc3)c3ccc4ccc5ccc6nc(-c7ccccc7)oc6c5c4c3)cccc21 ; CC1(C)c2ccccc2-c2ccc(N(c3ccccc3)c3ccc4ccc5ccc6nc(-c7ccccc7)oc6c5c4c3)cc21 ; CC1(C)c2ccccc2-c2cccc(N(c3ccccc3)c3ccc4ccc5ccc6nc(-c7ccccc7)oc6c5c4c3)c21 ; c1ccc(-c2cccc(N(c3ccc4sc5ccccc5c4c3)c3ccc4ccc5ccc6nc(-c7ccccc7)oc6c5c4c3)n2)cc1] 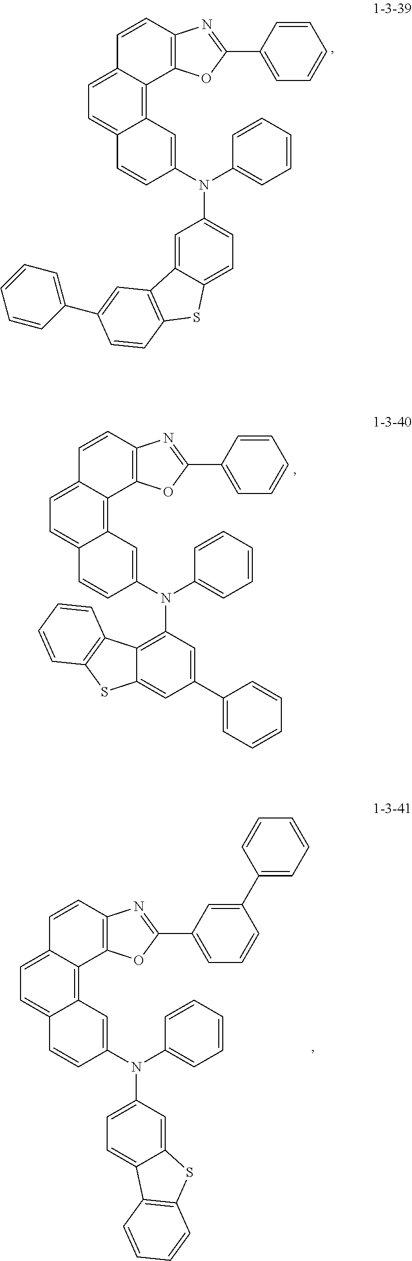 [SMILES: c1ccc(-c2ccc3sc4ccc(N(c5ccccc5)c5ccc6ccc7ccc8nc(-c9ccccc9)oc8c7c6c5)cc4c3c2)cc1 ; c1ccc(-c2cc(N(c3ccccc3)c3ccc4ccc5ccc6nc(-c7ccccc7)oc6c5c4c3)c3c(c2)sc2ccccc23)cc1 ; c1ccc(-c2cccc(-c3nc4ccc5ccc6ccc(N(c7ccccc7)c7ccc8c(c7)sc7ccccc78)cc6c5c4o3)c2)cc1] 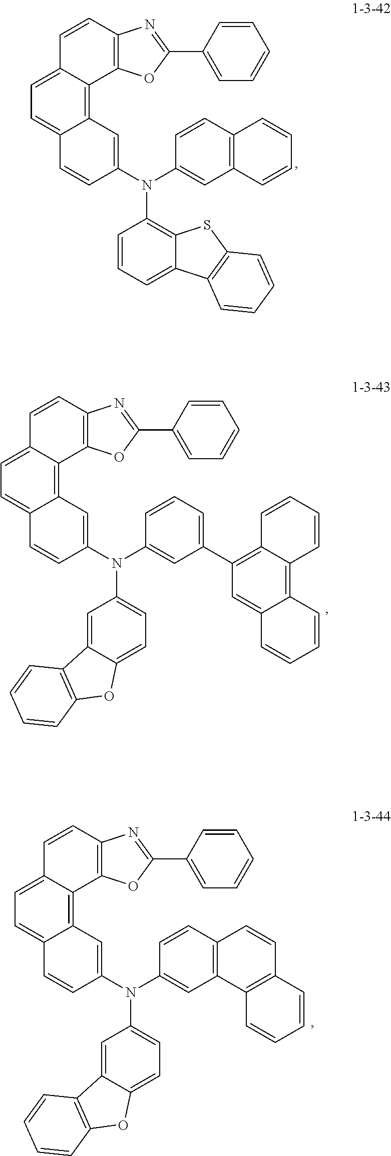 [SMILES: c1ccc(-c2nc3ccc4ccc5ccc(N(c6ccc7ccccc7c6)c6cccc7c6sc6ccccc67)cc5c4c3o2)cc1 ; c1ccc(-c2nc3ccc4ccc5ccc(N(c6cccc(-c7cc8ccccc8c8ccccc78)c6)c6ccc7oc8ccccc8c7c6)cc5c4c3o2)cc1 ; c1ccc(-c2nc3ccc4ccc5ccc(N(c6ccc7ccc8ccccc8c7c6)c6ccc7oc8ccccc8c7c6)cc5c4c3o2)cc1] 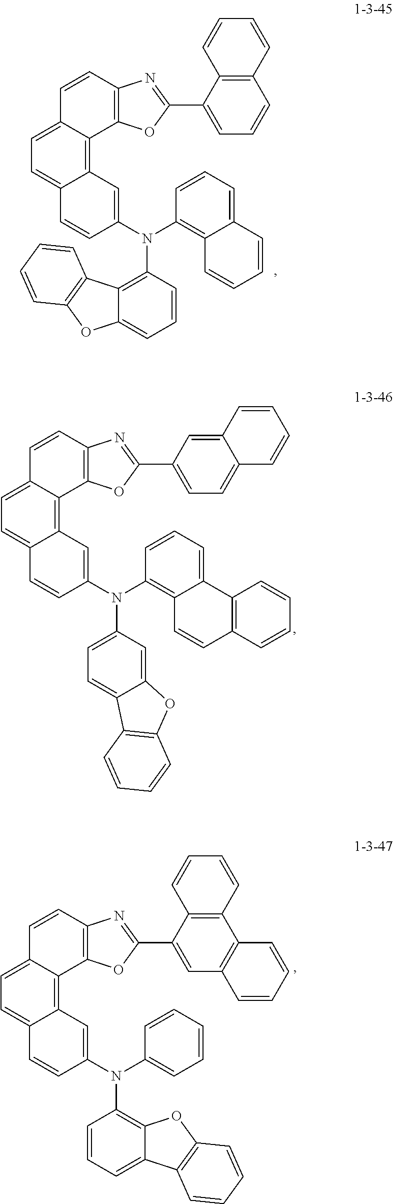 [SMILES: c1ccc2c(-c3nc4ccc5ccc6ccc(N(c7cccc8ccccc78)c7cccc8oc9ccccc9c78)cc6c5c4o3)cccc2c1 ; c1ccc2cc(-c3nc4ccc5ccc6ccc(N(c7ccc8c(c7)oc7ccccc78)c7cccc8c7ccc7ccccc78)cc6c5c4o3)ccc2c1 ; c1ccc(N(c2ccc3ccc4ccc5nc(-c6cc7ccccc7c7ccccc67)oc5c4c3c2)c2cccc3c2oc2ccccc23)cc1] 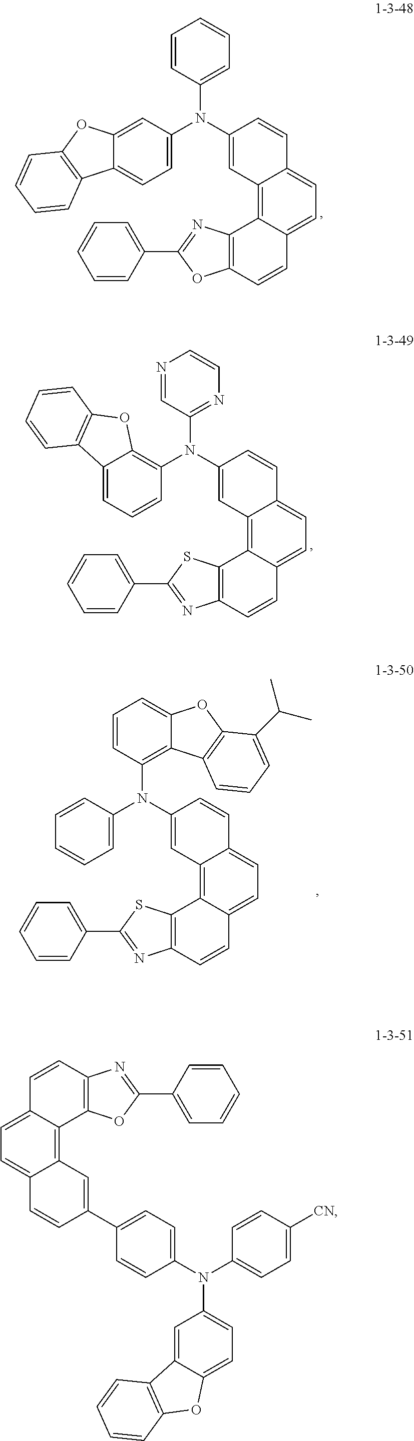 [SMILES: c1ccc(-c2nc3c(ccc4ccc5ccc(N(c6ccccc6)c6ccc7c(c6)oc6ccccc67)cc5c43)o2)cc1 ; c1ccc(-c2nc3ccc4ccc5ccc(N(c6cnccn6)c6cccc7c6oc6ccccc67)cc5c4c3s2)cc1 ; CC(C)c1cccc2c1oc1cccc(N(c3ccccc3)c3ccc4ccc5ccc6nc(-c7ccccc7)sc6c5c4c3)c12 ; N#Cc1ccc(N(c2ccc(-c3ccc4ccc5ccc6nc(-c7ccccc7)oc6c5c4c3)cc2)c2ccc3oc4ccccc4c3c2)cc1] 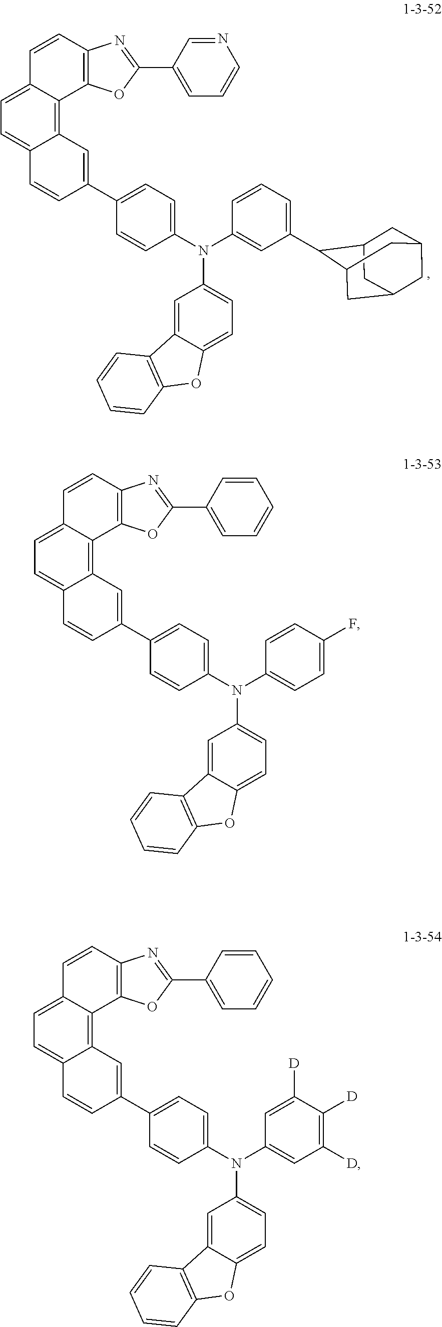 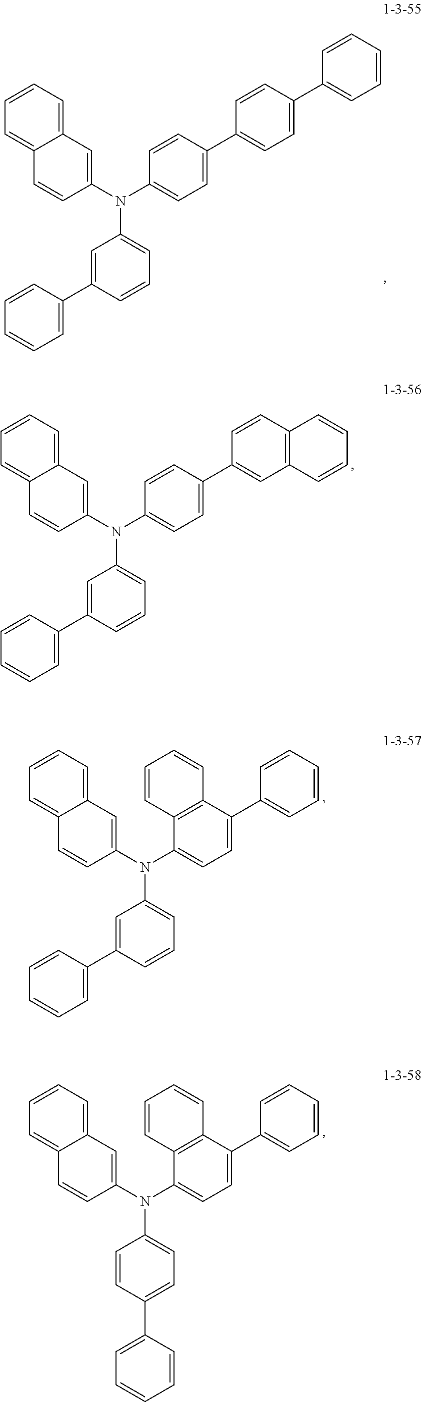 [SMILES: c1ccc(-c2ccc(-c3ccc(N(c4cccc(-c5ccccc5)c4)c4ccc5ccccc5c4)cc3)cc2)cc1 ; c1ccc(-c2cccc(N(c3ccc(-c4ccc5ccccc5c4)cc3)c3ccc4ccccc4c3)c2)cc1 ; c1ccc(-c2cccc(N(c3ccc4ccccc4c3)c3ccc(-c4ccccc4)c4ccccc34)c2)cc1 ; c1ccc(-c2ccc(N(c3ccc4ccccc4c3)c3ccc(-c4ccccc4)c4ccccc34)cc2)cc1] 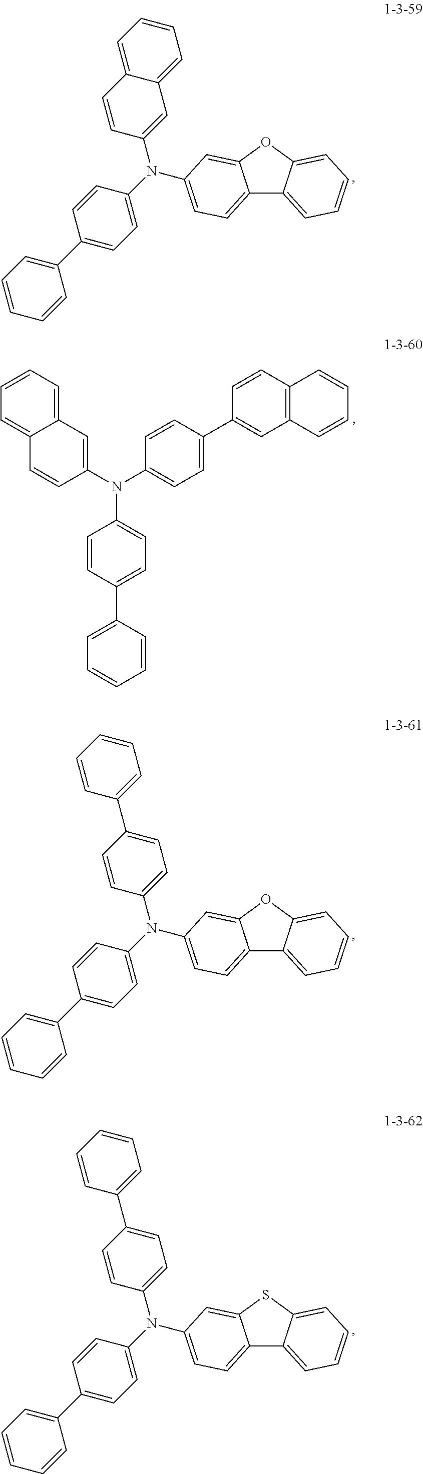 [SMILES: c1ccc(-c2ccc(N(c3ccc4ccccc4c3)c3ccc4c(c3)oc3ccccc34)cc2)cc1 ; c1ccc(-c2ccc(N(c3ccc(-c4ccc5ccccc5c4)cc3)c3ccc4ccccc4c3)cc2)cc1 ; c1ccc(-c2ccc(N(c3ccc(-c4ccccc4)cc3)c3ccc4c(c3)oc3ccccc34)cc2)cc1 ; c1ccc(-c2ccc(N(c3ccc(-c4ccccc4)cc3)c3ccc4c(c3)sc3ccccc34)cc2)cc1] 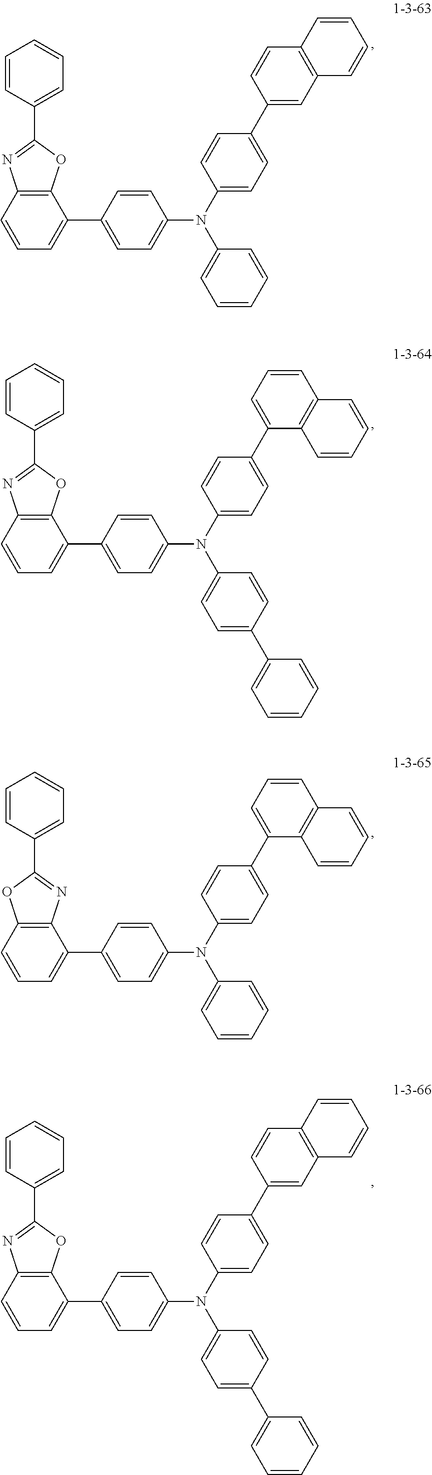 [SMILES: c1ccc(-c2nc3cccc(-c4ccc(N(c5ccccc5)c5ccc(-c6ccc7ccccc7c6)cc5)cc4)c3o2)cc1 ; c1ccc(-c2ccc(N(c3ccc(-c4cccc5ccccc45)cc3)c3ccc(-c4cccc5nc(-c6ccccc6)oc45)cc3)cc2)cc1 ; c1ccc(-c2nc3c(-c4ccc(N(c5ccccc5)c5ccc(-c6cccc7ccccc67)cc5)cc4)cccc3o2)cc1 ; c1ccc(-c2ccc(N(c3ccc(-c4ccc5ccccc5c4)cc3)c3ccc(-c4cccc5nc(-c6ccccc6)oc45)cc3)cc2)cc1] 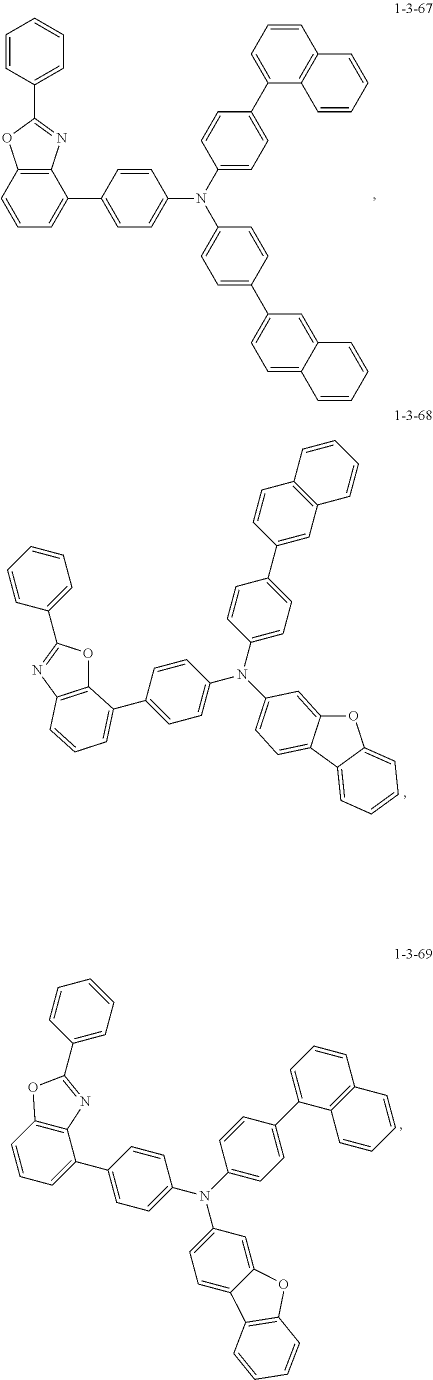 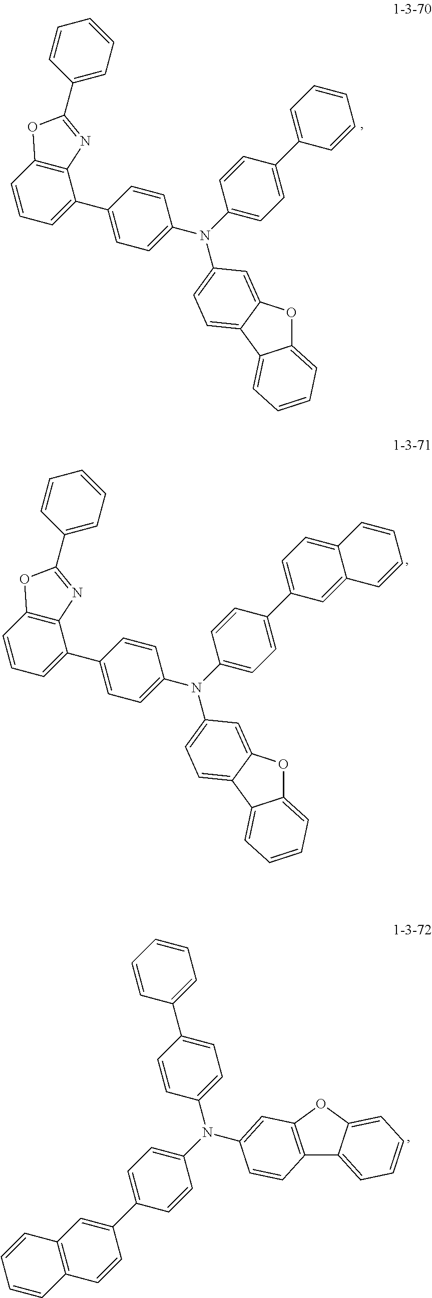 [SMILES: c1ccc(-c2ccc(N(c3ccc(-c4cccc5oc(-c6ccccc6)nc45)cc3)c3ccc4c(c3)oc3ccccc34)cc2)cc1 ; c1ccc(-c2nc3c(-c4ccc(N(c5ccc(-c6ccc7ccccc7c6)cc5)c5ccc6c(c5)oc5ccccc56)cc4)cccc3o2)cc1 ; c1ccc(-c2ccc(N(c3ccc(-c4ccc5ccccc5c4)cc3)c3ccc4c(c3)oc3ccccc34)cc2)cc1] 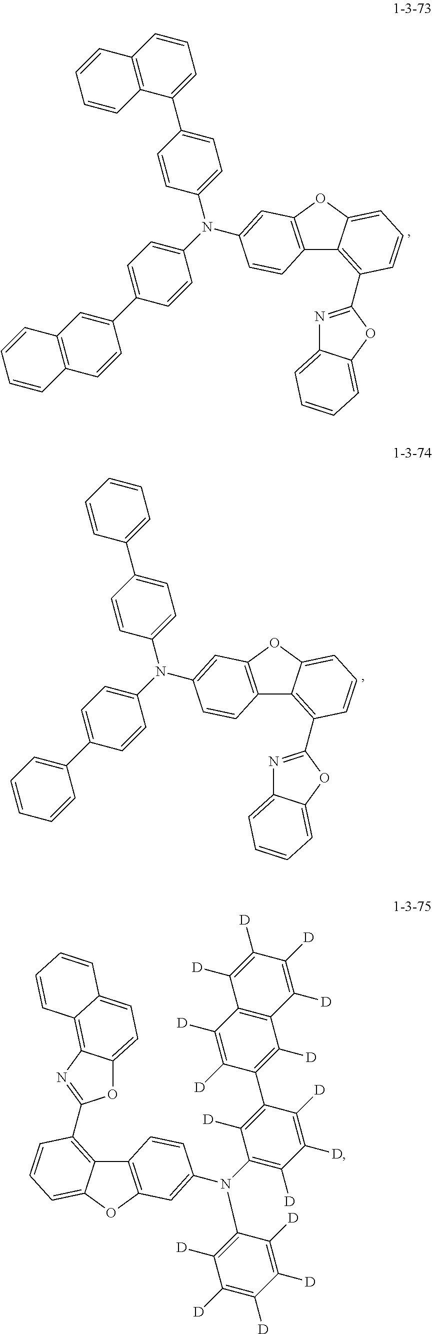 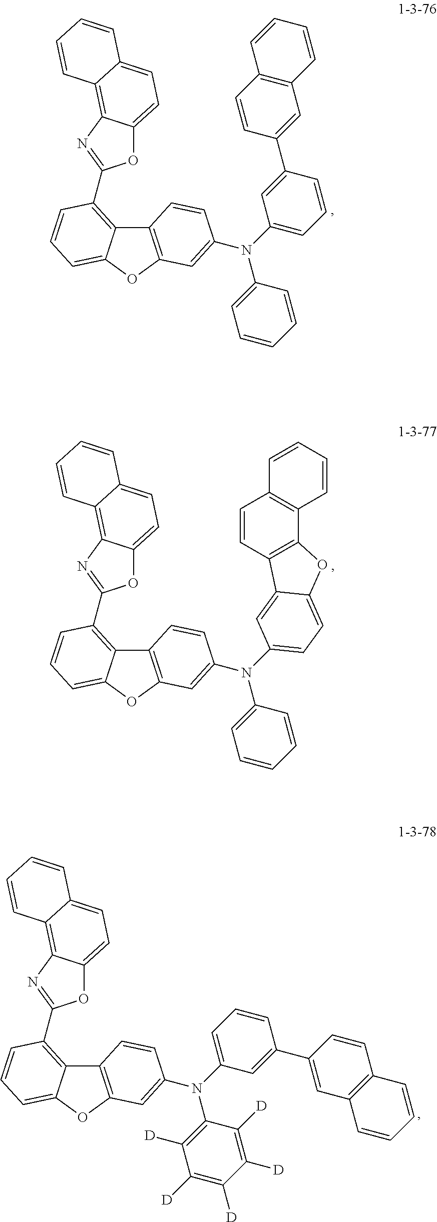 [SMILES: c1ccc(N(c2cccc(-c3ccc4ccccc4c3)c2)c2ccc3c(c2)oc2cccc(-c4nc5c(ccc6ccccc65)o4)c23)cc1 ; c1ccc(N(c2ccc3c(c2)oc2cccc(-c4nc5c(ccc6ccccc65)o4)c23)c2ccc3oc4c5ccccc5ccc4c3c2)cc1 ; [2H]c1c([2H])c([2H])c(N(c2cccc(-c3ccc4ccccc4c3)c2)c2ccc3c(c2)oc2cccc(-c4nc5c(ccc6ccccc65)o4)c23)c([2H])c1[2H]] 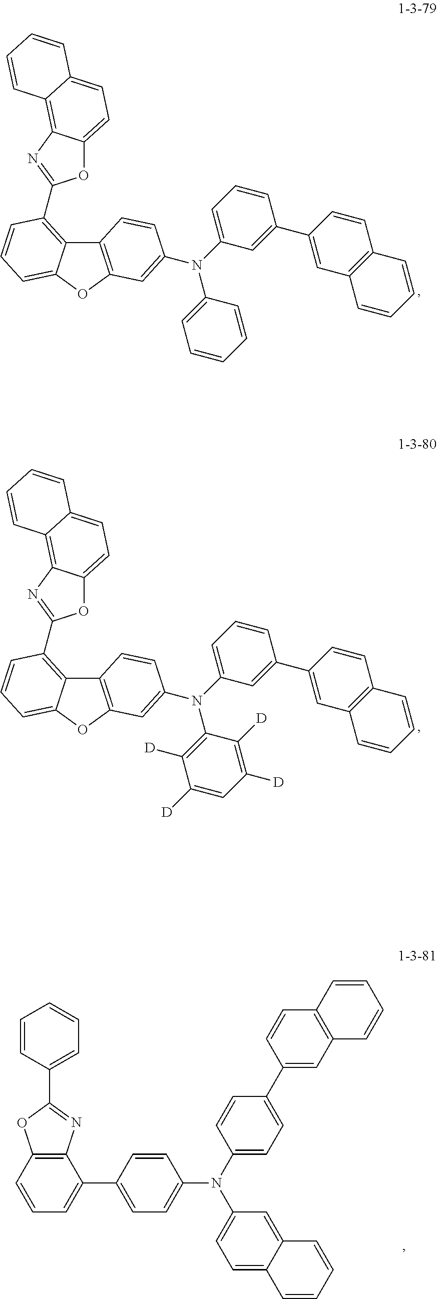 [SMILES: c1ccc(N(c2cccc(-c3ccc4ccccc4c3)c2)c2ccc3c(c2)oc2cccc(-c4nc5c(ccc6ccccc65)o4)c23)cc1 ; [2H]c1cc([2H])c([2H])c(N(c2cccc(-c3ccc4ccccc4c3)c2)c2ccc3c(c2)oc2cccc(-c4nc5c(ccc6ccccc65)o4)c23)c1[2H] ; c1ccc(-c2nc3c(-c4ccc(N(c5ccc(-c6ccc7ccccc7c6)cc5)c5ccc6ccccc6c5)cc4)cccc3o2)cc1] 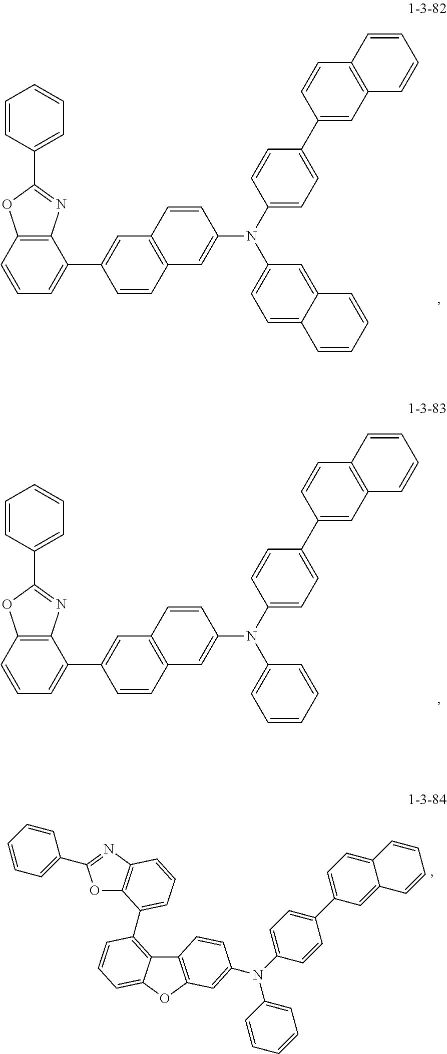 [SMILES: c1ccc(-c2nc3c(-c4ccc5cc(N(c6ccc(-c7ccc8ccccc8c7)cc6)c6ccc7ccccc7c6)ccc5c4)cccc3o2)cc1 ; c1ccc(-c2nc3c(-c4ccc5cc(N(c6ccccc6)c6ccc(-c7ccc8ccccc8c7)cc6)ccc5c4)cccc3o2)cc1 ; c1ccc(-c2nc3cccc(-c4cccc5oc6cc(N(c7ccccc7)c7ccc(-c8ccc9ccccc9c8)cc7)ccc6c45)c3o2)cc1] 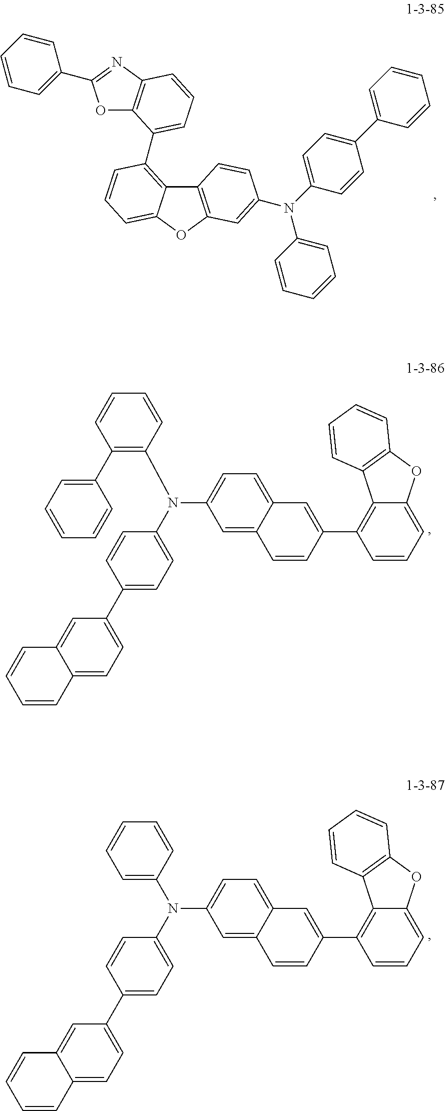 [SMILES: c1ccc(-c2ccc(N(c3ccccc3)c3ccc4c(c3)oc3cccc(-c5cccc6nc(-c7ccccc7)oc56)c34)cc2)cc1 ; c1ccc(-c2ccccc2N(c2ccc(-c3ccc4ccccc4c3)cc2)c2ccc3cc(-c4cccc5oc6ccccc6c45)ccc3c2)cc1 ; c1ccc(N(c2ccc(-c3ccc4ccccc4c3)cc2)c2ccc3cc(-c4cccc5oc6ccccc6c45)ccc3c2)cc1] 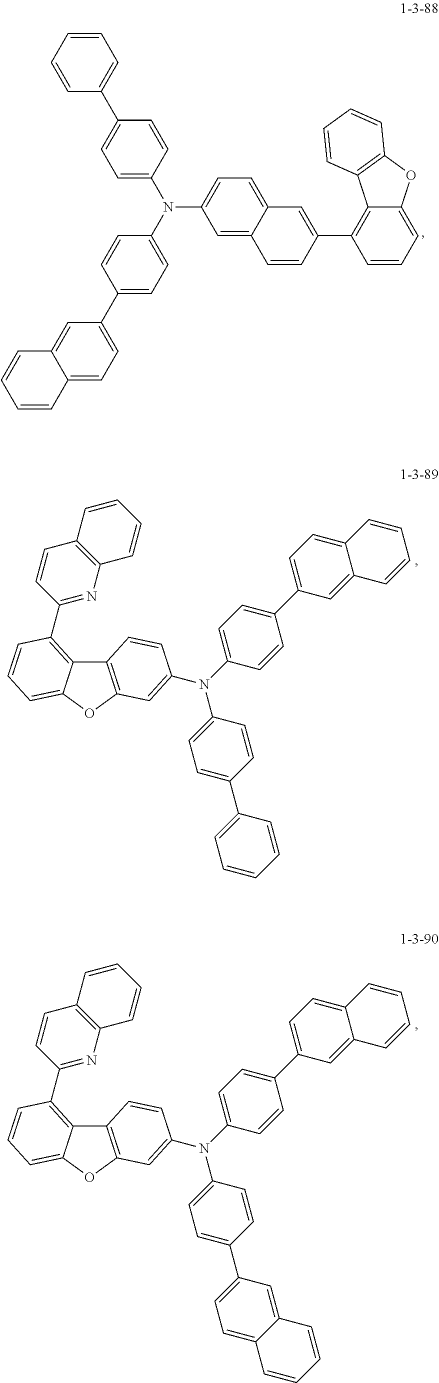 [SMILES: c1ccc(-c2ccc(N(c3ccc(-c4ccc5ccccc5c4)cc3)c3ccc4cc(-c5cccc6oc7ccccc7c56)ccc4c3)cc2)cc1 ; c1ccc(-c2ccc(N(c3ccc(-c4ccc5ccccc5c4)cc3)c3ccc4c(c3)oc3cccc(-c5ccc6ccccc6n5)c34)cc2)cc1 ; c1ccc2cc(-c3ccc(N(c4ccc(-c5ccc6ccccc6c5)cc4)c4ccc5c(c4)oc4cccc(-c6ccc7ccccc7n6)c45)cc3)ccc2c1] 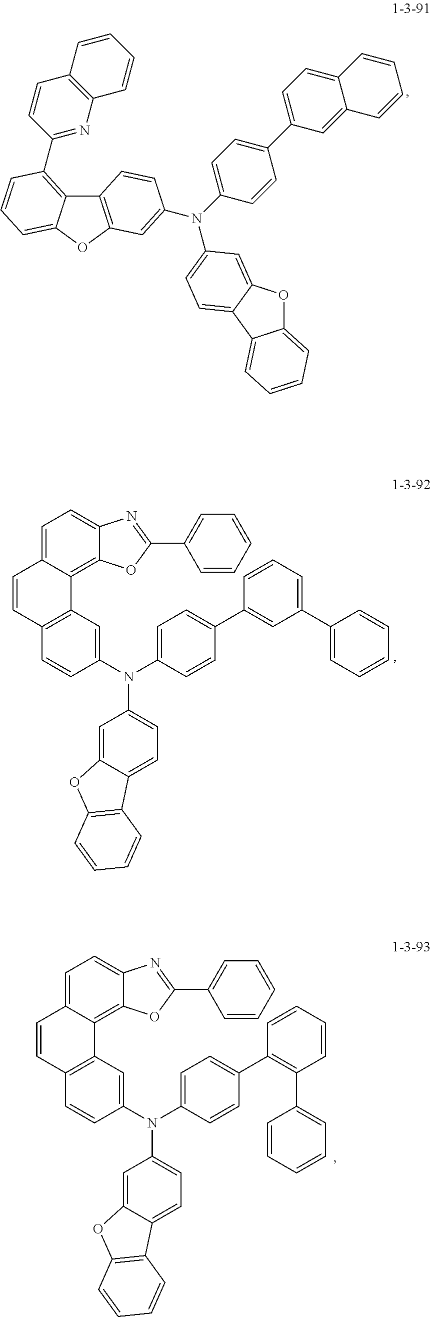 [SMILES: c1ccc2cc(-c3ccc(N(c4ccc5c(c4)oc4ccccc45)c4ccc5c(c4)oc4cccc(-c6ccc7ccccc7n6)c45)cc3)ccc2c1 ; c1ccc(-c2cccc(-c3ccc(N(c4ccc5c(c4)oc4ccccc45)c4ccc5ccc6ccc7nc(-c8ccccc8)oc7c6c5c4)cc3)c2)cc1 ; c1ccc(-c2nc3ccc4ccc5ccc(N(c6ccc(-c7ccccc7-c7ccccc7)cc6)c6ccc7c(c6)oc6ccccc67)cc5c4c3o2)cc1] 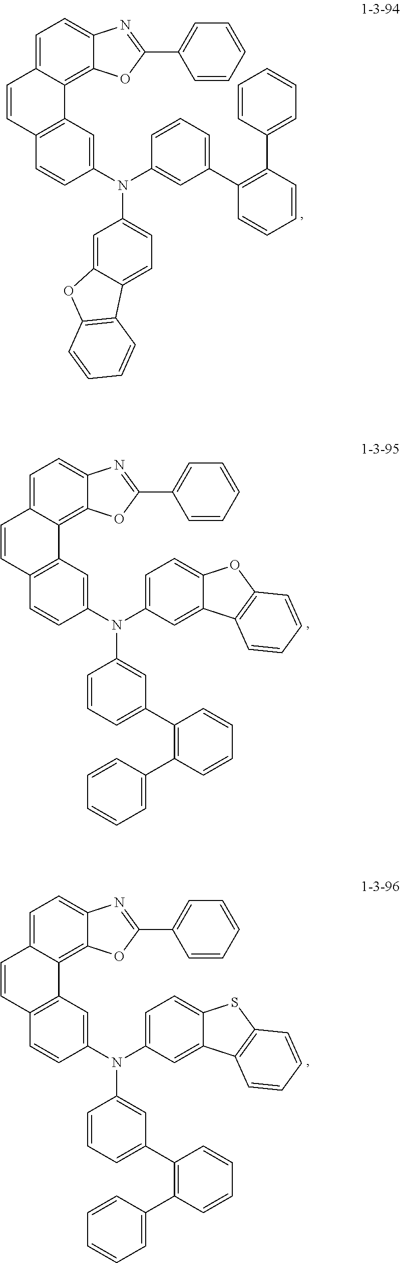 [SMILES: c1ccc(-c2nc3ccc4ccc5ccc(N(c6cccc(-c7ccccc7-c7ccccc7)c6)c6ccc7c(c6)oc6ccccc67)cc5c4c3o2)cc1 ; c1ccc(-c2nc3ccc4ccc5ccc(N(c6cccc(-c7ccccc7-c7ccccc7)c6)c6ccc7oc8ccccc8c7c6)cc5c4c3o2)cc1 ; c1ccc(-c2nc3ccc4ccc5ccc(N(c6cccc(-c7ccccc7-c7ccccc7)c6)c6ccc7sc8ccccc8c7c6)cc5c4c3o2)cc1] 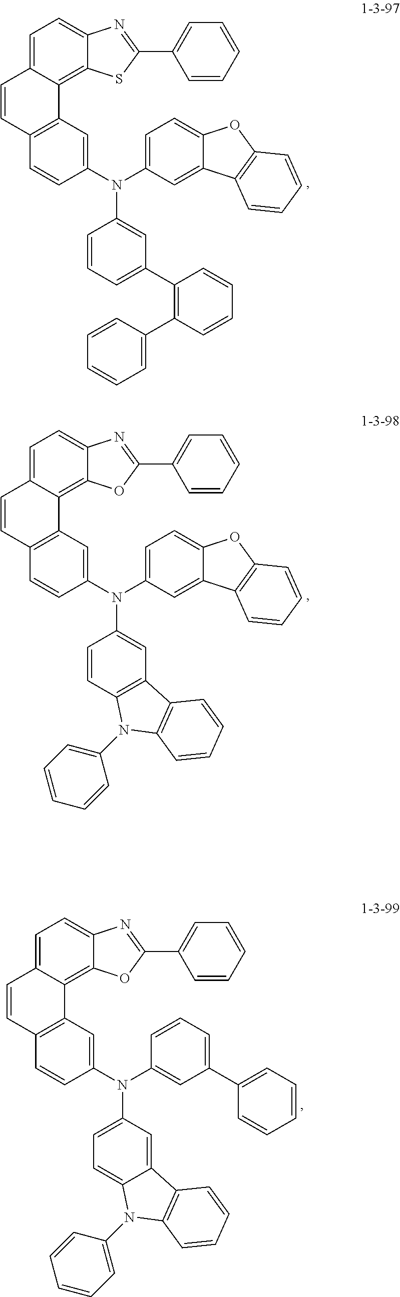 [SMILES: c1ccc(-c2nc3ccc4ccc5ccc(N(c6cccc(-c7ccccc7-c7ccccc7)c6)c6ccc7oc8ccccc8c7c6)cc5c4c3s2)cc1 ; c1ccc(-c2nc3ccc4ccc5ccc(N(c6ccc7oc8ccccc8c7c6)c6ccc7c(c6)c6ccccc6n7-c6ccccc6)cc5c4c3o2)cc1 ; c1ccc(-c2cccc(N(c3ccc4ccc5ccc6nc(-c7ccccc7)oc6c5c4c3)c3ccc4c(c3)c3ccccc3n4-c3ccccc3)c2)cc1] 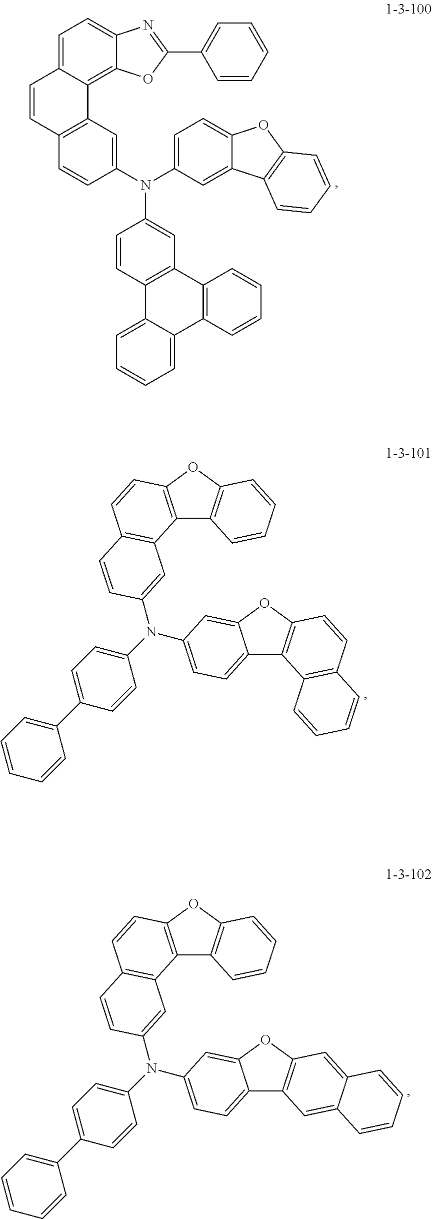 [SMILES: c1ccc(-c2nc3ccc4ccc5ccc(N(c6ccc7oc8ccccc8c7c6)c6ccc7c8ccccc8c8ccccc8c7c6)cc5c4c3o2)cc1 ; c1ccc(-c2ccc(N(c3ccc4c(c3)oc3ccc5ccccc5c34)c3ccc4ccc5oc6ccccc6c5c4c3)cc2)cc1 ; c1ccc(-c2ccc(N(c3ccc4c(c3)oc3cc5ccccc5cc34)c3ccc4ccc5oc6ccccc6c5c4c3)cc2)cc1] 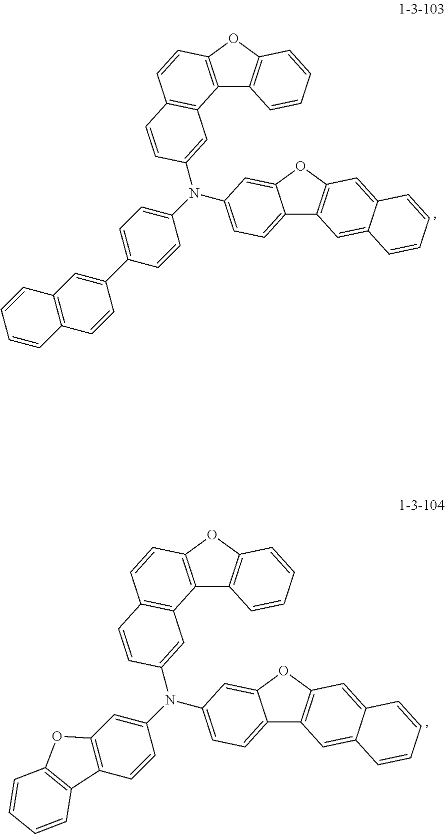 [SMILES: c1ccc2cc(-c3ccc(N(c4ccc5c(c4)oc4cc6ccccc6cc45)c4ccc5ccc6oc7ccccc7c6c5c4)cc3)ccc2c1 ; c1ccc2cc3c(cc2c1)oc1cc(N(c2ccc4c(c2)oc2ccccc24)c2ccc4ccc5oc6ccccc6c5c4c2)ccc13] 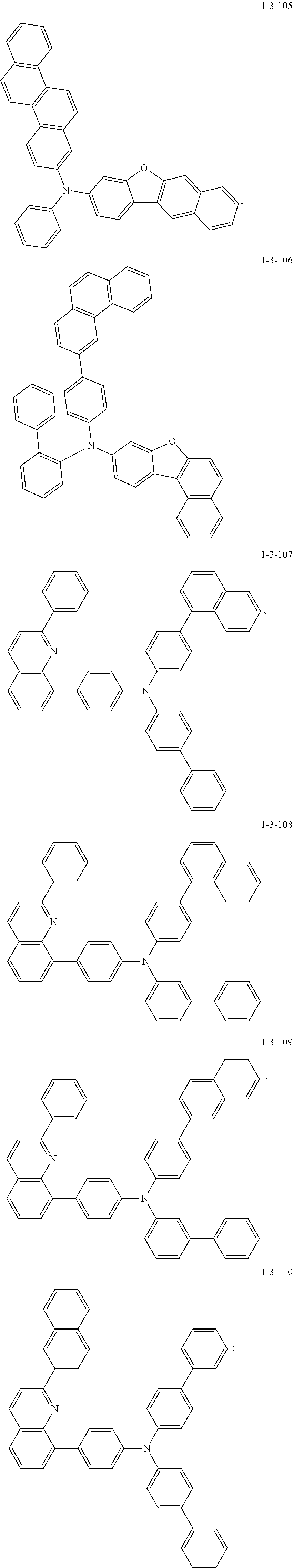
wherein optionally, hydrogens in Compound 1-1-1 to Compound 1-1-104, Compound 1-2-1 to Compound 1-2-100, and Compound 1-3-1 to Compound 1-3-110 can be partially or fully substituted with deuterium.

13. The organic electroluminescent device according to claim 11, wherein the emissive layer comprises a second host material;
preferably, the second host material has a structure represented by Formula NH-1: 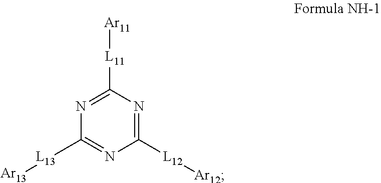
wherein L₁₁ to L₁₃ are, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 30 carbon atoms, substituted or unsubstituted heteroarylene having 3 to 30 carbon atoms, or a combination thereof; and
Ar₁₁ to Ar₁₃ are, at each occurrence identically or differently, selected from substituted or unsubstituted aryl having 6 to 30 carbon atoms, substituted or unsubstituted heteroaryl having 3 to 30 carbon atoms, or a combination thereof,
more preferably, the second host material is selected from the group consisting of Compound B-1 to Compound B-243: 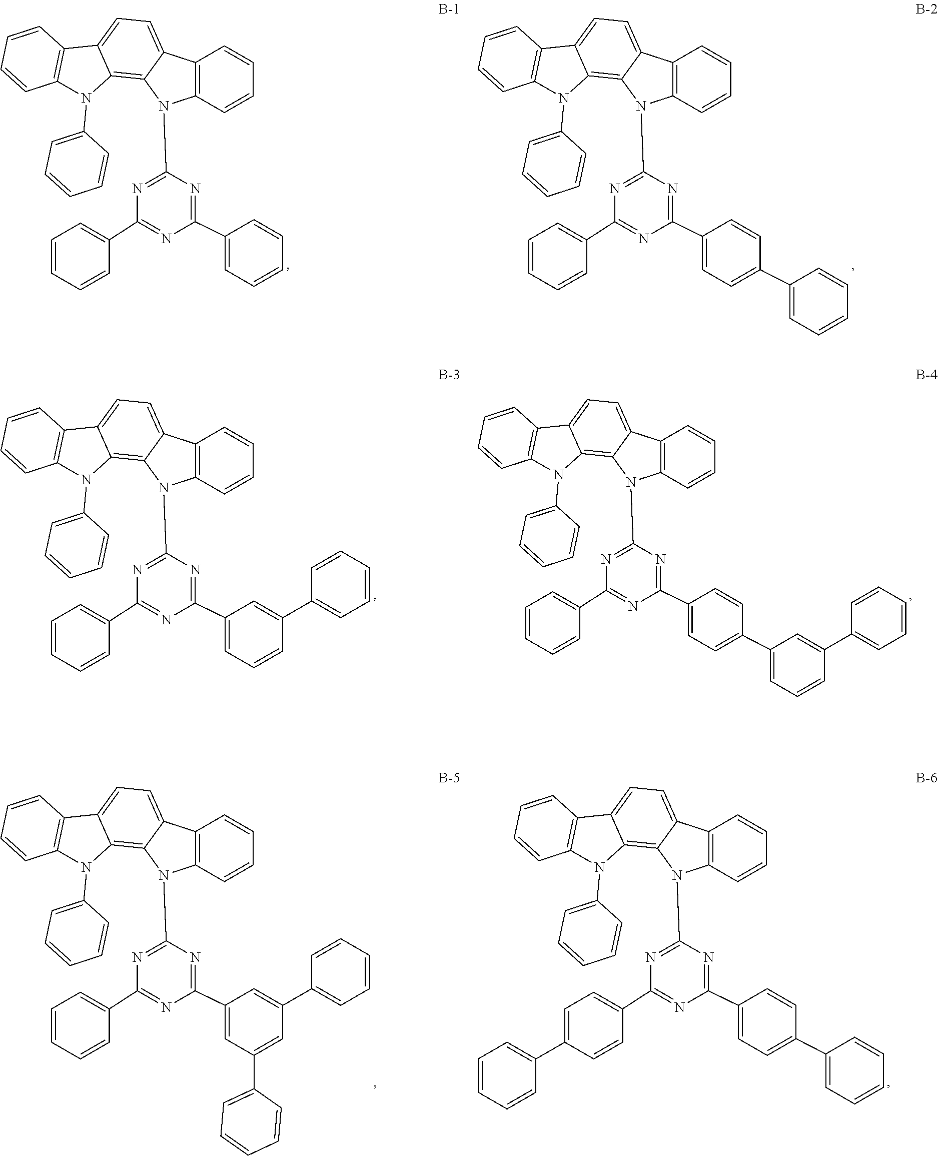 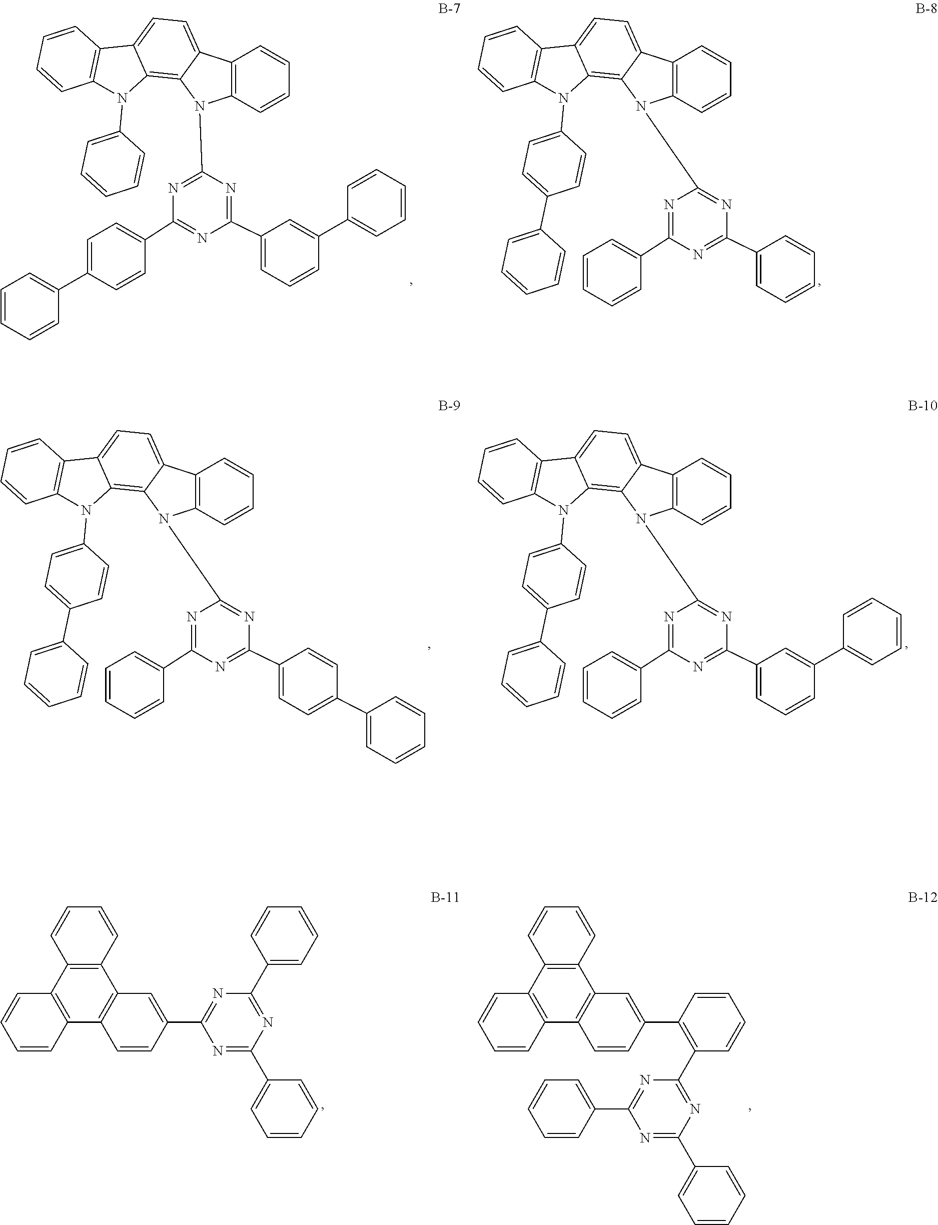 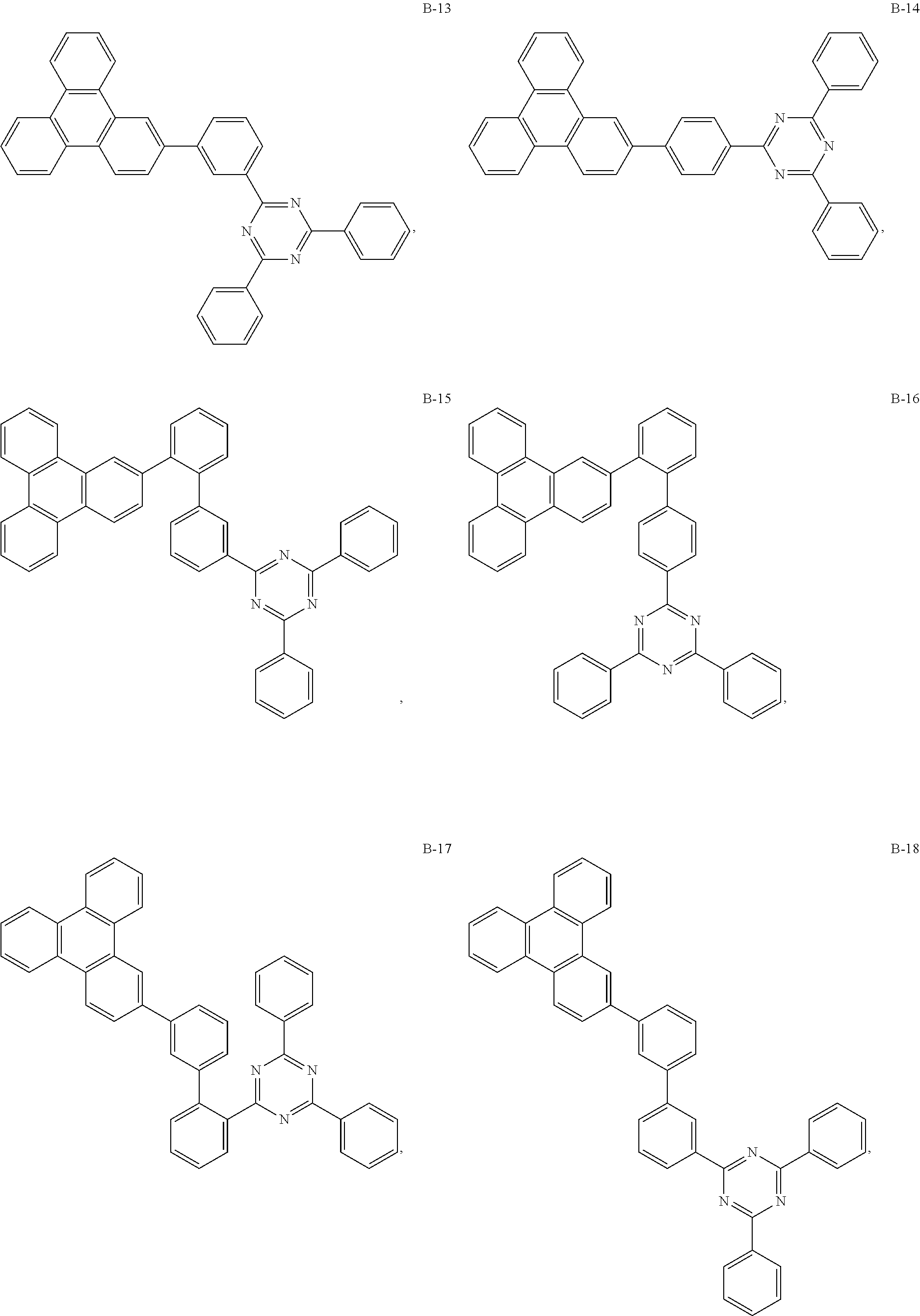 [SMILES: c1ccc(-c2nc(-c3ccccc3)nc(-c3cccc(-c4ccc5c6ccccc6c6ccccc6c5c4)c3)n2)cc1 ; c1ccc(-c2nc(-c3ccccc3)nc(-c3ccc(-c4ccc5c6ccccc6c6ccccc6c5c4)cc3)n2)cc1 ; c1ccc(-c2nc(-c3ccccc3)nc(-c3cccc(-c4ccccc4-c4ccc5c6ccccc6c6ccccc6c5c4)c3)n2)cc1 ; c1ccc(-c2nc(-c3ccccc3)nc(-c3ccc(-c4ccccc4-c4ccc5c6ccccc6c6ccccc6c5c4)cc3)n2)cc1 ; c1ccc(-c2nc(-c3ccccc3)nc(-c3ccccc3-c3cccc(-c4ccc5c6ccccc6c6ccccc6c5c4)c3)n2)cc1 ; c1ccc(-c2nc(-c3ccccc3)nc(-c3cccc(-c4cccc(-c5ccc6c7ccccc7c7ccccc7c6c5)c4)c3)n2)cc1] 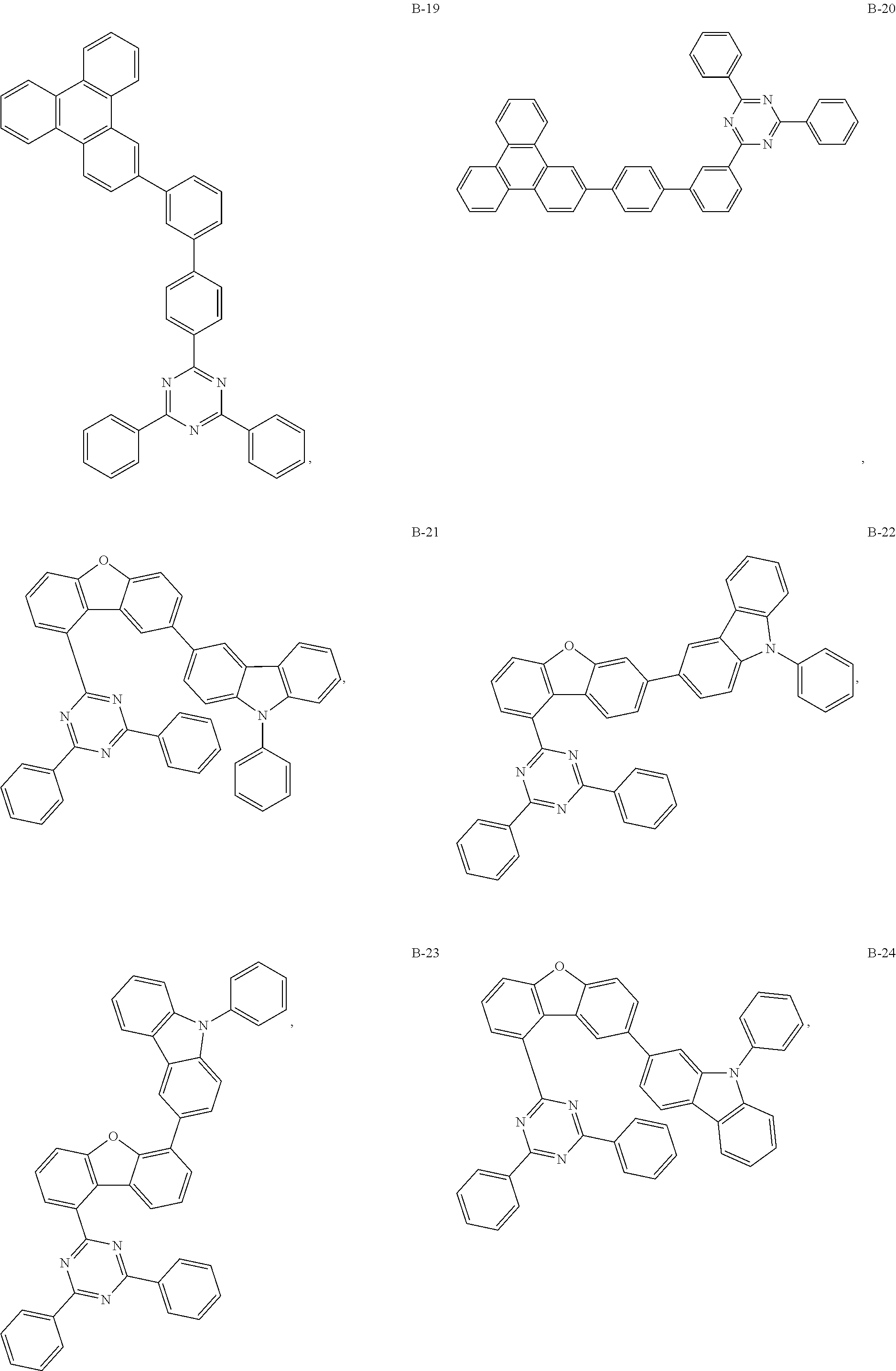 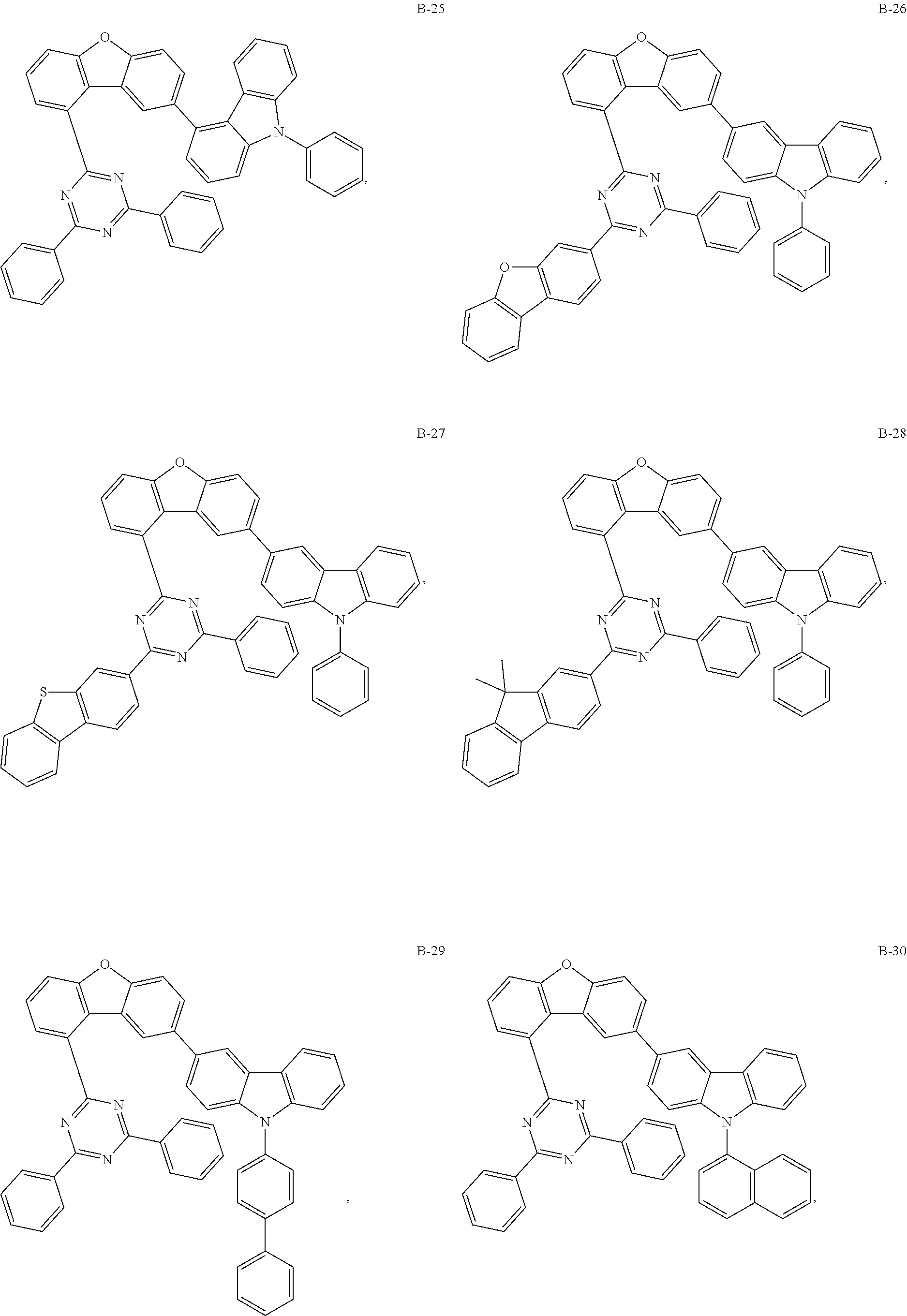 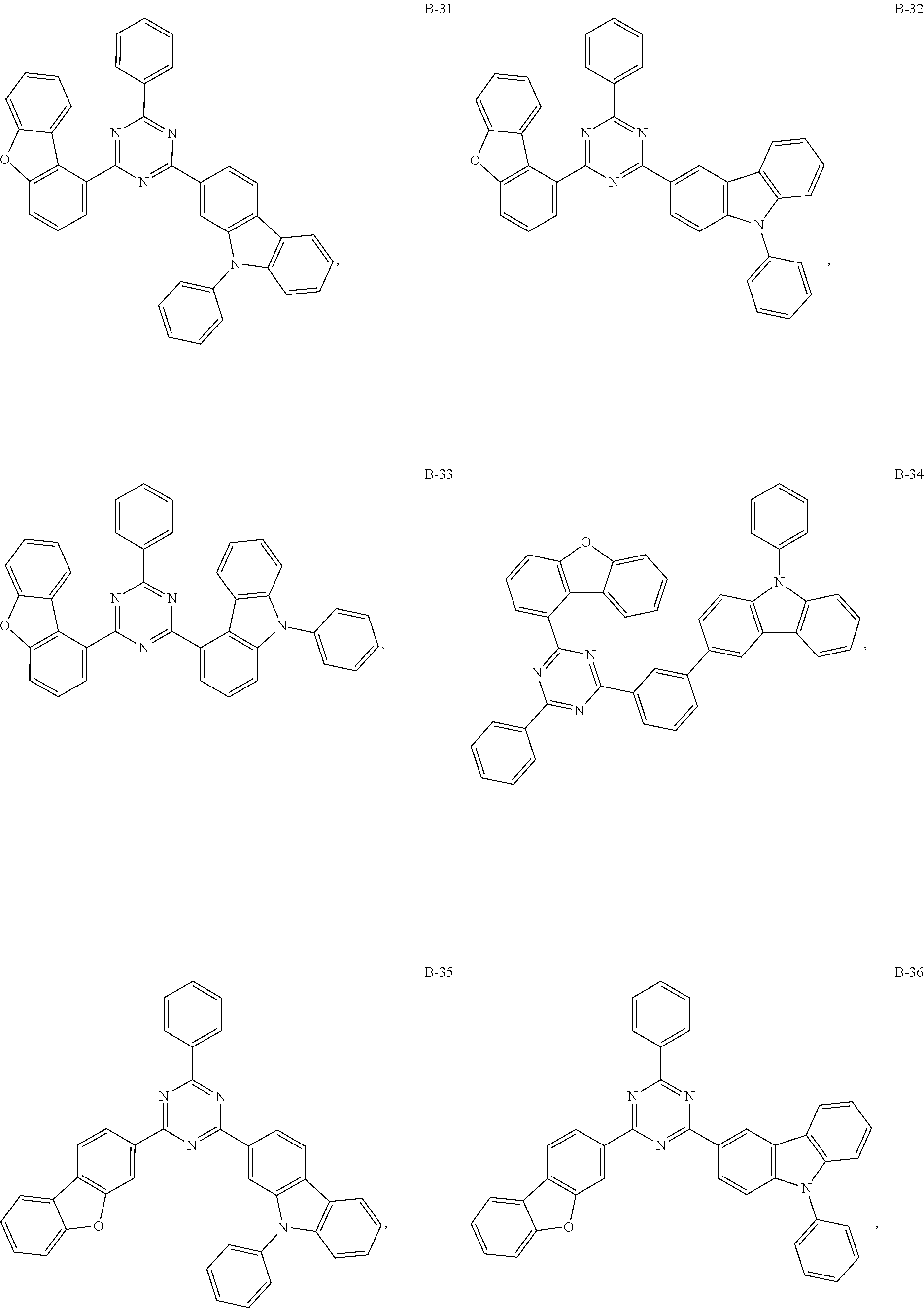 [SMILES: c1ccc(-c2nc(-c3ccc4c5ccccc5n(-c5ccccc5)c4c3)nc(-c3cccc4oc5ccccc5c34)n2)cc1 ; c1ccc(-c2nc(-c3ccc4c(c3)c3ccccc3n4-c3ccccc3)nc(-c3cccc4oc5ccccc5c34)n2)cc1 ; c1ccc(-c2nc(-c3cccc4oc5ccccc5c34)nc(-c3cccc4c3c3ccccc3n4-c3ccccc3)n2)cc1 ; c1ccc(-c2nc(-c3cccc(-c4ccc5c(c4)c4ccccc4n5-c4ccccc4)c3)nc(-c3cccc4oc5ccccc5c34)n2)cc1 ; c1ccc(-c2nc(-c3ccc4c(c3)oc3ccccc34)nc(-c3ccc4c5ccccc5n(-c5ccccc5)c4c3)n2)cc1 ; c1ccc(-c2nc(-c3ccc4c(c3)oc3ccccc34)nc(-c3ccc4c(c3)c3ccccc3n4-c3ccccc3)n2)cc1] 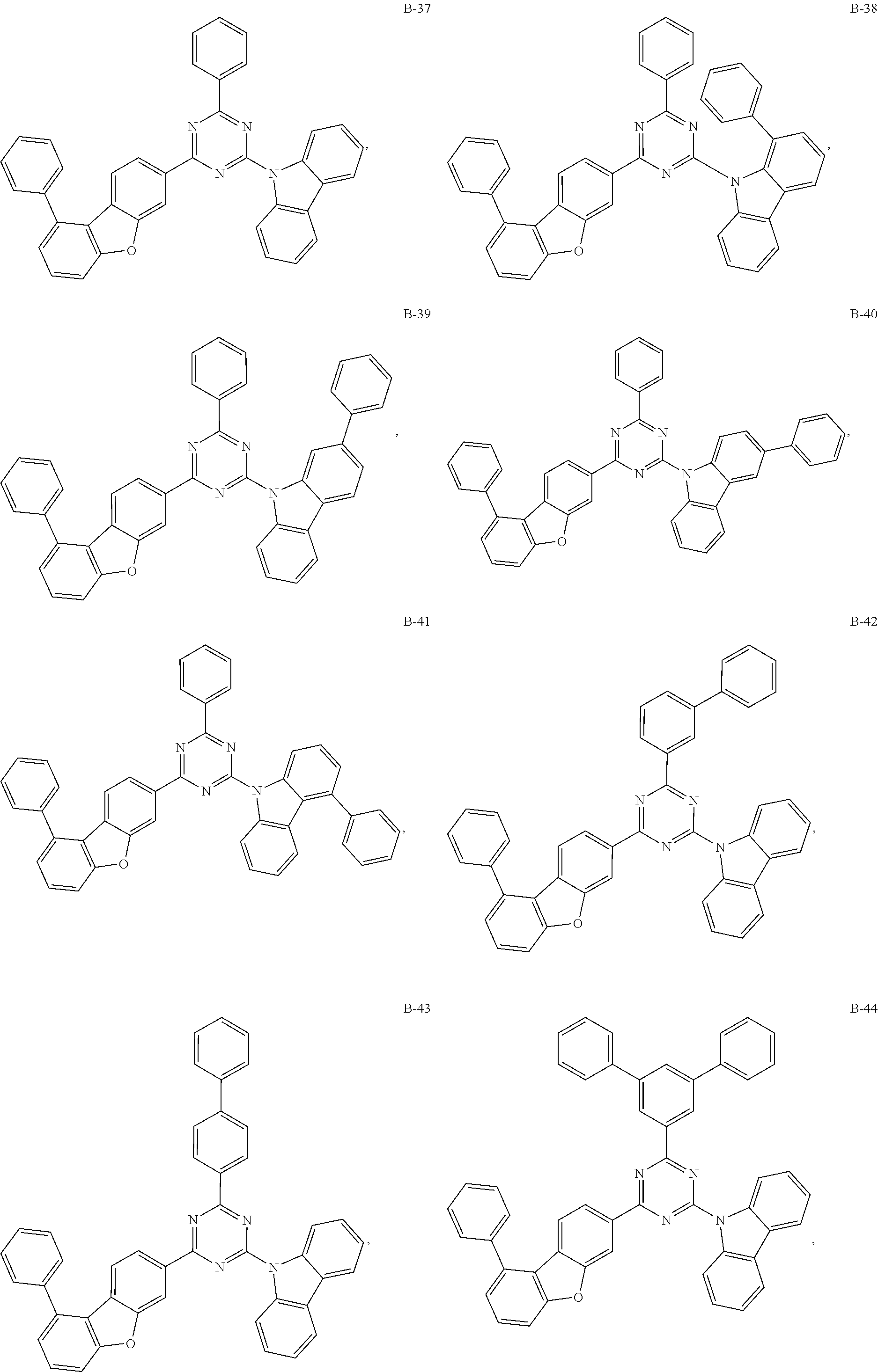 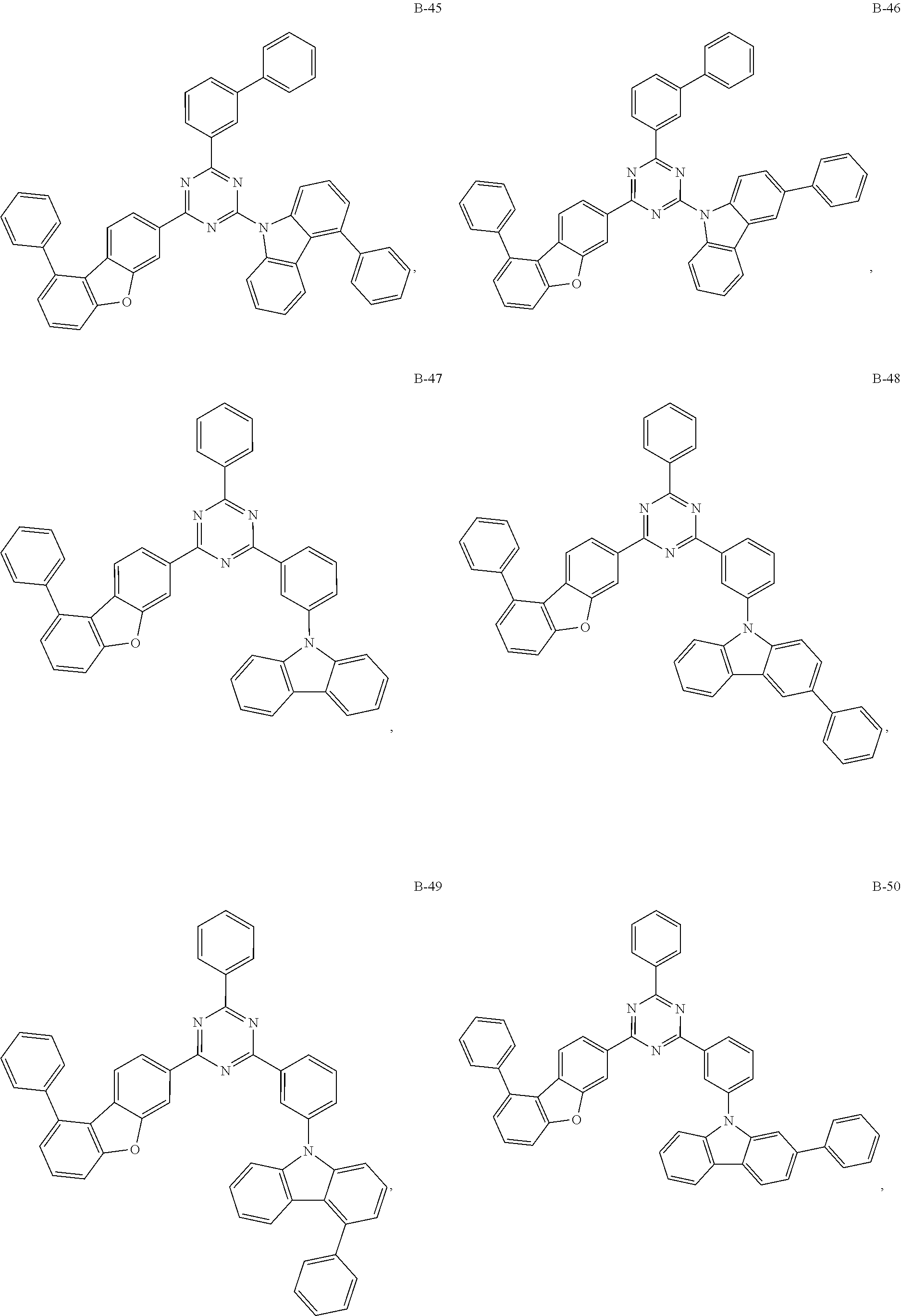 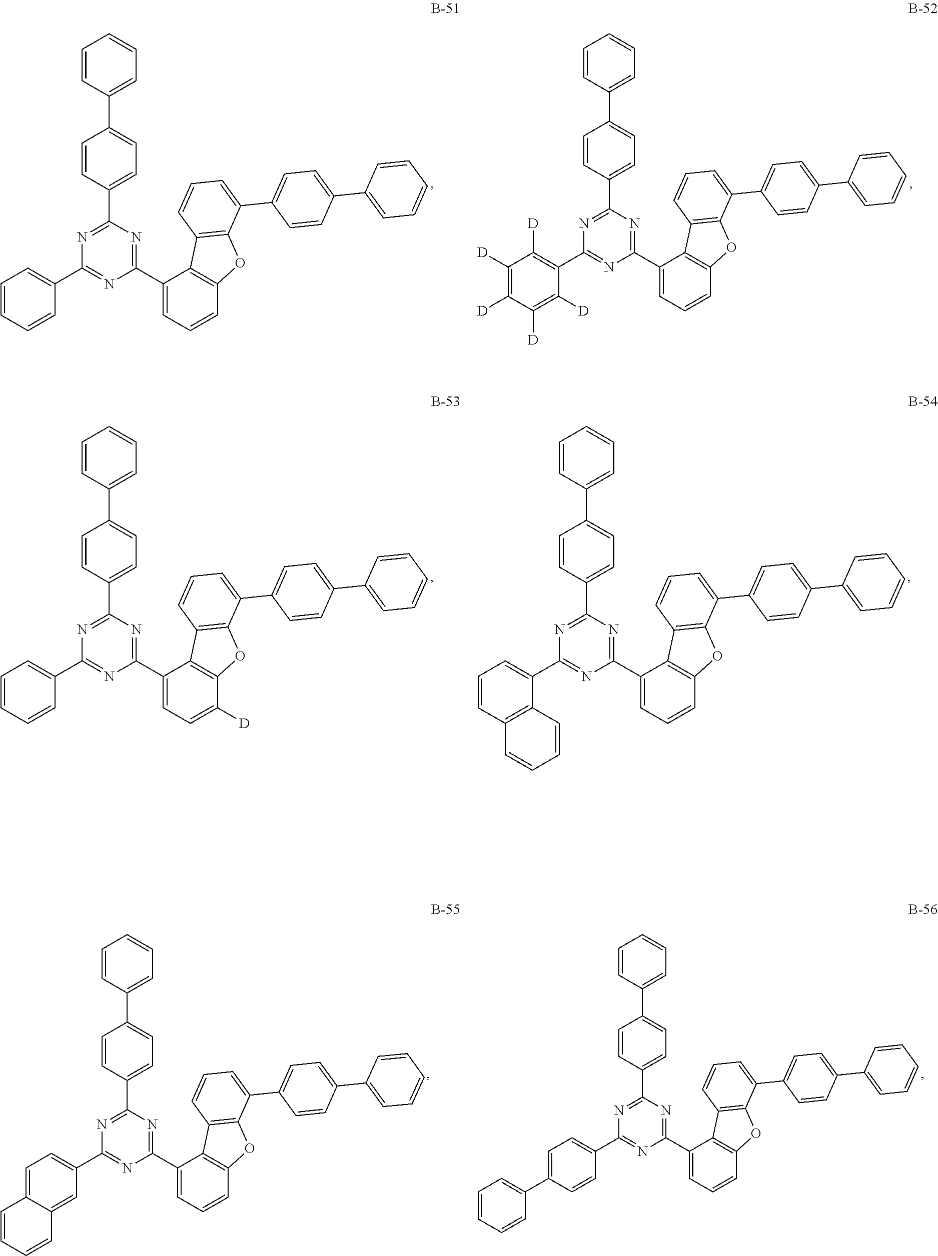 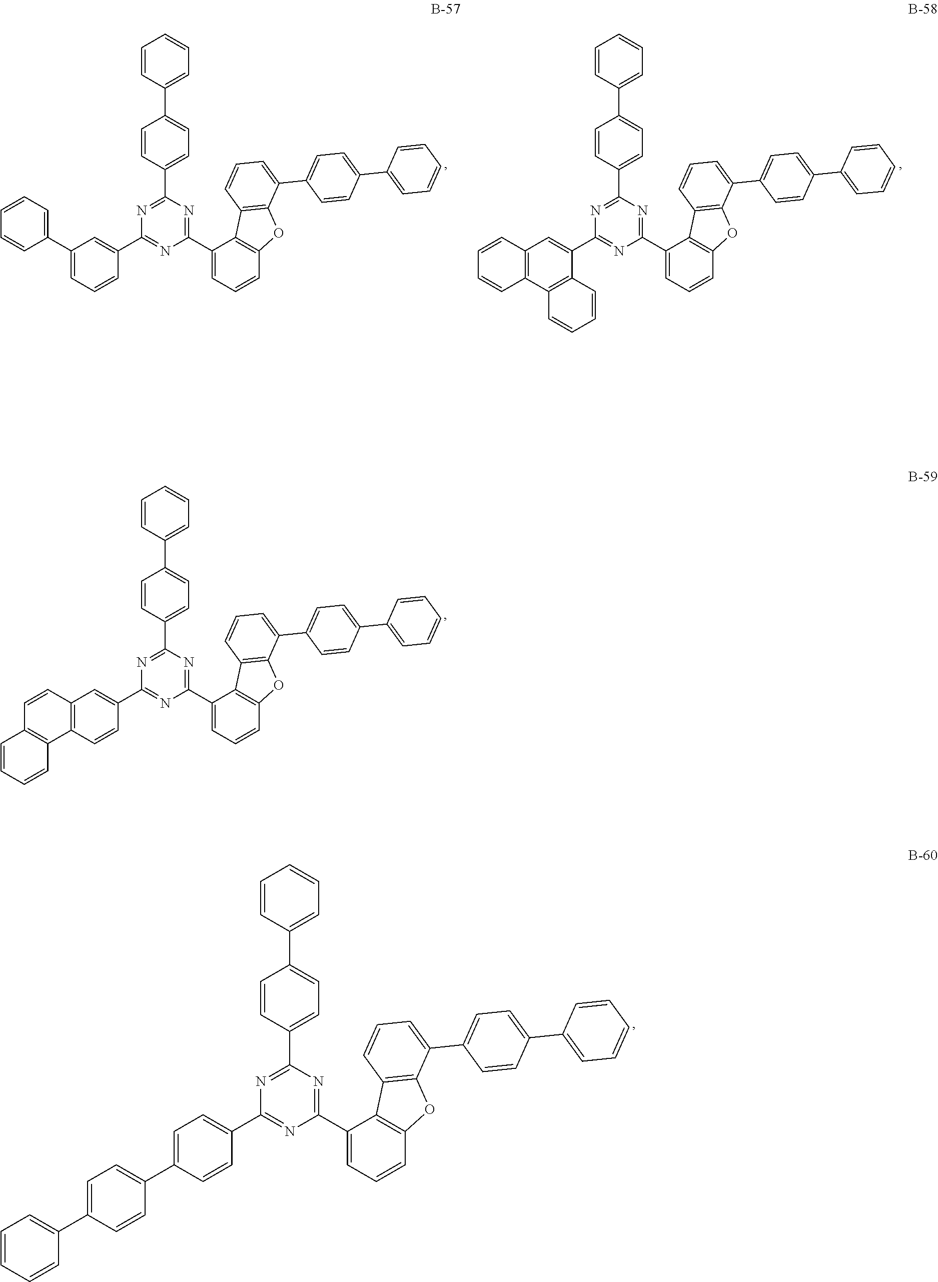 [SMILES: c1ccc(-c2ccc(-c3nc(-c4cccc(-c5ccccc5)c4)nc(-c4cccc5oc6c(-c7ccc(-c8ccccc8)cc7)cccc6c45)n3)cc2)cc1 ; c1ccc(-c2ccc(-c3nc(-c4cc5ccccc5c5ccccc45)nc(-c4cccc5oc6c(-c7ccc(-c8ccccc8)cc7)cccc6c45)n3)cc2)cc1 ; c1ccc(-c2ccc(-c3nc(-c4ccc5c(ccc6ccccc65)c4)nc(-c4cccc5oc6c(-c7ccc(-c8ccccc8)cc7)cccc6c45)n3)cc2)cc1 ; c1ccc(-c2ccc(-c3ccc(-c4nc(-c5ccc(-c6ccccc6)cc5)nc(-c5cccc6oc7c(-c8ccc(-c9ccccc9)cc8)cccc7c56)n4)cc3)cc2)cc1] 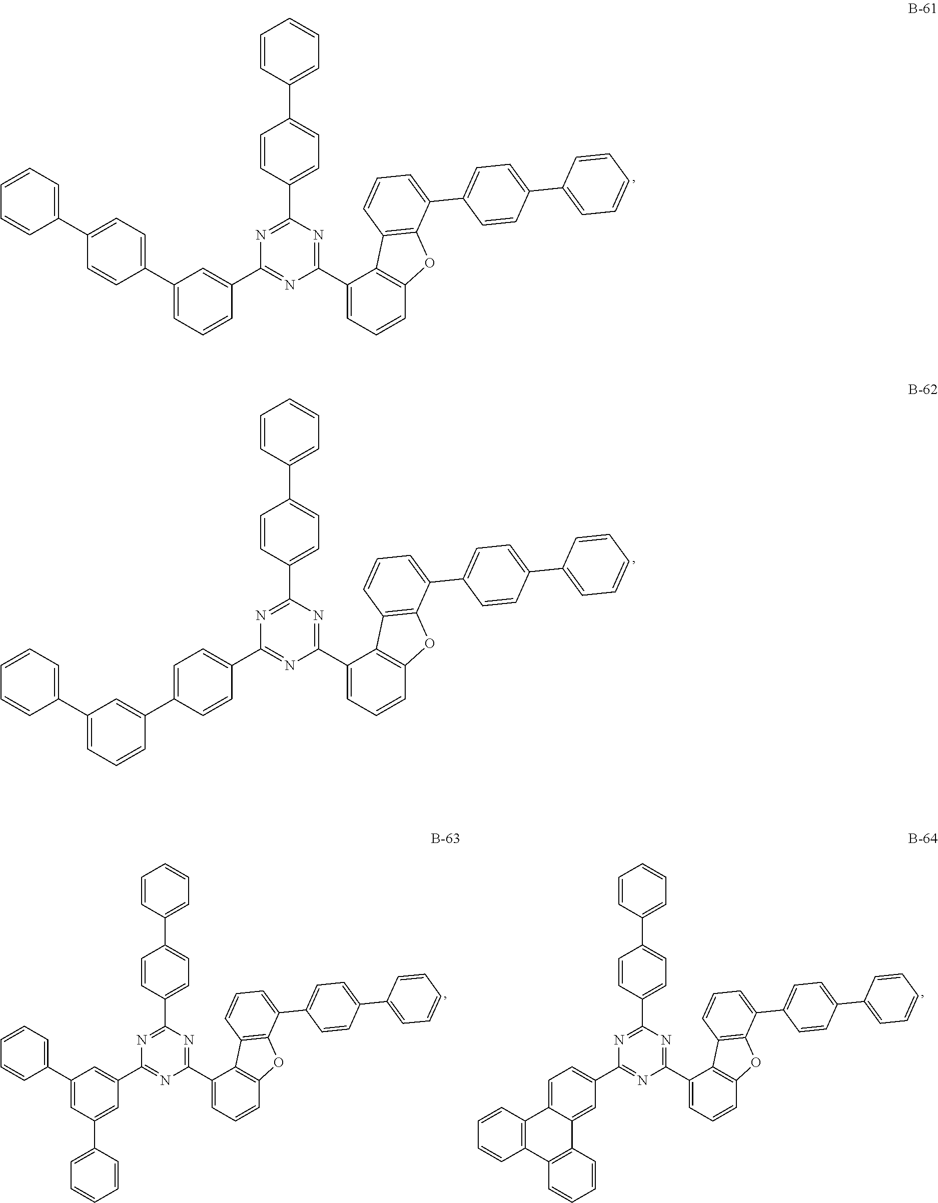 [SMILES: c1ccc(-c2ccc(-c3cccc(-c4nc(-c5ccc(-c6ccccc6)cc5)nc(-c5cccc6oc7c(-c8ccc(-c9ccccc9)cc8)cccc7c56)n4)c3)cc2)cc1 ; c1ccc(-c2ccc(-c3nc(-c4ccc(-c5cccc(-c6ccccc6)c5)cc4)nc(-c4cccc5oc6c(-c7ccc(-c8ccccc8)cc7)cccc6c45)n3)cc2)cc1 ; c1ccc(-c2ccc(-c3nc(-c4cc(-c5ccccc5)cc(-c5ccccc5)c4)nc(-c4cccc5oc6c(-c7ccc(-c8ccccc8)cc7)cccc6c45)n3)cc2)cc1 ; c1ccc(-c2ccc(-c3nc(-c4ccc5c6ccccc6c6ccccc6c5c4)nc(-c4cccc5oc6c(-c7ccc(-c8ccccc8)cc7)cccc6c45)n3)cc2)cc1] 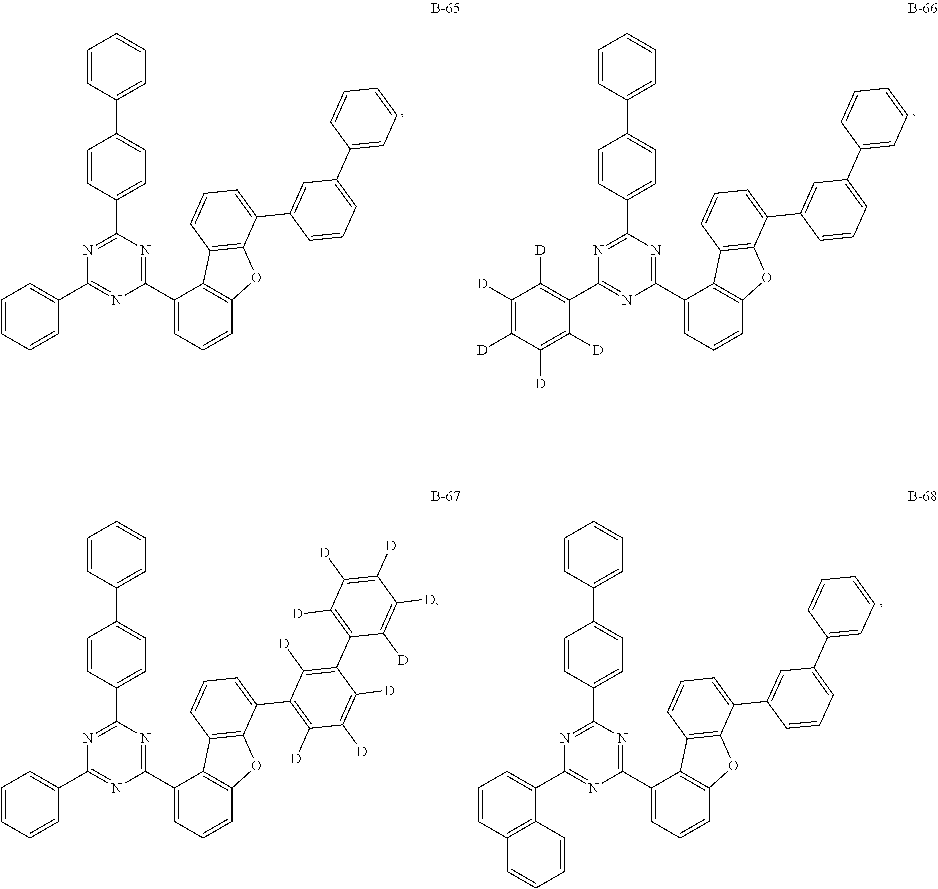 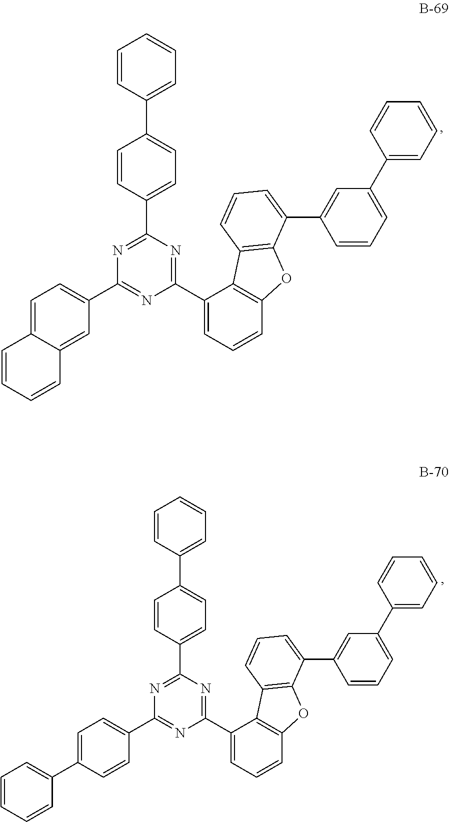 [SMILES: c1ccc(-c2ccc(-c3nc(-c4ccc5ccccc5c4)nc(-c4cccc5oc6c(-c7cccc(-c8ccccc8)c7)cccc6c45)n3)cc2)cc1 ; c1ccc(-c2ccc(-c3nc(-c4ccc(-c5ccccc5)cc4)nc(-c4cccc5oc6c(-c7cccc(-c8ccccc8)c7)cccc6c45)n3)cc2)cc1] 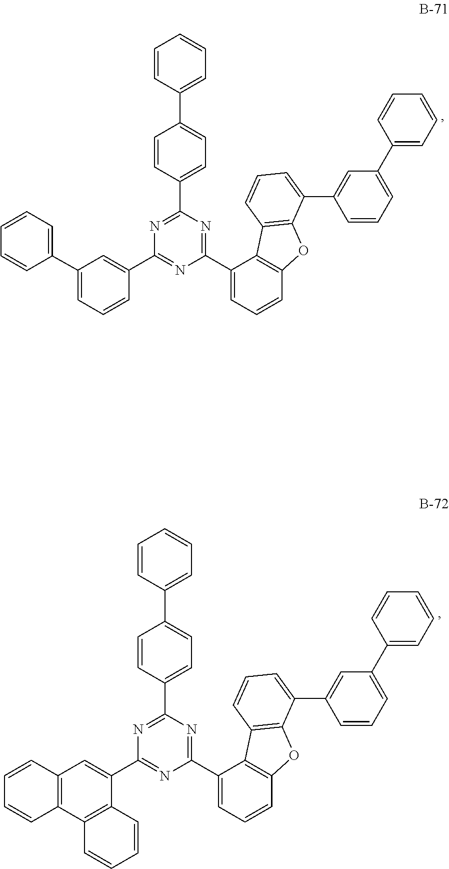 [SMILES: c1ccc(-c2ccc(-c3nc(-c4cccc(-c5ccccc5)c4)nc(-c4cccc5oc6c(-c7cccc(-c8ccccc8)c7)cccc6c45)n3)cc2)cc1 ; c1ccc(-c2ccc(-c3nc(-c4cc5ccccc5c5ccccc45)nc(-c4cccc5oc6c(-c7cccc(-c8ccccc8)c7)cccc6c45)n3)cc2)cc1] 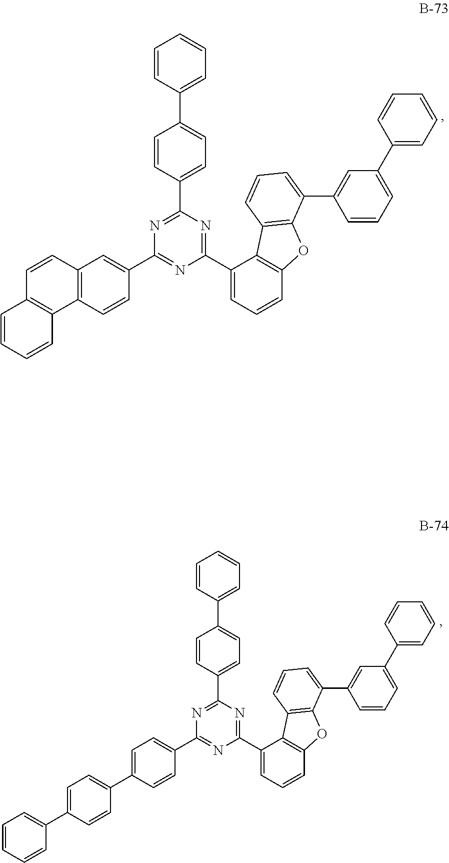 [SMILES: c1ccc(-c2ccc(-c3nc(-c4ccc5c(ccc6ccccc65)c4)nc(-c4cccc5oc6c(-c7cccc(-c8ccccc8)c7)cccc6c45)n3)cc2)cc1 ; c1ccc(-c2ccc(-c3ccc(-c4nc(-c5ccc(-c6ccccc6)cc5)nc(-c5cccc6oc7c(-c8cccc(-c9ccccc9)c8)cccc7c56)n4)cc3)cc2)cc1] 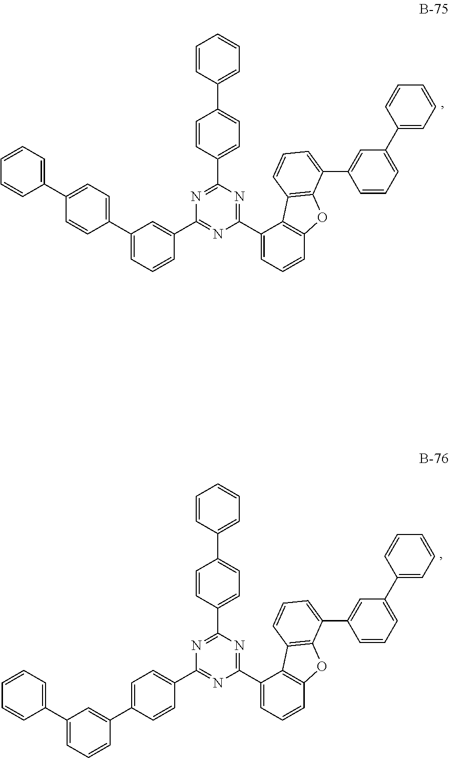 [SMILES: c1ccc(-c2ccc(-c3cccc(-c4nc(-c5ccc(-c6ccccc6)cc5)nc(-c5cccc6oc7c(-c8cccc(-c9ccccc9)c8)cccc7c56)n4)c3)cc2)cc1 ; c1ccc(-c2ccc(-c3nc(-c4ccc(-c5cccc(-c6ccccc6)c5)cc4)nc(-c4cccc5oc6c(-c7cccc(-c8ccccc8)c7)cccc6c45)n3)cc2)cc1] 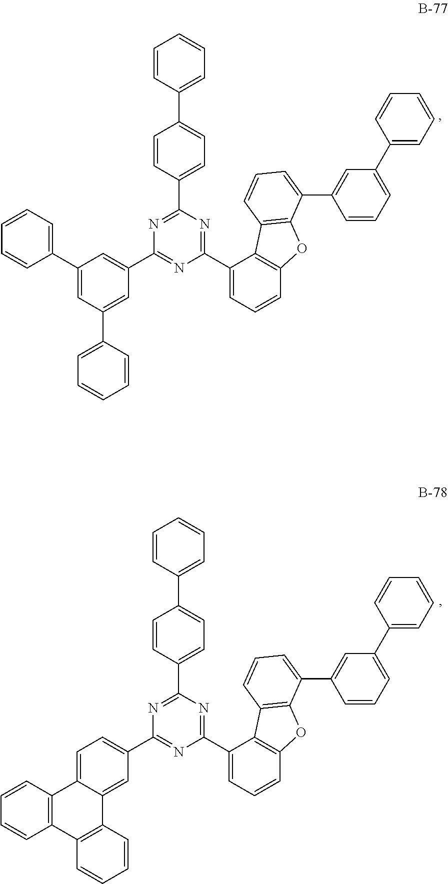 [SMILES: c1ccc(-c2ccc(-c3nc(-c4cc(-c5ccccc5)cc(-c5ccccc5)c4)nc(-c4cccc5oc6c(-c7cccc(-c8ccccc8)c7)cccc6c45)n3)cc2)cc1 ; c1ccc(-c2ccc(-c3nc(-c4ccc5c6ccccc6c6ccccc6c5c4)nc(-c4cccc5oc6c(-c7cccc(-c8ccccc8)c7)cccc6c45)n3)cc2)cc1] 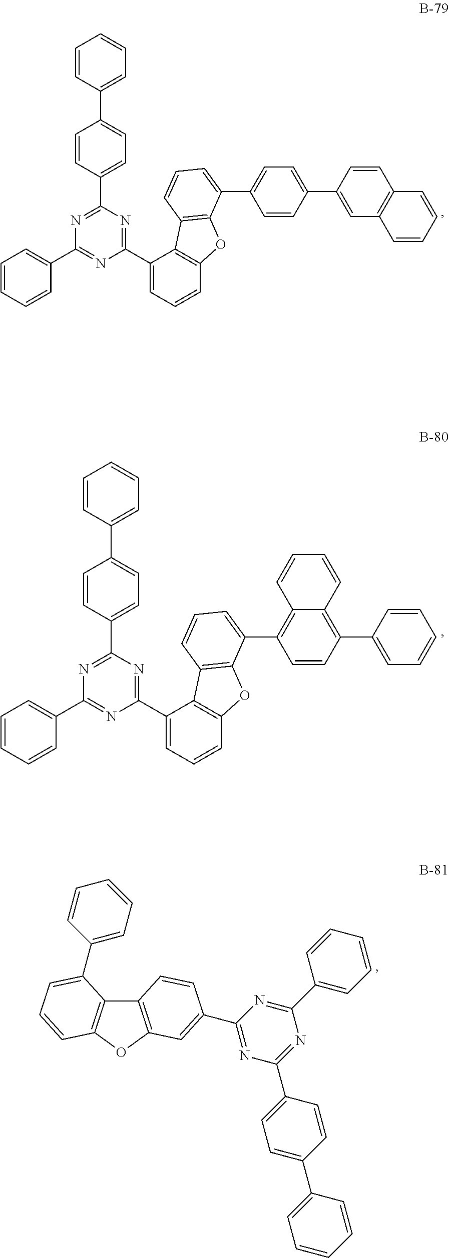 [SMILES: c1ccc(-c2ccc(-c3nc(-c4ccccc4)nc(-c4cccc5oc6c(-c7ccc(-c8ccc9ccccc9c8)cc7)cccc6c45)n3)cc2)cc1 ; c1ccc(-c2ccc(-c3nc(-c4ccccc4)nc(-c4cccc5oc6c(-c7ccc(-c8ccccc8)c8ccccc78)cccc6c45)n3)cc2)cc1 ; c1ccc(-c2ccc(-c3nc(-c4ccccc4)nc(-c4ccc5c(c4)oc4cccc(-c6ccccc6)c45)n3)cc2)cc1] 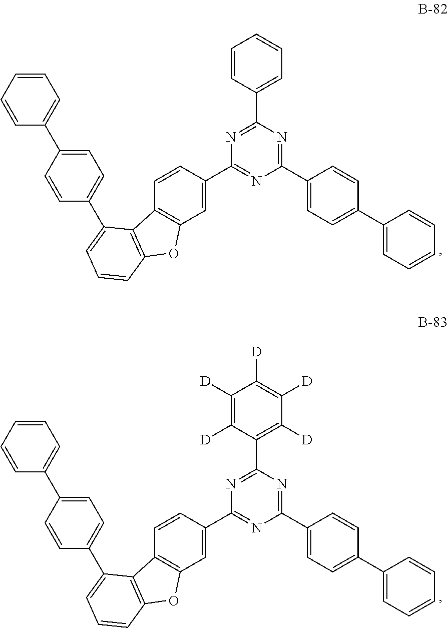 [SMILES: c1ccc(-c2ccc(-c3nc(-c4ccccc4)nc(-c4ccc5c(c4)oc4cccc(-c6ccc(-c7ccccc7)cc6)c45)n3)cc2)cc1 ; [2H]c1c([2H])c([2H])c(-c2nc(-c3ccc(-c4ccccc4)cc3)nc(-c3ccc4c(c3)oc3cccc(-c5ccc(-c6ccccc6)cc5)c34)n2)c([2H])c1[2H]] 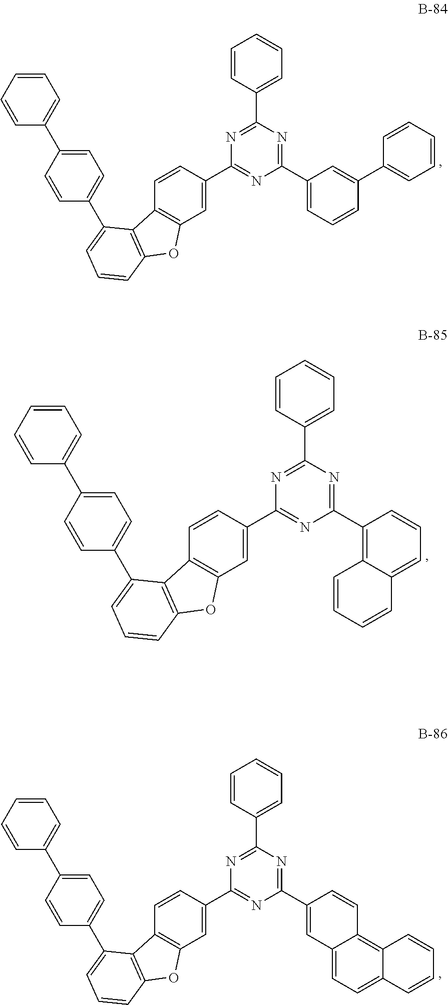 [SMILES: c1ccc(-c2ccc(-c3cccc4oc5cc(-c6nc(-c7ccccc7)nc(-c7cccc(-c8ccccc8)c7)n6)ccc5c34)cc2)cc1 ; c1ccc(-c2ccc(-c3cccc4oc5cc(-c6nc(-c7ccccc7)nc(-c7cccc8ccccc78)n6)ccc5c34)cc2)cc1 ; c1ccc(-c2ccc(-c3cccc4oc5cc(-c6nc(-c7ccccc7)nc(-c7ccc8c(ccc9ccccc98)c7)n6)ccc5c34)cc2)cc1] 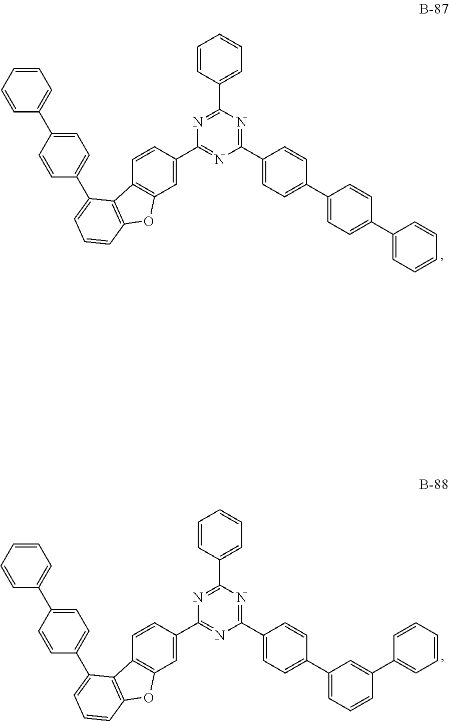 [SMILES: c1ccc(-c2ccc(-c3ccc(-c4nc(-c5ccccc5)nc(-c5ccc6c(c5)oc5cccc(-c7ccc(-c8ccccc8)cc7)c56)n4)cc3)cc2)cc1 ; c1ccc(-c2ccc(-c3cccc4oc5cc(-c6nc(-c7ccccc7)nc(-c7ccc(-c8cccc(-c9ccccc9)c8)cc7)n6)ccc5c34)cc2)cc1] 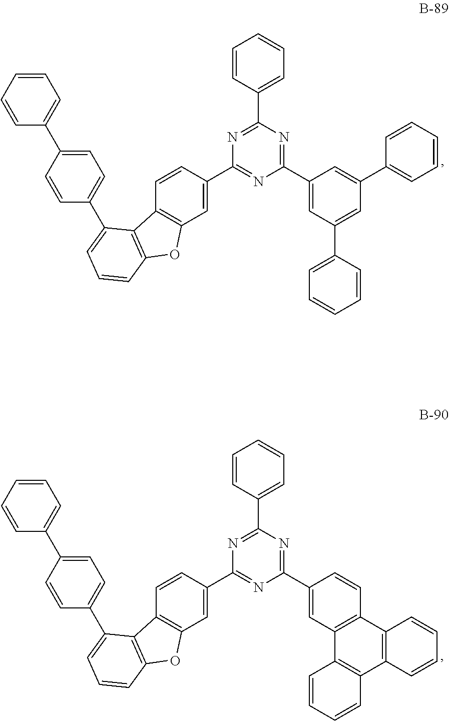 [SMILES: c1ccc(-c2ccc(-c3cccc4oc5cc(-c6nc(-c7ccccc7)nc(-c7cc(-c8ccccc8)cc(-c8ccccc8)c7)n6)ccc5c34)cc2)cc1 ; c1ccc(-c2ccc(-c3cccc4oc5cc(-c6nc(-c7ccccc7)nc(-c7ccc8c9ccccc9c9ccccc9c8c7)n6)ccc5c34)cc2)cc1] 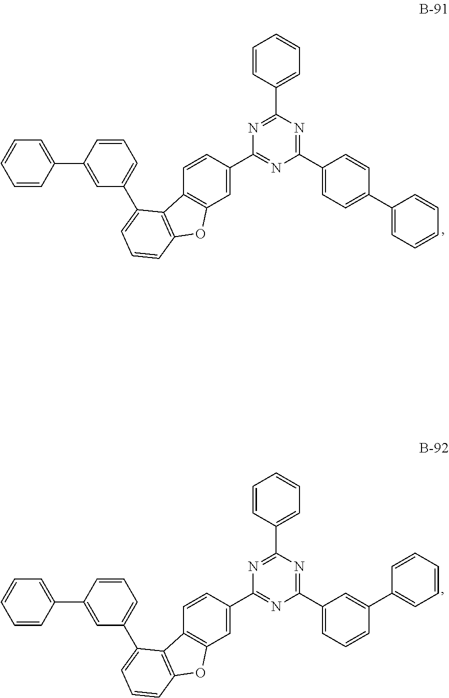 [SMILES: c1ccc(-c2ccc(-c3nc(-c4ccccc4)nc(-c4ccc5c(c4)oc4cccc(-c6cccc(-c7ccccc7)c6)c45)n3)cc2)cc1 ; c1ccc(-c2cccc(-c3nc(-c4ccccc4)nc(-c4ccc5c(c4)oc4cccc(-c6cccc(-c7ccccc7)c6)c45)n3)c2)cc1] 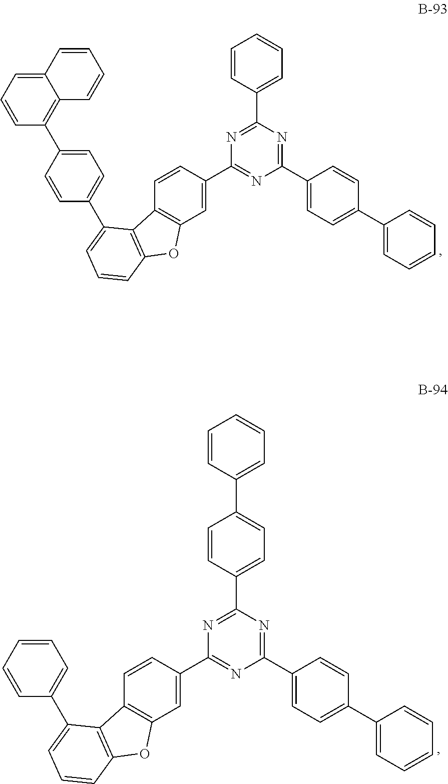 [SMILES: c1ccc(-c2ccc(-c3nc(-c4ccccc4)nc(-c4ccc5c(c4)oc4cccc(-c6ccc(-c7cccc8ccccc78)cc6)c45)n3)cc2)cc1 ; c1ccc(-c2ccc(-c3nc(-c4ccc(-c5ccccc5)cc4)nc(-c4ccc5c(c4)oc4cccc(-c6ccccc6)c45)n3)cc2)cc1] 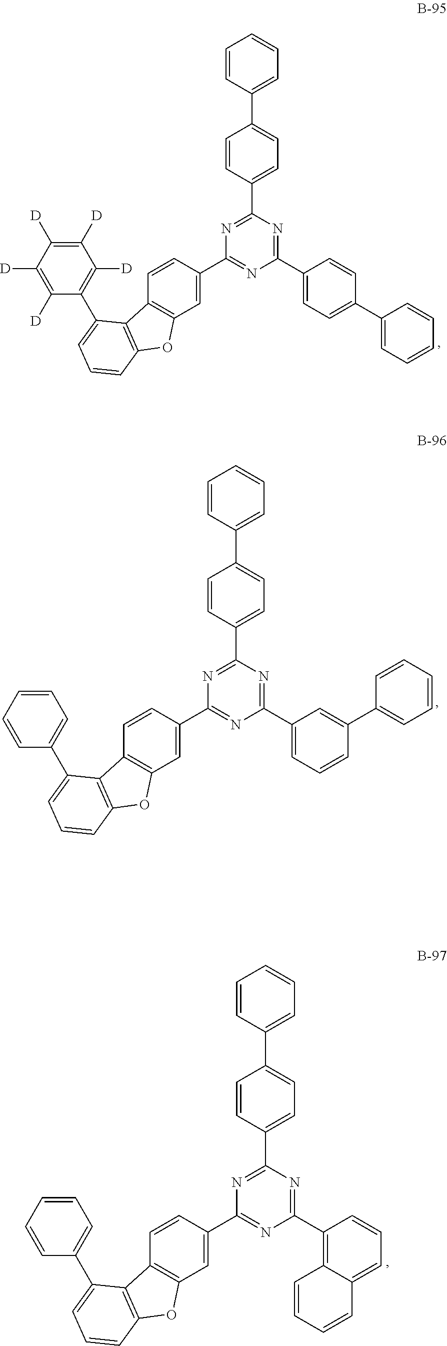 [SMILES: [2H]c1c([2H])c([2H])c(-c2cccc3oc4cc(-c5nc(-c6ccc(-c7ccccc7)cc6)nc(-c6ccc(-c7ccccc7)cc6)n5)ccc4c23)c([2H])c1[2H] ; c1ccc(-c2ccc(-c3nc(-c4cccc(-c5ccccc5)c4)nc(-c4ccc5c(c4)oc4cccc(-c6ccccc6)c45)n3)cc2)cc1 ; c1ccc(-c2ccc(-c3nc(-c4ccc5c(c4)oc4cccc(-c6ccccc6)c45)nc(-c4cccc5ccccc45)n3)cc2)cc1] 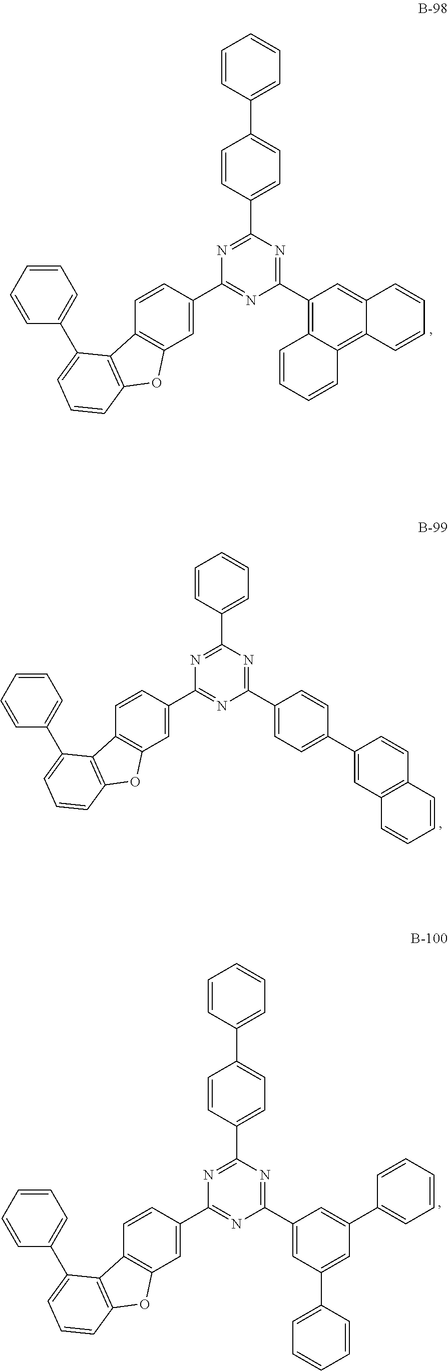 [SMILES: c1ccc(-c2ccc(-c3nc(-c4ccc5c(c4)oc4cccc(-c6ccccc6)c45)nc(-c4cc5ccccc5c5ccccc45)n3)cc2)cc1 ; c1ccc(-c2nc(-c3ccc(-c4ccc5ccccc5c4)cc3)nc(-c3ccc4c(c3)oc3cccc(-c5ccccc5)c34)n2)cc1 ; c1ccc(-c2ccc(-c3nc(-c4cc(-c5ccccc5)cc(-c5ccccc5)c4)nc(-c4ccc5c(c4)oc4cccc(-c6ccccc6)c45)n3)cc2)cc1] 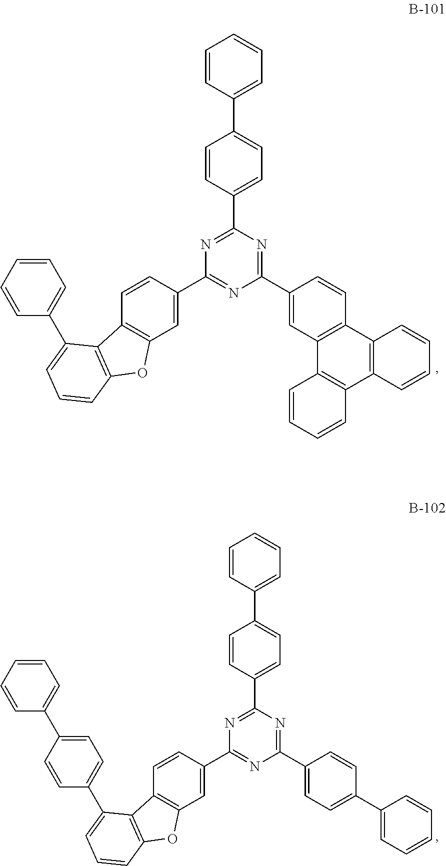 [SMILES: c1ccc(-c2ccc(-c3nc(-c4ccc5c(c4)oc4cccc(-c6ccccc6)c45)nc(-c4ccc5c6ccccc6c6ccccc6c5c4)n3)cc2)cc1 ; c1ccc(-c2ccc(-c3nc(-c4ccc(-c5ccccc5)cc4)nc(-c4ccc5c(c4)oc4cccc(-c6ccc(-c7ccccc7)cc6)c45)n3)cc2)cc1] 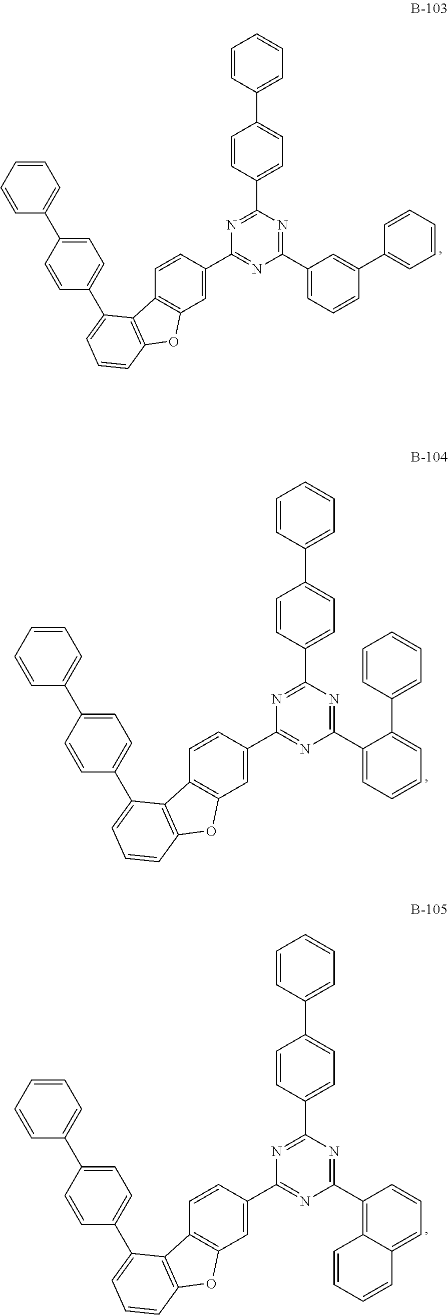 [SMILES: c1ccc(-c2ccc(-c3nc(-c4cccc(-c5ccccc5)c4)nc(-c4ccc5c(c4)oc4cccc(-c6ccc(-c7ccccc7)cc6)c45)n3)cc2)cc1 ; c1ccc(-c2ccc(-c3nc(-c4ccc5c(c4)oc4cccc(-c6ccc(-c7ccccc7)cc6)c45)nc(-c4ccccc4-c4ccccc4)n3)cc2)cc1 ; c1ccc(-c2ccc(-c3nc(-c4ccc5c(c4)oc4cccc(-c6ccc(-c7ccccc7)cc6)c45)nc(-c4cccc5ccccc45)n3)cc2)cc1] 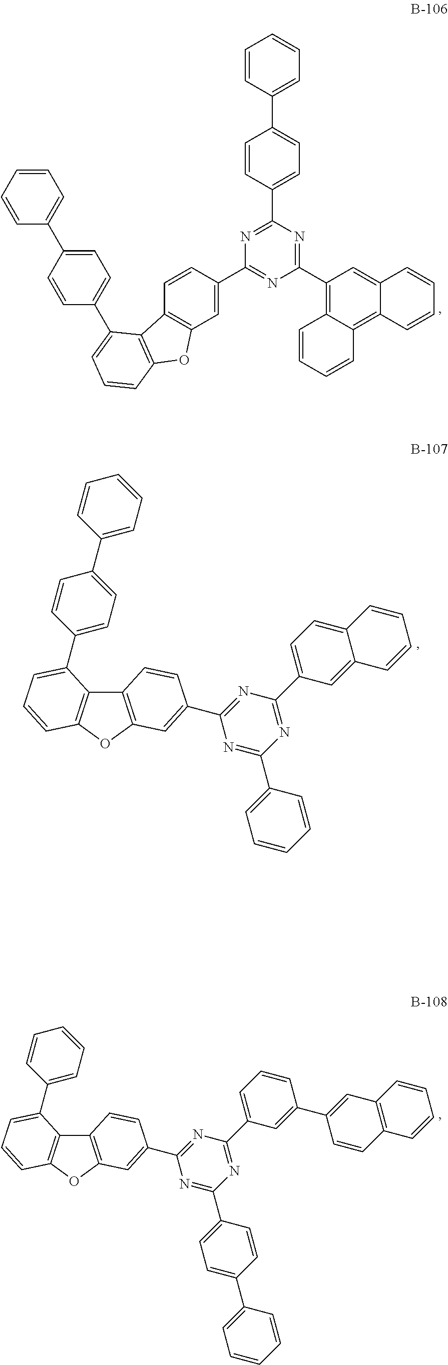 [SMILES: c1ccc(-c2ccc(-c3nc(-c4ccc5c(c4)oc4cccc(-c6ccc(-c7ccccc7)cc6)c45)nc(-c4cc5ccccc5c5ccccc45)n3)cc2)cc1 ; c1ccc(-c2ccc(-c3cccc4oc5cc(-c6nc(-c7ccccc7)nc(-c7ccc8ccccc8c7)n6)ccc5c34)cc2)cc1 ; c1ccc(-c2ccc(-c3nc(-c4cccc(-c5ccc6ccccc6c5)c4)nc(-c4ccc5c(c4)oc4cccc(-c6ccccc6)c45)n3)cc2)cc1] 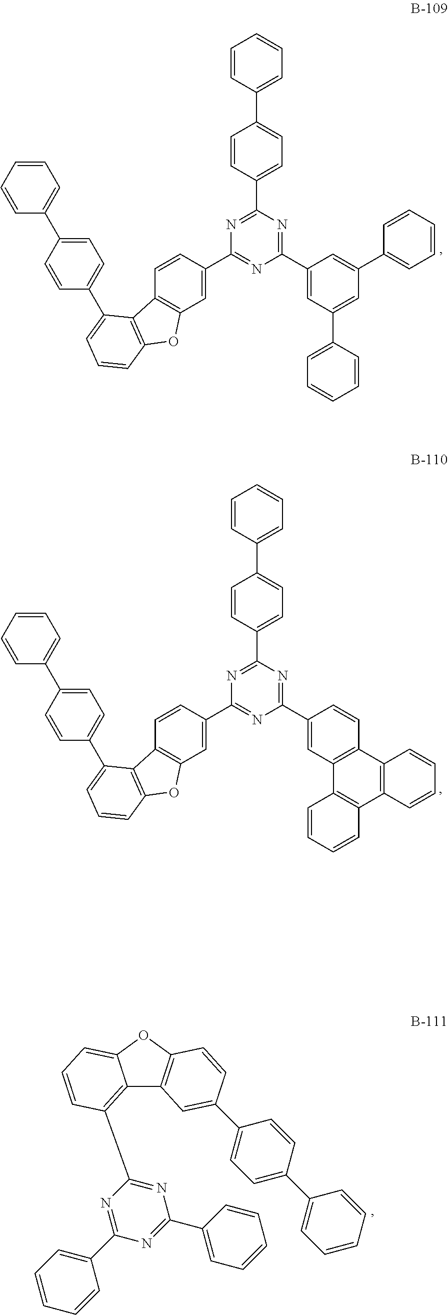 [SMILES: c1ccc(-c2ccc(-c3nc(-c4cc(-c5ccccc5)cc(-c5ccccc5)c4)nc(-c4ccc5c(c4)oc4cccc(-c6ccc(-c7ccccc7)cc6)c45)n3)cc2)cc1 ; c1ccc(-c2ccc(-c3nc(-c4ccc5c(c4)oc4cccc(-c6ccc(-c7ccccc7)cc6)c45)nc(-c4ccc5c6ccccc6c6ccccc6c5c4)n3)cc2)cc1 ; c1ccc(-c2ccc(-c3ccc4oc5cccc(-c6nc(-c7ccccc7)nc(-c7ccccc7)n6)c5c4c3)cc2)cc1] 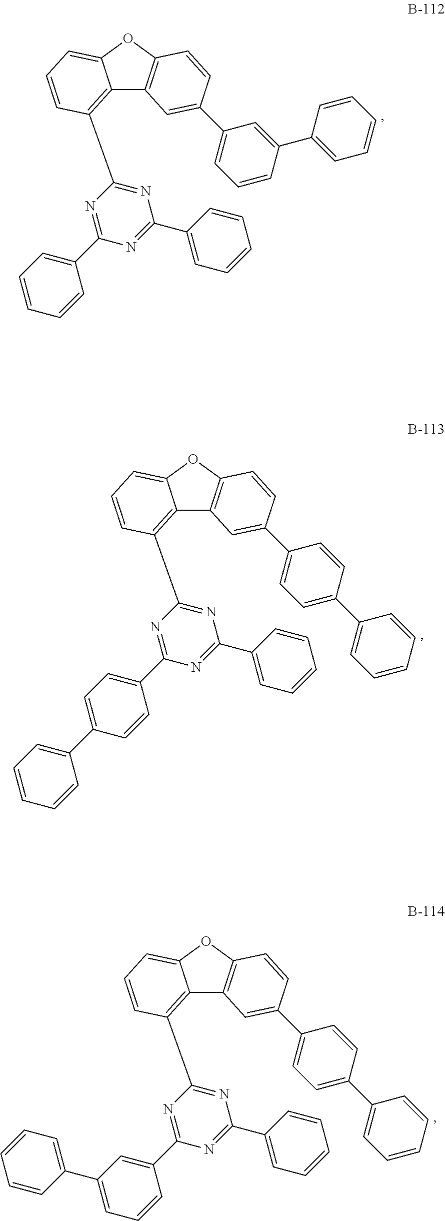 [SMILES: c1ccc(-c2cccc(-c3ccc4oc5cccc(-c6nc(-c7ccccc7)nc(-c7ccccc7)n6)c5c4c3)c2)cc1 ; c1ccc(-c2ccc(-c3ccc4oc5cccc(-c6nc(-c7ccccc7)nc(-c7ccc(-c8ccccc8)cc7)n6)c5c4c3)cc2)cc1 ; c1ccc(-c2ccc(-c3ccc4oc5cccc(-c6nc(-c7ccccc7)nc(-c7cccc(-c8ccccc8)c7)n6)c5c4c3)cc2)cc1] 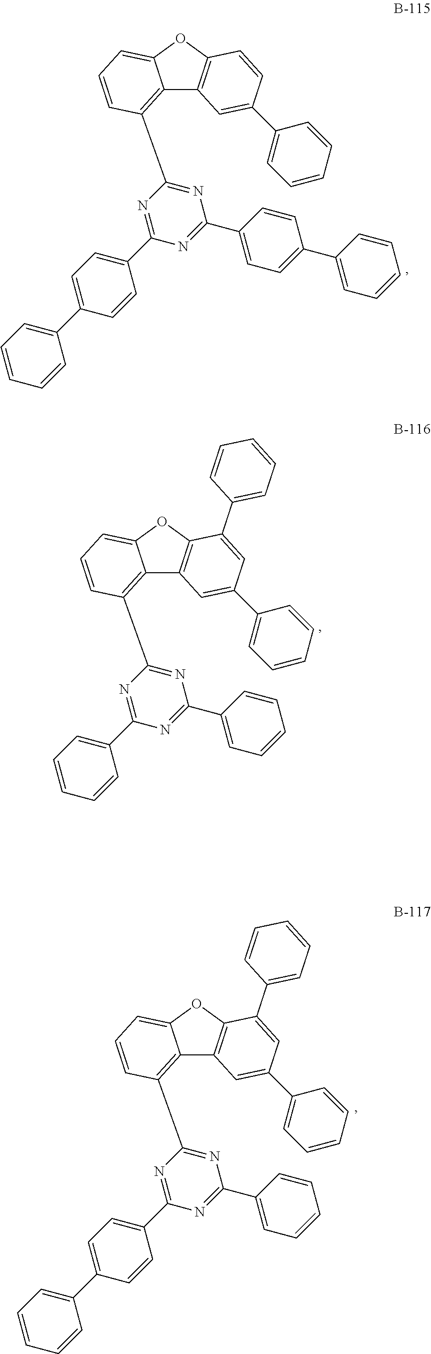 [SMILES: c1ccc(-c2ccc(-c3nc(-c4ccc(-c5ccccc5)cc4)nc(-c4cccc5oc6ccc(-c7ccccc7)cc6c45)n3)cc2)cc1 ; c1ccc(-c2cc(-c3ccccc3)c3oc4cccc(-c5nc(-c6ccccc6)nc(-c6ccccc6)n5)c4c3c2)cc1 ; c1ccc(-c2ccc(-c3nc(-c4ccccc4)nc(-c4cccc5oc6c(-c7ccccc7)cc(-c7ccccc7)cc6c45)n3)cc2)cc1] 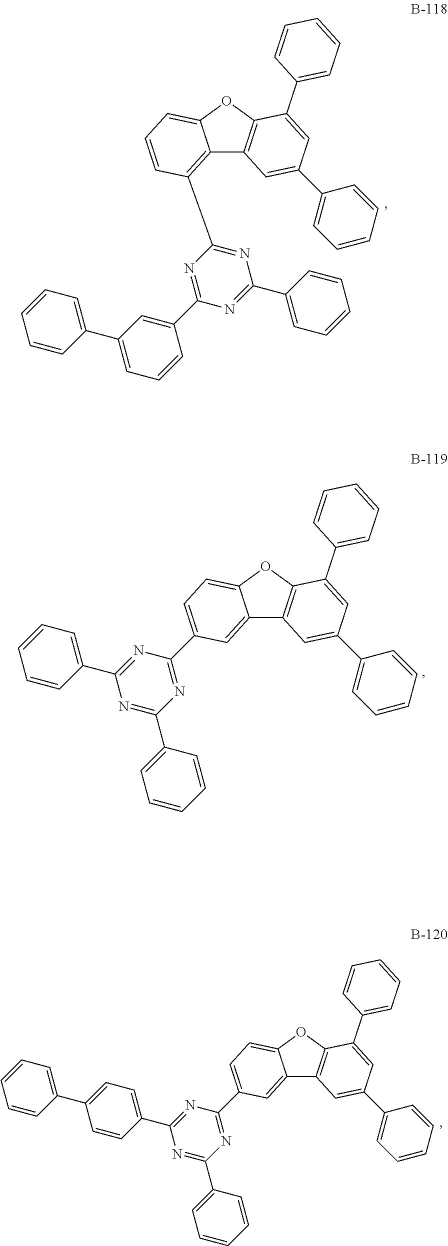 [SMILES: c1ccc(-c2cccc(-c3nc(-c4ccccc4)nc(-c4cccc5oc6c(-c7ccccc7)cc(-c7ccccc7)cc6c45)n3)c2)cc1 ; c1ccc(-c2cc(-c3ccccc3)c3oc4ccc(-c5nc(-c6ccccc6)nc(-c6ccccc6)n5)cc4c3c2)cc1 ; c1ccc(-c2ccc(-c3nc(-c4ccccc4)nc(-c4ccc5oc6c(-c7ccccc7)cc(-c7ccccc7)cc6c5c4)n3)cc2)cc1] 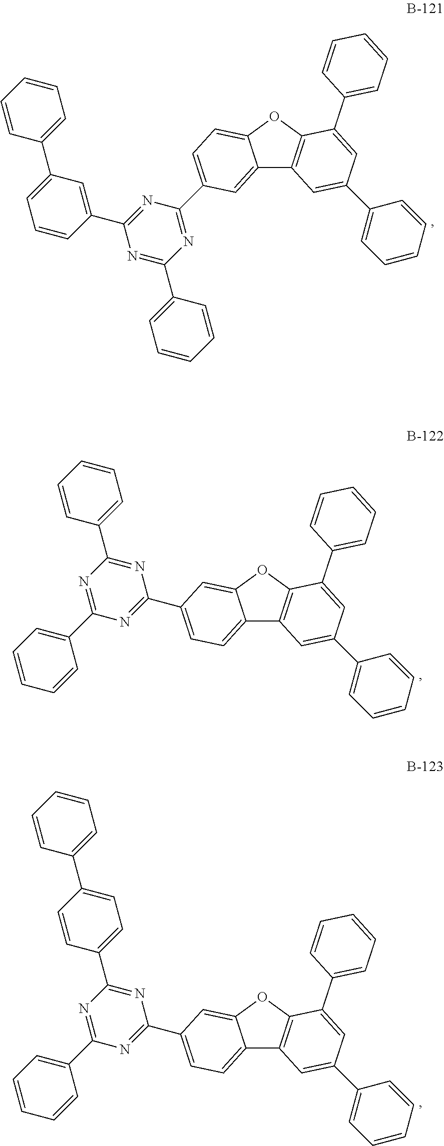 [SMILES: c1ccc(-c2cccc(-c3nc(-c4ccccc4)nc(-c4ccc5oc6c(-c7ccccc7)cc(-c7ccccc7)cc6c5c4)n3)c2)cc1 ; c1ccc(-c2cc(-c3ccccc3)c3oc4cc(-c5nc(-c6ccccc6)nc(-c6ccccc6)n5)ccc4c3c2)cc1 ; c1ccc(-c2ccc(-c3nc(-c4ccccc4)nc(-c4ccc5c(c4)oc4c(-c6ccccc6)cc(-c6ccccc6)cc45)n3)cc2)cc1] 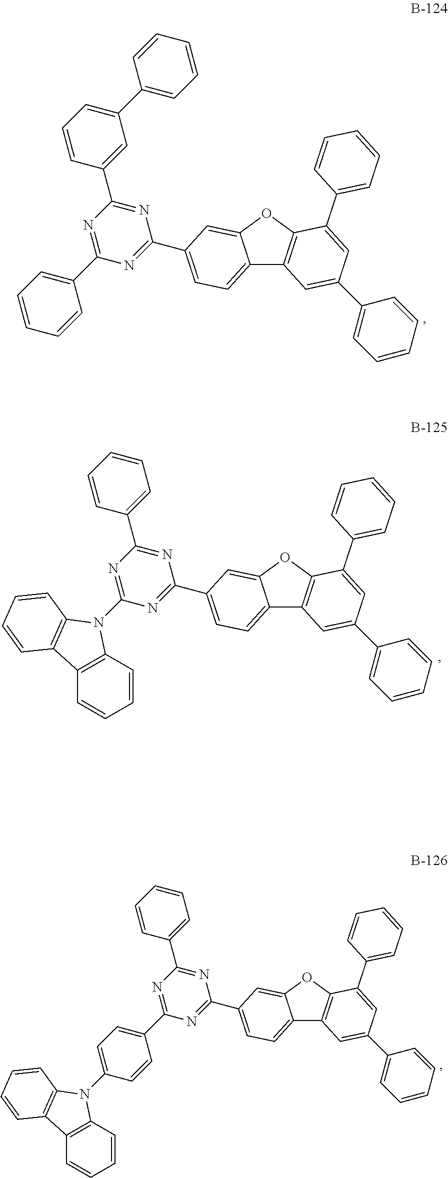 [SMILES: c1ccc(-c2cccc(-c3nc(-c4ccccc4)nc(-c4ccc5c(c4)oc4c(-c6ccccc6)cc(-c6ccccc6)cc45)n3)c2)cc1 ; c1ccc(-c2cc(-c3ccccc3)c3oc4cc(-c5nc(-c6ccccc6)nc(-n6c7ccccc7c7ccccc76)n5)ccc4c3c2)cc1 ; c1ccc(-c2cc(-c3ccccc3)c3oc4cc(-c5nc(-c6ccccc6)nc(-c6ccc(-n7c8ccccc8c8ccccc87)cc6)n5)ccc4c3c2)cc1] 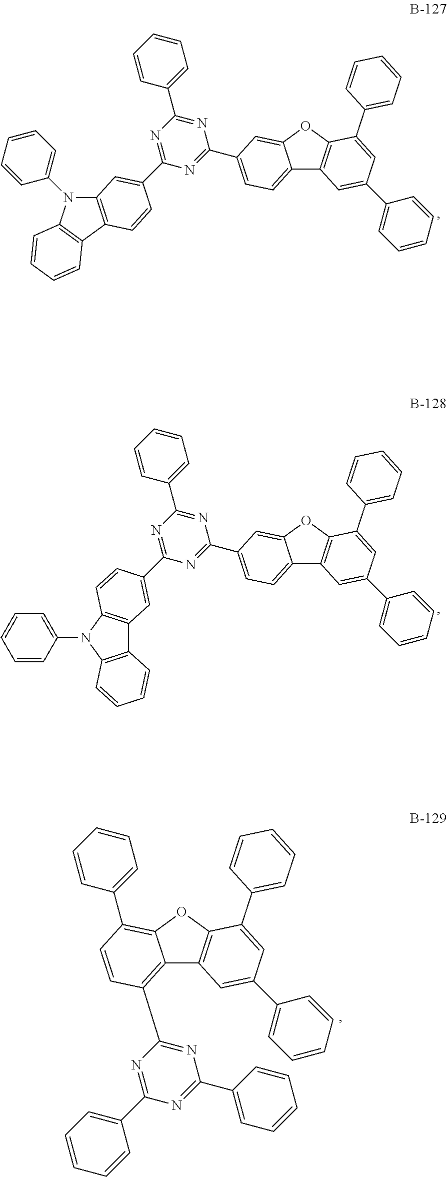 [SMILES: c1ccc(-c2cc(-c3ccccc3)c3oc4cc(-c5nc(-c6ccccc6)nc(-c6ccc7c8ccccc8n(-c8ccccc8)c7c6)n5)ccc4c3c2)cc1 ; c1ccc(-c2cc(-c3ccccc3)c3oc4cc(-c5nc(-c6ccccc6)nc(-c6ccc7c(c6)c6ccccc6n7-c6ccccc6)n5)ccc4c3c2)cc1 ; c1ccc(-c2cc(-c3ccccc3)c3oc4c(-c5ccccc5)ccc(-c5nc(-c6ccccc6)nc(-c6ccccc6)n5)c4c3c2)cc1] 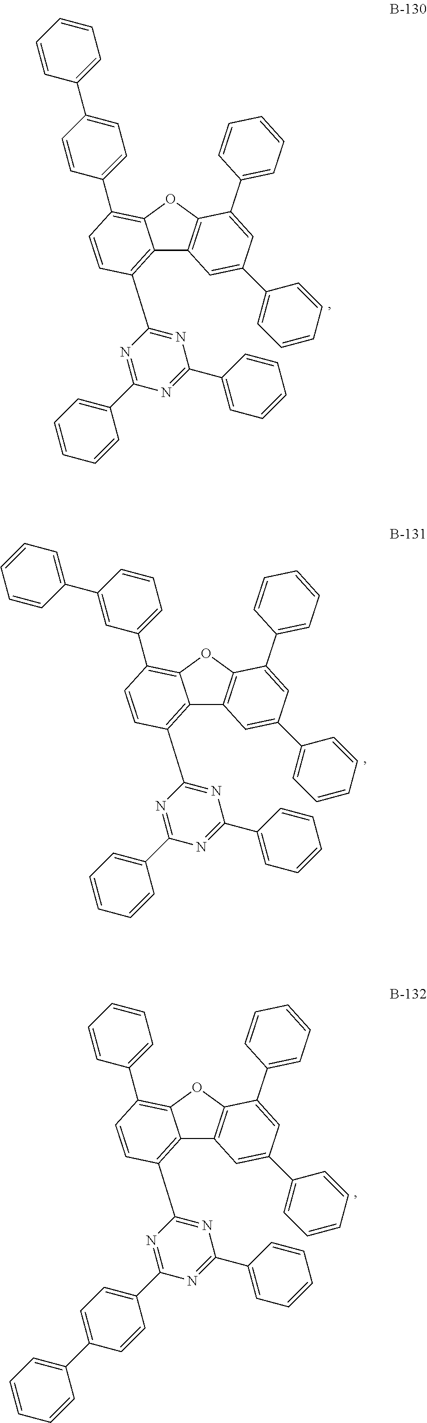 [SMILES: c1ccc(-c2ccc(-c3ccc(-c4nc(-c5ccccc5)nc(-c5ccccc5)n4)c4c3oc3c(-c5ccccc5)cc(-c5ccccc5)cc34)cc2)cc1 ; c1ccc(-c2cccc(-c3ccc(-c4nc(-c5ccccc5)nc(-c5ccccc5)n4)c4c3oc3c(-c5ccccc5)cc(-c5ccccc5)cc34)c2)cc1 ; c1ccc(-c2ccc(-c3nc(-c4ccccc4)nc(-c4ccc(-c5ccccc5)c5oc6c(-c7ccccc7)cc(-c7ccccc7)cc6c45)n3)cc2)cc1] 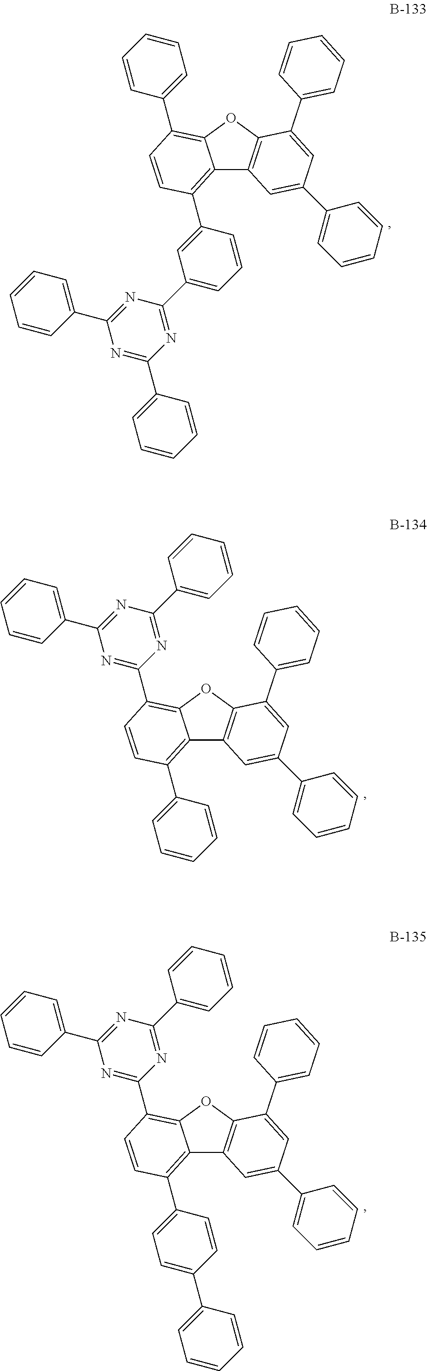 [SMILES: c1ccc(-c2cc(-c3ccccc3)c3oc4c(-c5ccccc5)ccc(-c5cccc(-c6nc(-c7ccccc7)nc(-c7ccccc7)n6)c5)c4c3c2)cc1 ; c1ccc(-c2cc(-c3ccccc3)c3oc4c(-c5nc(-c6ccccc6)nc(-c6ccccc6)n5)ccc(-c5ccccc5)c4c3c2)cc1 ; c1ccc(-c2ccc(-c3ccc(-c4nc(-c5ccccc5)nc(-c5ccccc5)n4)c4oc5c(-c6ccccc6)cc(-c6ccccc6)cc5c34)cc2)cc1] 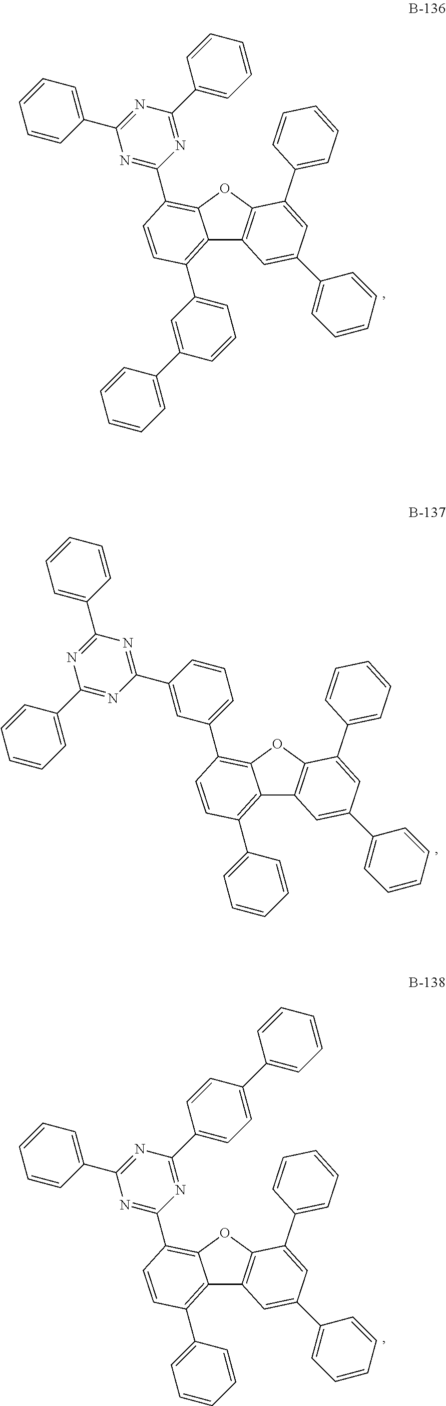 [SMILES: c1ccc(-c2cccc(-c3ccc(-c4nc(-c5ccccc5)nc(-c5ccccc5)n4)c4oc5c(-c6ccccc6)cc(-c6ccccc6)cc5c34)c2)cc1 ; c1ccc(-c2cc(-c3ccccc3)c3oc4c(-c5cccc(-c6nc(-c7ccccc7)nc(-c7ccccc7)n6)c5)ccc(-c5ccccc5)c4c3c2)cc1 ; c1ccc(-c2ccc(-c3nc(-c4ccccc4)nc(-c4ccc(-c5ccccc5)c5c4oc4c(-c6ccccc6)cc(-c6ccccc6)cc45)n3)cc2)cc1] 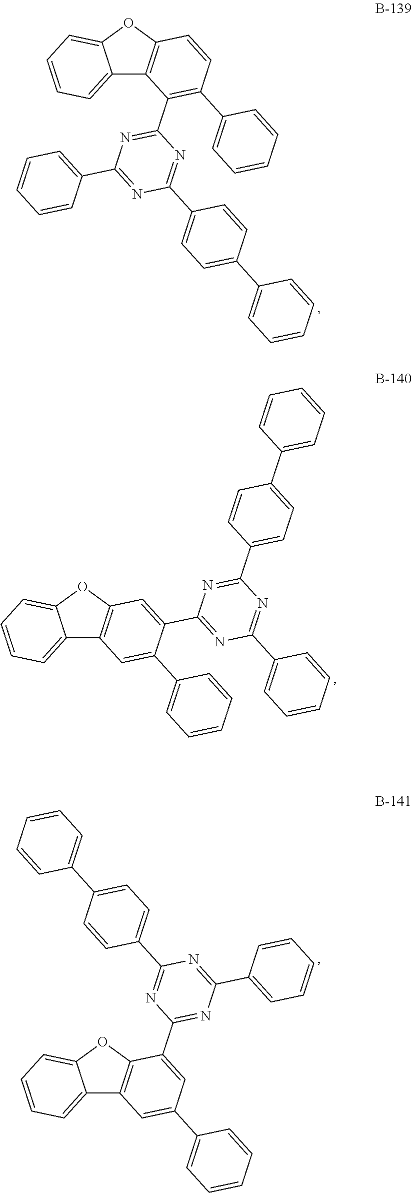 [SMILES: c1ccc(-c2ccc(-c3nc(-c4ccccc4)nc(-c4c(-c5ccccc5)ccc5oc6ccccc6c45)n3)cc2)cc1 ; c1ccc(-c2ccc(-c3nc(-c4ccccc4)nc(-c4cc5oc6ccccc6c5cc4-c4ccccc4)n3)cc2)cc1 ; c1ccc(-c2ccc(-c3nc(-c4ccccc4)nc(-c4cc(-c5ccccc5)cc5c4oc4ccccc45)n3)cc2)cc1] 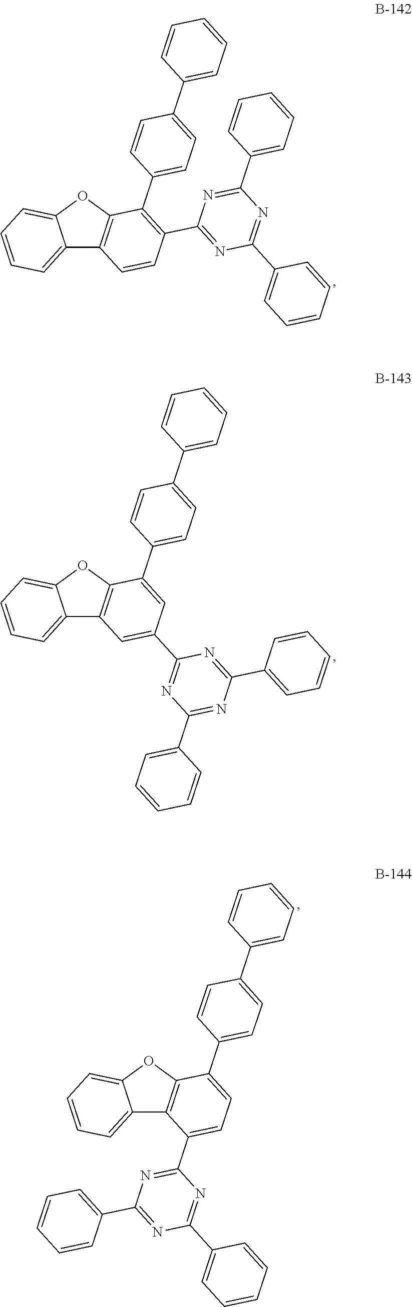 [SMILES: c1ccc(-c2ccc(-c3c(-c4nc(-c5ccccc5)nc(-c5ccccc5)n4)ccc4c3oc3ccccc34)cc2)cc1 ; c1ccc(-c2ccc(-c3cc(-c4nc(-c5ccccc5)nc(-c5ccccc5)n4)cc4c3oc3ccccc34)cc2)cc1 ; c1ccc(-c2ccc(-c3ccc(-c4nc(-c5ccccc5)nc(-c5ccccc5)n4)c4c3oc3ccccc34)cc2)cc1] 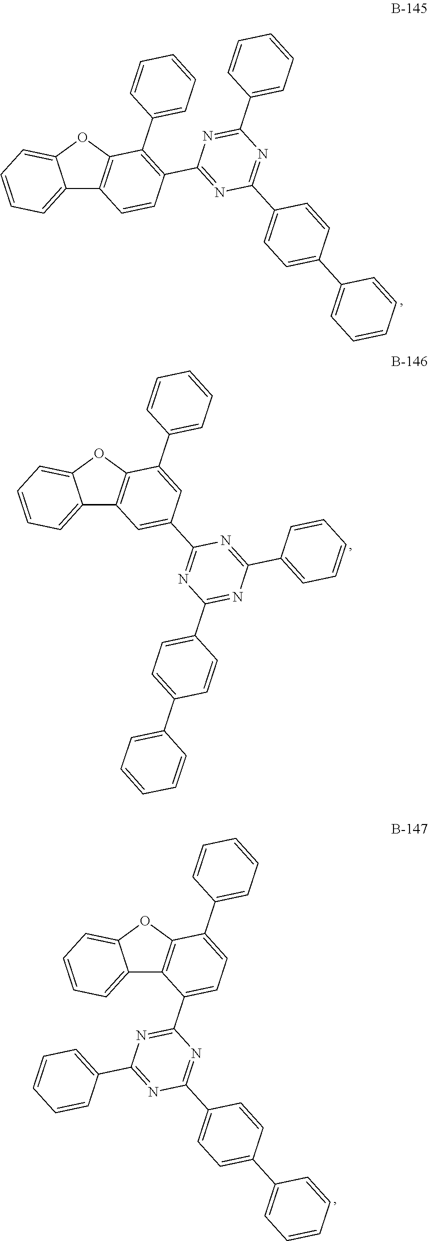 [SMILES: c1ccc(-c2ccc(-c3nc(-c4ccccc4)nc(-c4ccc5c(oc6ccccc65)c4-c4ccccc4)n3)cc2)cc1 ; c1ccc(-c2ccc(-c3nc(-c4ccccc4)nc(-c4cc(-c5ccccc5)c5oc6ccccc6c5c4)n3)cc2)cc1 ; c1ccc(-c2ccc(-c3nc(-c4ccccc4)nc(-c4ccc(-c5ccccc5)c5oc6ccccc6c45)n3)cc2)cc1] 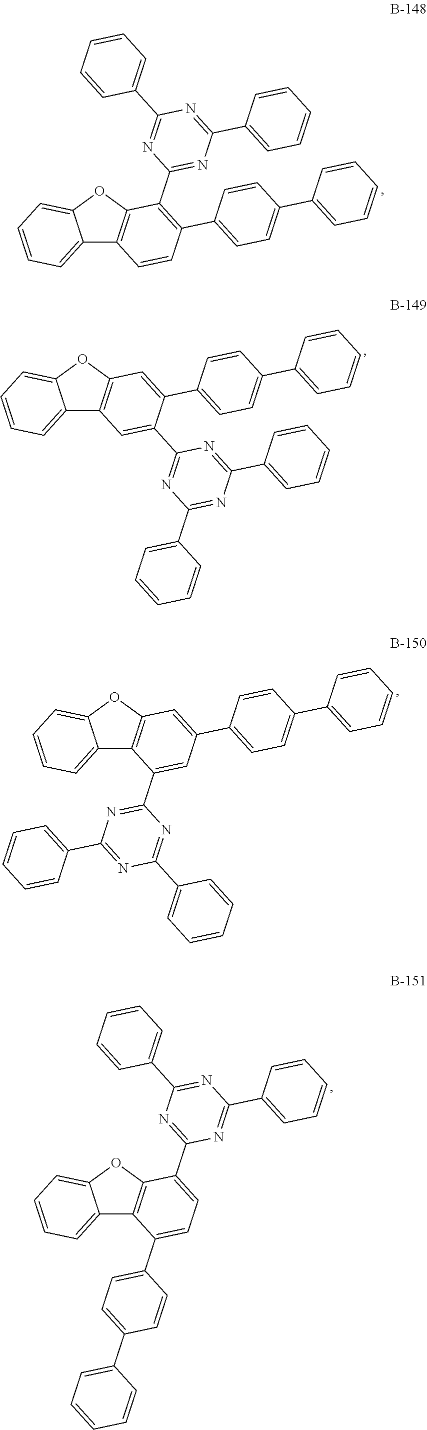 [SMILES: c1ccc(-c2ccc(-c3ccc4c(oc5ccccc54)c3-c3nc(-c4ccccc4)nc(-c4ccccc4)n3)cc2)cc1 ; c1ccc(-c2ccc(-c3cc4oc5ccccc5c4cc3-c3nc(-c4ccccc4)nc(-c4ccccc4)n3)cc2)cc1 ; c1ccc(-c2ccc(-c3cc(-c4nc(-c5ccccc5)nc(-c5ccccc5)n4)c4c(c3)oc3ccccc34)cc2)cc1 ; c1ccc(-c2ccc(-c3ccc(-c4nc(-c5ccccc5)nc(-c5ccccc5)n4)c4oc5ccccc5c34)cc2)cc1] 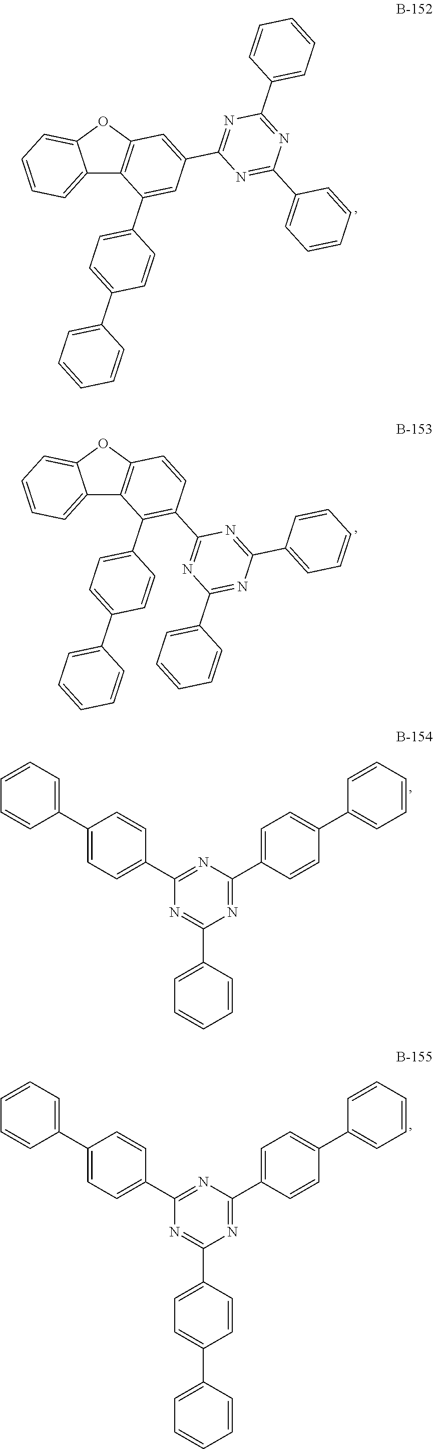 [SMILES: c1ccc(-c2ccc(-c3cc(-c4nc(-c5ccccc5)nc(-c5ccccc5)n4)cc4oc5ccccc5c34)cc2)cc1 ; c1ccc(-c2ccc(-c3c(-c4nc(-c5ccccc5)nc(-c5ccccc5)n4)ccc4oc5ccccc5c34)cc2)cc1 ; c1ccc(-c2ccc(-c3nc(-c4ccccc4)nc(-c4ccc(-c5ccccc5)cc4)n3)cc2)cc1 ; c1ccc(-c2ccc(-c3nc(-c4ccc(-c5ccccc5)cc4)nc(-c4ccc(-c5ccccc5)cc4)n3)cc2)cc1] 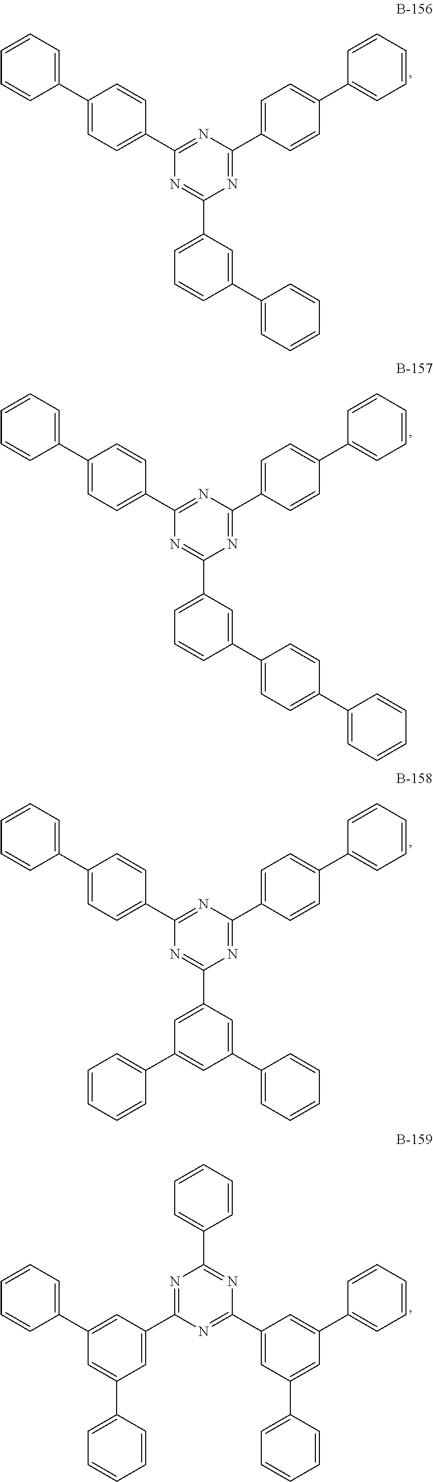 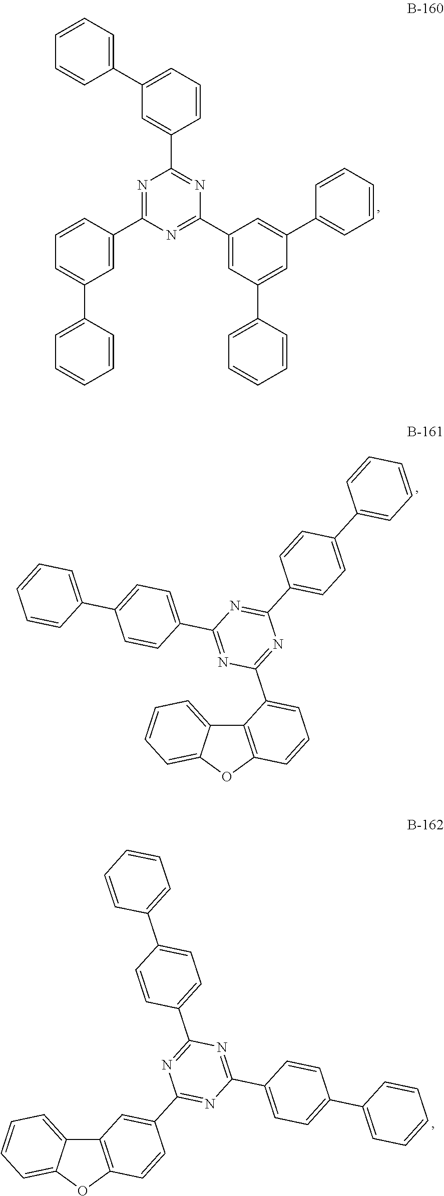 [SMILES: c1ccc(-c2cccc(-c3nc(-c4cccc(-c5ccccc5)c4)nc(-c4cc(-c5ccccc5)cc(-c5ccccc5)c4)n3)c2)cc1 ; c1ccc(-c2ccc(-c3nc(-c4ccc(-c5ccccc5)cc4)nc(-c4cccc5oc6ccccc6c45)n3)cc2)cc1 ; c1ccc(-c2ccc(-c3nc(-c4ccc(-c5ccccc5)cc4)nc(-c4ccc5oc6ccccc6c5c4)n3)cc2)cc1] 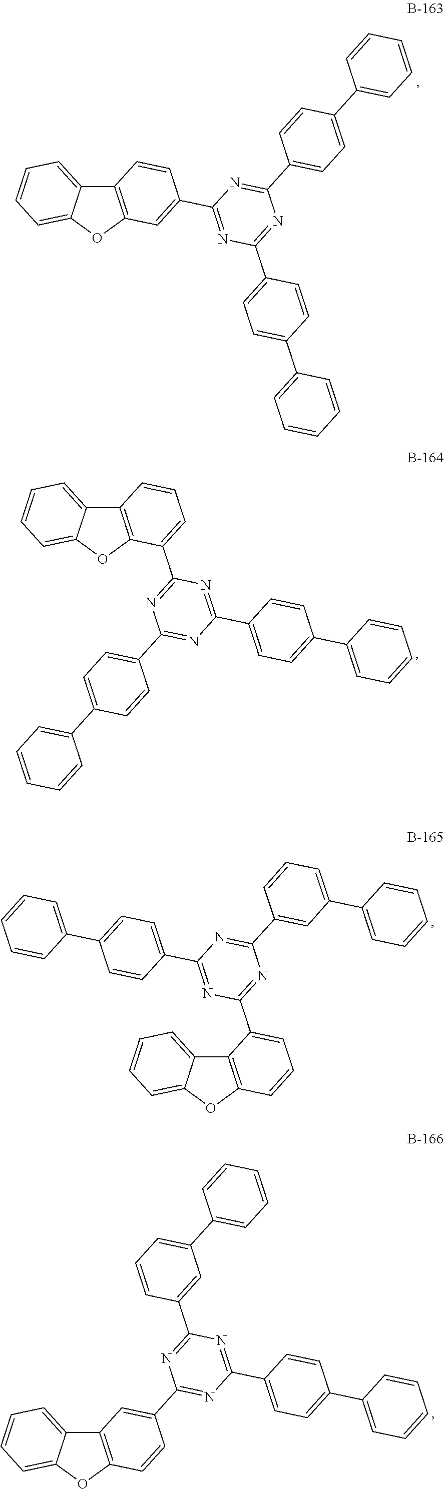 [SMILES: c1ccc(-c2ccc(-c3nc(-c4ccc(-c5ccccc5)cc4)nc(-c4ccc5c(c4)oc4ccccc45)n3)cc2)cc1 ; c1ccc(-c2ccc(-c3nc(-c4ccc(-c5ccccc5)cc4)nc(-c4cccc5c4oc4ccccc45)n3)cc2)cc1 ; c1ccc(-c2ccc(-c3nc(-c4cccc(-c5ccccc5)c4)nc(-c4cccc5oc6ccccc6c45)n3)cc2)cc1 ; c1ccc(-c2ccc(-c3nc(-c4cccc(-c5ccccc5)c4)nc(-c4ccc5oc6ccccc6c5c4)n3)cc2)cc1] 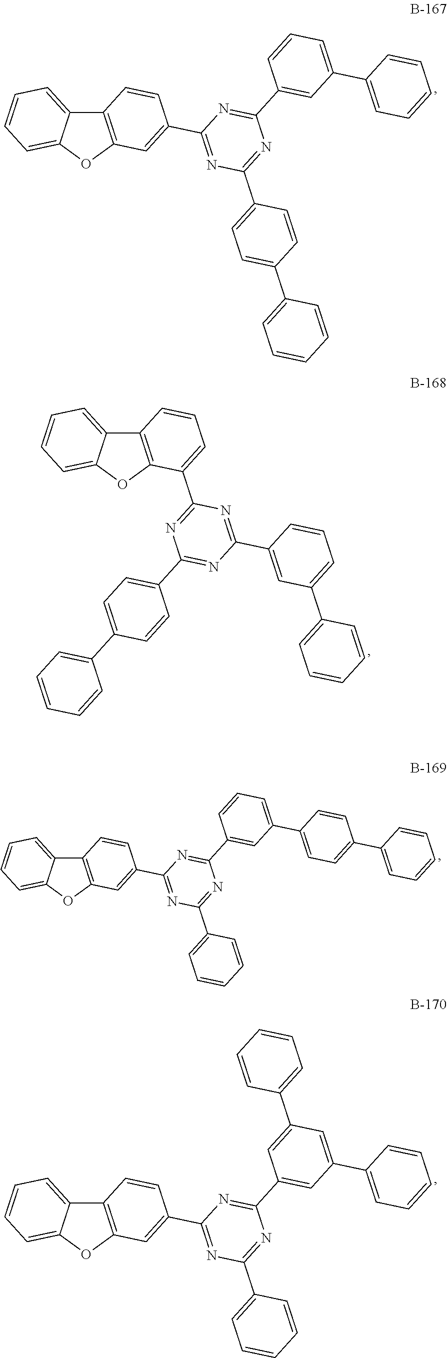 [SMILES: c1ccc(-c2ccc(-c3nc(-c4cccc(-c5ccccc5)c4)nc(-c4ccc5c(c4)oc4ccccc45)n3)cc2)cc1 ; c1ccc(-c2ccc(-c3nc(-c4cccc(-c5ccccc5)c4)nc(-c4cccc5c4oc4ccccc45)n3)cc2)cc1 ; c1ccc(-c2ccc(-c3cccc(-c4nc(-c5ccccc5)nc(-c5ccc6c(c5)oc5ccccc56)n4)c3)cc2)cc1 ; c1ccc(-c2cc(-c3ccccc3)cc(-c3nc(-c4ccccc4)nc(-c4ccc5c(c4)oc4ccccc45)n3)c2)cc1] 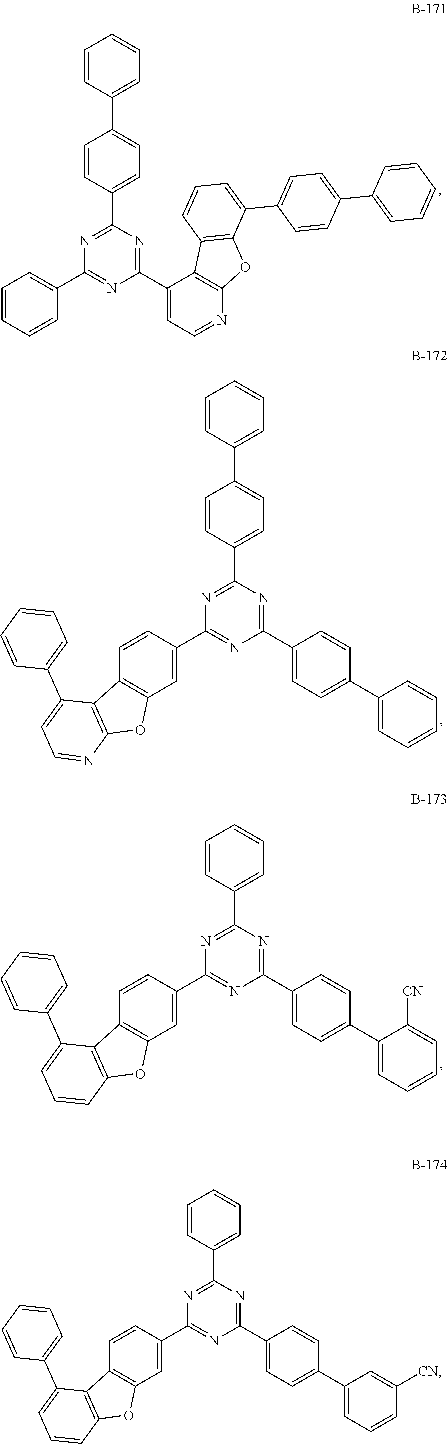 [SMILES: c1ccc(-c2ccc(-c3nc(-c4ccccc4)nc(-c4ccnc5oc6c(-c7ccc(-c8ccccc8)cc7)cccc6c45)n3)cc2)cc1 ; c1ccc(-c2ccc(-c3nc(-c4ccc(-c5ccccc5)cc4)nc(-c4ccc5c(c4)oc4nccc(-c6ccccc6)c45)n3)cc2)cc1 ; N#Cc1ccccc1-c1ccc(-c2nc(-c3ccccc3)nc(-c3ccc4c(c3)oc3cccc(-c5ccccc5)c34)n2)cc1 ; N#Cc1cccc(-c2ccc(-c3nc(-c4ccccc4)nc(-c4ccc5c(c4)oc4cccc(-c6ccccc6)c45)n3)cc2)c1] 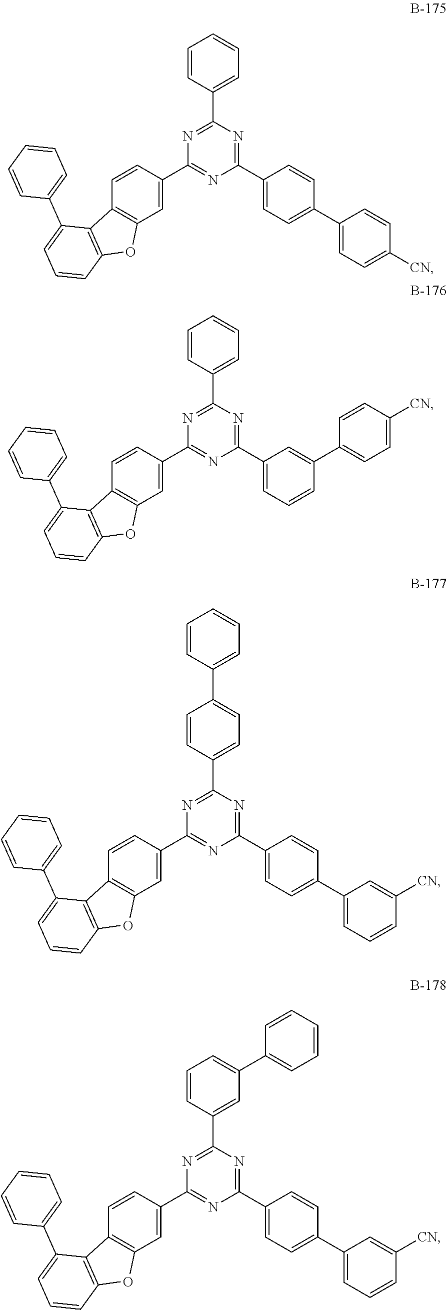 [SMILES: N#Cc1ccc(-c2ccc(-c3nc(-c4ccccc4)nc(-c4ccc5c(c4)oc4cccc(-c6ccccc6)c45)n3)cc2)cc1 ; N#Cc1ccc(-c2cccc(-c3nc(-c4ccccc4)nc(-c4ccc5c(c4)oc4cccc(-c6ccccc6)c45)n3)c2)cc1 ; N#Cc1cccc(-c2ccc(-c3nc(-c4ccc(-c5ccccc5)cc4)nc(-c4ccc5c(c4)oc4cccc(-c6ccccc6)c45)n3)cc2)c1 ; N#Cc1cccc(-c2ccc(-c3nc(-c4cccc(-c5ccccc5)c4)nc(-c4ccc5c(c4)oc4cccc(-c6ccccc6)c45)n3)cc2)c1] 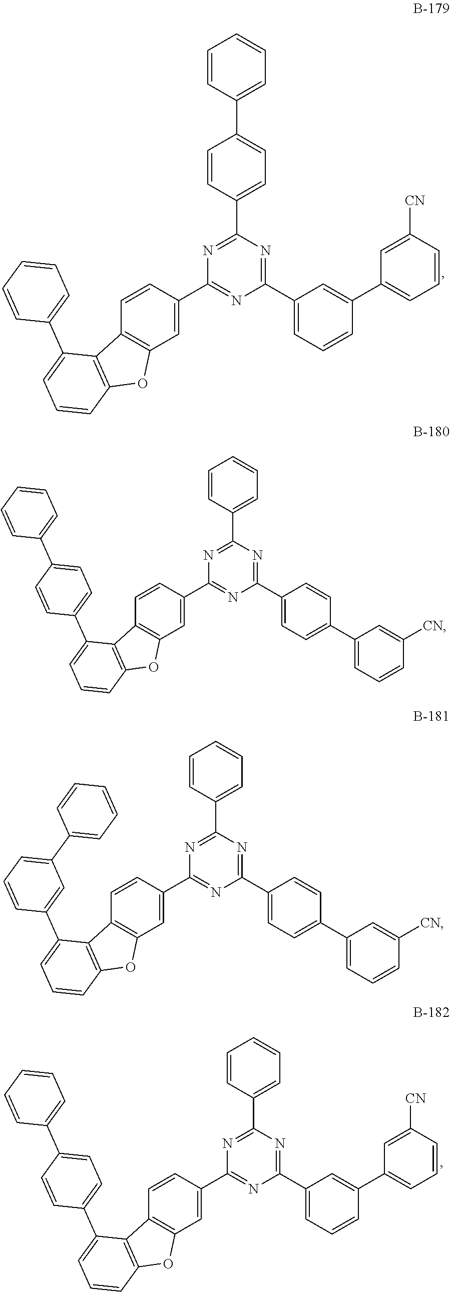 [SMILES: N#Cc1cccc(-c2cccc(-c3nc(-c4ccc(-c5ccccc5)cc4)nc(-c4ccc5c(c4)oc4cccc(-c6ccccc6)c45)n3)c2)c1 ; N#Cc1cccc(-c2ccc(-c3nc(-c4ccccc4)nc(-c4ccc5c(c4)oc4cccc(-c6ccc(-c7ccccc7)cc6)c45)n3)cc2)c1 ; N#Cc1cccc(-c2ccc(-c3nc(-c4ccccc4)nc(-c4ccc5c(c4)oc4cccc(-c6cccc(-c7ccccc7)c6)c45)n3)cc2)c1 ; N#Cc1cccc(-c2cccc(-c3nc(-c4ccccc4)nc(-c4ccc5c(c4)oc4cccc(-c6ccc(-c7ccccc7)cc6)c45)n3)c2)c1] 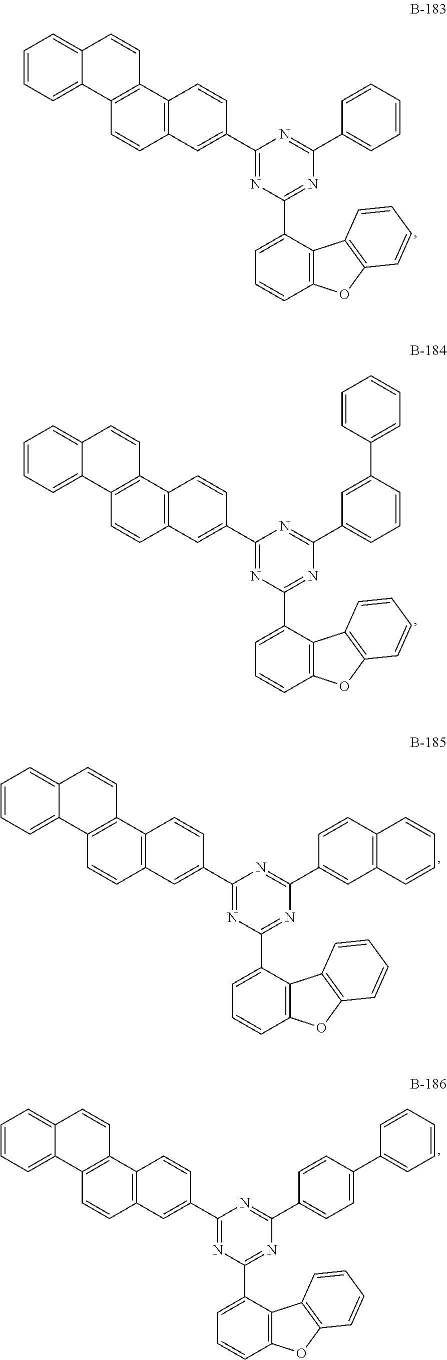 [SMILES: c1ccc(-c2nc(-c3ccc4c(ccc5c6ccccc6ccc45)c3)nc(-c3cccc4oc5ccccc5c34)n2)cc1 ; c1ccc(-c2cccc(-c3nc(-c4ccc5c(ccc6c7ccccc7ccc56)c4)nc(-c4cccc5oc6ccccc6c45)n3)c2)cc1 ; c1ccc2cc(-c3nc(-c4ccc5c(ccc6c7ccccc7ccc56)c4)nc(-c4cccc5oc6ccccc6c45)n3)ccc2c1 ; c1ccc(-c2ccc(-c3nc(-c4ccc5c(ccc6c7ccccc7ccc56)c4)nc(-c4cccc5oc6ccccc6c45)n3)cc2)cc1] 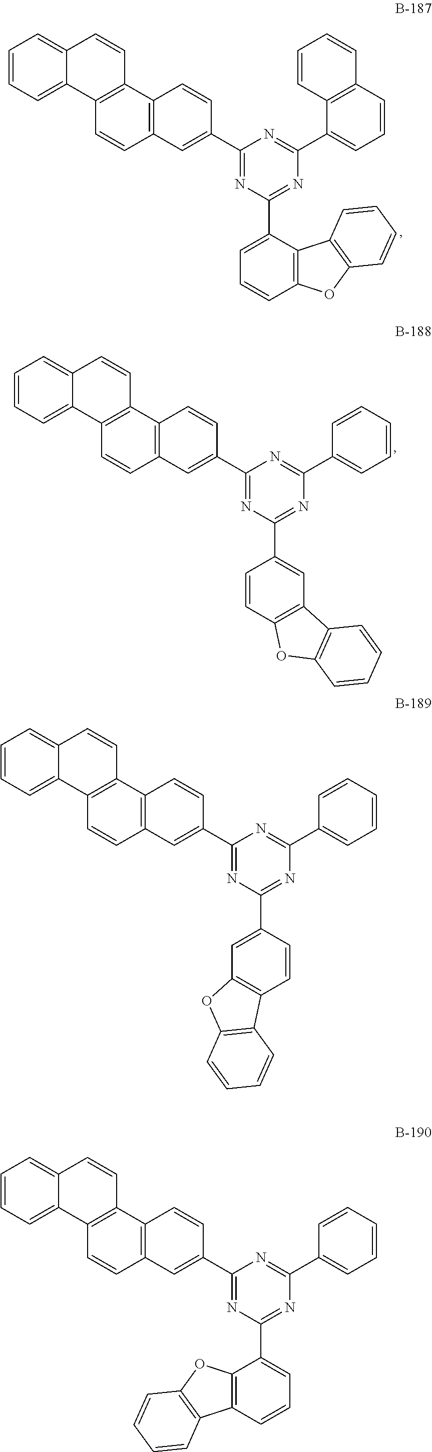 [SMILES: c1ccc2c(-c3nc(-c4ccc5c(ccc6c7ccccc7ccc56)c4)nc(-c4cccc5oc6ccccc6c45)n3)cccc2c1 ; c1ccc(-c2nc(-c3ccc4c(ccc5c6ccccc6ccc45)c3)nc(-c3ccc4oc5ccccc5c4c3)n2)cc1 ; c1ccc(-c2nc(-c3ccc4c(ccc5c6ccccc6ccc45)c3)nc(-c3ccc4c(c3)oc3ccccc34)n2)cc1 ; c1ccc(-c2nc(-c3ccc4c(ccc5c6ccccc6ccc45)c3)nc(-c3cccc4c3oc3ccccc34)n2)cc1] 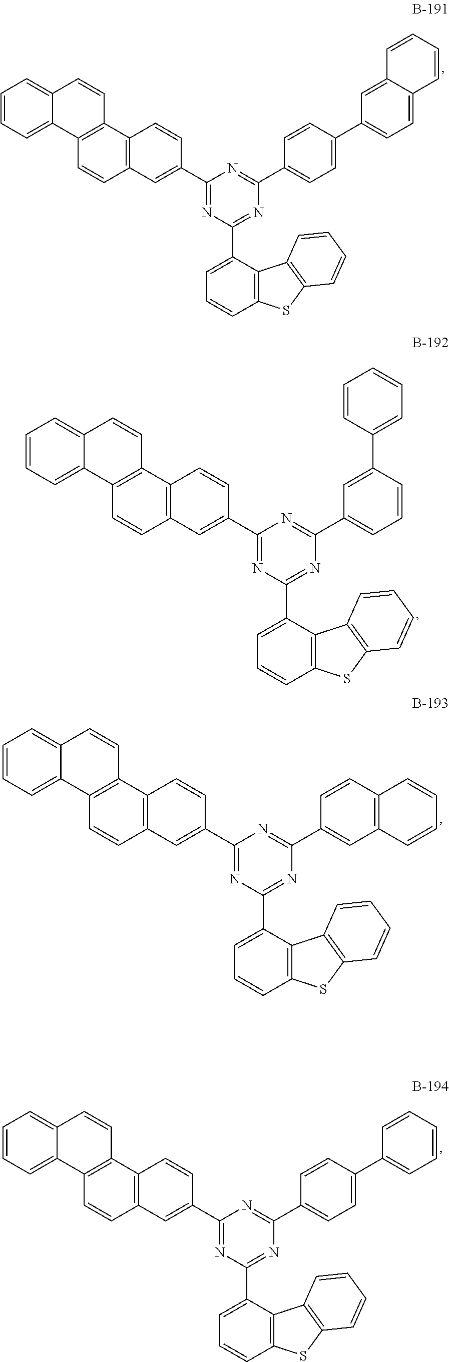 [SMILES: c1ccc2cc(-c3ccc(-c4nc(-c5ccc6c(ccc7c8ccccc8ccc67)c5)nc(-c5cccc6sc7ccccc7c56)n4)cc3)ccc2c1 ; c1ccc(-c2cccc(-c3nc(-c4ccc5c(ccc6c7ccccc7ccc56)c4)nc(-c4cccc5sc6ccccc6c45)n3)c2)cc1 ; c1ccc2cc(-c3nc(-c4ccc5c(ccc6c7ccccc7ccc56)c4)nc(-c4cccc5sc6ccccc6c45)n3)ccc2c1 ; c1ccc(-c2ccc(-c3nc(-c4ccc5c(ccc6c7ccccc7ccc56)c4)nc(-c4cccc5sc6ccccc6c45)n3)cc2)cc1] 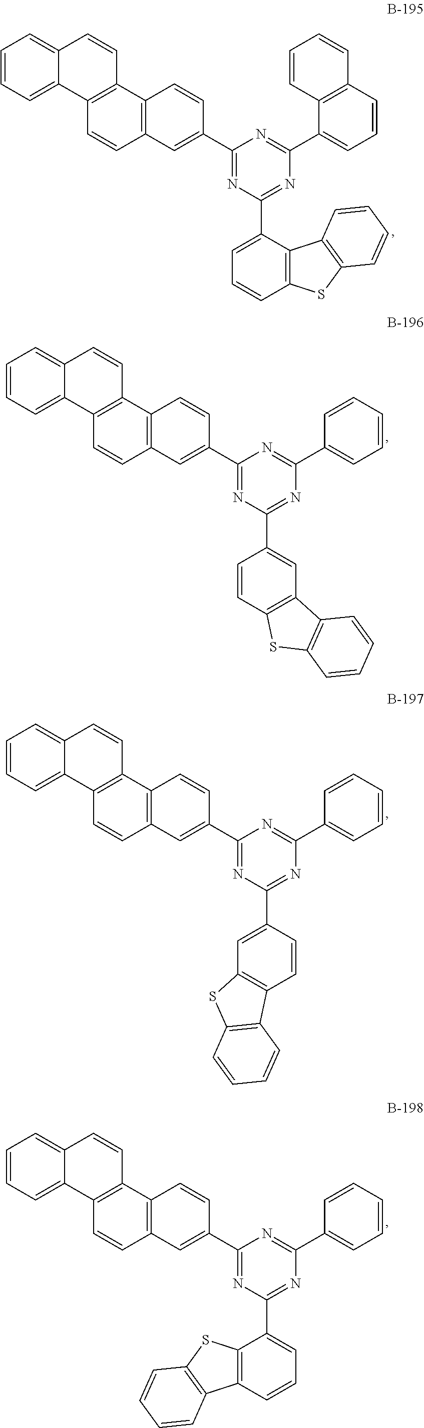 [SMILES: c1ccc2c(-c3nc(-c4ccc5c(ccc6c7ccccc7ccc56)c4)nc(-c4cccc5sc6ccccc6c45)n3)cccc2c1 ; c1ccc(-c2nc(-c3ccc4c(ccc5c6ccccc6ccc45)c3)nc(-c3ccc4sc5ccccc5c4c3)n2)cc1 ; c1ccc(-c2nc(-c3ccc4c(ccc5c6ccccc6ccc45)c3)nc(-c3ccc4c(c3)sc3ccccc34)n2)cc1 ; c1ccc(-c2nc(-c3ccc4c(ccc5c6ccccc6ccc45)c3)nc(-c3cccc4c3sc3ccccc34)n2)cc1] 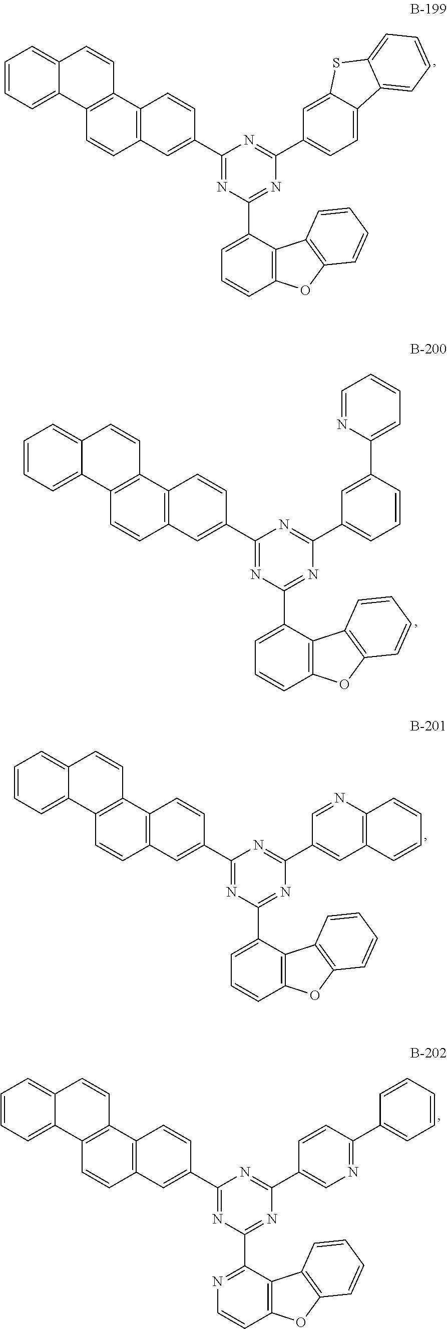 [SMILES: c1ccc2c(c1)ccc1c3ccc(-c4nc(-c5ccc6c(c5)sc5ccccc56)nc(-c5cccc6oc7ccccc7c56)n4)cc3ccc21 ; c1ccc(-c2cccc(-c3nc(-c4ccc5c(ccc6c7ccccc7ccc56)c4)nc(-c4cccc5oc6ccccc6c45)n3)c2)nc1 ; c1ccc2ncc(-c3nc(-c4ccc5c(ccc6c7ccccc7ccc56)c4)nc(-c4cccc5oc6ccccc6c45)n3)cc2c1 ; c1ccc(-c2ccc(-c3nc(-c4ccc5c(ccc6c7ccccc7ccc56)c4)nc(-c4nccc5oc6ccccc6c45)n3)cn2)cc1] 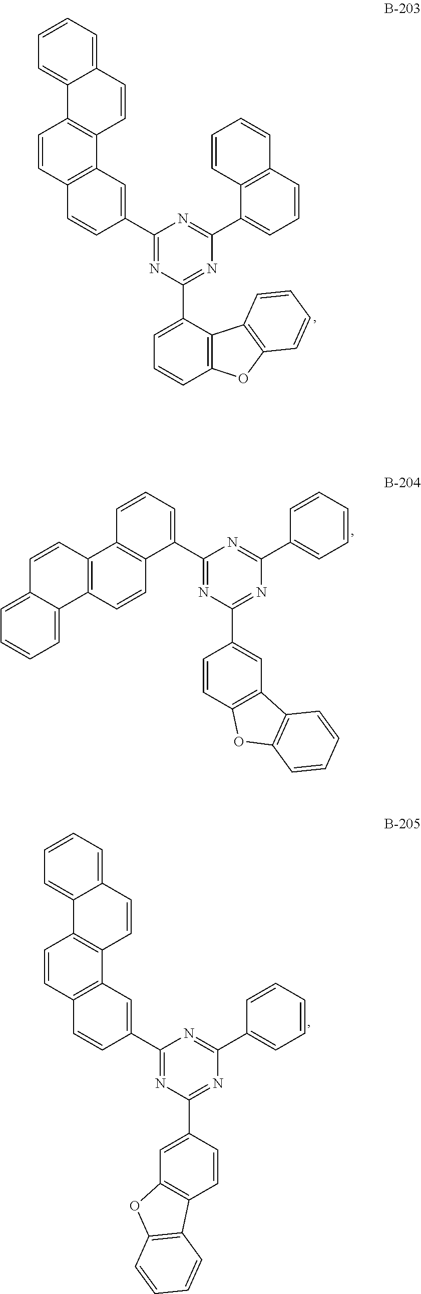 [SMILES: c1ccc2c(-c3nc(-c4ccc5ccc6c7ccccc7ccc6c5c4)nc(-c4cccc5oc6ccccc6c45)n3)cccc2c1 ; c1ccc(-c2nc(-c3ccc4oc5ccccc5c4c3)nc(-c3cccc4c3ccc3c5ccccc5ccc43)n2)cc1 ; c1ccc(-c2nc(-c3ccc4c(c3)oc3ccccc34)nc(-c3ccc4ccc5c6ccccc6ccc5c4c3)n2)cc1] 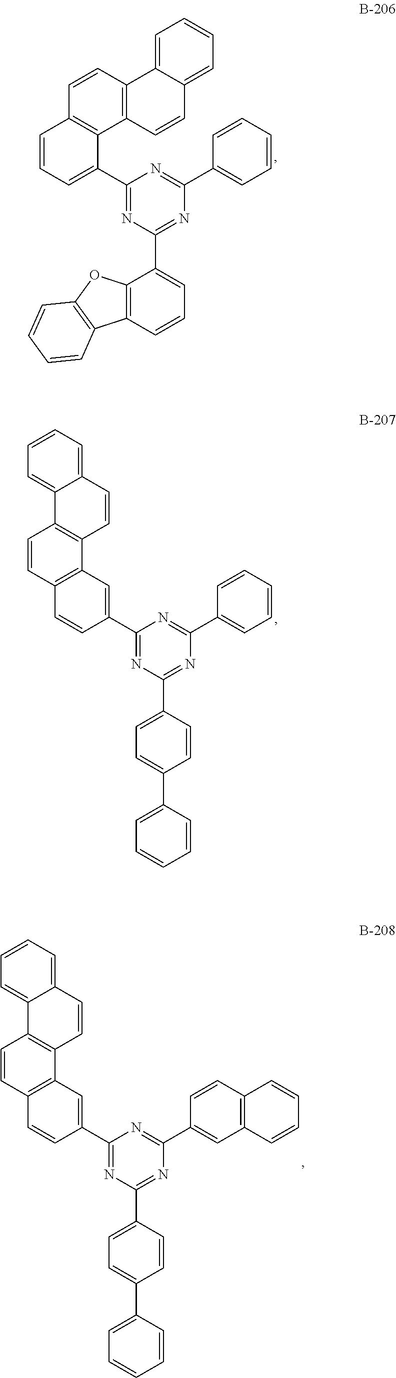 [SMILES: c1ccc(-c2nc(-c3cccc4c3oc3ccccc34)nc(-c3cccc4ccc5c6ccccc6ccc5c34)n2)cc1 ; c1ccc(-c2ccc(-c3nc(-c4ccccc4)nc(-c4ccc5ccc6c7ccccc7ccc6c5c4)n3)cc2)cc1 ; c1ccc(-c2ccc(-c3nc(-c4ccc5ccccc5c4)nc(-c4ccc5ccc6c7ccccc7ccc6c5c4)n3)cc2)cc1] 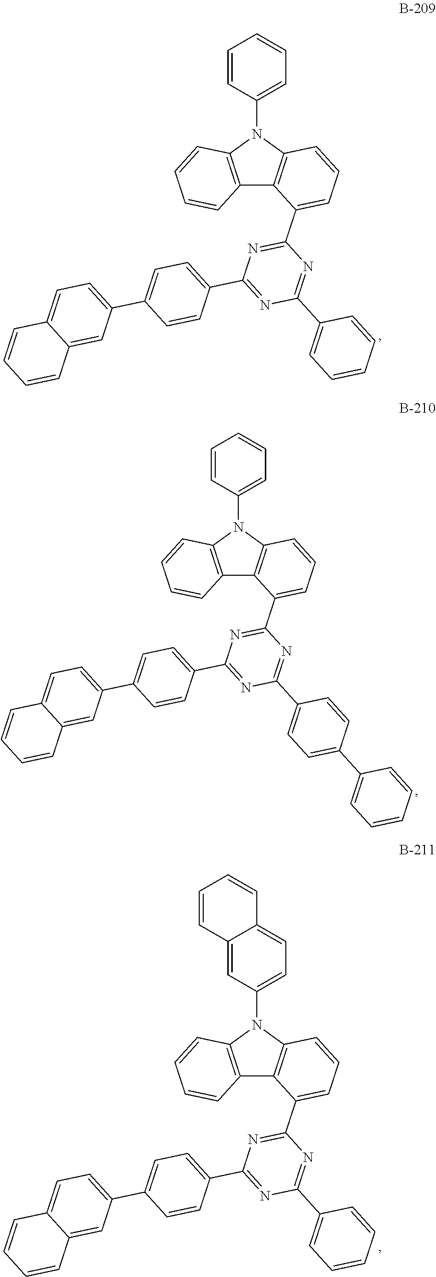 [SMILES: c1ccc(-c2nc(-c3ccc(-c4ccc5ccccc5c4)cc3)nc(-c3cccc4c3c3ccccc3n4-c3ccccc3)n2)cc1 ; c1ccc(-c2ccc(-c3nc(-c4ccc(-c5ccc6ccccc6c5)cc4)nc(-c4cccc5c4c4ccccc4n5-c4ccccc4)n3)cc2)cc1 ; c1ccc(-c2nc(-c3ccc(-c4ccc5ccccc5c4)cc3)nc(-c3cccc4c3c3ccccc3n4-c3ccc4ccccc4c3)n2)cc1] 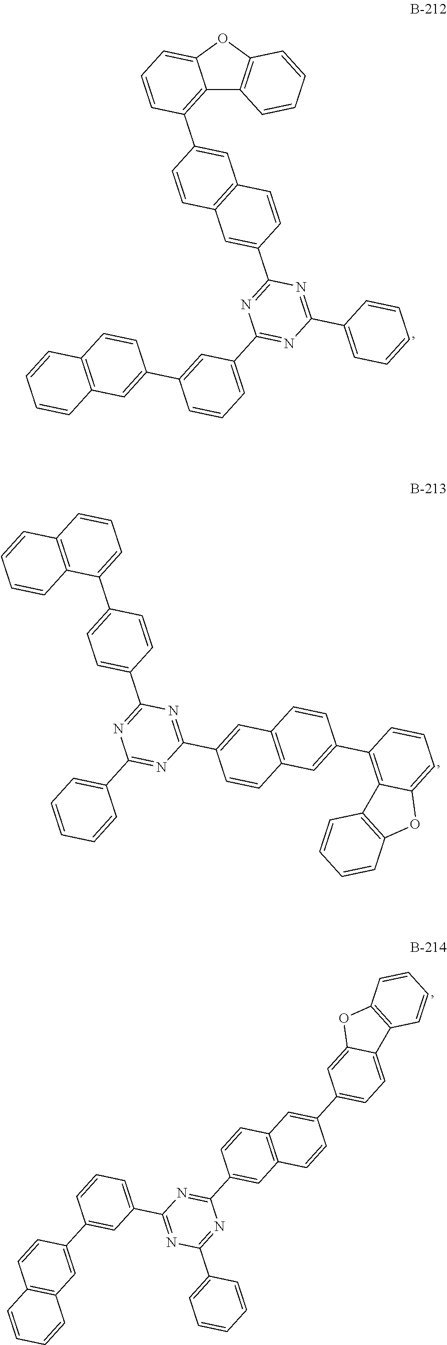 [SMILES: c1ccc(-c2nc(-c3cccc(-c4ccc5ccccc5c4)c3)nc(-c3ccc4cc(-c5cccc6oc7ccccc7c56)ccc4c3)n2)cc1 ; c1ccc(-c2nc(-c3ccc(-c4cccc5ccccc45)cc3)nc(-c3ccc4cc(-c5cccc6oc7ccccc7c56)ccc4c3)n2)cc1 ; c1ccc(-c2nc(-c3cccc(-c4ccc5ccccc5c4)c3)nc(-c3ccc4cc(-c5ccc6c(c5)oc5ccccc56)ccc4c3)n2)cc1] 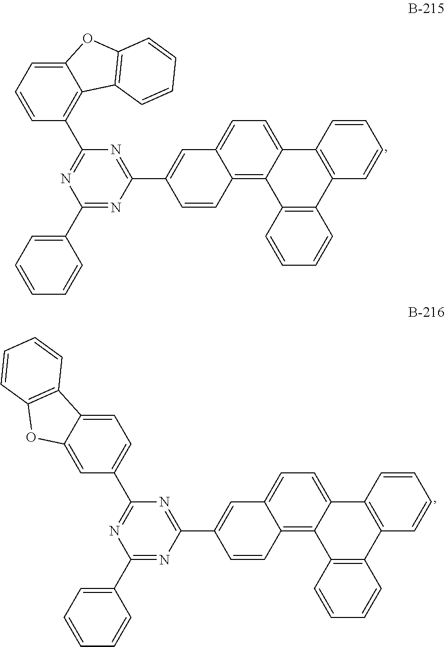 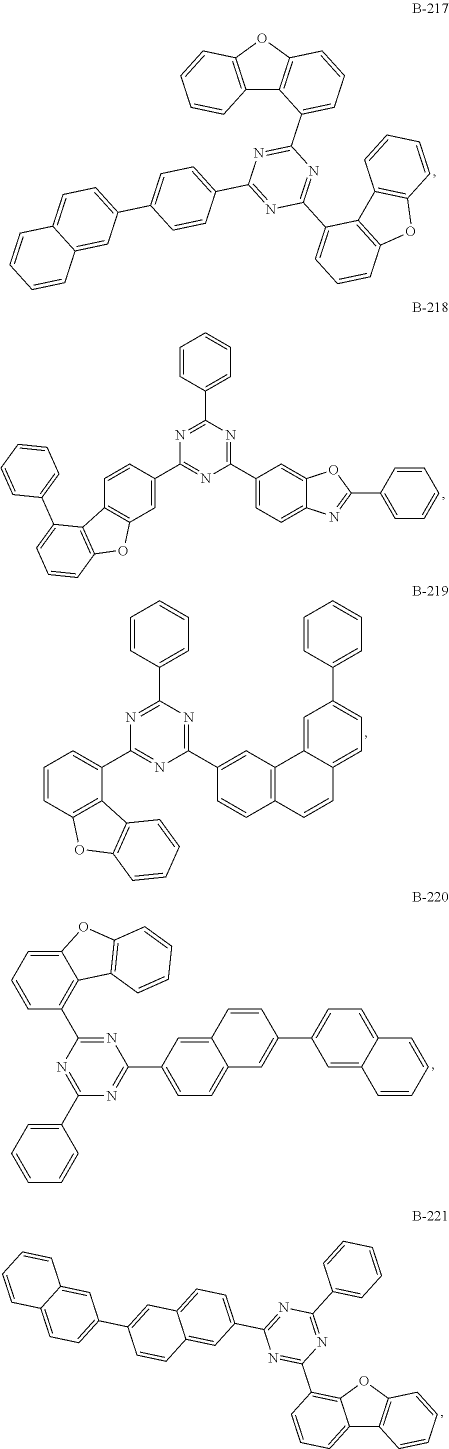 [SMILES: c1ccc2cc(-c3ccc(-c4nc(-c5cccc6oc7ccccc7c56)nc(-c5cccc6oc7ccccc7c56)n4)cc3)ccc2c1 ; c1ccc(-c2nc(-c3ccc4nc(-c5ccccc5)oc4c3)nc(-c3ccc4c(c3)oc3cccc(-c5ccccc5)c34)n2)cc1 ; c1ccc(-c2ccc3ccc4ccc(-c5nc(-c6ccccc6)nc(-c6cccc7oc8ccccc8c67)n5)cc4c3c2)cc1 ; c1ccc(-c2nc(-c3ccc4cc(-c5ccc6ccccc6c5)ccc4c3)nc(-c3cccc4oc5ccccc5c34)n2)cc1 ; c1ccc(-c2nc(-c3ccc4cc(-c5ccc6ccccc6c5)ccc4c3)nc(-c3cccc4c3oc3ccccc34)n2)cc1] 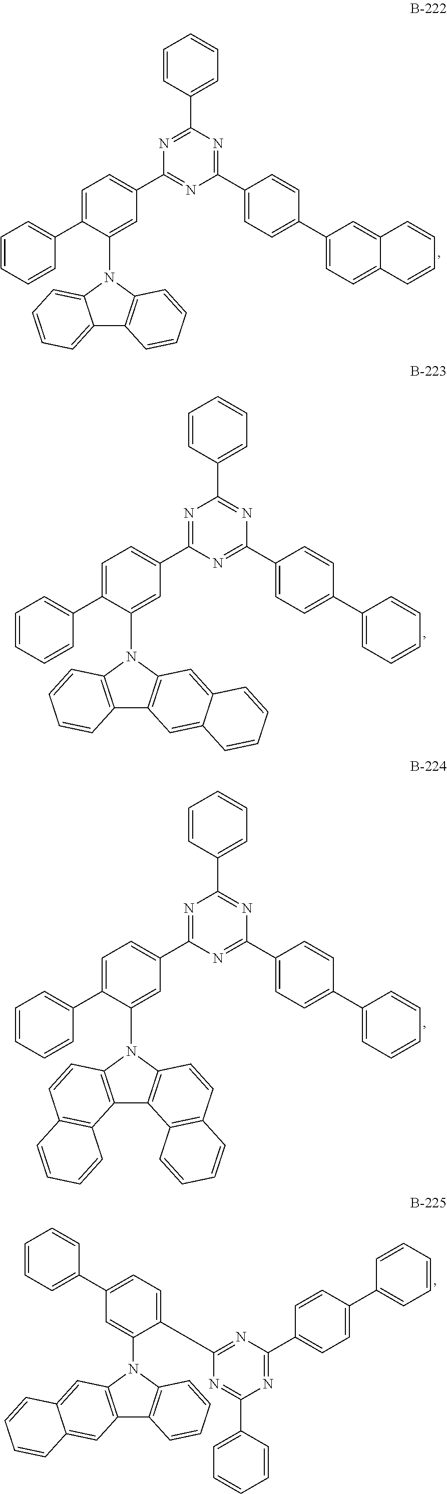 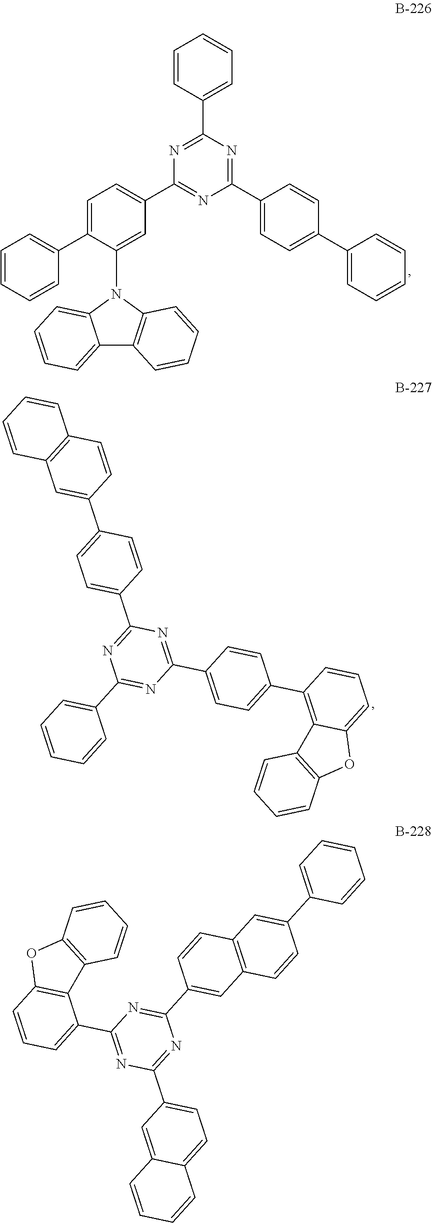 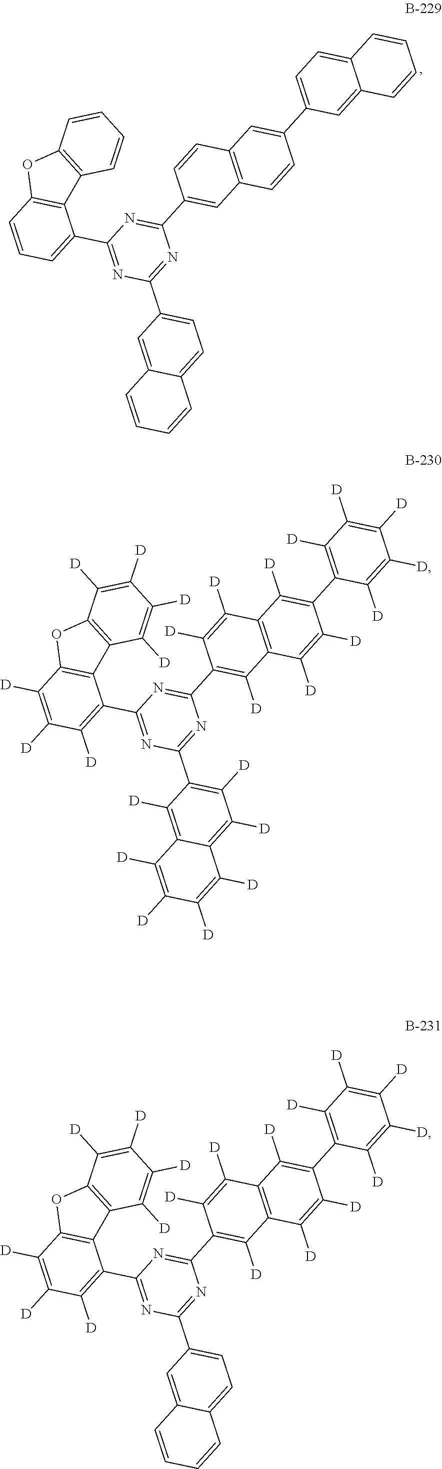 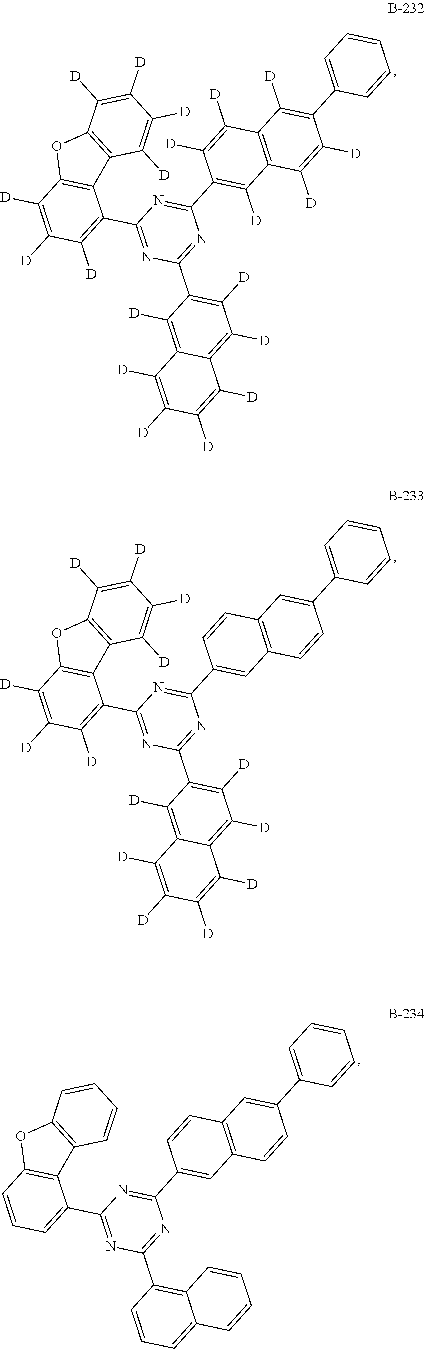 [SMILES: [2H]c1c([2H])c([2H])c2c(oc3c([2H])c([2H])c([2H])c(-c4nc(-c5c([2H])c([2H])c6c([2H])c([2H])c([2H])c([2H])c6c5[2H])nc(-c5c([2H])c([2H])c6c([2H])c(-c7ccccc7)c([2H])c([2H])c6c5[2H])n4)c32)c1[2H] ; [2H]c1c([2H])c([2H])c2c(oc3c([2H])c([2H])c([2H])c(-c4nc(-c5ccc6cc(-c7ccccc7)ccc6c5)nc(-c5c([2H])c([2H])c6c([2H])c([2H])c([2H])c([2H])c6c5[2H])n4)c32)c1[2H] ; c1ccc(-c2ccc3cc(-c4nc(-c5cccc6ccccc56)nc(-c5cccc6oc7ccccc7c56)n4)ccc3c2)cc1] 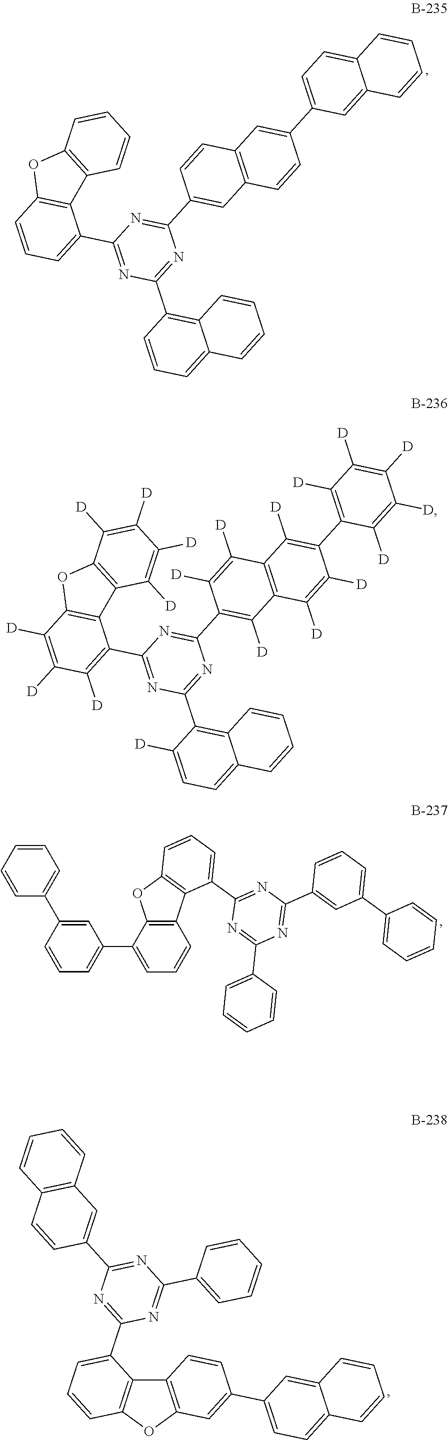 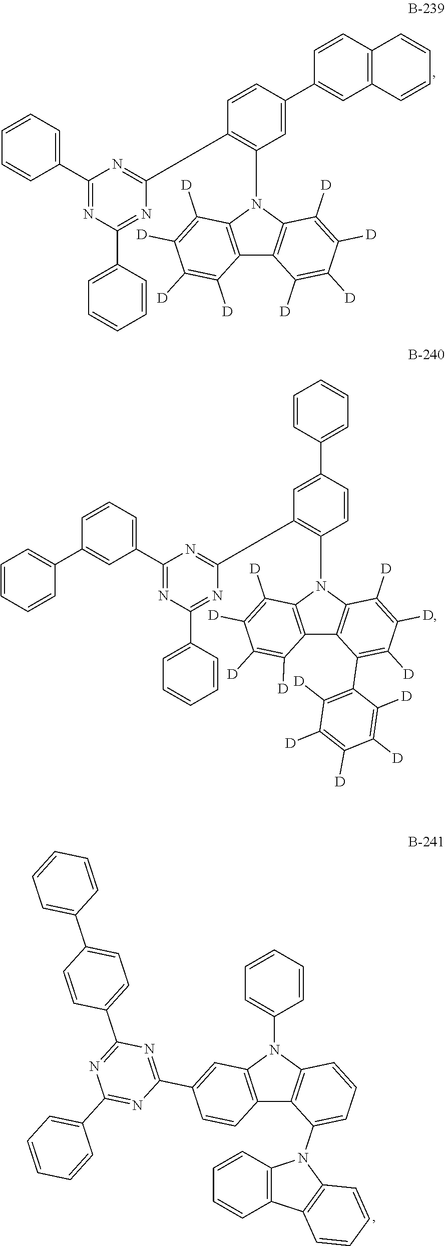 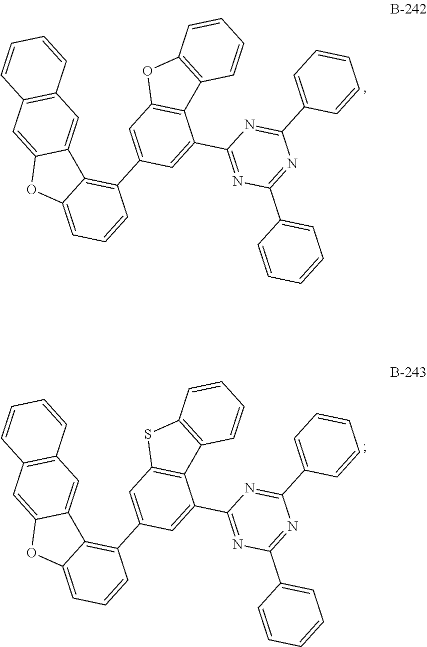 [SMILES: c1ccc(-c2nc(-c3ccccc3)nc(-c3cc(-c4cccc5oc6cc7ccccc7cc6c45)cc4oc5ccccc5c34)n2)cc1 ; c1ccc(-c2nc(-c3ccccc3)nc(-c3cc(-c4cccc5oc6cc7ccccc7cc6c45)cc4sc5ccccc5c34)n2)cc1]
wherein optionally, hydrogens in the structures of Compound B-1 to Compound B-243 can be partially or fully substituted with deuterium.

14. The organic electroluminescent device according to claim 11, wherein the emissive layer comprises at least one metal complex;
preferably, the metal complex is a phosphorescent sensitizer;
more preferably, the metal complex has a general formula of M(Lₐ)ₘ(L_{b})ₙ(L_{c})_{q};
the metal M is selected from a metal with a relative atomic mass greater than 40;
Lₐ, L_{b}, and Lₑ are a first ligand, a second ligand, and a third ligand coordinated to the metal M, respectively, and Lₐ, L_{b}, and Lₑ can be optionally joined to form a multidentate ligand;
Lₐ, L_{b}, and Lₑ are identical or different; m is 1, 2, or 3, n is 0, 1, or 2, q is 0, 1, or 2, and m+n+q is equal to the oxidation state of the metal M; when m is greater than or equal to 2, multiple Lₐ are identical or different; when n is 2, two L_{b} are identical or different; when q is 2, two Lₑ are identical or different;
Lₐ is, at each occurrence identically or differently, selected from a structure represented by Formula 2: 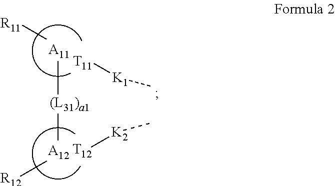
wherein
the ring A₁₁ and the ring A₁₂ are each selected from an aromatic ring having 6 to 30 ring atoms, a heteroaromatic ring having 5 to 30 ring atoms, or a combination thereof;
T₁₁ and T₁₂ are, at each occurrence identically or differently, selected from C or N;
K₁₁ and K₁₂ are, at each occurrence identically or differently, selected from a single bond, O, S, or NRₖ;
L₃₁ is, at each occurrence identically or differently, selected from the group consisting of: a single bond, BR_{L11}, CR_{L11}R_{L11}, NR_{L11}, O, SiR_{L11}R_{L11}, PR_{L11}, S, GeR_{L11}R_{L11}, Se, substituted or unsubstituted vinylene, ethynylene, substituted or unsubstituted arylene having 6 to 30 carbon atoms, substituted or unsubstituted heteroarylene having 3 to 30 carbon atoms, and combinations thereof; when two R_{L11} are present at the same time, the two R_{L11} are identical or different;
a₁ is selected from 0 or 1;
R₁₁ and R₁₂ represent, at each occurrence identically or differently, mono-substitution, multiple substitutions, or non-substitution;
R_{L11}, R₁₁, R₁₂, and Rₖ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof; and
adjacent substituents R_{L11}, R₁₁, and R₁₂ can be optionally joined to form a ring;
L_{b} and L_{c} are, at each occurrence identically or differently, selected from any one of the following structures: 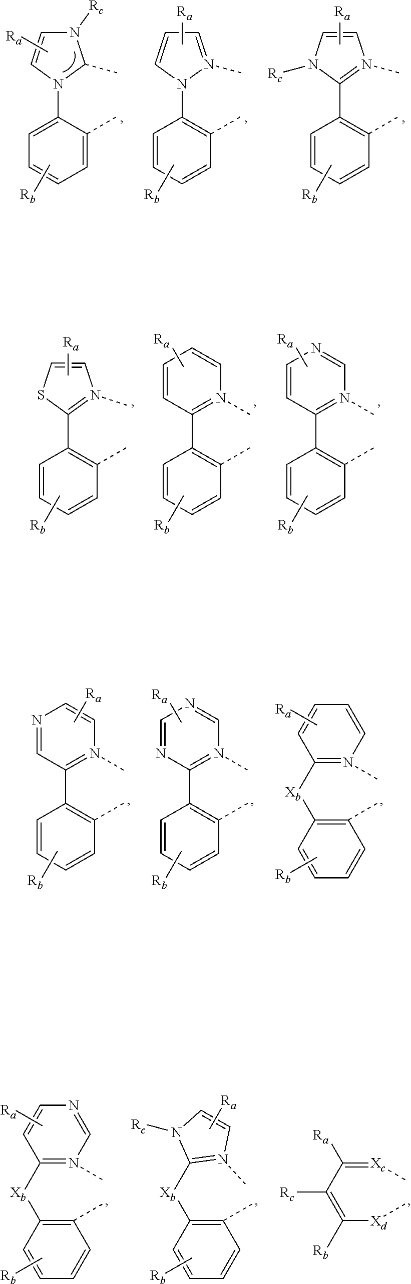
wherein
Rₐ, R_{b}, and R_{c} represent, at each occurrence identically or differently, mono-substitution, multiple substitutions, or non-substitution;
X_{b} is, at each occurrence identically or differently, selected from the group consisting of: O, S, Se, NR_{N1}, and CR_{C1}R_{C2};
X_{c} and X_{d} are, at each occurrence identically or differently, selected from the group consisting of: O, S, Se, and NR_{N2};
Rₐ, R_{b}, R_{c}, R_{N1}, R_{N2}, R_{C1}, and R_{C2} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof; and
adjacent substituents Rₐ, R_{b}, R_{c}, R_{N1}, R_{N2}, R_{C1}, and R_{C2} can be optionally joined to form a ring;
most preferably, the metal complex is selected from the group consisting of Compound RD1 to Compound RD214 and Compound GD1 to Compound GD184: 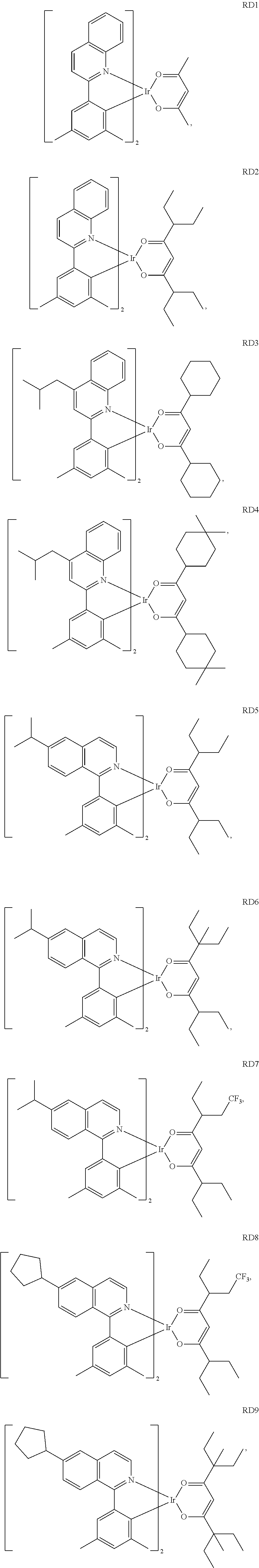 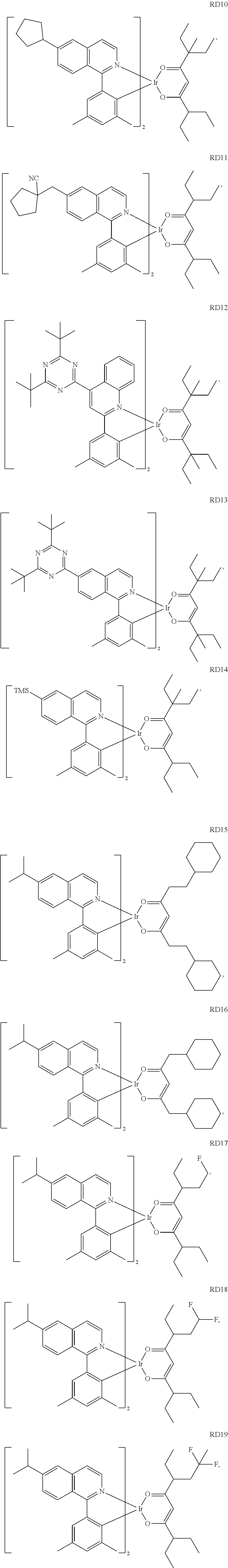 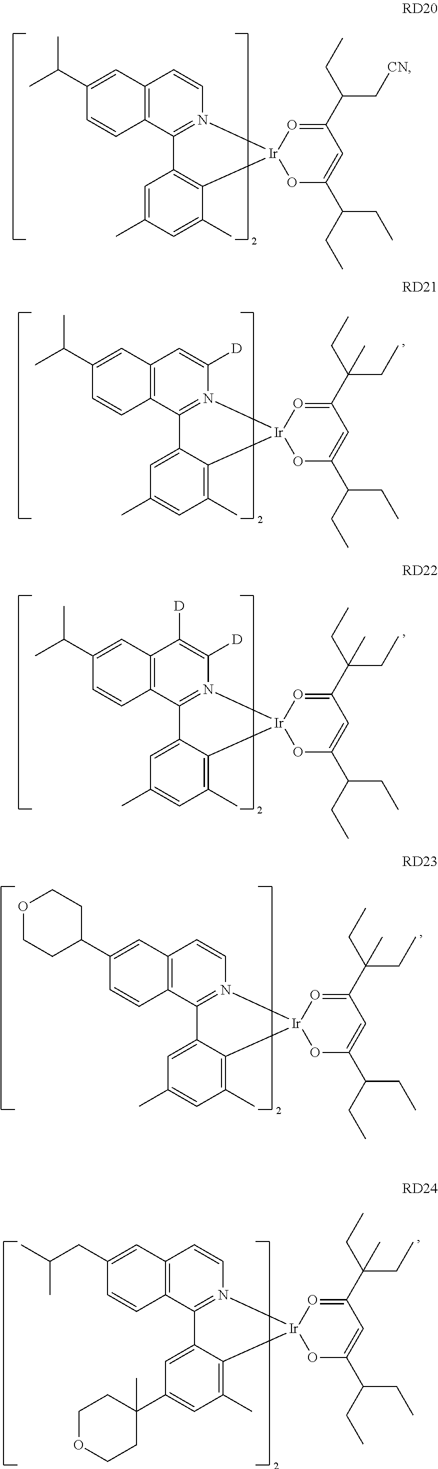 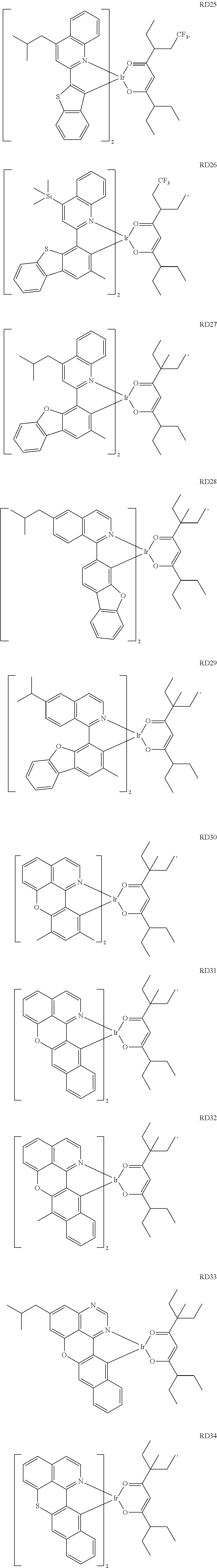 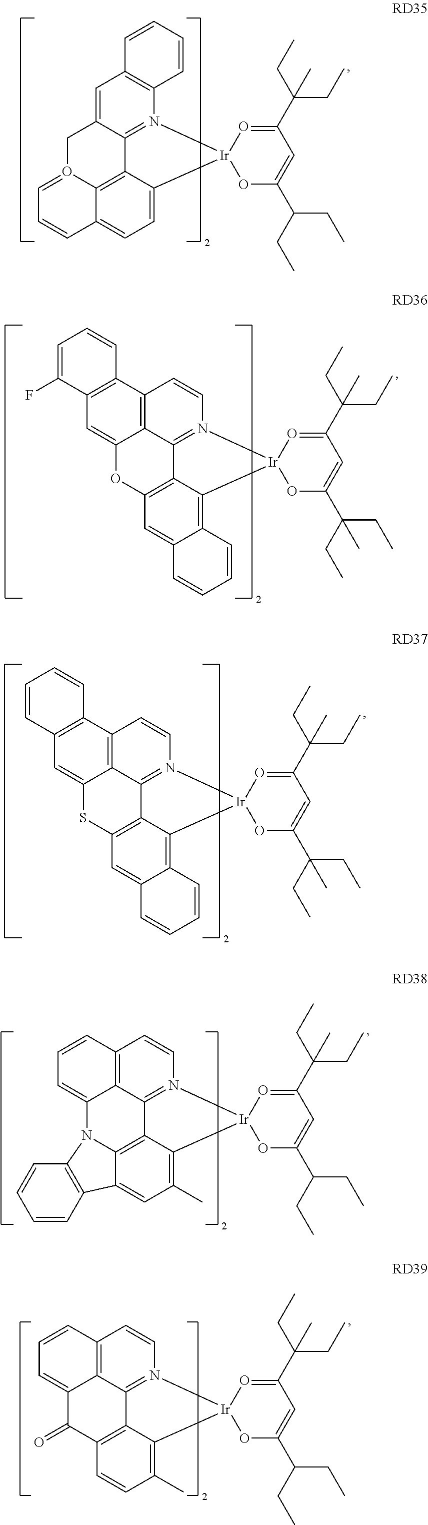 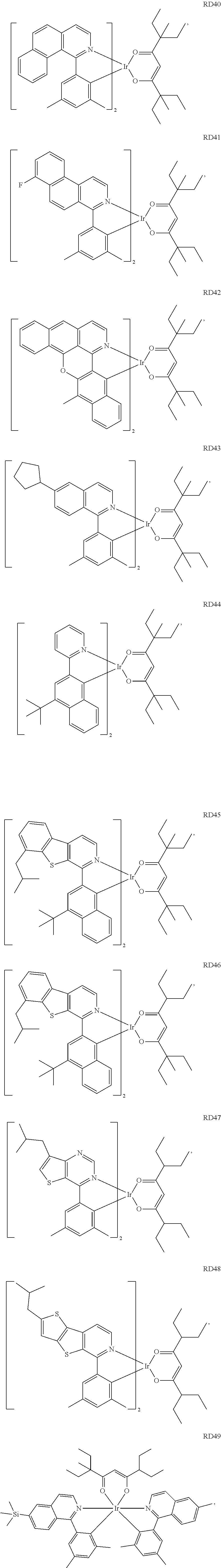 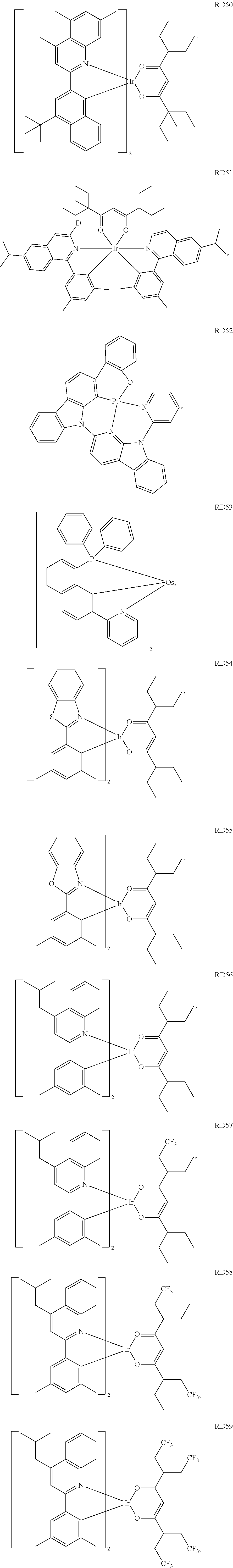 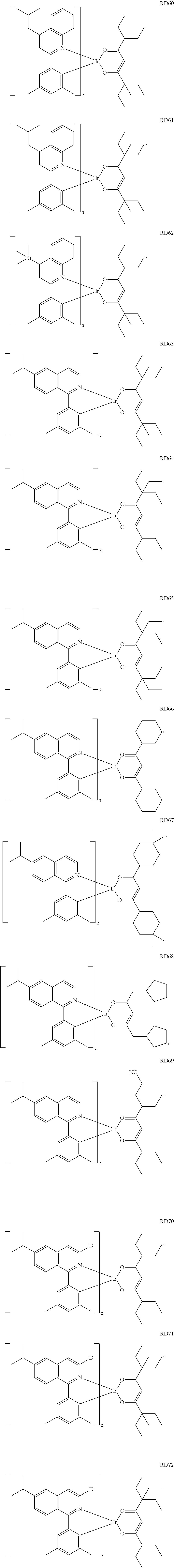 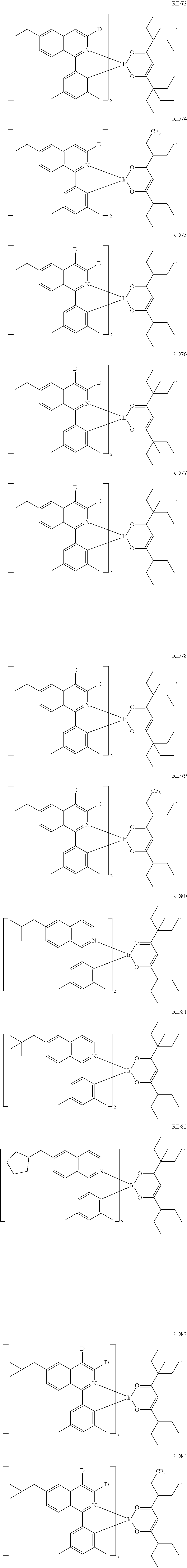 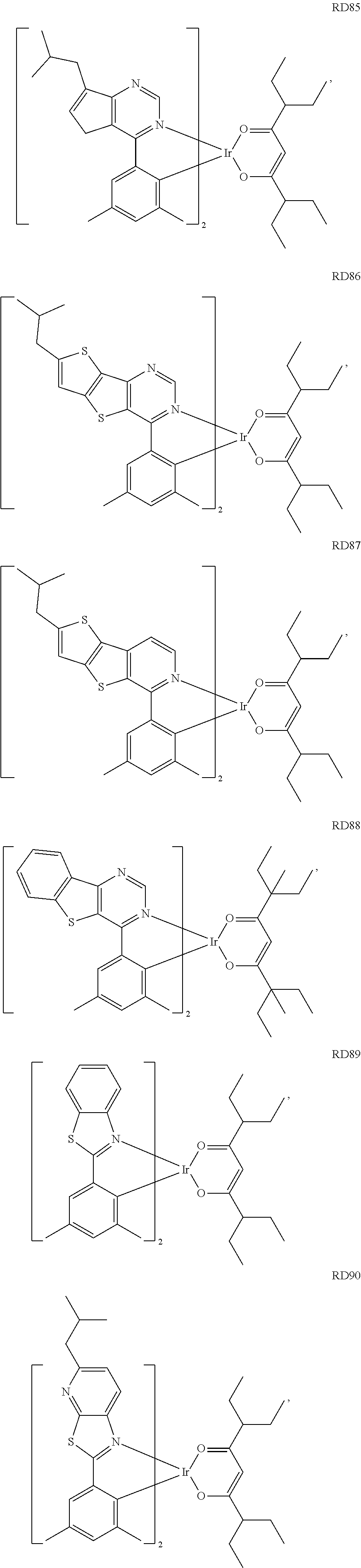 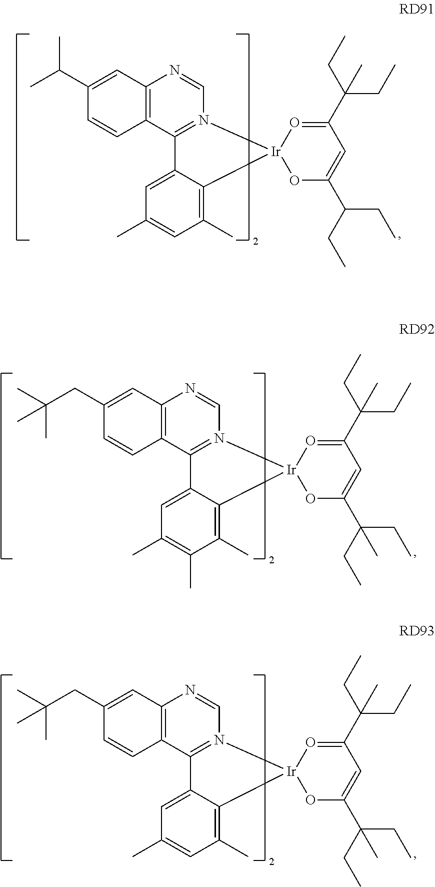 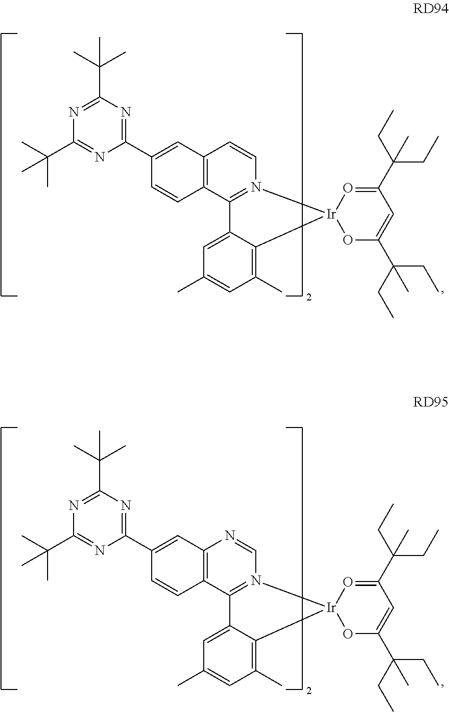 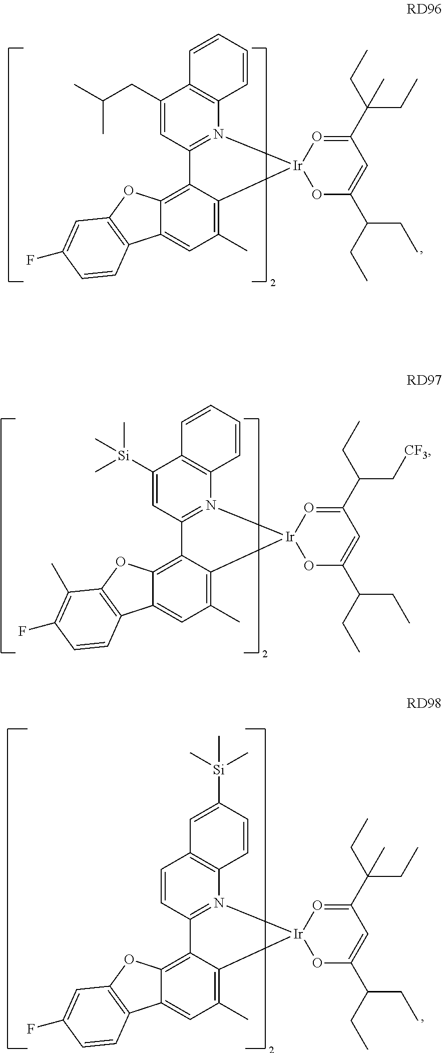 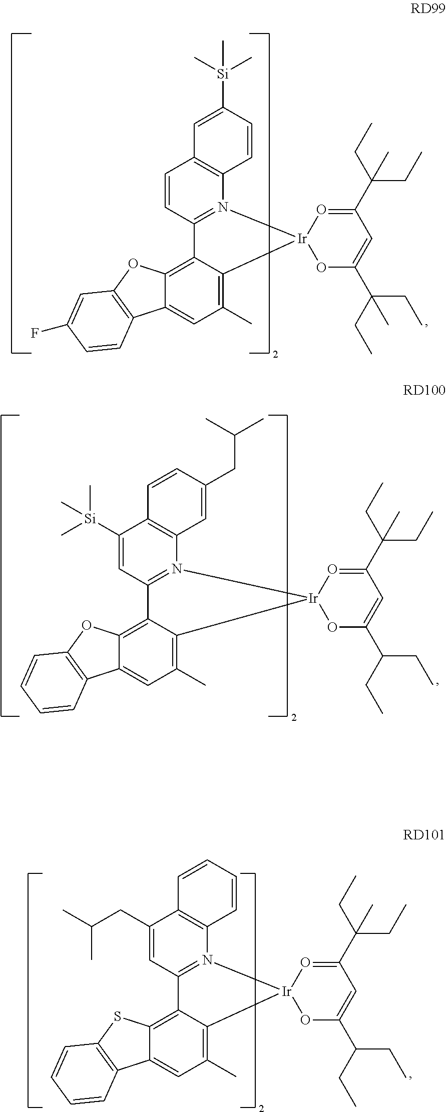 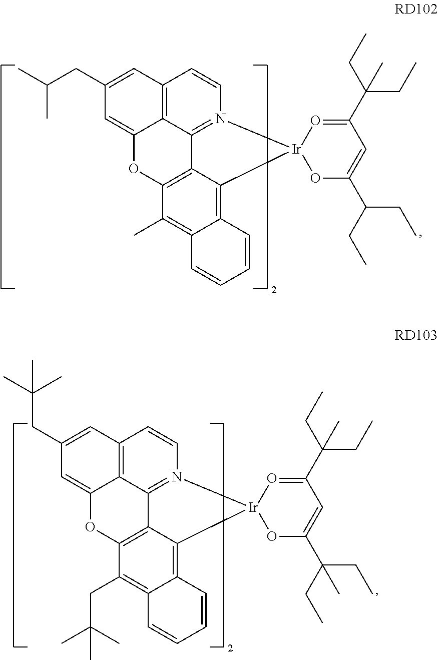 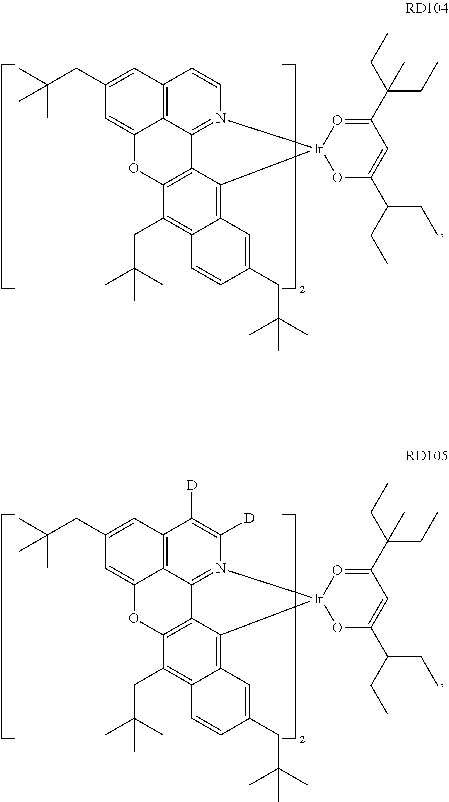 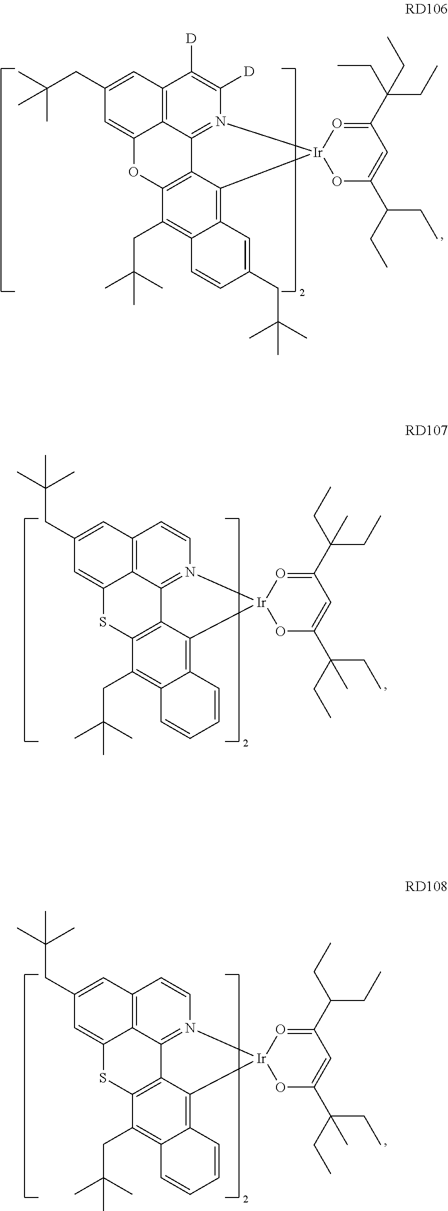 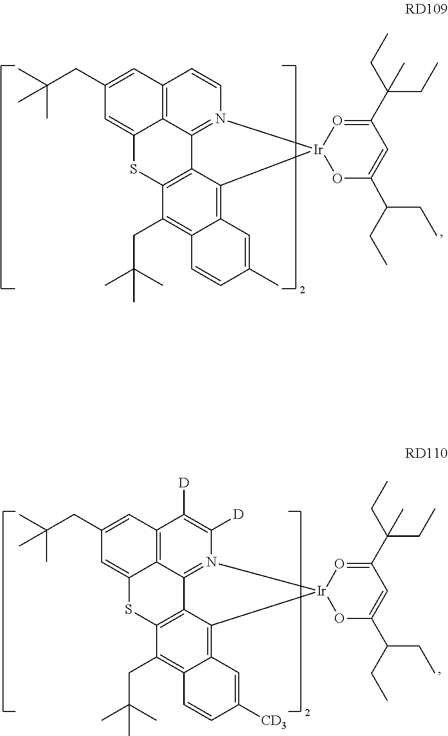 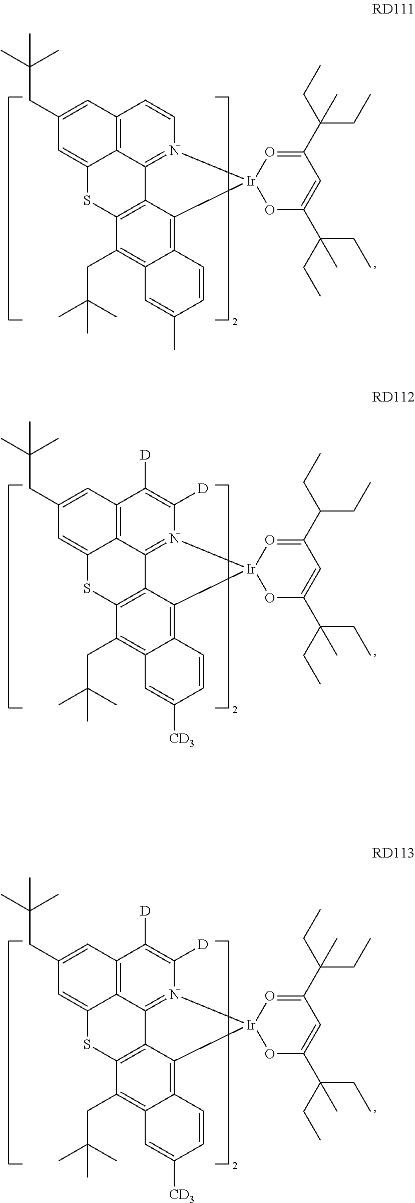 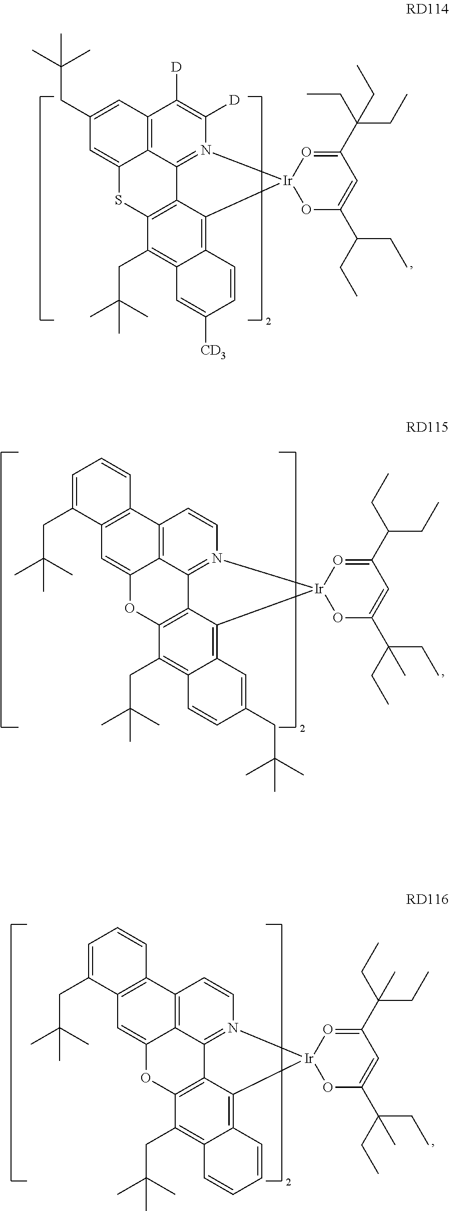 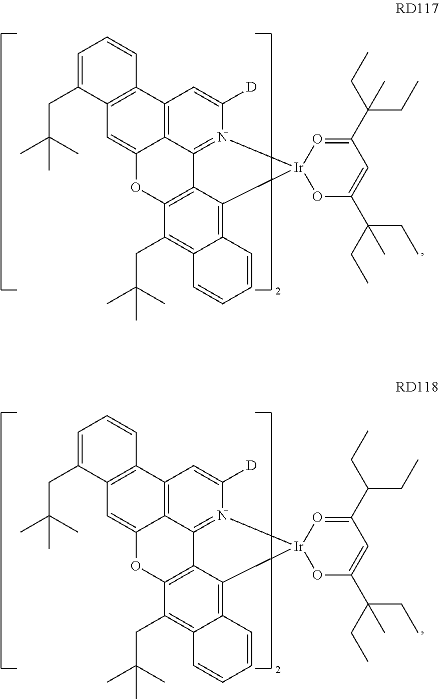 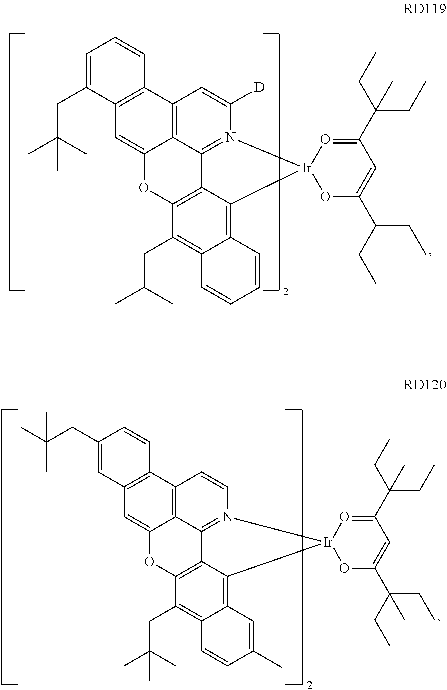 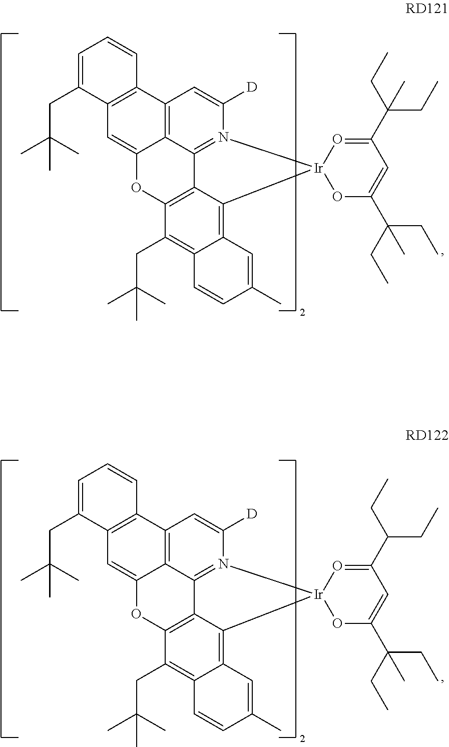 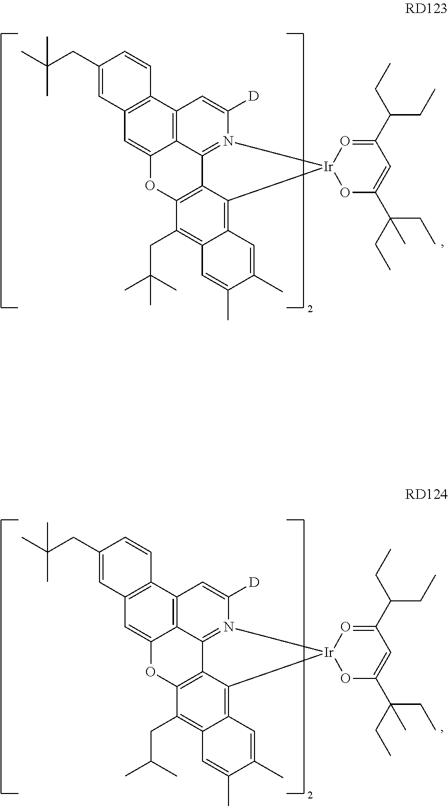 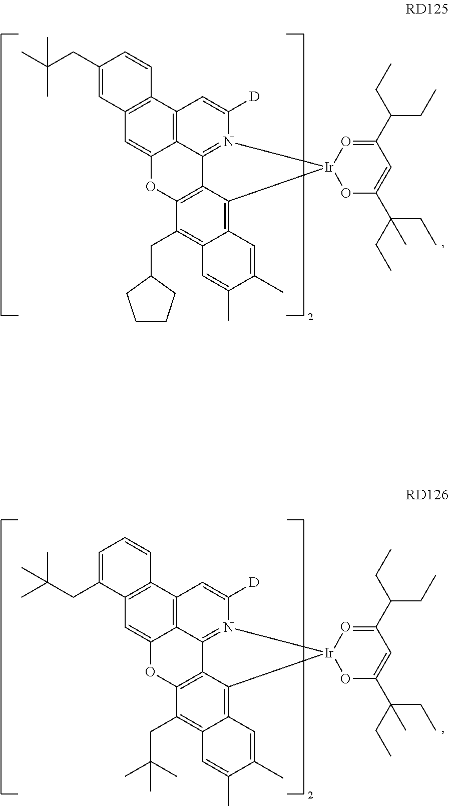 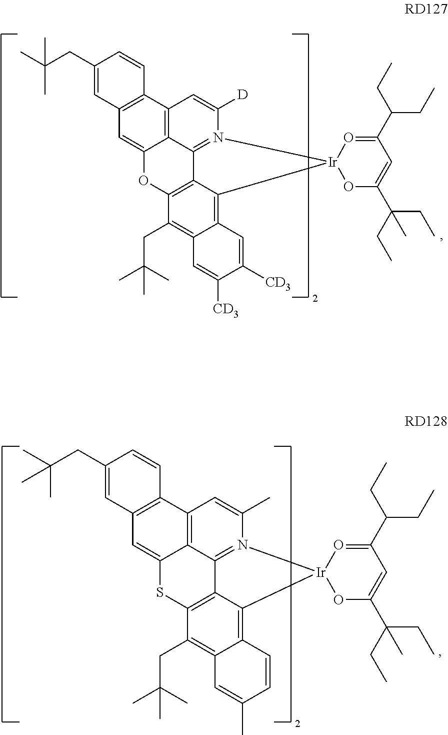 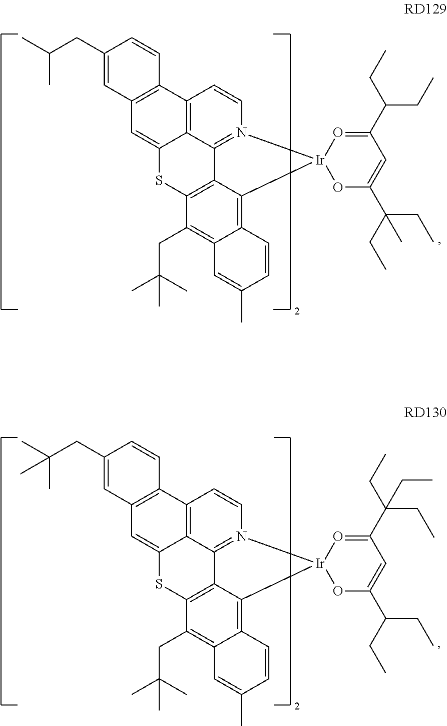 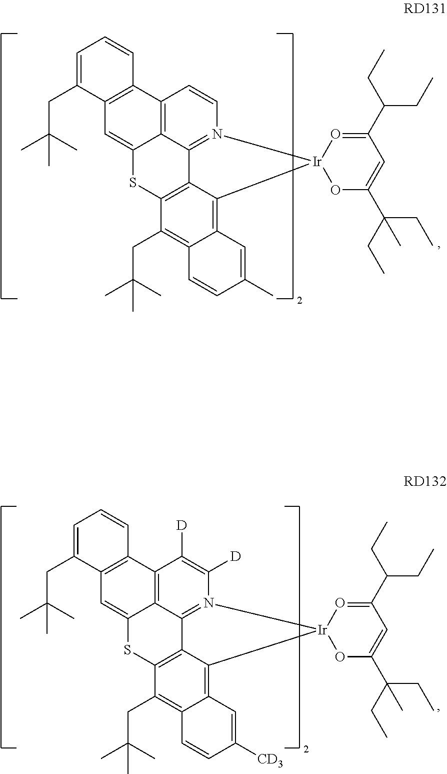 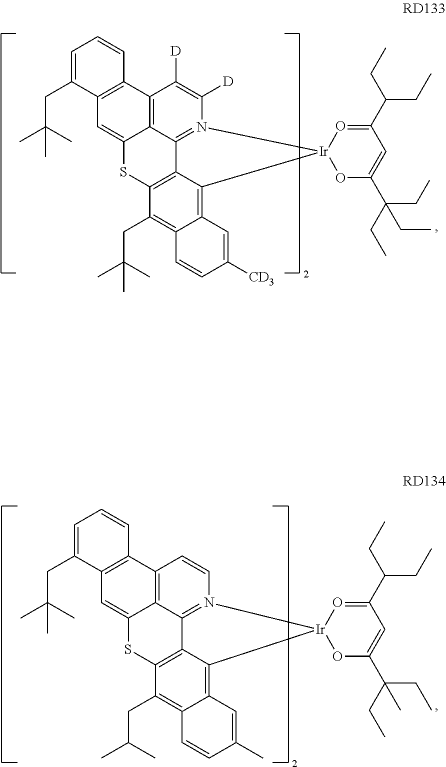 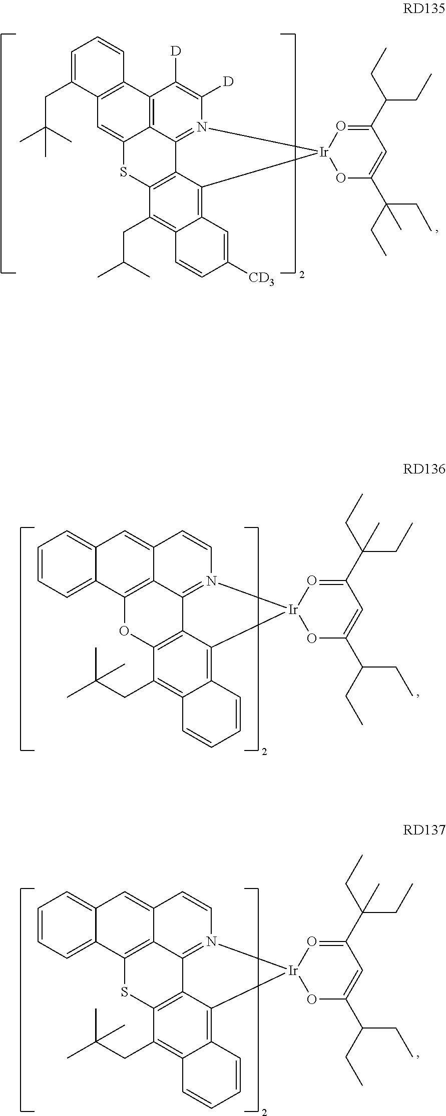 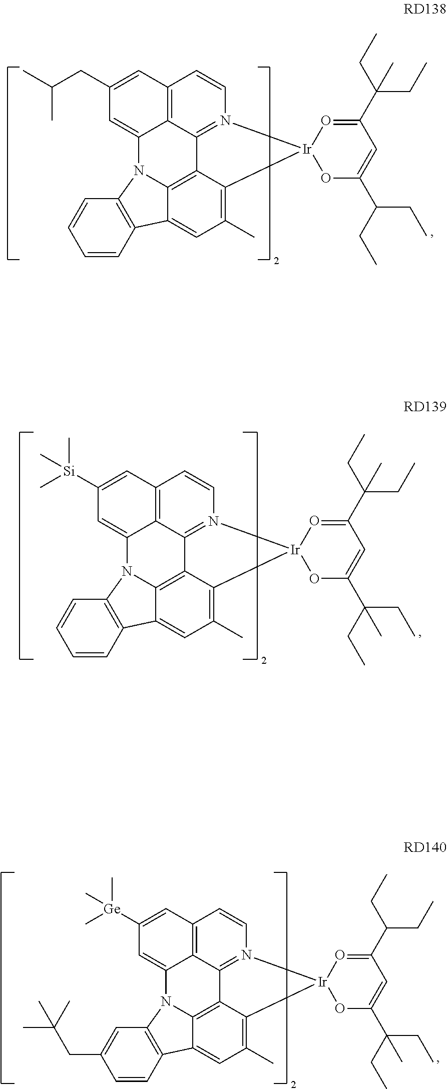 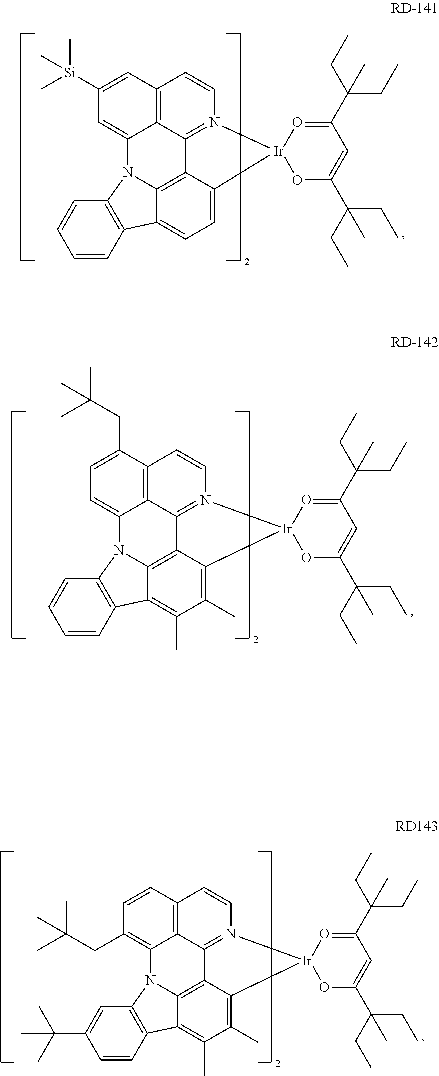 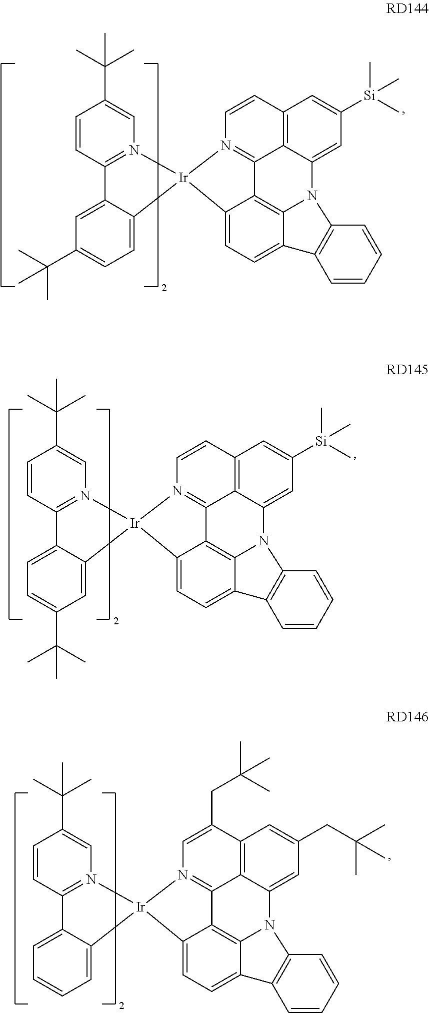 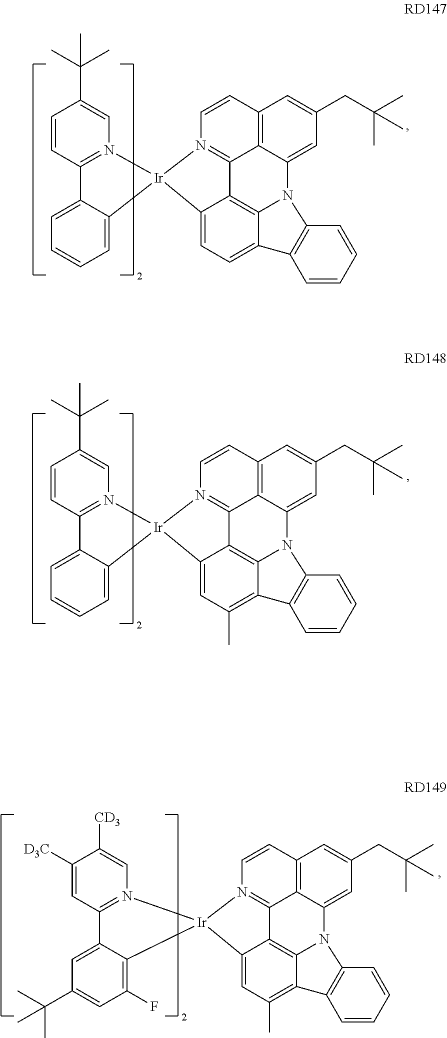 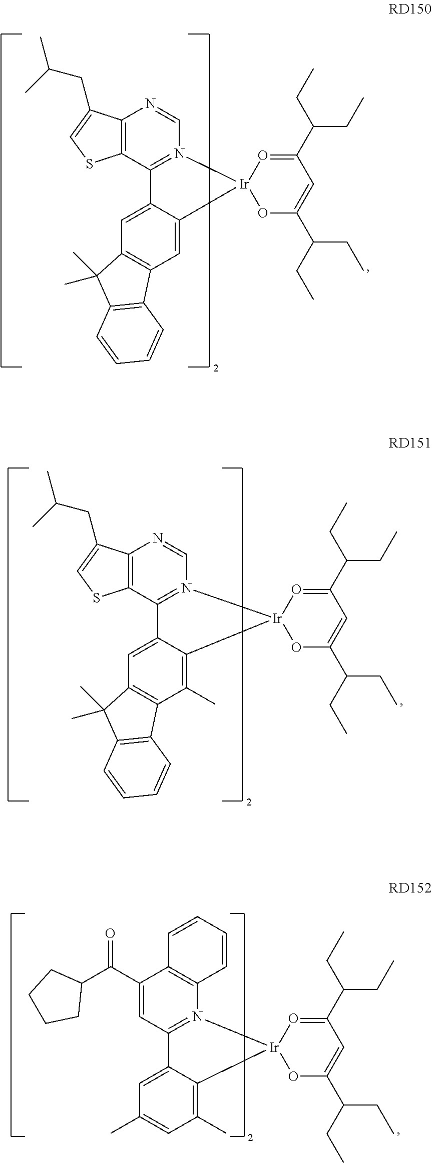 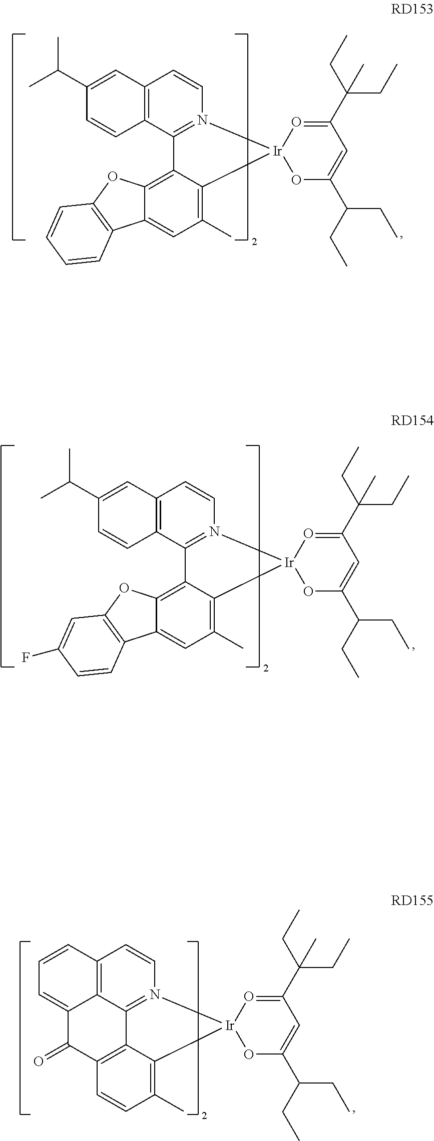 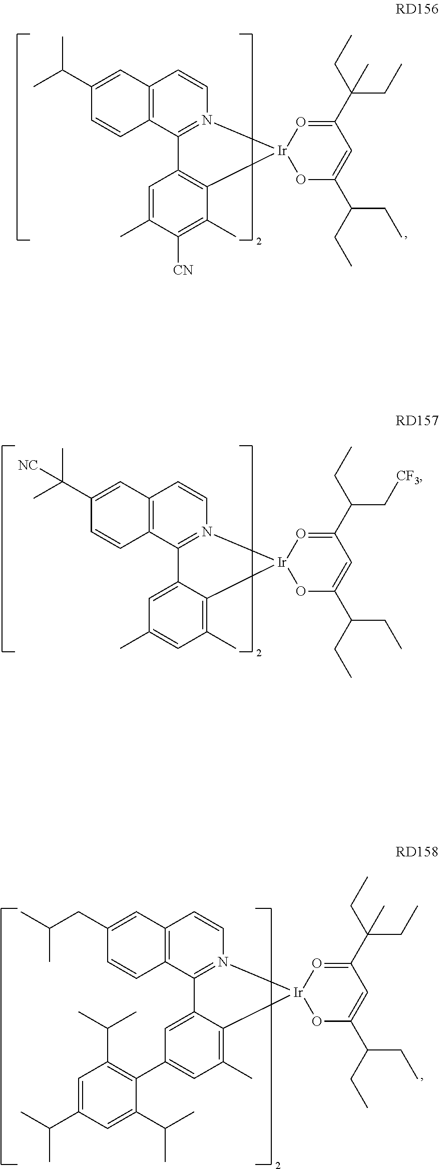 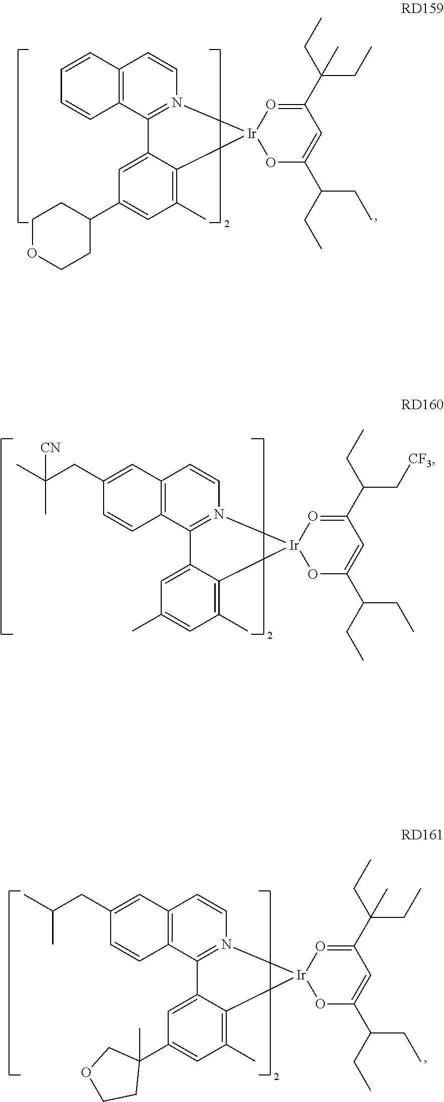 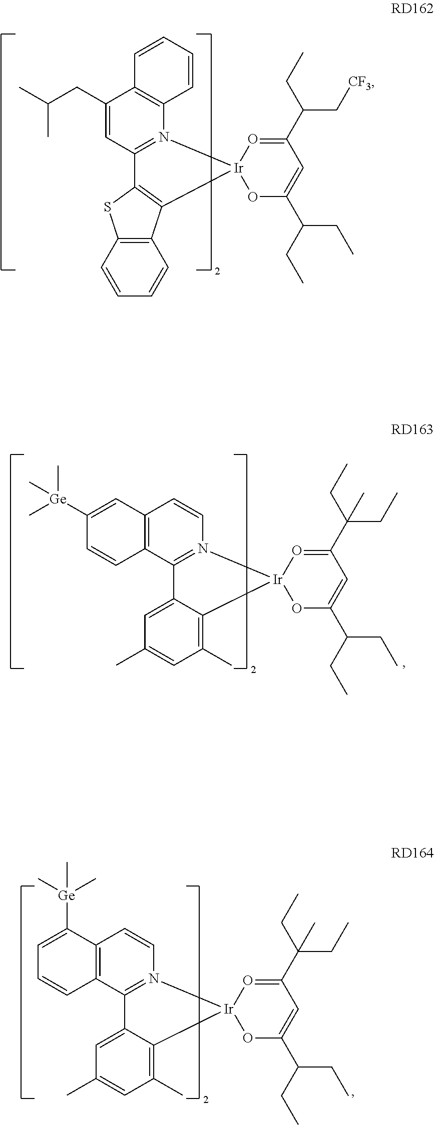 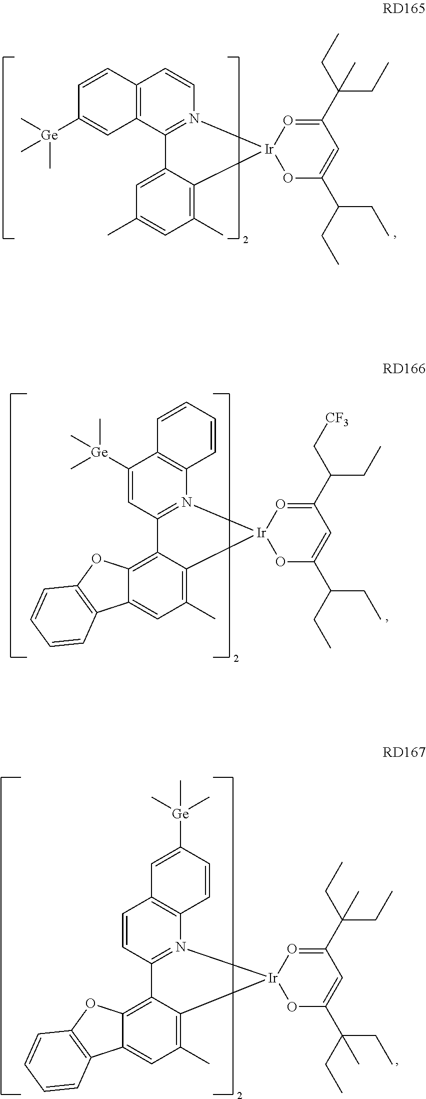 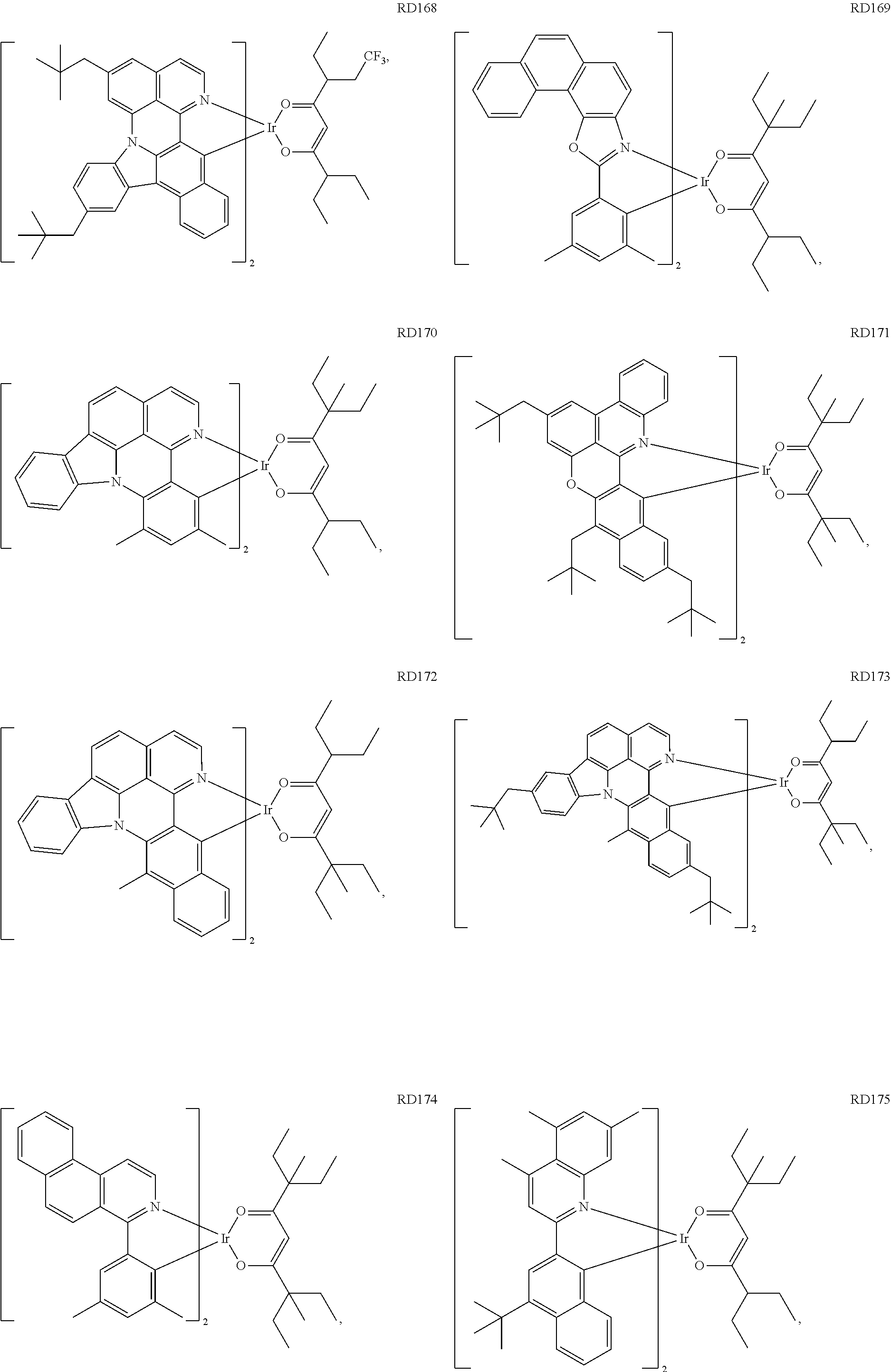 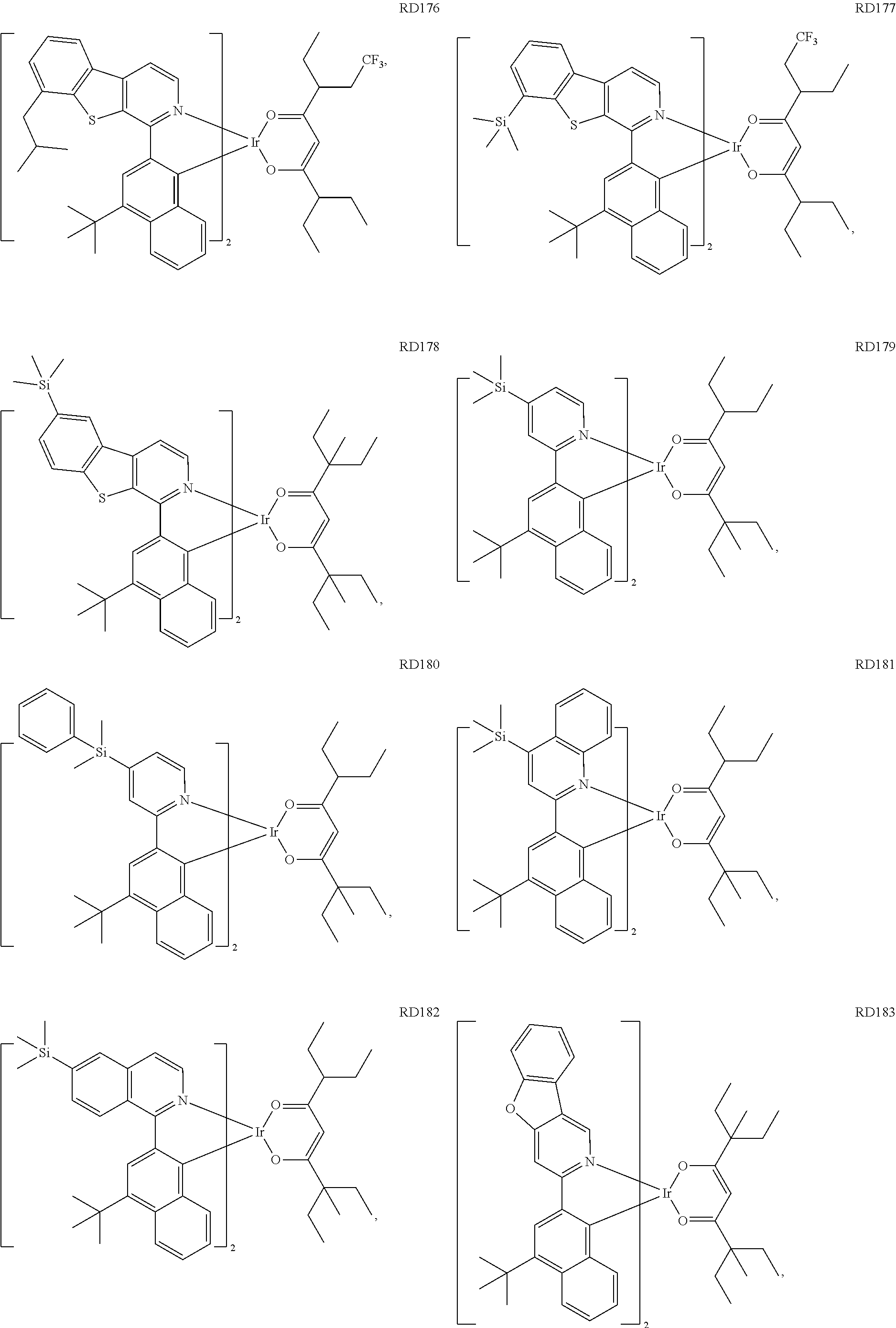 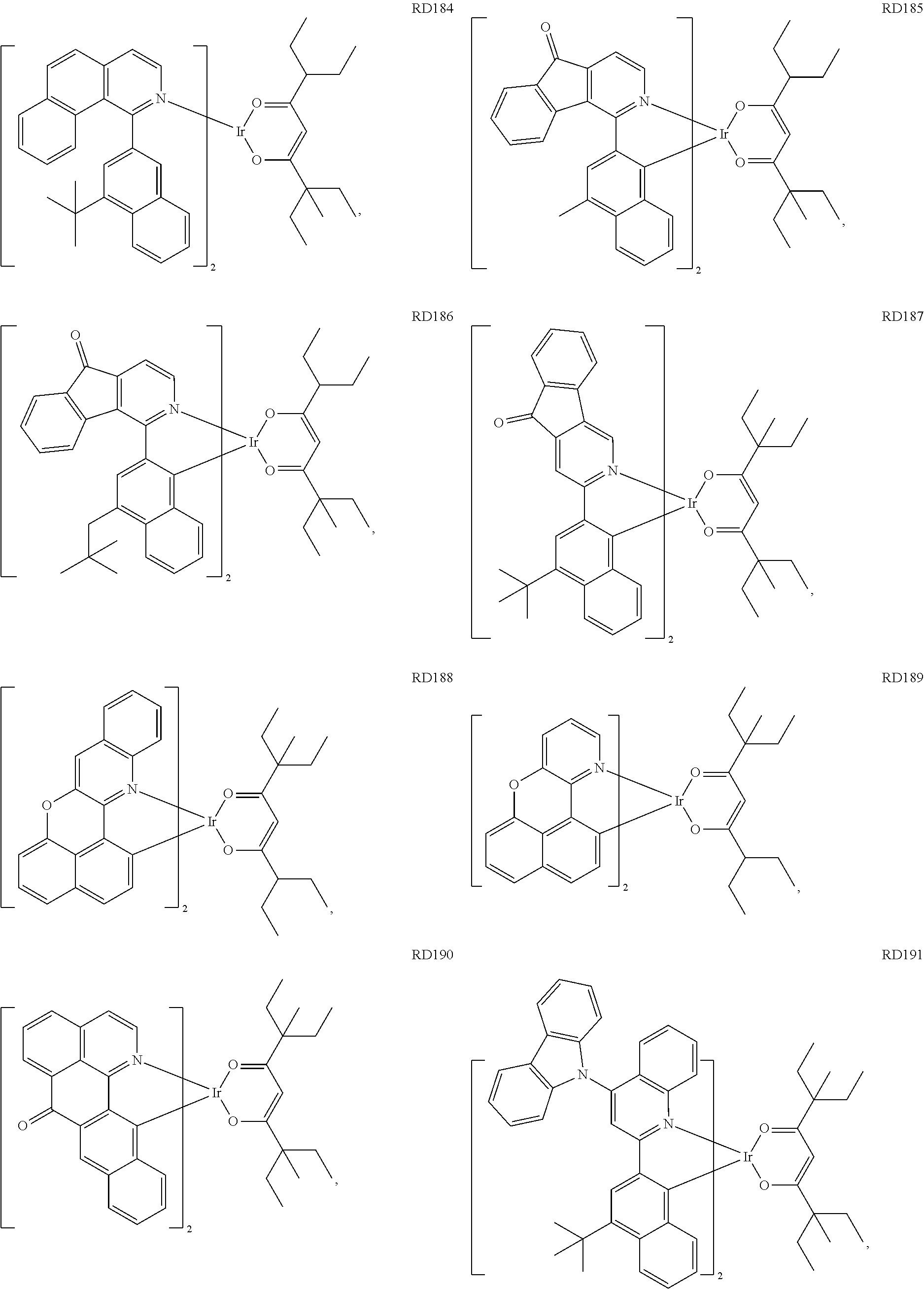 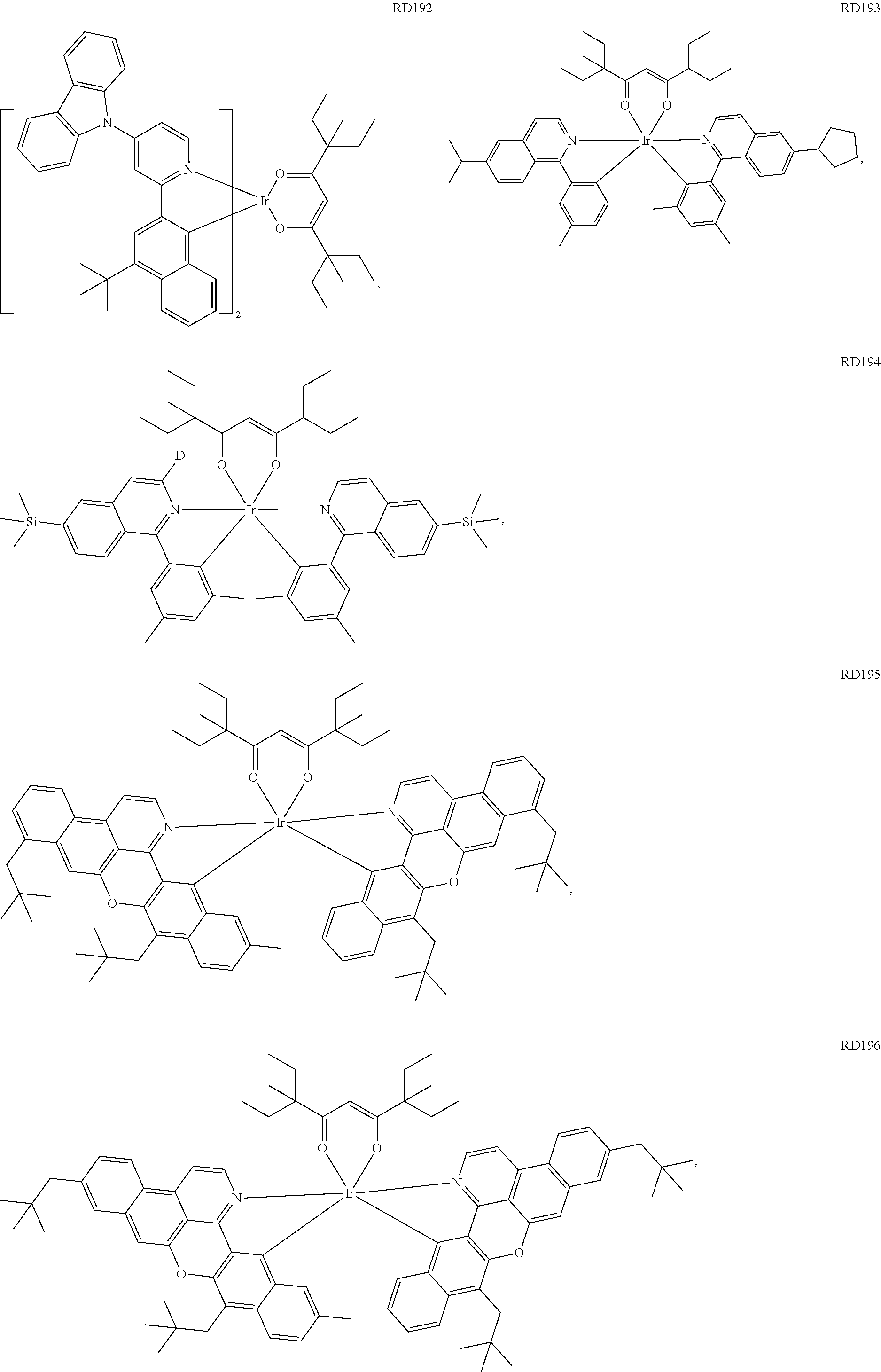 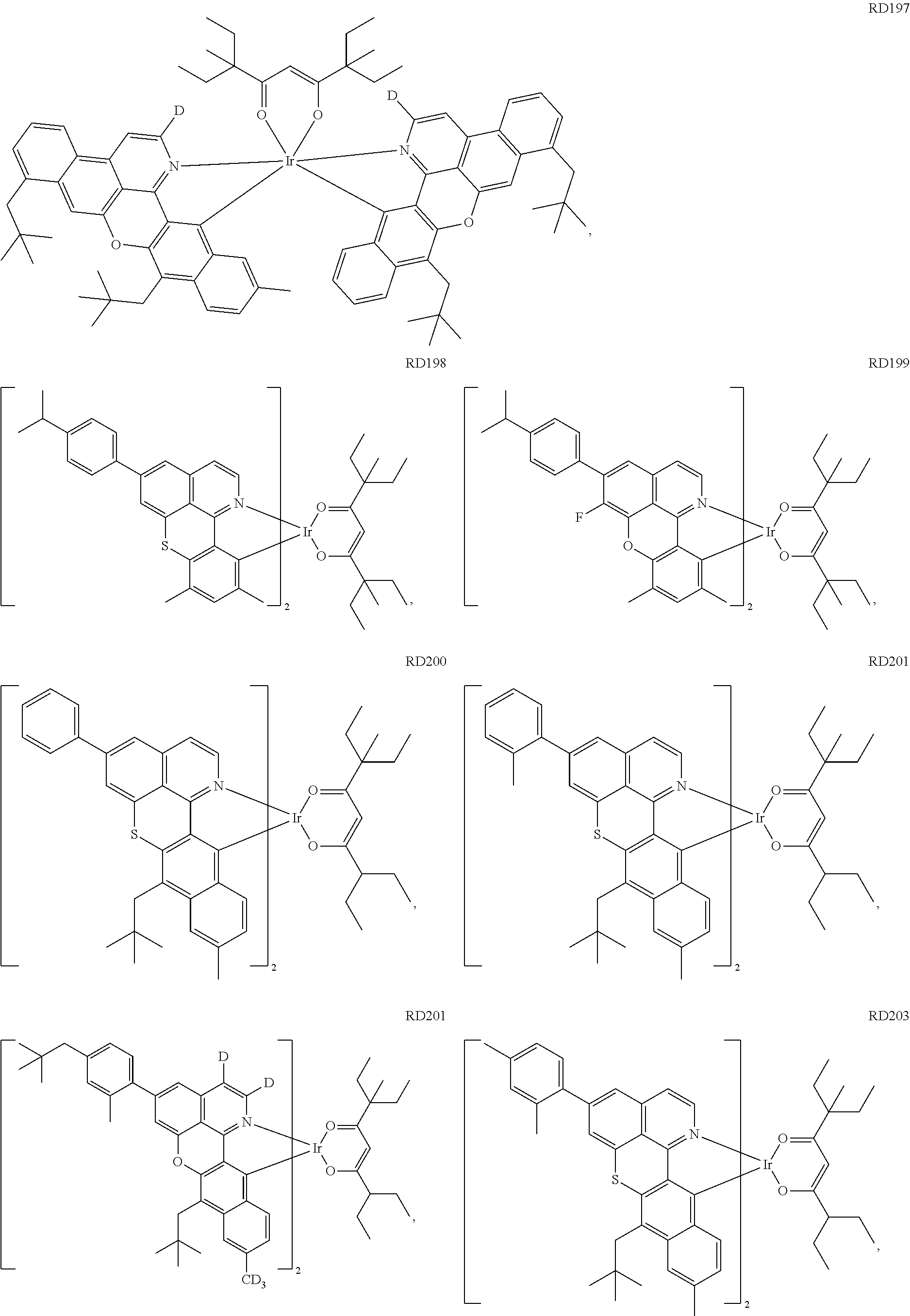 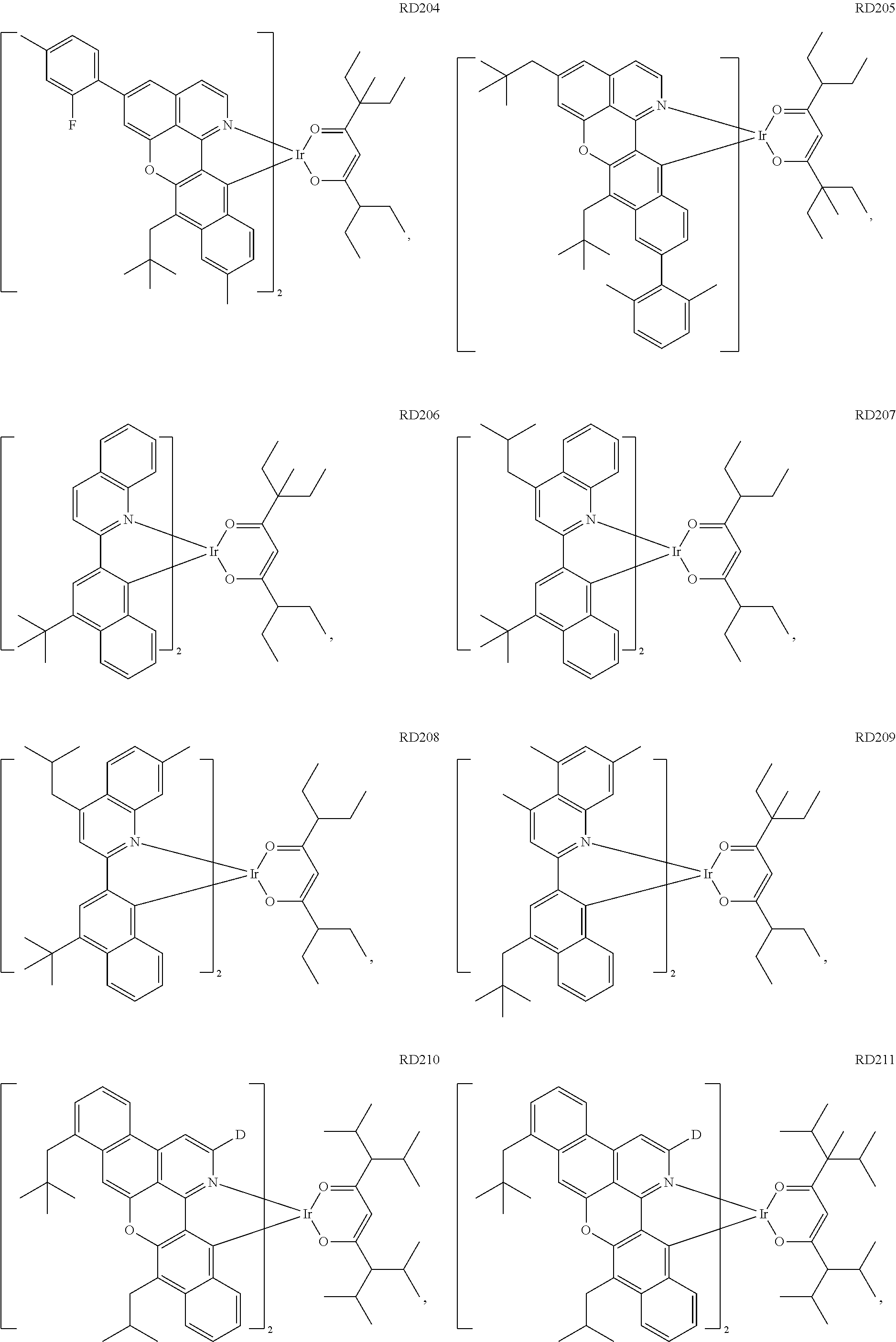 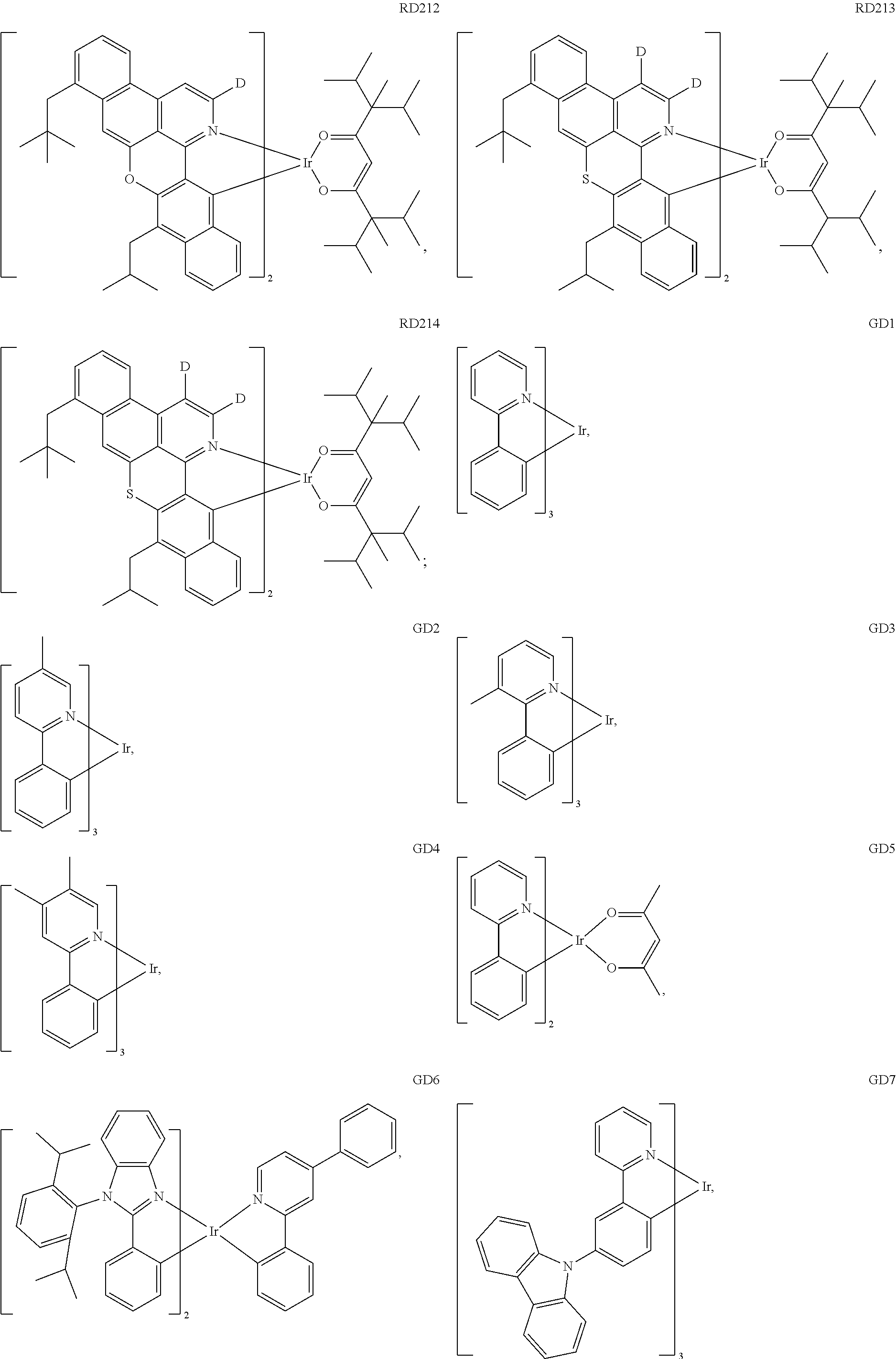 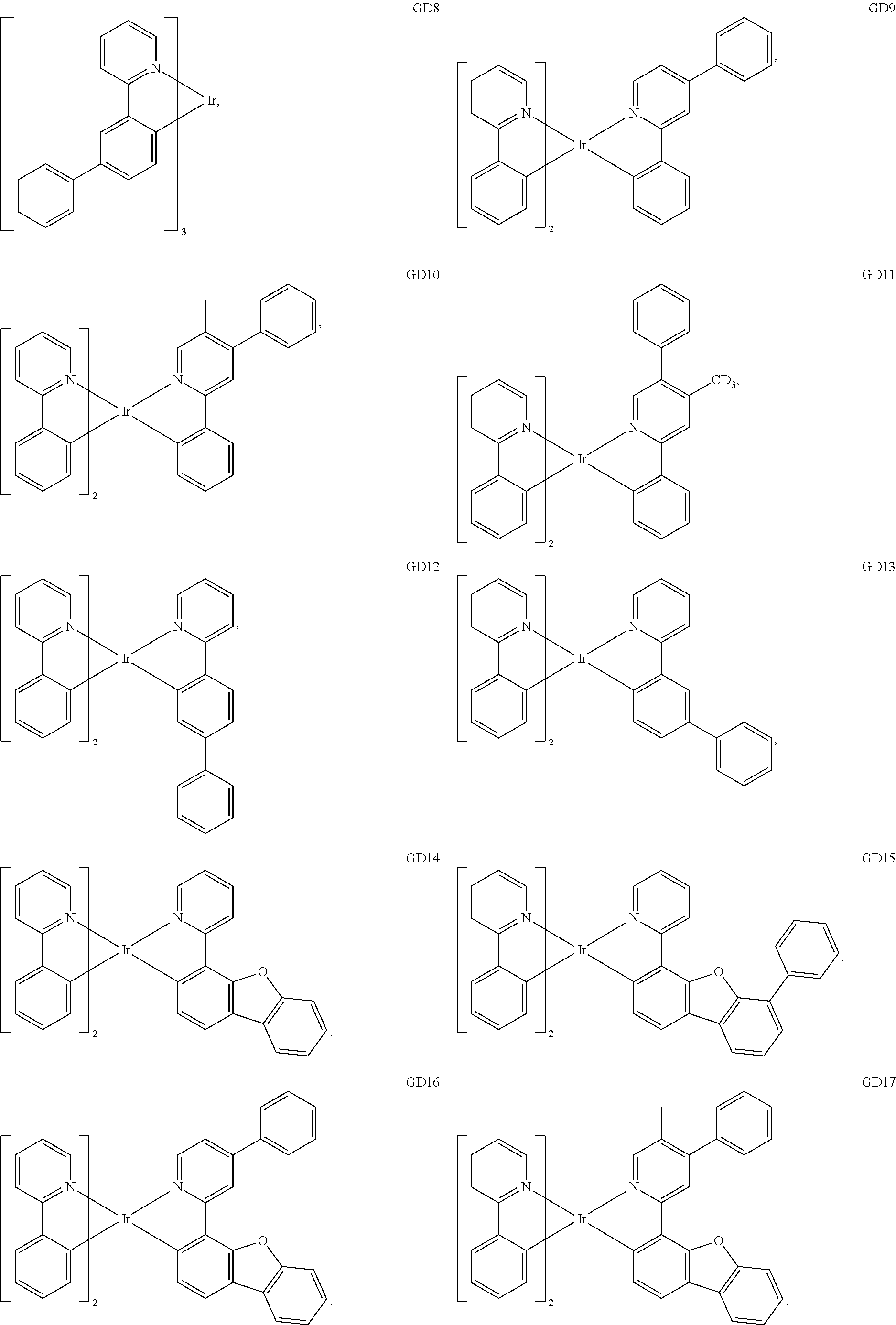 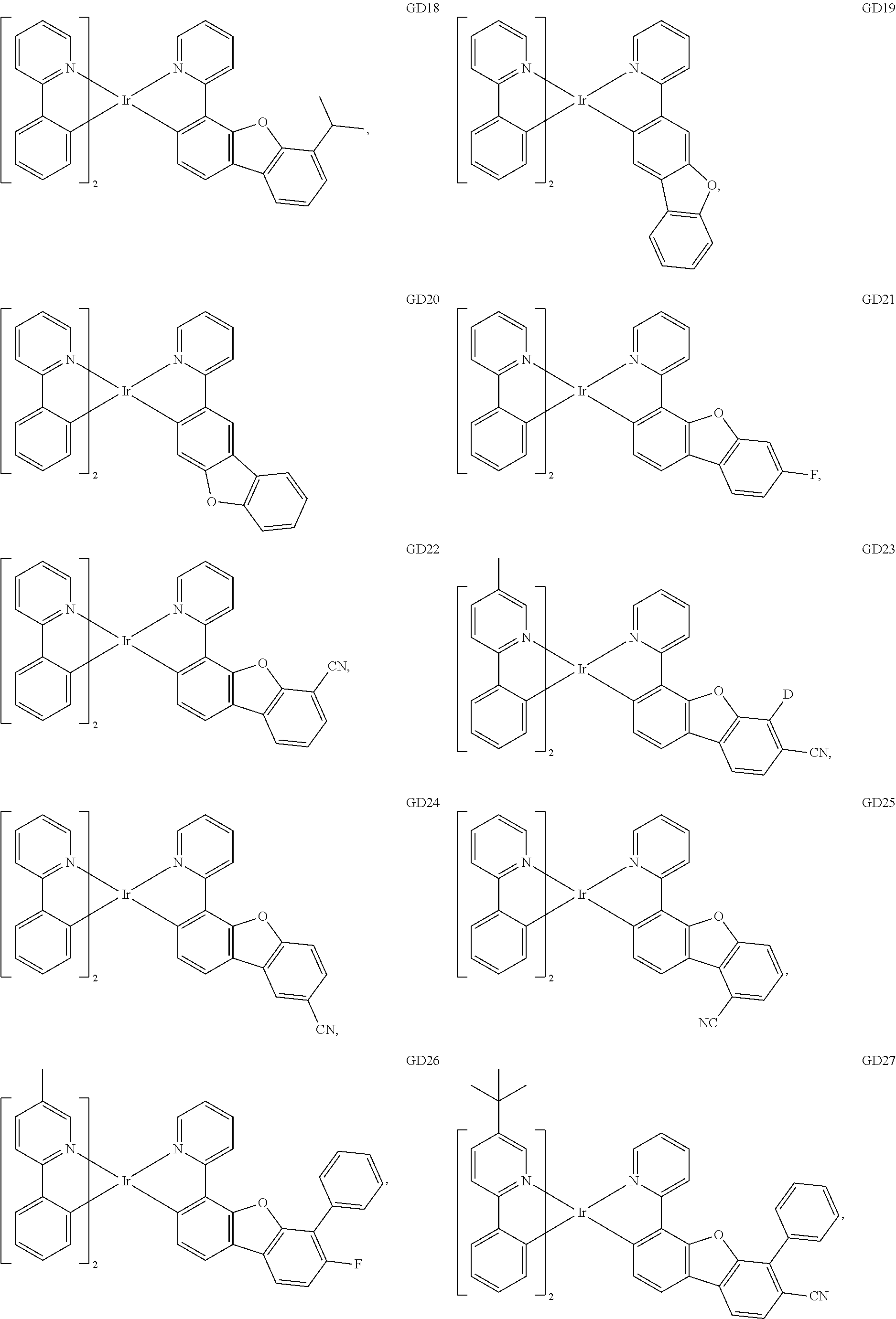 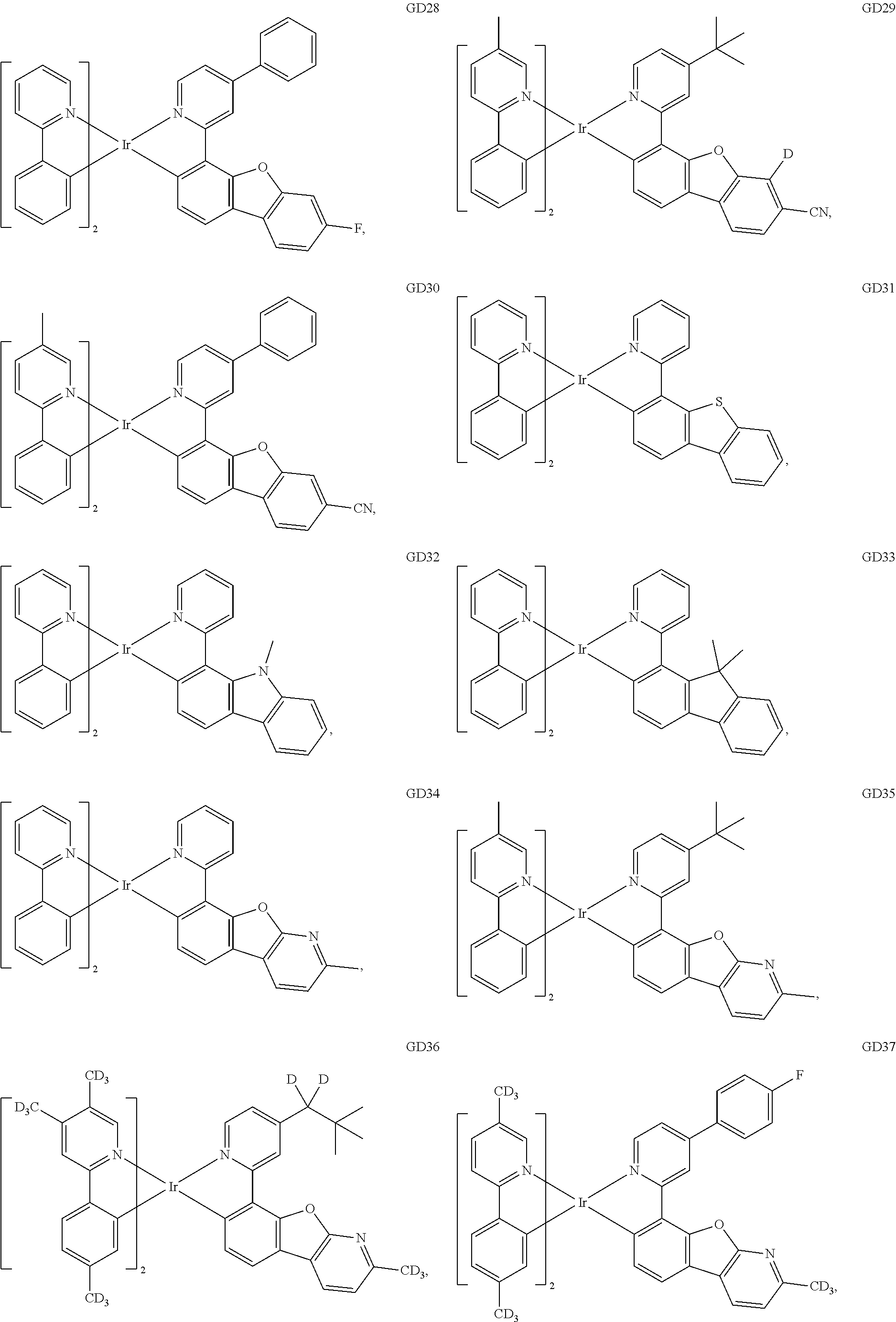 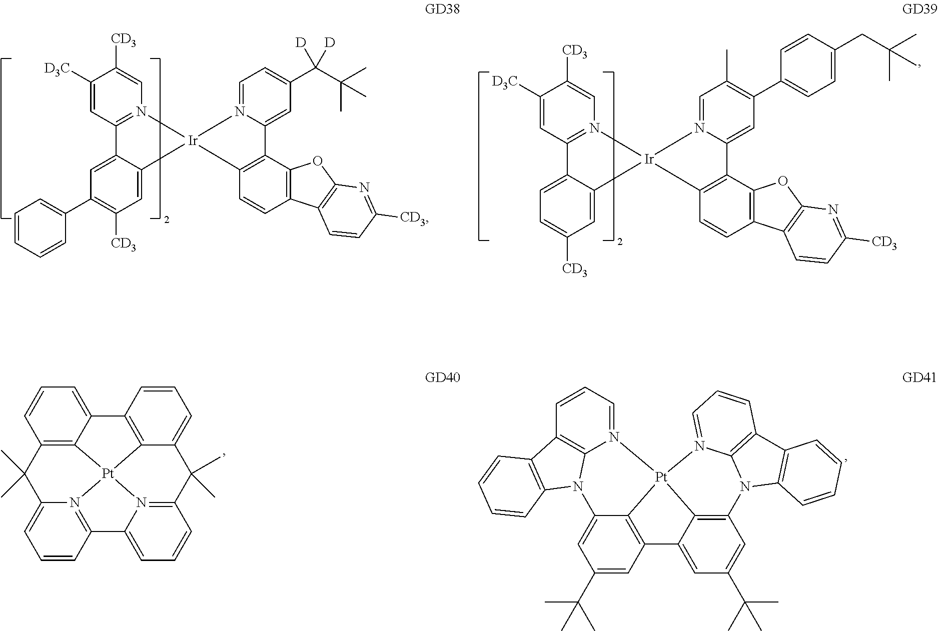 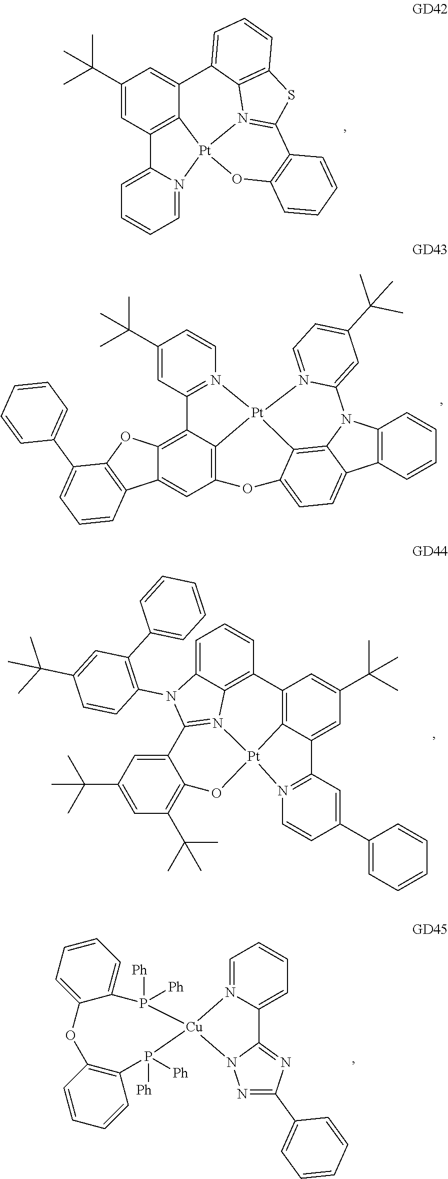 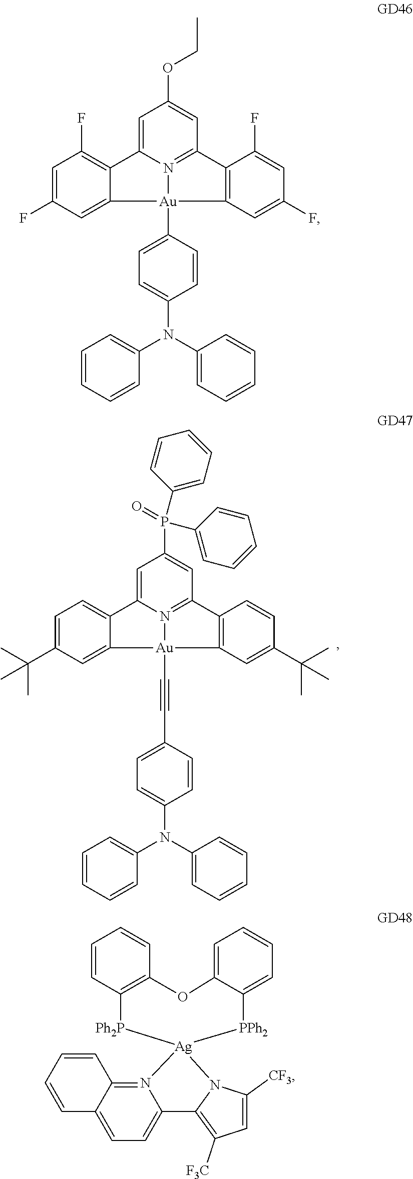 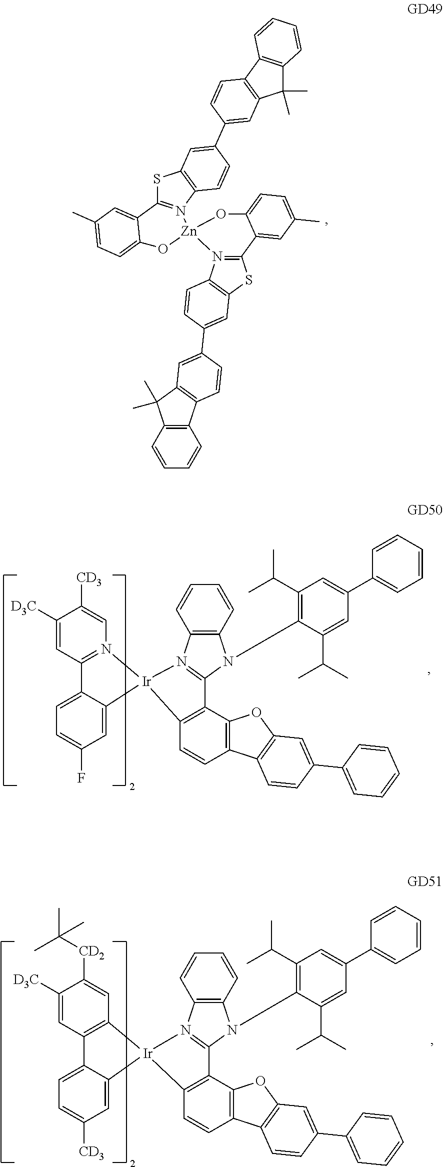 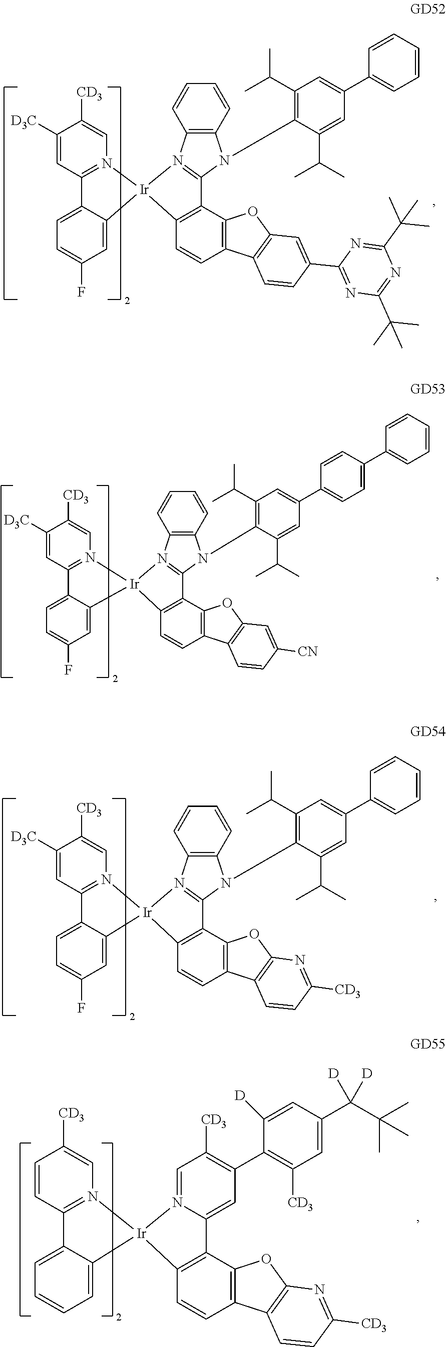 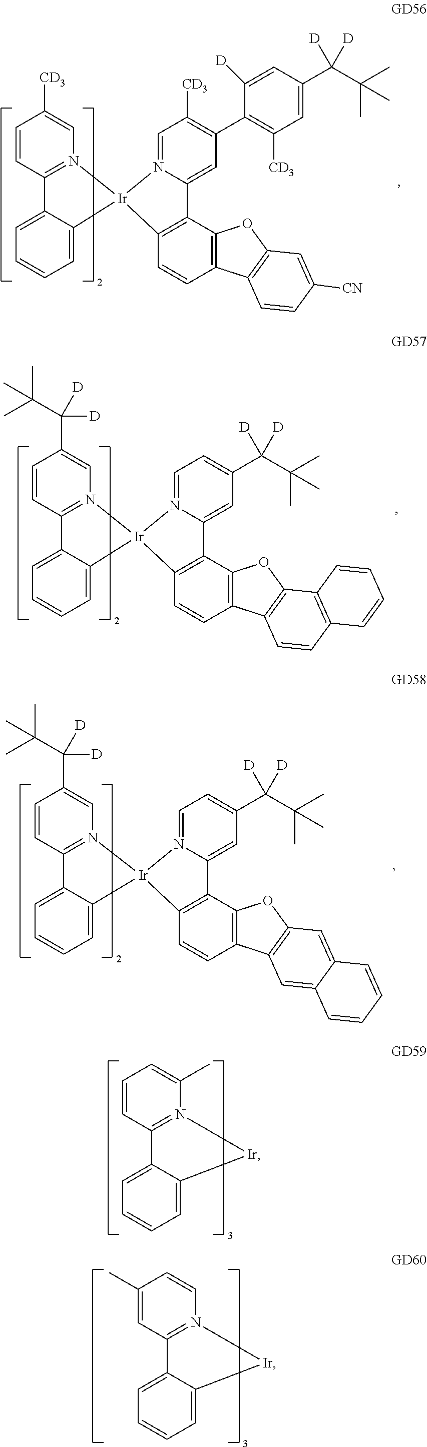 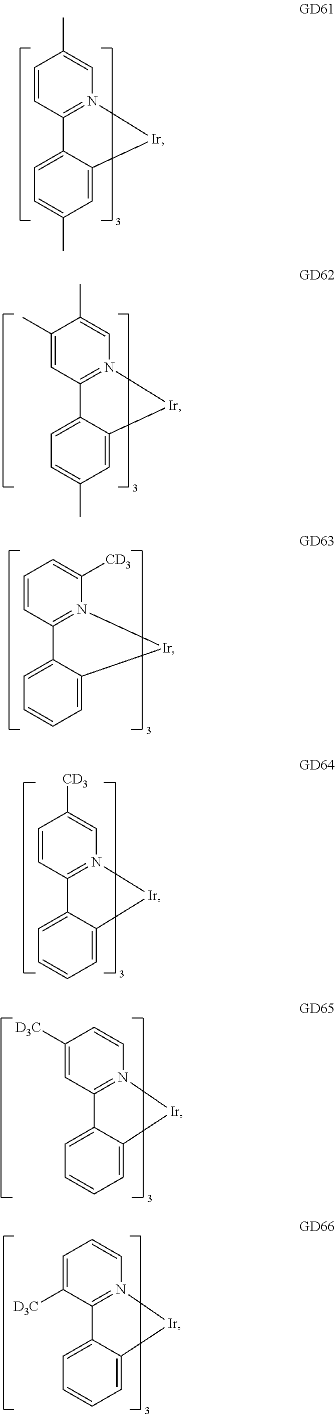 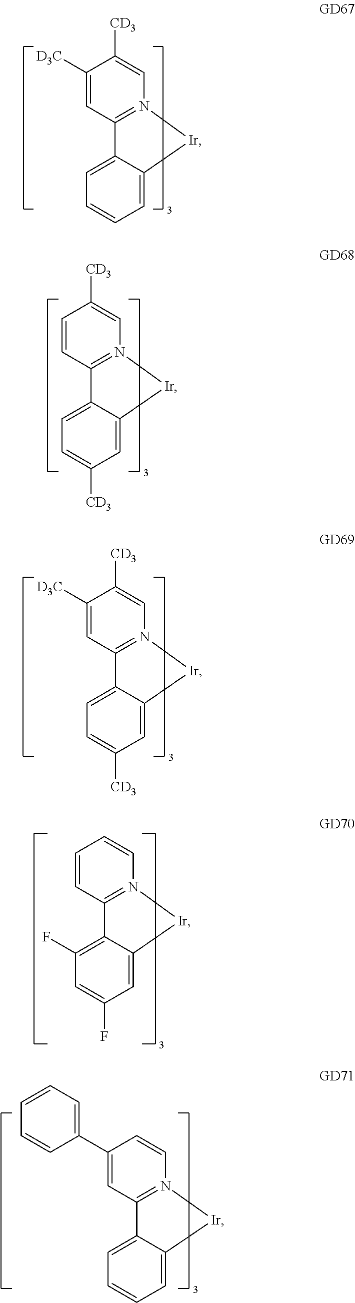 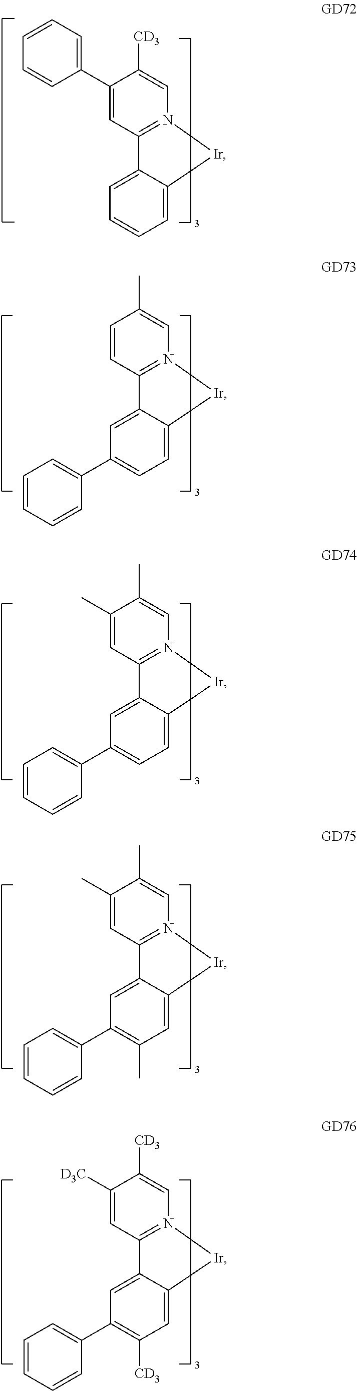 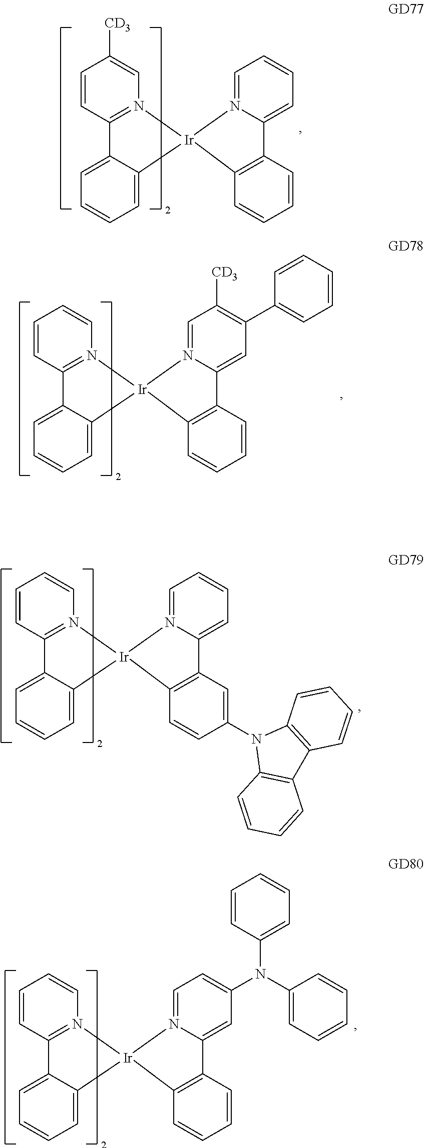 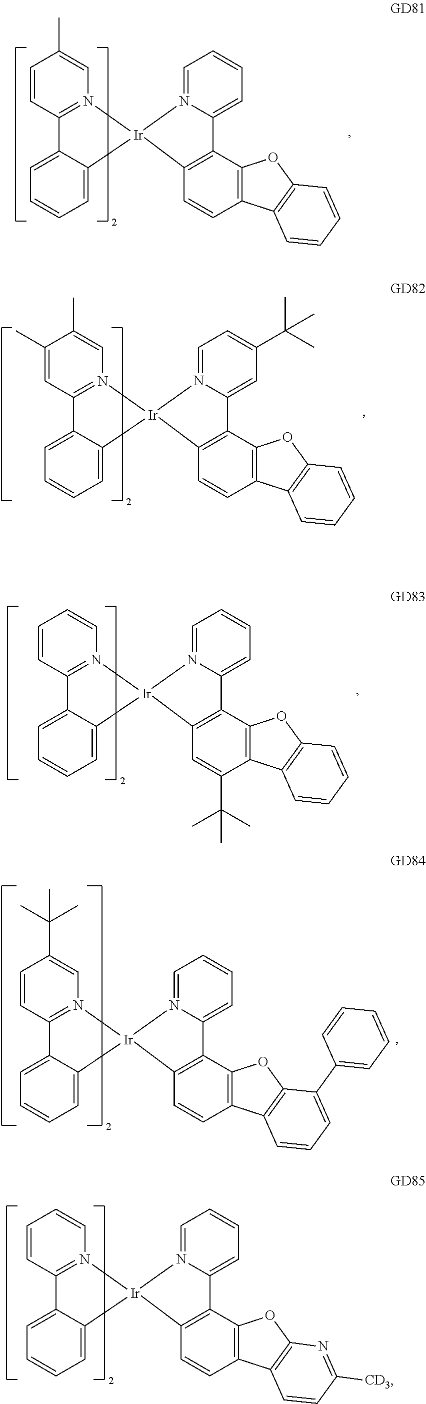 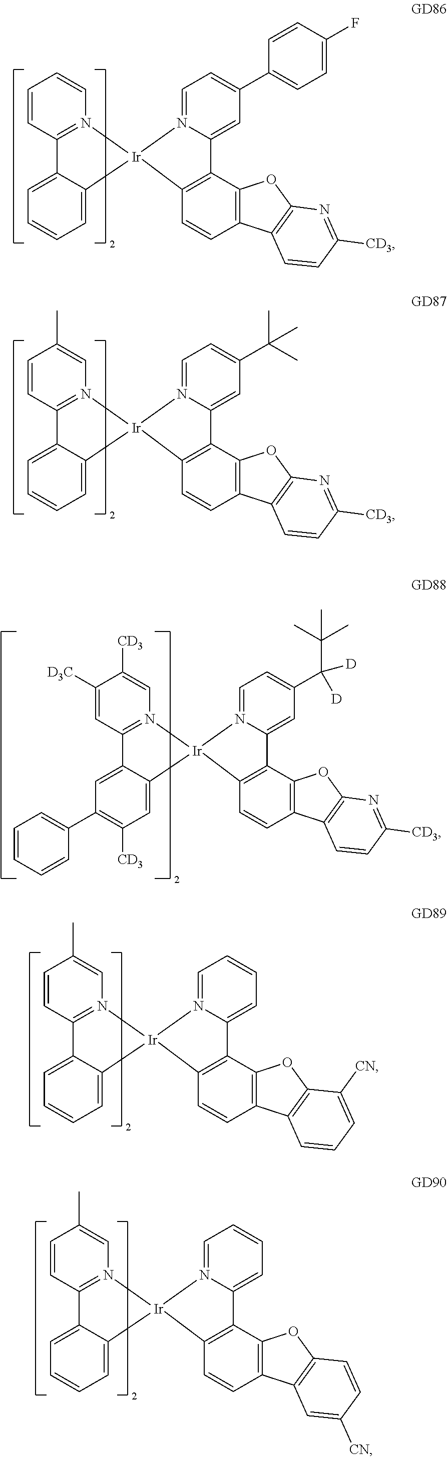 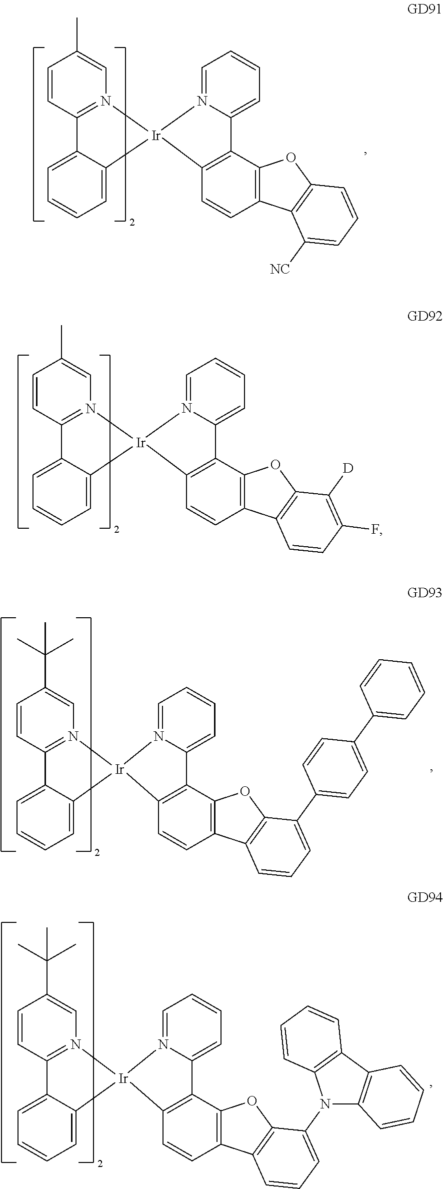 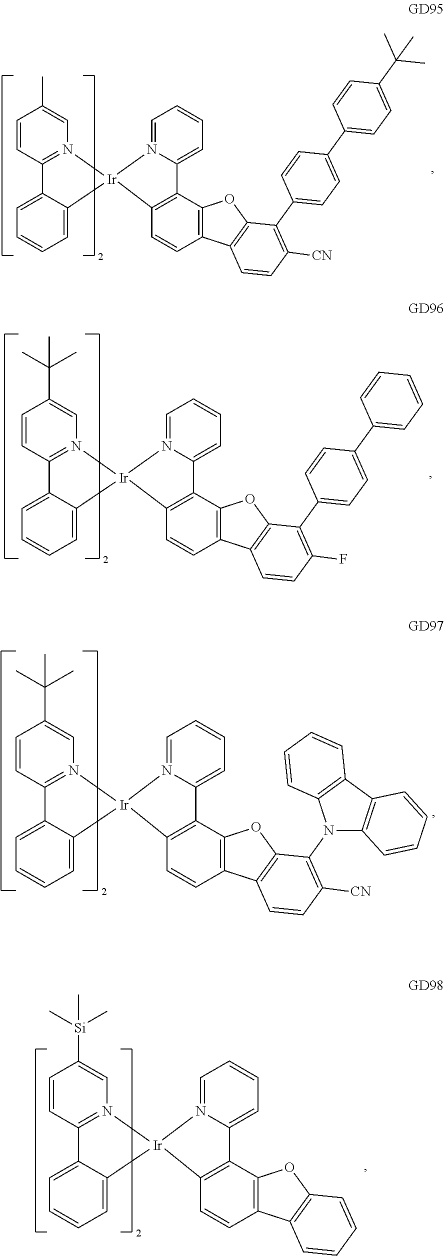 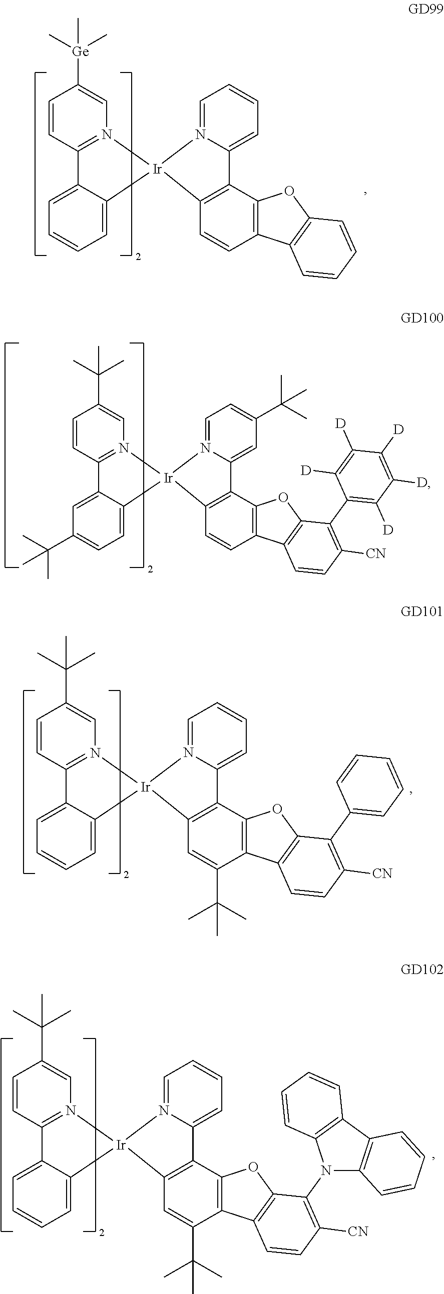 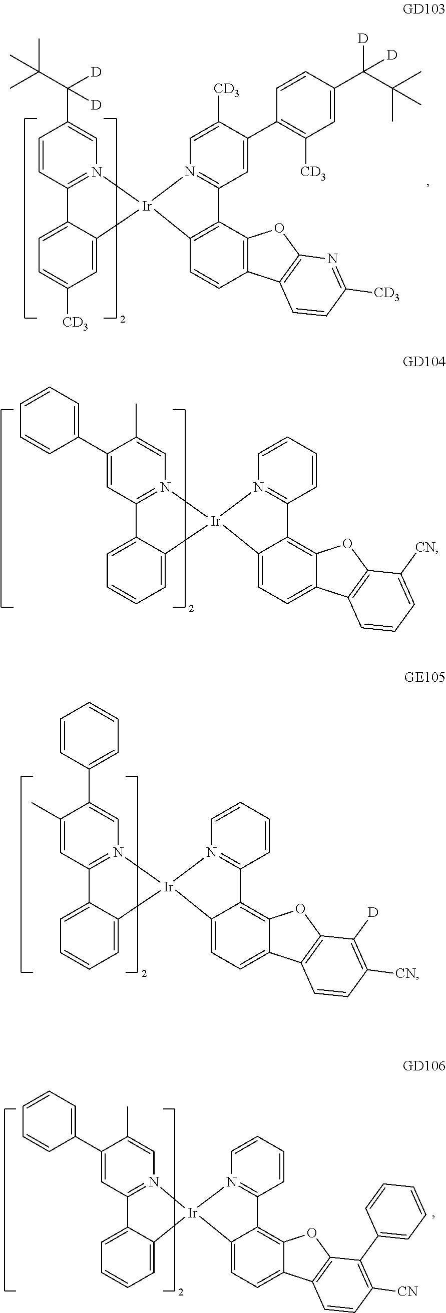 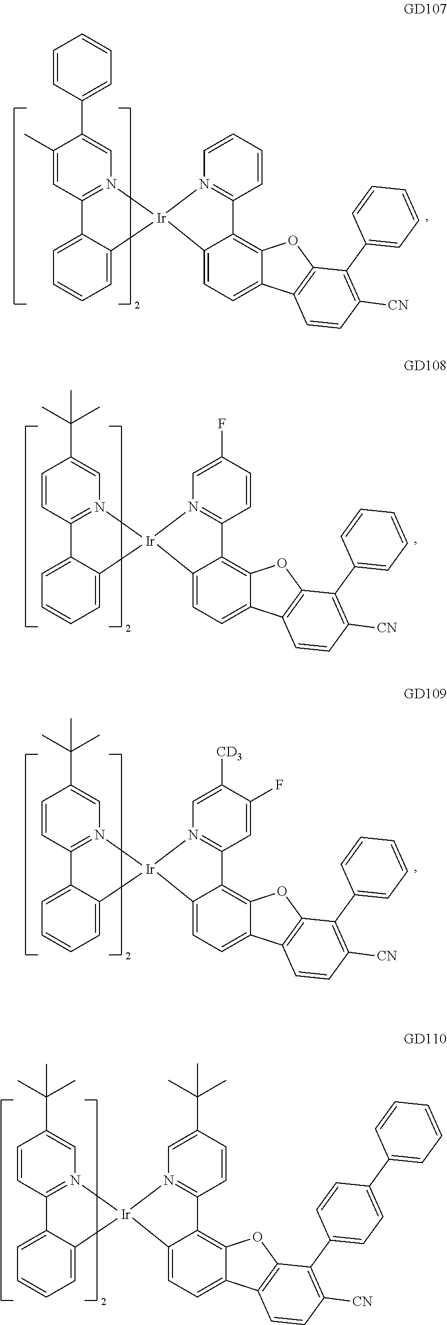 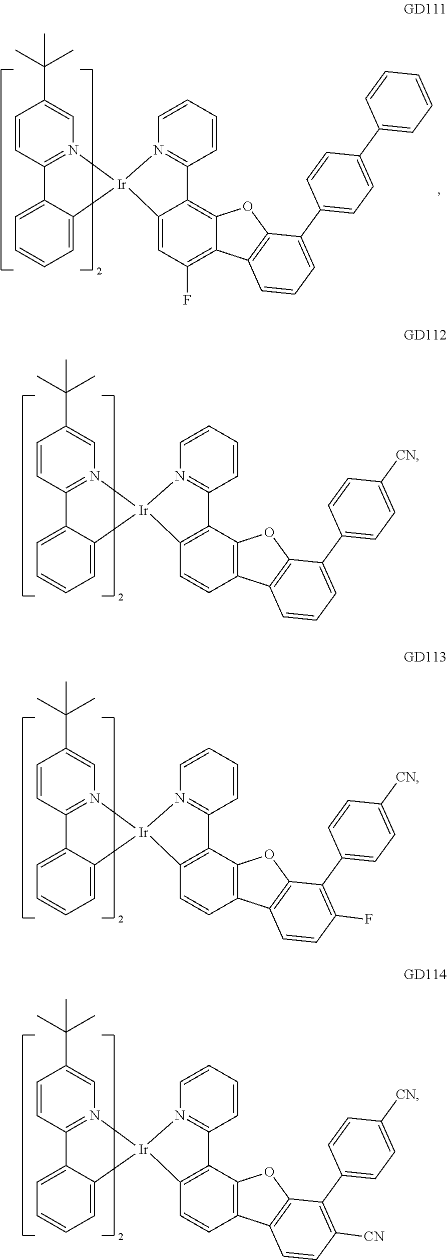 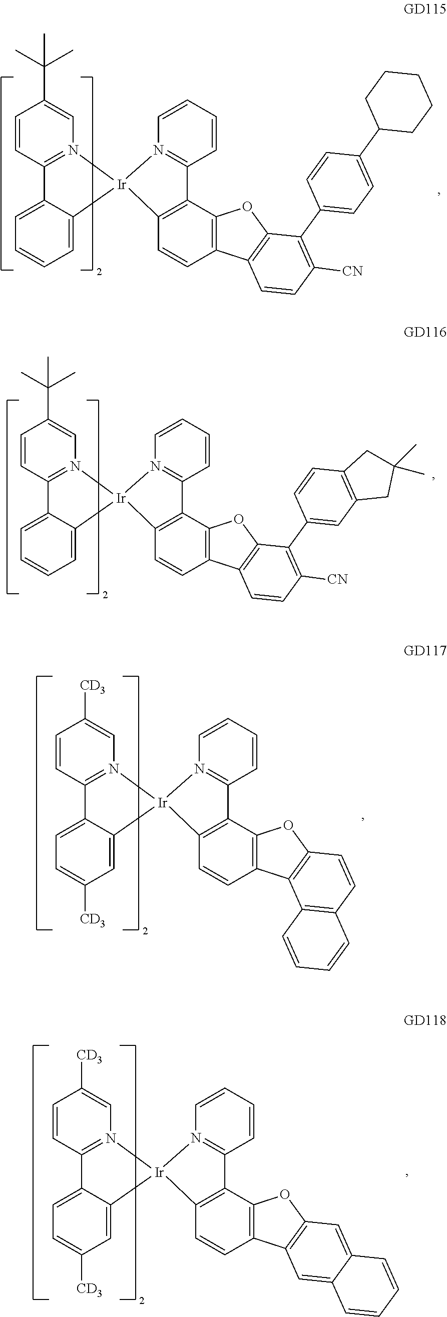 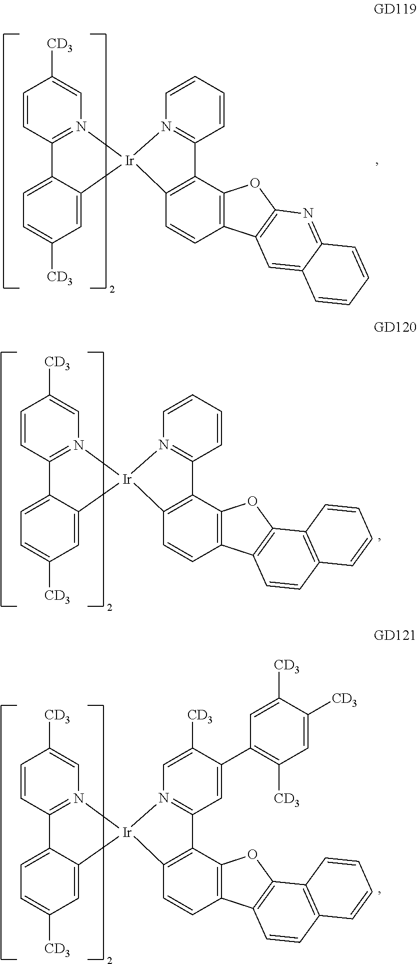 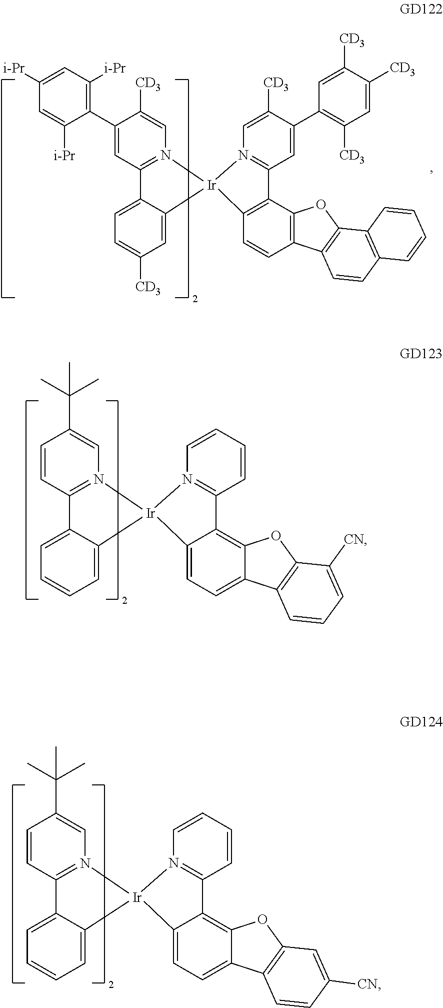 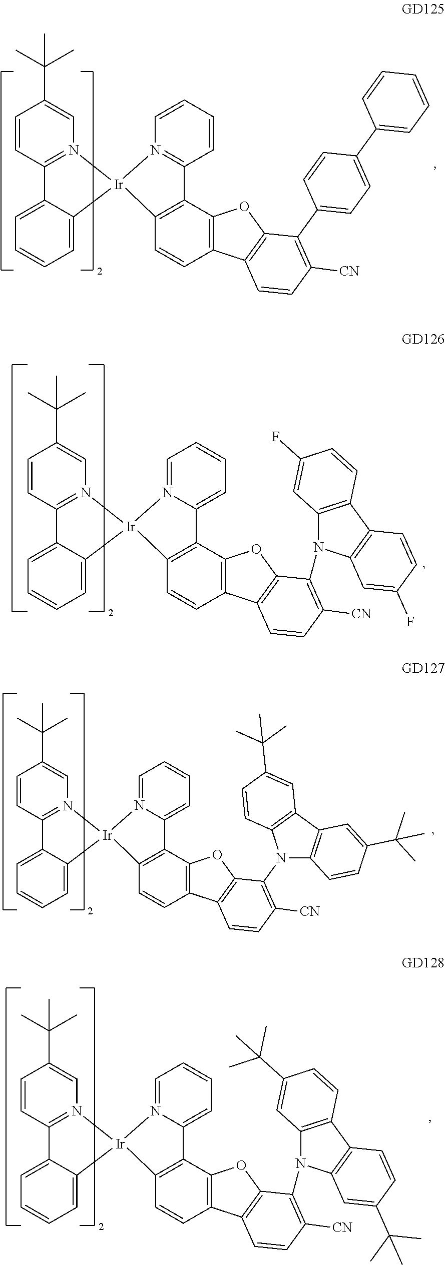 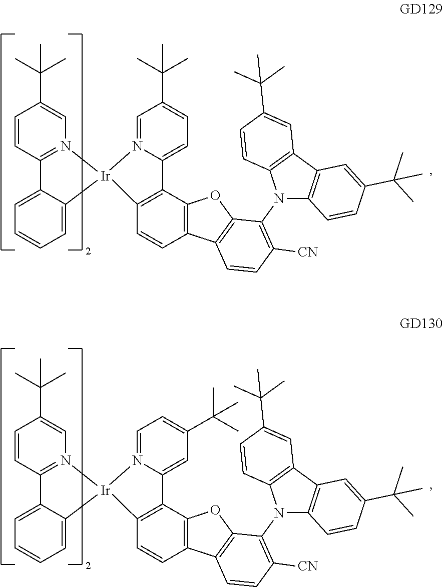 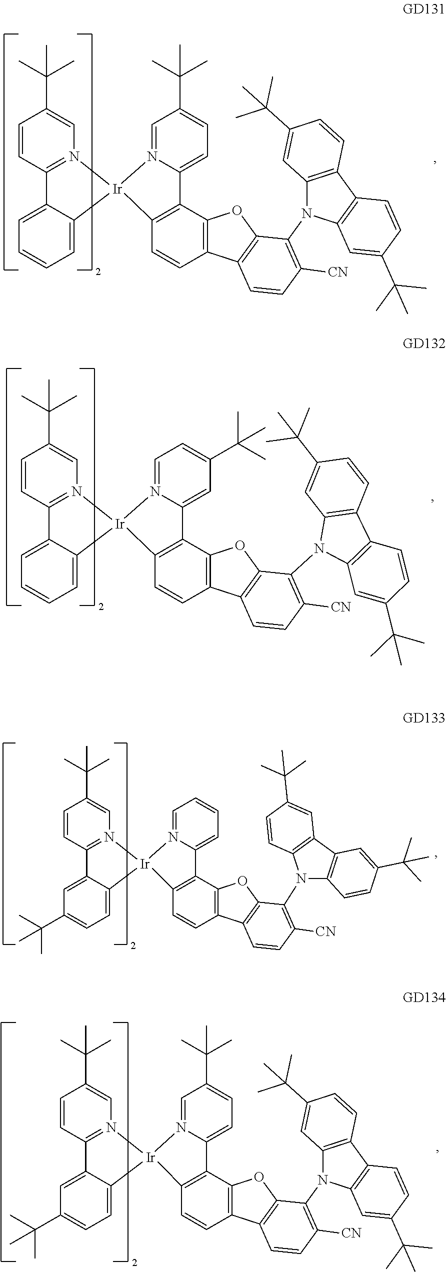 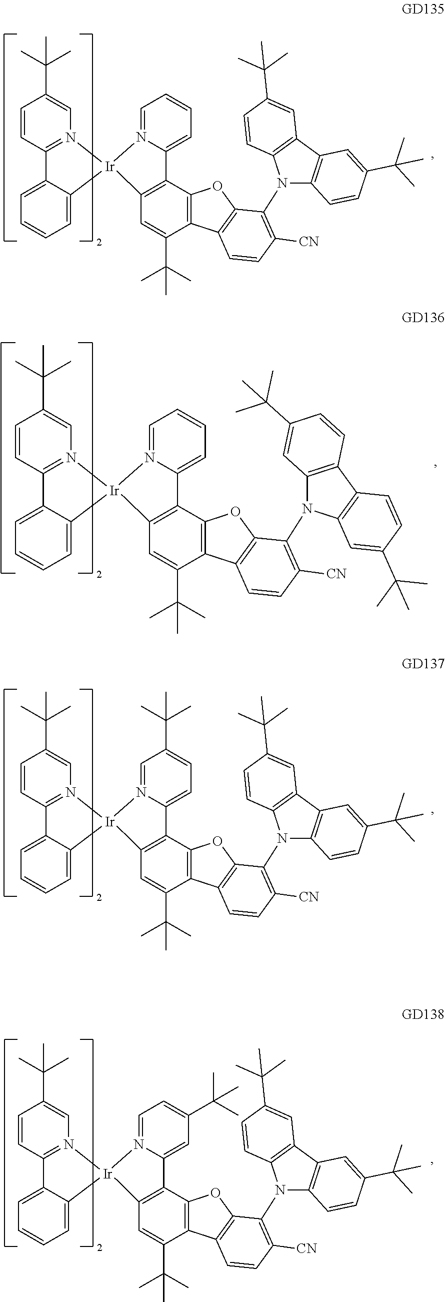 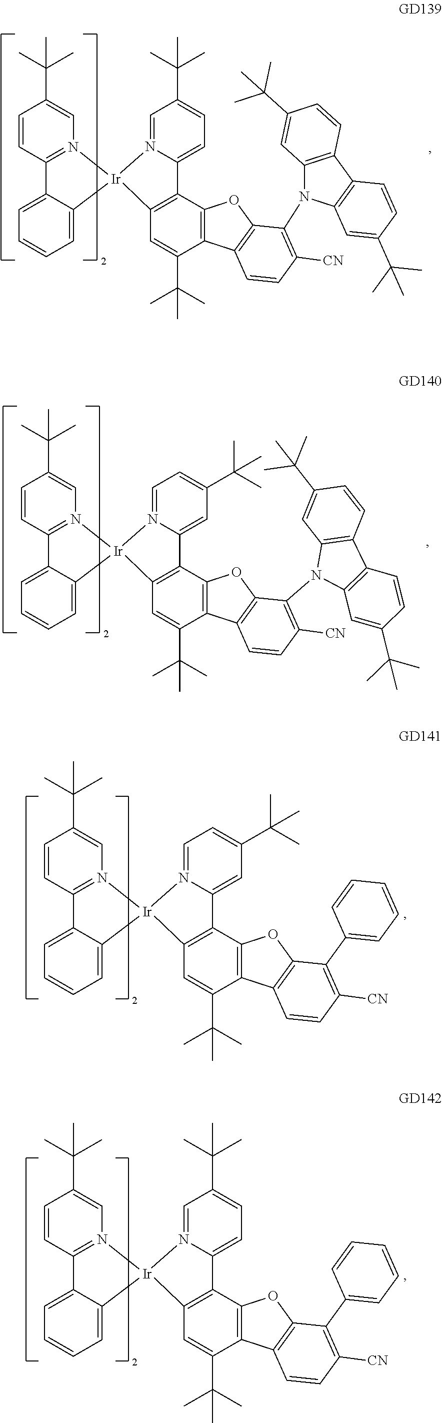 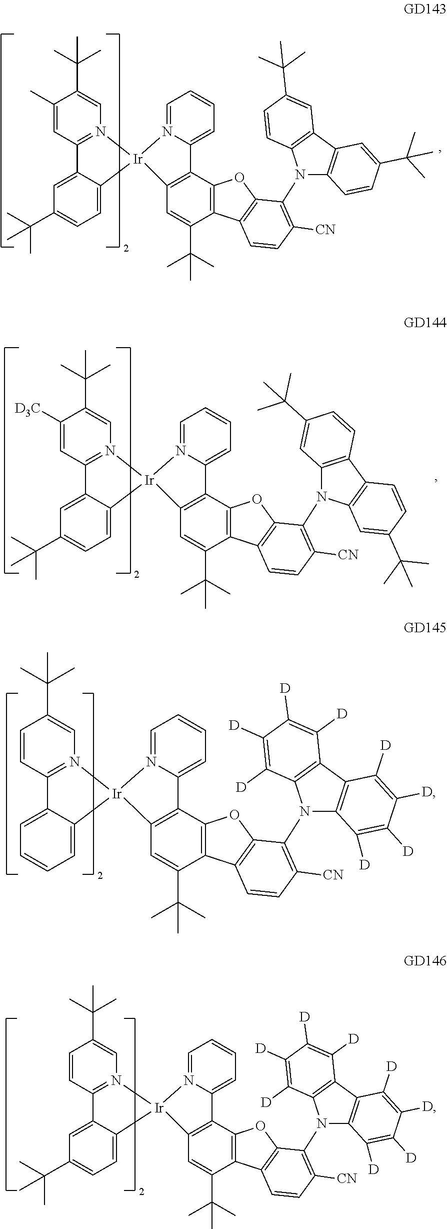 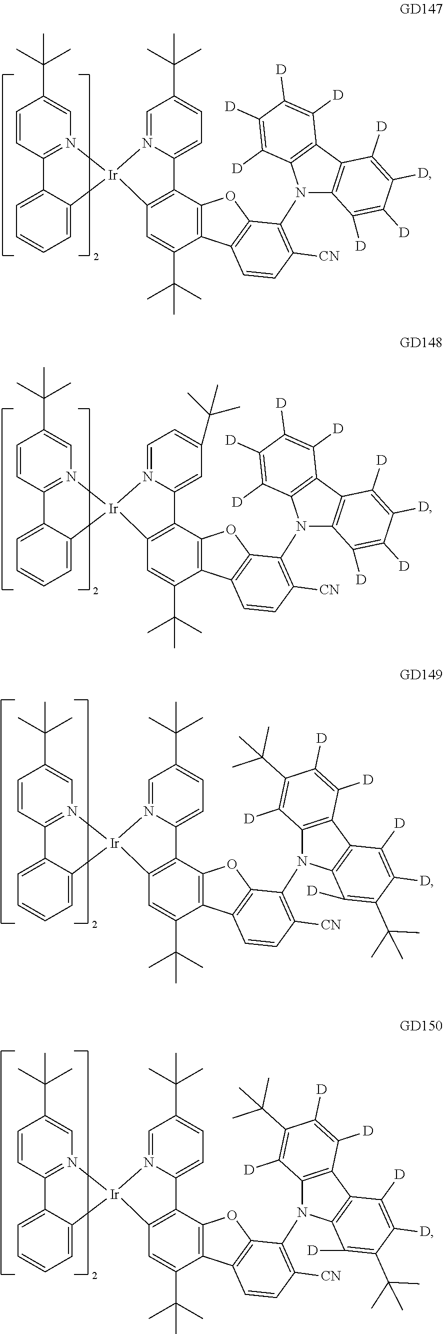 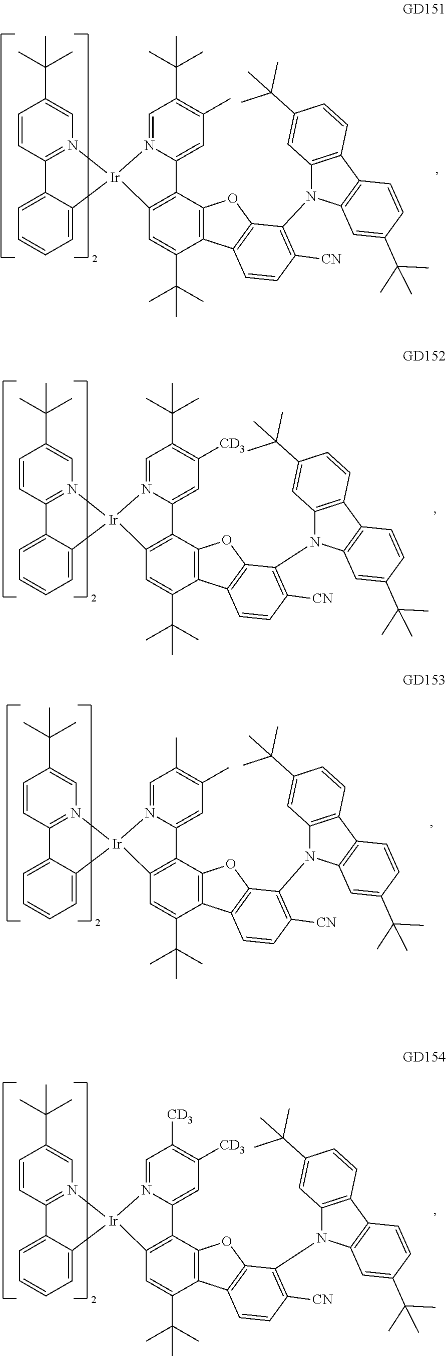 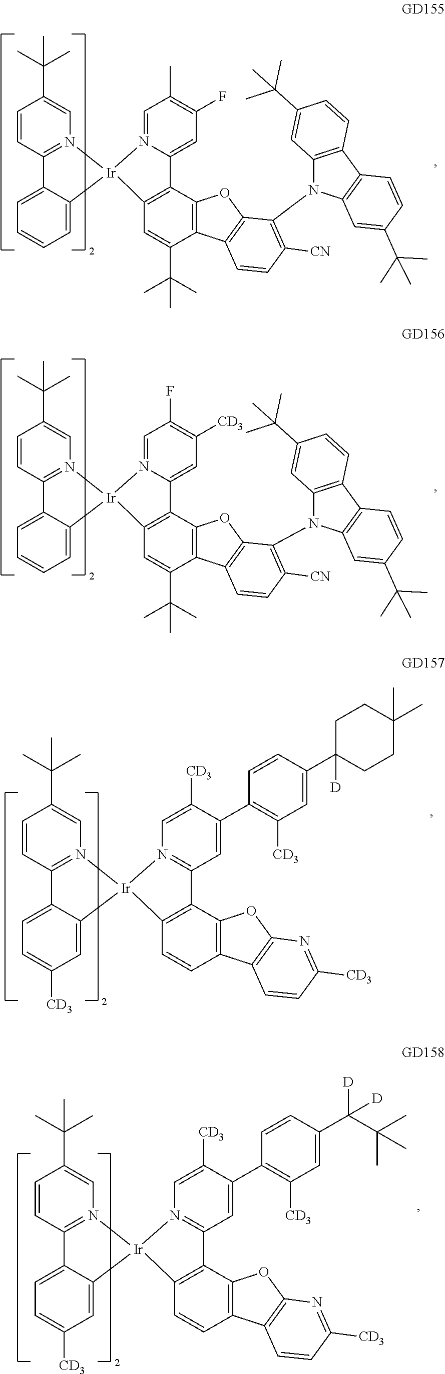 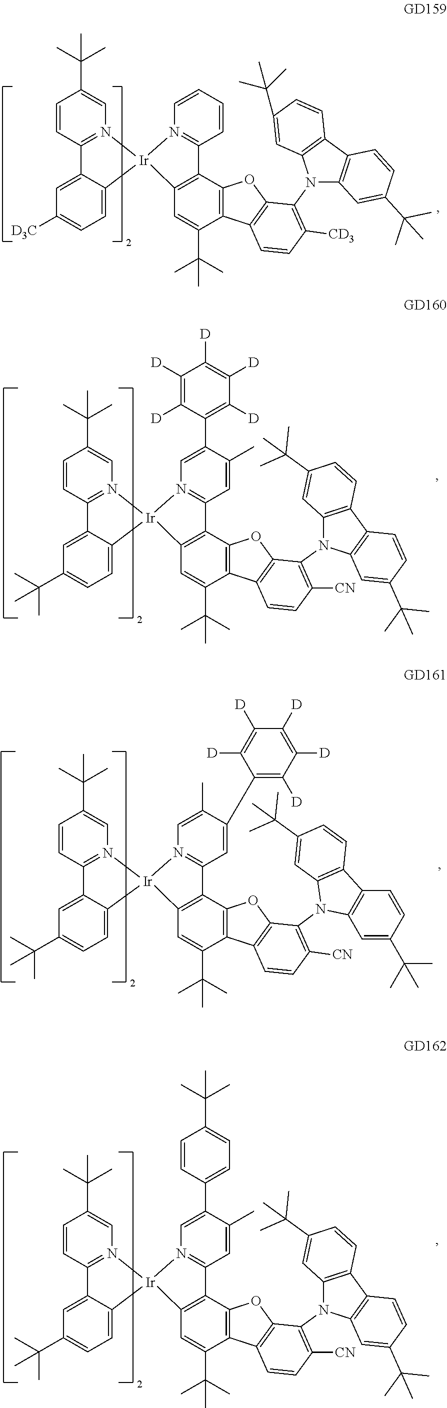 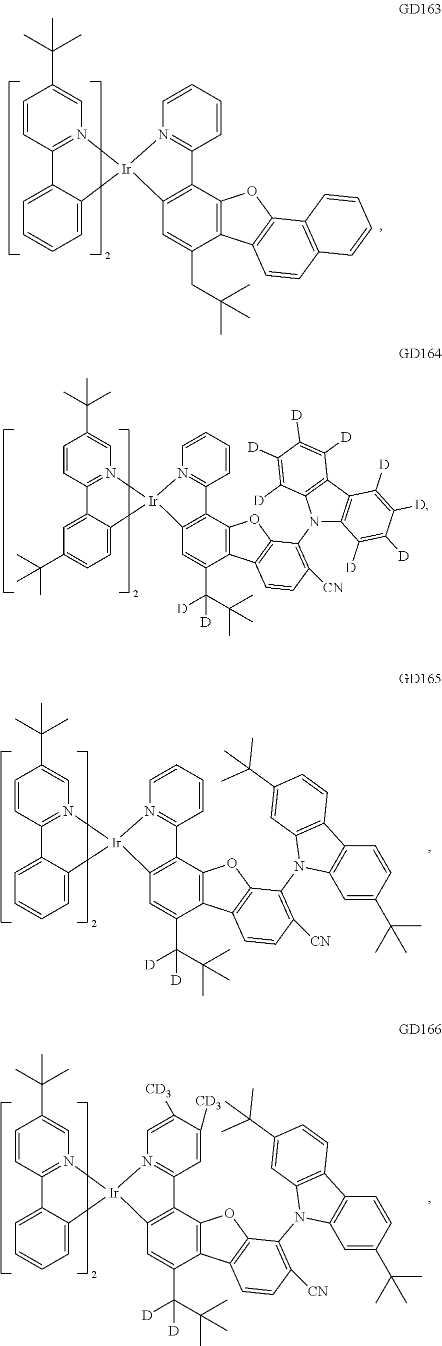 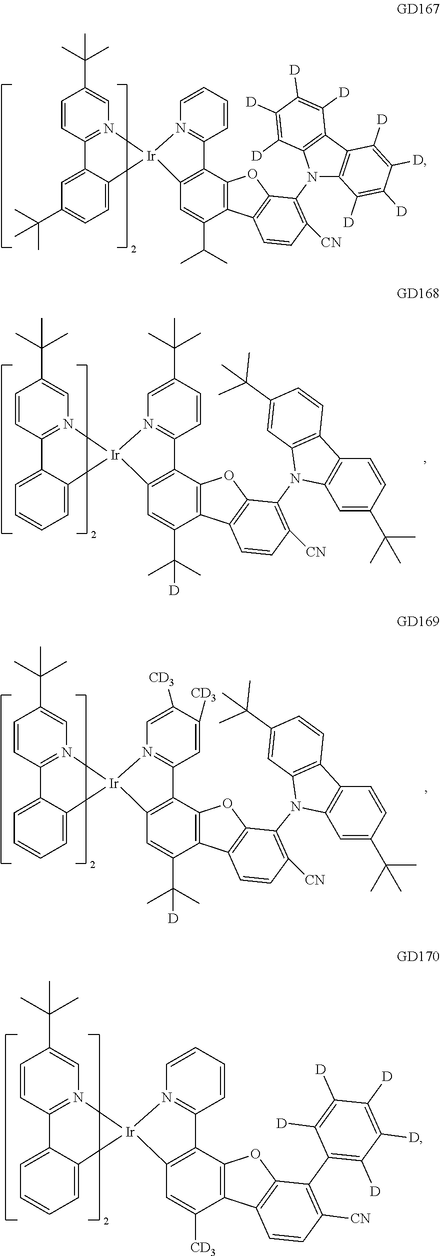 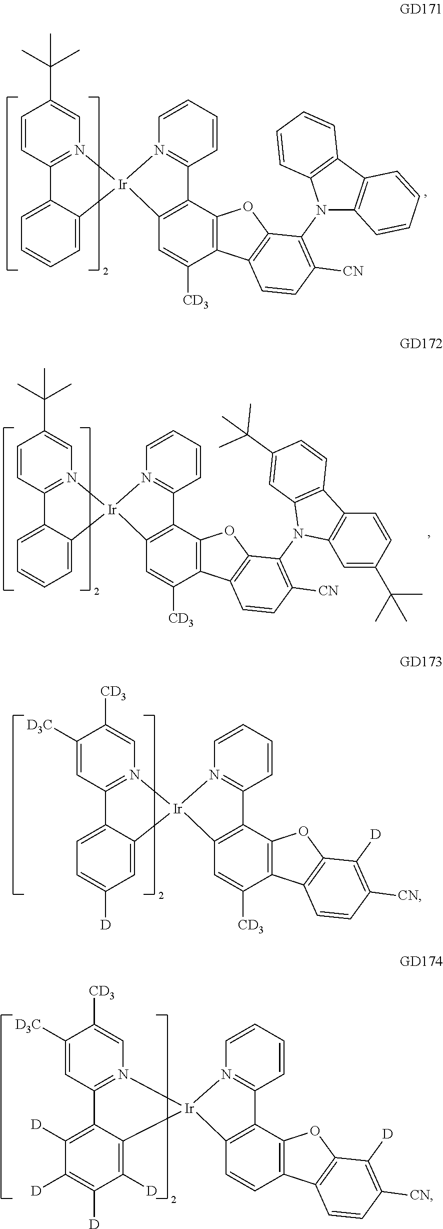 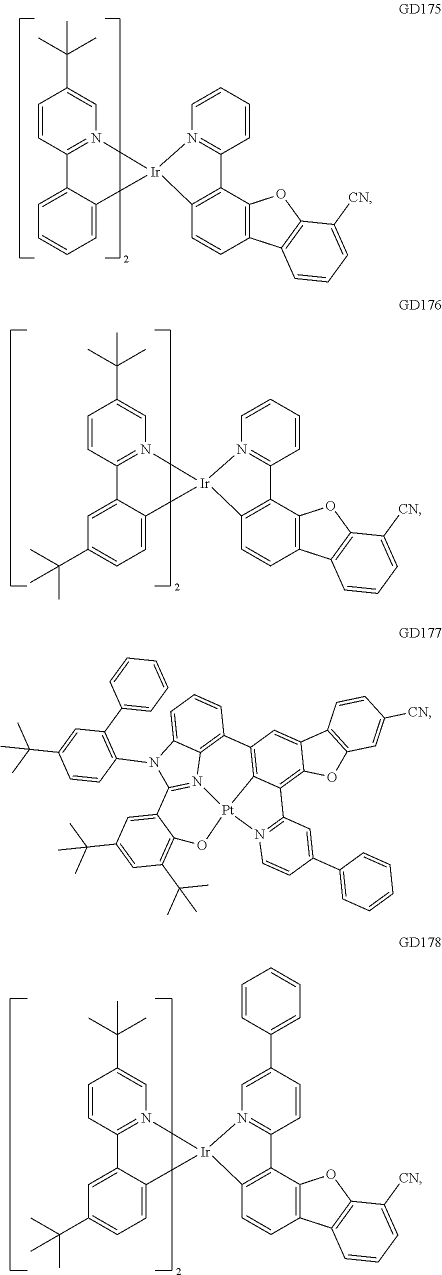 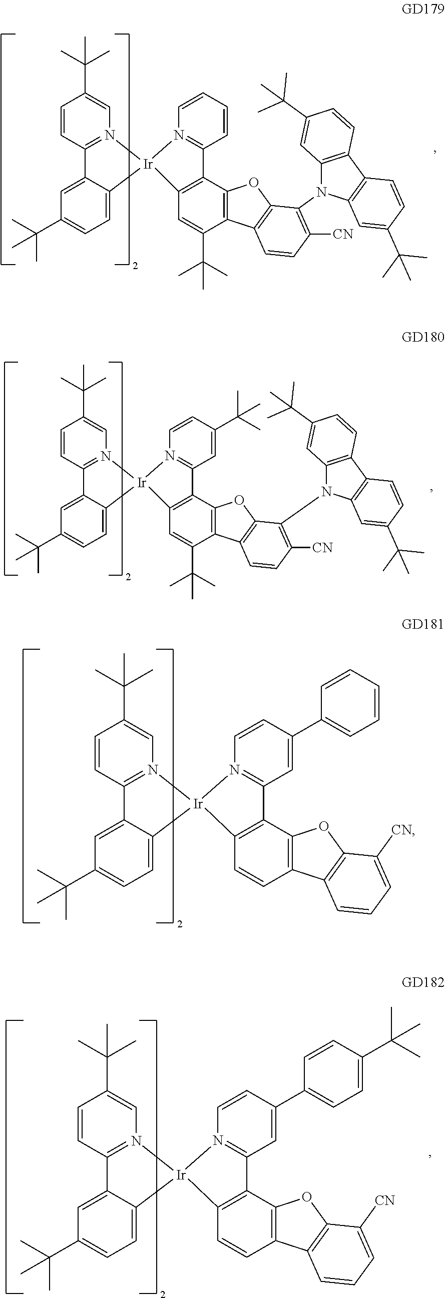 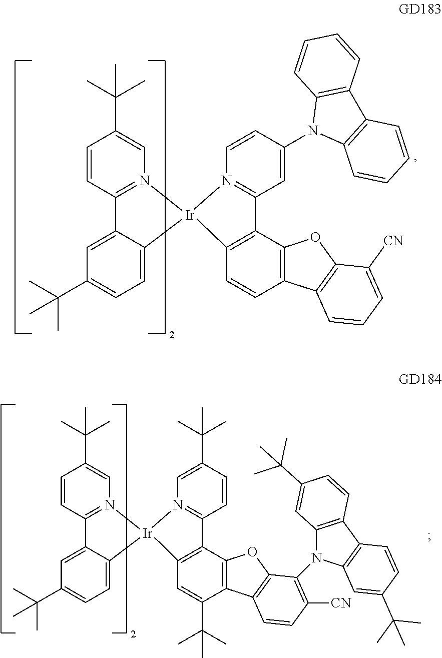
wherein optionally, hydrogens in the structures of Compound RD1 to Compound RD214 and Compound GD1 to Compound GD184 can be partially or fully substituted with deuterium.

15. A compound composition, comprising the compound according to claim 1.
